(19) United States
(12) Patent Application Publication
(10) Pub. No.: US 2016/0250051 A1
(43) Pub. Date: Sep. 1, 2016

(54) SET COMPRISING A CATHETER AND A VALVE SUPPORTING IMPLANT

(71) Applicant: TRANSCATHETER TECHNOLOGIES GMBH, Regensburg (DE)

(72) Inventors: Hou-Sen Lim, Singapore (SG); Wolfgang Gotz, Regensburg (DE)

(21) Appl. No.: 14/909,074

(22) PCT Filed: Jul. 31, 2014
(86) PCT No.: PCT/EP2014/066537

§ 371 Date: Jan. 29, 2016

(30) Foreign Application Priority Data
Jul. 31, 2013 (EP) 13178715.2
Jul. 31, 2013 (EP) 13178717.8
Jul. 31, 2013 (EP) 13178718.6
Jul. 31, 2013 (EP) 13178719.4
Mar. 14, 2014 (EP) 14160061.9
Mar. 14, 2014 (EP) 14160065.0

(51) Int. Cl. A61F 2/95; A61F 2/24

(52) U.S. Cl. CPC A61F 2/95; A61F 2/2439; A61F 2002/9511; A61F 2230/0069

(57) ABSTRACT

The present invention relates to a set comprising at least one expandable and/or decollapsible or unfoldable medical implant wherein at least one implantation device is configured for detachably receiving the implant or a device comprising at least the implant (3), wherein the implant may comprise at least a valve, wherein the valve may configured so as to fulfill a check valve function with one conducting direction (CD) and one blocking direction (BD), in which a blocking element may move for opening and/or closing the valve (50), wherein the valve may be arranged on the implantation device or on the device with at least one conducting side being located at a smaller distance from the distal end or from the distal tip of the implantation device than one blocking side. The present invention further relate to a method of detachably attaching an implant or a device comprising the implant with an implantation device and to a method for releasing an implant from an implantation device and/or from a device comprising the implant.

The present invention relates to a set according to the preamble of claim 1. The invention further relates to a method used for detachably attachment or interconnection of a medical implant with an implantation device according to claim 10 and to a method for releasing a medical implant at an implantation site according to claim 12.

In medicine, stents, or implants in general, are used for keeping vessels (especially: blood vessels) or tubes (especially trachea, esophagus, stomach, intestine, urethra, ureter) open or for replacing the function of a defect valve. They are inserted or advanced, respectively, to the implantation site in a folded or collapsed, respectively, or crimped manner by using an implantation device, for example a catheter. At this site, the unfolding or decollapsing, respectively, of folded or collapsed stents is effected for example by removing an outer sleeve, which is arranged over or about the folded or collapsed implant, by means of reset or restoring forces; or by means of a balloon arranged inside the implant, which expands the implant encircling or encompassing the balloon when being inflated or blown up, respectively.

The object of the present invention is to propose another medical set or arrangement for inserting and/or manipulating an implant or a device comprising such a medical implant, in particular a valve supporting implant, for example a heart valve, to and/or at an implantation site.

The object according to the invention is solved by the feature combination of claim 1. This object is also solved by the feature combination of claims 10 and 12.

Thus, according to the invention, there is proposed a medical set or arrangement comprising at least one expandable and/or decollapsible or unfoldable medical implant and at least one implantation device, for example a catheter, for detachably receiving the implant or a device comprising at least such an implant. Thereby, the implant may comprise at least a valve and/or may be designed, configured as and/or composed from at least from a valve supporting implant. Further, the valve may be designed or configured so as to fulfill a check valve function, for example the valve may be configured as a check valve with at least a conducting direction (CD) and at least a blocking direction (BD). Further, the valve may comprise a blocking element which may move for opening and/or, in particular reversibly, closing and/or opening the valve. Further on, the valve may be arranged, connected and/or fixed on, in or onto the implantation device and/or the implant such that at least one or the conducting side of the check valve is located, arranged or positioned at a smaller distance from the distal end or from the distal tip of the implantation device than one of the blocking side.

In other words, the valve may be arranged with its flowing through direction being orientated from the distal end to the proximal end of the implantation device.

That may mean that at least one or the conducting side of the check valve may be located or arranged before at least one or the blocking side of the valve in a direction from a distal end to a proximal end of the implantation device.

In an alternative embodiment of the present invention, there is proposed a medical set or arrangement comprising at least one expandable and/or decollapsible or unfoldable medical implant and at least one implantation device, for example a catheter, for detachably receiving the implant or a device comprising at least such an implant. Thereby, the implant may comprise at least a valve and/or may be designed or configured as or composed at least from a valve supporting implant. Further, the valve may be designed or configured so as to fulfill a check valve function, for example the valve may be configured as a check valve with at least a conducting direction and at least a blocking direction. Further, the valve may comprise a blocking element which may move for opening and/or closing the valve. Further on, the valve may be arranged and/or connected and/or fixed on, in or onto the implantation device such that at least one or the blocking side of the check valve is located or arranged or positioned at a smaller distance from the distal end or from the distal tip of the implantation device than one or the conducting side of the valve.

In other words, the valve may be arranged with its flowing through direction being orientated from the proximal end to the distal end of the implantation device.

That means that at least one or the blocking side of the check valve may be located or arranged before at least one or the conducting side of the valve in a direction from a distal end to a proximal end of the implantation device.

Further, according to the invention, a method for detachably attach, temporarily fix and/or detachably interconnect a medical implant on or with an implantation device may comprise at least the steps of providing an implantation device and a medical implant, wherein the implant may comprise at least a valve configured to fulfill a check valve function and of detachably attaching, fixing or interconnecting the implant on or with the implantation device. Thereby, the at least one or the conducting side of the valve may be located or arranged before at least one or the blocking side of the valve in a direction from a proximal end to a distal end of the implantation device, or, in other words, such that at least one or the conducting side of the check valve may be located, arranged or positioned at a smaller distance from the distal end or from the distal tip of the implantation device than the blocking side.

In an alternative embodiment of the present invention, there is proposed a method for detachably attach, temporarily fix and/or detachably interconnect a medical implant on or with an implantation device. The method may comprise at least the steps of providing an implantation device and an implant, wherein the implant may comprise at least a valve configured to fulfill a check valve function and of detachably attaching, fixing or interconnecting the implant on or with the implantation device. Thereby, the at least one or the blocking side of the valve may be located or arranged before at least one or the conducting side of the valve in a direction from a distal end to a proximal end of the implantation device, or in, other words, such that one or the blocking side of the check valve may be located arranged or positioned at a smaller distance from the proximal end or from the proximal tip of the implantation device than the conducting side.

Finally, the present invention relates to a method for releasing an implant from an implantation device at an implantation site using a set according to the present invention. The method comprises at least the steps of expanding, unfolding and/or decollapsing, respectively, a crimped or folded implant from a first diameter to a second diameter and disconnecting a device comprising the implant and/or the implant at a site that is different from a connection site at which the implant or the device comprising the implant has been connected to or attached on the implantation device.

Further advantageous embodiments and developments of the present invention are each subject-matter of the dependent claims.

Embodiments of the present invention can include additionally or alternatively one or more of the preceding and/or following features independently of any other feature, i. e., without having to comprise any other feature in combination.

Whenever the expression “can”, “may be” or “may have”, and so on is used herein, it is to be understood synonymously with “in exemplary embodiments is” or “in exemplary embodiments has”, “preferably is” or “preferably has” respectively, and so on, and is intended to illustrate exemplary embodiments.

In the following, the expression “distal end” may be understood as the end of the implantation device or of a receiving device for the implant which is intended to be inserted. The expression “proximal end” may be understood as the end of the implantation device or receiving device opposite to the distal end, in other words, the end which will be orientated to and manipulated by a surgeon or operator.

Whenever numerical values are mentioned herein such as “one”, “two” and the like, they have to be understood as values representing the lower threshold of numerical ranges. A long as this does not result in a contradiction in the eyes of the skilled one, numerical values, such as “one” shall be understood as comprising also “at least one”. This interpretation or understanding is as well encompassed by the present invention as the understanding according to which a numerical value such as “one” may be understood as “exactly one” whenever this appears technically possible to the skilled person. Both understandings are covered by the present invention. This applies to any numerical value stated herein.

Whenever reference is made within the present specification to a check valve, it is to be noted that the term “check valve” is used by way of an example for a valve having at least the function of and/or being at least configured for permitting a fluid, like gas or liquid, to flow through the valve in a particular direction and to prohibit or prevent such a flow in another, in particular reversed direction.

Whenever reference is made within the present specification to an implantation device, it is to be noted that this term is used by way of example for a delivery implement, catheter or device for delivering the implant to an implantation site. However, the present invention is not to be understood to relate only to catheters—rather, any suitable device for advancing an implant to its implantation site is also contemplated by the inventors.

According to the invention, expanding, unfolding or decollapsing, respectively, is understood as enlarging or increasing the diameter of the implant. Thereby, the non-expanded or non-unfolded or non-decollapsed, respectively, diameter (which can also be referred to as a first diameter, wherein also another diameter which is smaller than the second diameter mentioned below can be understood as a first diameter in the sense of the present invention) can be a diameter of the implant immediately before its insertion into the patient's body. In returning the diameter back from a second diameter (which is larger than the first diameter) to an arbitrary reduced diameter (the first diameter), the diameter is diminished or reduced, respectively. Returning (back) can be accomplished by a (completely or partly) re-folding or a reversed expanding procedure. According to the invention, during the returning procedure, the implant does not necessarily have to be brought into a shape that it occupied or passed through during unfolding or expanding.

According to the invention, altering the shape of the implant may mean reducing or increasing a diameter, particularly an external diameter, of the implant. Such an alteration can or cannot involve an alteration of the implant's length or any other kind of alteration.

In some embodiments according to the invention, the set comprises the devices that are necessary and provided or configured for executing the method(s) according to the invention. This especially applies for the devices mentioned in relation with the method(s) disclosed herein. A set may comprise—as any other device according to the present invention and/or any other device comprised in the set or parts thereof—at least one or more devices suited and/or configured and/or adapted such that one, more or all steps mentioned therein can be executed by the respective device.

In some embodiments of the present invention, folding the implant may mean reducing the diameter of the implant.

According to the present invention, the check valve may at least have a conducting direction and at least a blocking direction. A fluid flowing in the conducting direction of the check valve will be able to pass through the conducting side of the check valve wherein a fluid flowing in the reverse or in the blocking direction of the check valve will not be able to pass through the check valve, but will be retained or stopped, at least partly, from the blocking side or element of the check valve.

In certain embodiments of the present invention the blocking direction is a direction opposite to the conducting direction.

In some embodiments, the diameter of the implant is arranged in one plane perpendicular to a main flow direction of the implant in case fluids flow through the implant after its implantation.

In case a diameter of the implant could not be determined, expanding, unfolding or decollapsing, respectively, is understood as an increase in a direction or dimension of the implant which effects an elongation of a periphery or circumference, respectively, of the implant in a plane perpendicular to the longitudinal direction of the implant explained further below.

According to the invention, receiving an implant or a device comprising an implant by the implantation device is understood as any functional connection between the implant and the implantation device. Thereby, a transmission of power or forces, respectively, can take place, but does not have to take place. The connection can be provided as a frictional or form closure connection or neither as a frictional nor as a form closure connection.

According to the invention, “detachably receiving” is understood as a separable or dividable conjunction or association between the implantation device and the implant. An example for a separable or dividable conjunction is crimping a stent onto an implantation device for advancing or inserting the stent to the implantation site.

In some embodiments of the present invention, the implantation device comprises at least one means or an apparatus for controlling the expansion, the folding and/or the unfolding, respectively, of the implant, in particular from a first diameter to a second diameter and/or for returning back the implant from the second diameter to the first diameter; alternatively the implantation device is prepared for receiving such a means.

According to the invention, “controlling” also includes adjusting or setting or regulating, respectively. Thereby, it can be adjusted or set or regulated, respectively, to a voltage value, a pressure value or the like.

According to the invention the implantation device advantageously allows for a controlled unfolding or decollapsing, respectively, and re-folding or re-collapsing, respectively, (which can also encompass or include expanding and returning back to a reduced diameter) of the implant, for example, when being arranged inside the implant. Thus, it is advantageously possible to return or bring the implant back to a smaller diameter again and thus re-implant it after its expansion or unfolding, i. e., to shift it at the implantation site. If there should be detected during implantation that an implant of wrong size or design or construction, respectively, had been chosen, the implant could advantageously be replaced even after its expansion/unfolding.

In certain embodiments of the present invention, the implantation device may be connected to or communicate, respectively, with the implant via the means for controlling.

In particular embodiments, the unfolding or decollapsing, respectively, and the folding or collapsing, respectively, of the implant can be performed without the aid of an outer sleeve.

In some embodiments of the present invention, the implantation device may comprise a plastic or synthetic material or a copolymer or can be manufactured by means of two- or multiple-component technologies. According to the invention, the implantation device may comprise a metal (steel or alloy). The implantation device may be stiff; however, it may also be designed flexible or bendable, in order to be able to adapt it to or align it with defined or certain conditions. Thereby, the implantation device may be manually bendable or it can be controlled for bending by means of a mechanism which can, e. g., be integrated in the implantation device. The implantation device may be bendable in a passive manner, e. g., by advancing or inserting it along the vessel or body lumen alone.

In certain embodiments of the present invention, the implantation device may comprise a mechanically enforced or reinforced section, in particular in a tip area of the implantation device, and in particular in a section which also comprises at least one passage means.

In some embodiments of the present invention, the implantation device may have a circular or oval or rectangular cross-section. The implantation device can also have a non-circular, a non-oval or a non-rectangular cross-section. Furthermore, the cross-section of the implantation device can be unchanged across the whole implantation device. However, it can also have two or three or more different cross-sections along its longitudinal axis and in particular in the area for receiving the implant.

In certain embodiments of the present invention, the unfolding and folding of the implant which is controlled by the implantation device can take place outside and/or inside a patient's body.

In some embodiments of the present invention, the implantation device can be designed such as described in, e.g., US 2007/0100427 A1 by Perouse or in US 2005/0075731 A1 by Artof et al. By way of reference, the contents thereof are each subject-matter of the present invention. This particularly applies for the materials and (part) geometries given therein.

In certain embodiments of the present invention, the implantation device can be a one-lumen implantation device; it can have no lumen and it can be a multi-lumen implantation device. If it is a multi-lumen implantation device, the implantation device can be a two- or three- or multi-lumen implantation device having equally or differently sized lumina in or regarding its cross-section.

In some embodiments of the present invention, the implantation device has a plurality of lumina in longitudinal direction or channels (hereinafter also designated in short: channel or channels) for guiding tension threads or reins. The lumina or channels may serve for organizing and/or arranging or aligning, respectively, the tension threads. They can advantageously assure that the physician is always able to determine which one of the optionally same looking tension threads he holds in his hand or wants to operate. Hereto, he has only to orientate from which channel the respective tension thread comes out or leaves, respectively, or into which channel the tension thread enters.

In certain embodiments of the present invention, the channels can serve for avoiding any disorientation or tangling, entangling, knotting or interloping, respectively, etc. of the tension threads with each other.

In some embodiments of the present invention, tension threads serving for the same or a common purpose during operation can be combined in the respective channels. Thus, tension threads which all have to be operated for effecting a certain behavior of the implant and/or of a device comprising the implant and/or of the implantation device can be guided through one channel. Tension threads which have to be operated for effecting another behavior of the implant and/or of a device comprising the implant and/or of the implantation device can be guided, through another channel. Obviously, using the tension threads facilitates the operation of the implant and/or of a device comprising the implant and/or of the implantation device by the physician.

In certain embodiments of the present invention, tension threads running to the implant can also be separated from tension threads running back from the implant.

In some embodiments of the present invention, providing the tension threads in a plurality of channels and their guidance therein can avoid a mutual interaction or interference, respectively, or the risk thereof. With the corresponding arrangement of the tension threads in separate channels it can, for example, be assured that, by pulling one tension thread running in a first channel, another tension thread is not unintendedly operated due to friction or any other interaction of the pulled tension thread with this other rein.

In certain embodiments of the present invention, providing a plurality of channels for separately guiding tension threads can advantageously enable a separation of tension threads and other means such as, for example, a guiding wire. The function of the tension threads is thus not impacted or influenced, respectively, by further means and functions of the implantation device, but—and this is not less advantageous—also vice-versa; i. e., it can also advantageously be avoided that the further means such as a guiding wire is impacted or influenced, respectively, by the presence or operation of the tension threads of the implantation device.

In some embodiments of the present invention, providing a plurality of channels for separately guiding tension threads can thus advantageously increase the precision during operation of the tension threads and thus the use of the implantation device and/or of the implant.

In certain embodiments of the present invention, the implantation device comprises at least one passage means for letting pass one or more tension threads.

In particular embodiments, the tension threads can serve for influencing the expansion, folding and/or unfolding of the implant by changing a tension or stress, respectively, that is applied on the implant by the tension thread.

According to the invention, “letting pass” is also understood as “passing” or “guiding through”.

In some embodiments, a passage means may be a passage opening, an eye or loop, respectively, a deflecting or diverting section or the like.

In particular embodiments of the present invention, a tension thread can comprise a polymer, a metal or a biological fiber material or can consist thereof. The tension thread or the tension threads can be optionally absorbable.

According to the invention, a tension or a stress, respectively, which is applied to the implant by the tension thread, is also understood as a strain or in general any effect of the tension thread on the implant.

Whenever the present application refers to a tension thread, there can be meant more than one tension thread, for example, two, three, four, five or more tension threads.

In some embodiments of the present invention, the tension threads can also be provided functionally separated from the implantation device.

In certain embodiments of the present invention, the implantation device can comprise an inner guiding means for the at least one tension thread.

In some embodiments of the present invention, the tension threads can leave or get out of the implantation device at one side and/or at one end of the implantation device through one or more passage means (especially, when those are designed as passage openings). Those passage means can be present in or on one or several planes perpendicular to the longitudinal axis of the implantation device.

In certain embodiments of the present invention, the implantation device can comprise a device for cutting or tearing through the tension threads.

In some embodiments of the present invention, the implant may be configured as a valve supporting implant or may be composed of at least one valve.

In certain embodiments, the implant may comprise at least one foldable or collapsible or crimpable and unfoldable or expandable structure on or around or over a portion or outer surface of the implantation device or of part thereof.

In some embodiments the implant can be a stent or comprises a stent, in particular a valve supporting stent, or may be configured as a cardiac valve, a heart valve prosthesis, substitute or replacement thereof.

In particular embodiments, the implant comprises flexible leaflets.

In certain embodiments of the present invention, an implant, in particular a stent, is proposed, which comprises at least one means which is provided or prepared with the means of an implantation device for controlling the expansion, folding and/or unfolding of the implant, in particular from a first diameter to a second diameter and/or the return back from the second diameter to the first diameter in order to control the change of the diameter.

In some embodiments of the present invention, the implant comprises at least one guiding means which is prepared and suited for guiding at least one tension thread by which at least one section of the implant is expandable and/or decollapsible or unfoldable, respectively, from the first diameter to the second diameter and/or is returnable back from the second diameter to the first diameter by changing the tension or stress, respectively, or the strain applied to the tension thread.

In certain embodiments of the present invention, the implant can be self-expanding, for example, it can be formed from or with a memory material, in particular nitinol, or materials which comprise nitinol. However, the implant can also be partly self-expanding, partly expandable by the use of an expanding means. The implant can exclusively be non-self-expanding. The implant can be foldable; the implant can be non-foldable.

In some embodiments of the present invention, the implant can comprise a biocompatible material, in particular a biocompatible stainless steel. The material can be bio-absorbable.

In certain embodiments of the present invention, the implant can be designed with or without a means for encompassing or sandwiching parts of native valve sections (in particular heart valve leaflets). In particular, the implant can be designed with or without sections rising up or lowering down due to temperature and memory effect.

In some embodiments of the present invention, the one or several guiding means of the implant can be designed in form of guiding holes, guiding rings, eyes or loops, respectively, hooks or, generally spoken, guiding structures. They confer guidance to the rein which can be understood in the sense of directing the rein in one direction.

According to the invention, guidance can also be understood such that the rein experiences stabilization along its extension. Thereby, the rein can be guided or directed, respectively, by the guiding means from an interior of the implant or stent, in particular from the implantation device, to the implant structure (in particular to the exterior of the implant). The guiding means of the implant can be symmetrical (in particular circular, oval or square) or asymmetrical. The guiding means can be located on one plane, on several planes or on a spiral plane of the implant. Several guiding means can be designed equally or can be present in at least two different designs.

In certain embodiments of the present invention, the implant or the stent can comprise a circular guiding means. The said can be designed in form of a channel which is, relative to the implant, open or closed to the outside, to the top or to the bottom. The guiding means can be closed or open and can have a symmetrical or asymmetrical form. The guiding means can be designed in form of a grid structure, a meander structure, a sinus wave structure, in particular one comprising 18 wave tips along a periphery, or the like. The guiding means can have a structure without a grid and/ok without a meander and/or without a sinus wave structure. However, it can also comprise a sinus wave structure having a number of wave tips other than 18.

In some embodiments of the present invention, the implant can be a valve supporting stent and made from steel such as described in the U.S. Pat. No. 5,411,552, U.S. Pat. No. 5,840,081 and U.S. Pat. No. 6,168,614 B1 by Andersen et al. The stent can, however, also be a valve supporting self-expanding stent according to the disclosure of U.S. Pat. No. 7,018,406 B2 by Seguin et al. or of US 2005/0075731 A1 by Artof et al. The contents of the afore-mentioned documents are by way of reference herewith also subject-matter of the present invention or application, respectively. This particularly applies for material and (part) geometries of the implants and stents disclosed therein.

In certain embodiments of the present invention, the tension threads can be guided or directed, respectively, along an interior of the implantation device and can leave or get out of the implantation device through the passage means. Then, the tension threads can be guided through the guiding units at or on the implant. The tension threads can be guided along a periphery or parts of a periphery in the guiding unit along the circumference of the implant. Then, the tension threads are guided back from outside through the passage means to the interior of the implantation device. The tension threads can leave the implantation device at its end proximal to the surgeon. The tension threads can leave the implantation device at its end distal to the surgeon. The effect and in particular the strain of the tension threads and thus the controlled unfolding and the re-folding or reduction in the diameter of the stent can be controlled by means of a controlling means.

In some embodiments of the present invention, a tension thread can leave the implantation device via a passage means arranged at the implantation device and can be guided back into the implantation device via the same passage means or via another passage means on the same or on another plane.

In certain embodiments of the present invention, the tension threads can leave the implantation device with their both ends.

In some embodiments of the present invention, the tension threads can leave the implantation device with one end and can be connected to the implantation device with the other end.

In certain embodiments of the present invention, the tension threads can be pulled back from the implant or stent and removed after cutting or tearing through or disengaging or notching, respectively, or by using any other kind of detaching or loosening the tension threads.

In some embodiments of the present invention, the tension threads can be cut or torn through by means of a device in the implantation device, a device inside or outside the implantation device (in particular a knife, a pair of scissors, by means of electrical voltage, by means of heat). A suited device can be arranged at or on the implantation device.

In certain embodiments of the present invention, the stent is unfolded or expanded with little or without any tension of the tension threads. With tension being present, the stent is reduced in its diameter or is partly or completely re-folded.

In particular embodiments of the set, the implantation device is arranged in the center or substantially in the center of a cross-section or of any cross-section of the implant and/or of the valve.

In some embodiments of the present invention, this center or central arrangement can relate to a state of use. It can relate to a non-unfolded or non-expanded state. It can, however, also relate to a completely expanded or unfolded state.

In certain embodiments of the present invention, the state of use can be a state during positioning the implant—in particular during its rotation around a rotation center thereof—in order to precisely form the desired positional relation between the implant and the anatomic site at which the implant shall be implanted or come to rest in the body.

In some embodiments of the present invention, arranging the implantation device in the center of the implant can involve several advantages wherein a uniformly expanding of the implant is among those. Furthermore, with a centralized implantation device, the implant can be controlled and positioned in a better way. The following example may point this out: In the case of a heart valve prosthesis as implant, it can be necessary to align or orientate, respectively, it relative to the commissures of the aortic root during the insertion of the prosthesis (the implant) supported by the implantation device in such a way that the orientation thereof and the orientation of the valve leaflets arranged at the prosthesis are concerted or coordinated, respectively. For this purpose, the physician rotates the implantation device around its longitudinal axis and, with a centrally arranged implantation device, this rotation brings the heart valve prosthesis into a desired position relative to its orientation in its rotational direction, too. If the implantation device is thereby not arranged in the center of the rotational-symmetrically constructed heart valve prosthesis, the heart valve prosthesis experiences a displacement in a radial or lateral direction. This displacement not only complicates achieving the desired orientation in the rotational direction, but also effects an undesired force and stress of circumjacent structures such as of the aortic root, of an already inserted receiving means for receiving and anchoring the prosthesis and the like. Centralizing the implantation device in the center of the implant can advantageously counteract this. The relative relation between the implantation device and the implant can remain unchanged. The occurrence of undesired displacements and forces can advantageously be avoided.

In certain embodiments of the present invention, centralizing the implantation device in the center of the implant or the arrangement thereof in the center of the implant, respectively, can advantageously be used for checking the valve function with an implantation device still being connected to the implant. The valve leaflets can unfold and close for a functional check without being hindered by the centrally arranged implantation device; however, this would not be possible if the implantation device would have been arranged at the edge, relative to a cross-section of the implant or of the valve. A position of the implantation device other than the central position could result in a non-uniform opening and closing of the valve leaflets and complicate a functional check, falsify or distort, respectively, the result thereof or make it impossible. Such a functional check with an implantation device still being connected to the valve is, however, of great importance and of great utility, because a revision or a re-positioning of the valve shall be possible when an unsatisfying position has been determined.

In some embodiments of the present invention, a central position of the implantation device, relative to the cross-section of the implant or of the valve, can also be advantageous in a folded or non-expanded state of the implant, because this position can allow a simplification of folding, crimping or the like of the implant. Thereby, the space requirement can advantageously be reduced, a damage of the implant or sections thereof (such as, in some embodiments, valve leaflets) can advantageously be avoided, etc.

In certain embodiments of the present invention, a central position of the implantation device, relative to the cross-section of the implant and/or of the valve, can furthermore advantageously enable to control the position of the implant in such a way that the implant uniformly contacts the structure into which it shall be inserted at its periphery. Tension or force peaks which can damage the receiving tissue or complicate the insertion process of the implant can thus advantageously be avoided. The risk of damaging the implant (for example, valve leaflets) or injuring tissue can hereby advantageously be diminished.

In some embodiments of the present invention, the implant is being crimped, is crimped and/or is configured to be crimpable onto the implantation device and/or a device for supporting or receiving the implant, and released or configured to be releasable from the latter at the implantation site.

In certain embodiments of the present invention, the implant is intended to be detachably fixed, attached or crimped on a portion or a surface of an implement device, such as a catheter, for being delivered to an implantation site.

In particular embodiments, the implant has a longitudinal axis, or an inner space or inner volume longitudinally extending within the implant, and has a radial direction perpendicular to the longitudinal axis, space or volume.

In some embodiments of the present invention, the implant comprises a first structural element having a first portion and a second structural element having a second portion.

In certain embodiments of the present invention, the implant comprises one or more interconnecting elements arranged between the first and the second structural elements.

In some embodiments of the present invention, the first and/or the second portions of the implant are located less radially as regards the longitudinal axis, the inner space or volume than a third portion of the one or more interconnecting elements.

Further the present invention relates to a method of detachably attaching, fixing, arranging, crimping and/or connecting an implant or a device comprising at least an implant on or with an implantation device as defined in claim 11.

In some embodiments, the method of detachably attaching, fixing, arranging, crimping or connecting an implant on or with an implantation device comprises the optional additional step of crimping or folding the implant such that there remains a first gap between one, more or all of the interconnecting elements and an outer surface of the implantation device.

In certain embodiments of the present invention, the implant may be of an expandable and again foldable or collapsible, respectively, type. Such implants may, for example, be changed in its diameter by means of strings guided around certain portions of the implant that can be tightened or released. The features required to be amendable in diameter are not in the main focus of the present invention. Since they are further explained in great detail in WO 2008/029296 A2 (“Minimally invasive heart valve replacement”, filed on Feb. 15, 2007) to the inventors of the present invention, and also in WO 2009/109348 A1 (“Stent, welcher vom expandierten Zustand kontrolliert erneut im Durchmesser verringerbar ist”, filed on Mar. 2, 2009) also to the inventors of the present invention, for the sake of avoiding repetition it is referred to those documents as regards those features. The respective disclosure is herewith incorporated into the present application by way of reference. The same applies to any material mentioned in either of both applications.

In certain embodiments, “radial” or “radially” may be understood as “lateral” or “laterally”, both indicating that a first structure that is arranged radially or laterally with respect to a second structure is more distant to, e.g., a central axis or a medial element than the second structure is. Consequently, “less radial” means more central or medial.

In some embodiments, the first structural element is a proximal structure, with the second structural element being a distal structure.

In particular embodiments, the interconnecting elements are two or more, in particular three, equidistantly or equi-angularly spaced posts. The posts may be equally spaced from each other. The posts may be located at 120° to one another.

In certain embodiments, one, more or all of the interconnecting elements is or forms a mesh. In some embodiments, the mesh is designed such that it can be changed in its diameter. Preferably, the mesh can be changed in diameter without (at all or significantly) changing its longitudinal extension. That is, the mesh can be prepared not to lengthen or to foreshorten upon changing the implant's diameter.

In some embodiments, the at least one or more interconnecting elements interconnect the first and the second structural elements with each other.

In certain embodiments, the interconnecting element or elements are provided for maintaining a distance between the first and the second structural elements.

In some embodiments, the interconnection of the first and the second structural element can be of a direct or indirect manner.

In certain embodiments, the interconnecting elements are posts arranged at or within the implant such that in at least one state, or in any state, of the implant the posts extend in parallel to the longitudinal axis, the inner space or volume.

In some embodiments, “in parallel to the longitudinal axis” simply means that there is a plane comprising a post at issue and another plane comprising the longitudinal axis, with those planes not intersecting with each other.

A state, as referred to above, may be a fully expanded state of the implant, e.g., after completion of the implantation process. A state may be a fully crimped state of the implant. A state may be the crimped state of the implant upon delivery to the implantation site by means of the implantation device.

In certain embodiments, the implant, in particularly the first structural element and/or the second structural element has at least a fourth portion, and possibly also a fifth portion, and maybe also further portions, located more radially or laterally as regards the longitudinal axis or the inner space/volume than a particular third or any third portion of the one or more interconnecting elements.

This may be the case in particular embodiments in a state in which the diameter or the radial expansion of the inner space is reduced due to external pressure put on the implant, or in all states the implant may take on or actually takes on during normal use of the implant.

In some embodiments, the fourth portion (and maybe also further portions also located more radially or laterally as regards the longitudinal axis or the inner space/volume than a particular third or any third portion of the one or more interconnecting elements) rises above the lateral or radial level of the third portion during the crimping process. In certain embodiments, for example, the first and/or the second structural element is at least temporarily deformed by the crimping process such that the fourth portion “stands up” (i.e., rises) during crimping. It is well possible that the fourth portion looses its prominent position again after expansion of the implant as is the case in some embodiments.

In particular embodiments, the third portion is provided more radially as regards to the inner space or volume than the first and/or the second portion in a state in which the diameter or the radial expansion of the inner space or volume is reduced due to external pressure put or applied, respectively, on the implant.

In certain embodiments, the state in which the diameter or the radial expansion of the inner space is reduced due to external pressure put on the implant is a fully crimped state or a state into which the implant has been crimped or would be crimped upon crimping for being implanted.

In some embodiments, the state in which the diameter or the radial expansion of the inner space is reduced due to external pressure put on the implant is a crimped state of the implant in which the first or the second portions or both portions contact an outer surface of the implantation device.

In certain embodiments, the first structural element and/or the second structural element is/are (a) guiding structure(s) for guiding strings for amending—both increasing and/or decreasing —the diameter of the implant once brought into a part of a body vessel or a part of the patient's heart.

In some embodiments, the implant is designed such that after having been crimped on the implantation device there remains a first gap between one, more or all of the interconnecting elements and an outer surface of the implantation device.

In certain embodiments, the gap or parts thereof are mainly or partly tube-shaped.

In some embodiments, the implant is designed such that after having been crimped on the implantation device there remains a second gap between one, more or all of the interconnecting elements and an inner surface of a sleeve covering the implant.

Again, in certain embodiments, the second gap is tube-shaped—mainly or partly.

In some embodiments, the first gap and/or the second gap is/are intended to accommodate structures like leaflets, commissures or the like, or sections thereof. Its size may be tailored to needs.

In certain embodiments, in a crimped or delivery state of the implant, the width of the first gap and/or of the second gap is at least 2 mm, or at least 3 mm, or at least 5 mm.

In particular embodiments, the implant comprises at least one sleeve. One of the sleeves may cover the implant on its outer surface.

In some embodiments, the method of detachably attaching, fixing, arranging, crimping and/or connecting an implant on or with an implantation device may comprise optionally the covering of the crimped implant with a sleeve, in particular such that there remains a second gap between one, more or all of the interconnecting elements and an inner surface of a sleeve covering the implant.

In certain embodiments, the method of detachably attaching, fixing, arranging, crimping and/or connecting an implant on or with an implantation device may comprise optionally crimping or folding until the final crimping state before implantation is reached. Therefore, wherever it is above referred to crimping of the implant such that there remains a first gap between one, more or all of the interconnecting elements and an outer surface of the implantation device, the final crimping state may be meant.

Along with advantages that are obvious to the skilled one and previous discussed advantages, the embodiments may provide one or more of the following advantages.

Although crimping of implants, in particularly stents, is well-known in the art and probably the most often used method for temporarily fixing an implant on an implantation device, according to the findings of the inventors the implant or structures comprised by the implant are frequently adversely compressed and sometimes even damaged. Those damages have hitherto not been realized neither by the skilled ones nor by the public. The present inventors, however, realized a problem resulting from applying undue pressure on, e.g., the leaflets of a heart valve replacement such as the one described in above mentioned WO 2008/029296 A2. It appears that the damages observed resulted from a pressure applied on the leaflet and the commissures upon crimping between the interconnecting elements or posts and the sleeve, respectively, on the one side, and the crimping surface (outer surface) of the implantation device on the other side.

In some embodiments, the implant design provides for space (the first gap) between the interconnecting elements to allow, e.g., the commissures of above implant of the figures or other structures to be located between the interconnecting elements and the surface of the implantation device without being pressed or even damaged.

Further, in certain embodiments, a design of the implant provides for sufficient space (second gap) for structures such as the leaflets of the implant of WO 2008/029296 A2 between the sleeve (if provided) or the vessel wall during delivery of the implant, and the surface of the relatively hard and inelastic implantation device.

In some embodiments, crushing of leaflets of a valve replacement comprised by the implant may be advantageously avoided.

In certain embodiments, a disruption of collagen fibres within leaflets of a valve replacement of natural origin (bovine, for example) after having been crimped can advantageously be prevented.

In certain embodiments, the means or the apparatus configured for controlling the expansion, folding and/or unfolding may fold or unfold an implant by using at least a tension thread.

In some embodiments, the means for controlling the expansion, folding and/or unfolding includes a shaft having a reception area for receiving the implant.

In certain embodiments, the means for controlling the expansion, folding and/or unfolding and/or the implantation device further includes at least one tensioning device for altering a shape of the foldable and/or unfoldable implant by means of the tension thread.

In some embodiments, the means for controlling the expansion, folding and/or unfolding and/or the implantation device includes a separation device for separating at least one tension thread from the implant and/or for cutting or cutting through the tension thread.

In particular embodiments, the at least one tension thread is a thread. The thread may be similar to a surgical suture thread. The thread may have the shape of a rope, a filament or of a cord. The thread may optionally be designed as a chain having a plurality of engaging chain links.

Whenever the present application refers to a thread or tension thread, there may be meant a plurality of threads or tension threads, for example, two, three, four, five or more threads, whenever a person skilled in the art recognizes the exchangeability of the terms.

In certain embodiments, the shaft of the means for controlling the expansion, folding and/or unfolding and/or of the implantation device is rigid. In some embodiments, the shaft is flexible in one or more directions (i.e., in a′longitudinal direction or in a direction of the width of the shaft, respectively, in both directions or in other directions). In certain embodiments, the shaft is elongatable. In some embodiments, the shaft is stiff.

In some embodiments, during its implanted implantation state and/or in its implanted state, the implant is able to be penetrated by fluids or is permeable for fluids, respectively, in its longitudinal direction. The terms “permeable” or “able to be penetrated” hereby refer to the ability of the implant to be penetrated or flown through by fluids.

In certain embodiments, the implant is—at least transiently or temporarily—mounted or loosely arranged on or at the reception area of the means for controlling the expansion, folding and/or unfolding or of the implantation device at the moment of unfolding or folding.

In some embodiments, the implant is arranged on or at the reception area or is interconnected with the reception area only by tension threads.

In certain embodiments, the tensioning device includes at least one pulling device. The pulling device is arranged and/or provided in such a way that it can indirectly or directly apply a tension on the implant for altering the shape of the implant by means of the tension thread.

Alternatively or additionally, in some embodiments, the pulling device is arranged and/or provided in such a way that it can reduce a tension applied on the implant by means of the tension thread.

In certain embodiments, the pulling device is arranged and/or provided such that it can interact with the tension thread in order to transfer force or tension.

In some embodiments, the pulling device and the tension thread are intricate with each other at at least one site.

In certain embodiments, the term “intricate” may be used to indicate that the tension thread is movable in at least one direction of space or in two directions of space relative to the pulling device.

According to some embodiments of the invention, the term “movable” may be understood as “slidable”.

According to certain embodiments of the present invention, the term “intricate” may mean that the tension thread is movably arranged relative to the pulling device like a first link of a chain is movably arranged relative to an adjacent second link of this chain to which the first link is usually connected in a chain.

In some embodiments, the term “intricate” may indicate that the tension thread is simply crossed once with or wrapped around the pulling device or sections thereof.

In some embodiments, the transfer (or the transmittal, respectively) of force or tension between the pulling device and the tension thread is achieved by a non-form closure connection.

In certain embodiments, the transfer of force or tension between the pulling device and the tension thread is achieved by a frictional connection.

In some embodiments, the pulling device is embodied as at least one pulling thread or consists of at least one pulling thread.

In certain embodiments, the tension thread and/or the pulling thread includes or constitutes at least one bundle or a plurality of threads or thread elements or consists thereof.

In some embodiments, the separation device includes at least one cutting device for cutting (or cutting through) the tension thread or consists thereof.

In certain embodiments, the separation device is not a wire or a thread (or a multitude thereof, respectively).

In some embodiments, the separation device is not embodied as a lock wire or lock thread that is withdrawn from the apparatus so as to allow for removing tension threads from the apparatus after final placement of the implant.

In certain embodiments, the separation device does not comprise hooks and/or does not comprise rings for guiding or limiting tension strings or threads.

In some embodiments, the separation device is embodied and/or intended to stay with the apparatus even after termination of the implantation of the implant.

In certain embodiments, the separation device may not be separated from the apparatus except for cleaning or the like.

In some embodiments, the separation device is embodied such that it is intended to be used only within the patient's body.

In certain embodiments, the separation device is not intended to be used for separating the tension threads from implant from outside the patient's body.

In some embodiments, the separation device can not be used to separate the tension strings without the presence and/or the support of the apparatus.

In certain embodiments, the apparatus includes at least one sleeve. The sleeve can preferably be made tube-like (that is, it can have a hollow inside), like a hollow cylinder, like a ring or the like. The sleeve can be designed symmetrically or asymmetrically, both relative to its opening direction and in another direction, particularly in a direction or plane perpendicular to the opening direction or the fluid passage direction, as well.

In some embodiments, the separation device is designed as a—preferably integral—part of a sleeve aperture in one wall of the sleeve of the apparatus. The sleeve aperture may connect an exterior of the sleeve with an interior of the sleeve. The sleeve aperture may in particular be designed as a passage opening or a through opening. It can thus be as thick as the wall of the sleeve is in a radial direction of the apparatus.

In certain embodiments, the sleeve aperture includes at least one first recess. At least one tension thread can be led or guided through—or by means of—this first recess.

In some embodiments the first recess includes a first portion or first area which includes a cutting device for cutting through the tension thread.

In certain embodiments, the cutting device is or comprises a cutting edge. In some embodiments, the cutting device is attached to the sleeve. In certain embodiments, the cutting device is integrally formed with the sleeve.

In some embodiments, the first recess includes a second portion or second area which does not include a cutting device.

In certain embodiments, the sleeve aperture includes at least one second recess in which at least one tension thread can be led or guided, preferably in the same way as with the first recess.

In some embodiments, the first recess and the second recess can be separated from each other by means of a bar in such a way that at least two tension threads can be led or guided in the recesses while being spaced apart from another.

In some embodiments, providing a distance between the two recesses can allow the leading of one or more tension threads having been distributed to the two recesses without touching each other in the area of the recesses. Thus, the leading of several tension threads through one sleeve aperture is advantageously possible without the tension threads constricting or hindering themselves. Additionally, the separated leading of tension threads being favored by providing several recesses advantageously allows for a separate treatment or use of each single tension thread: tension threads being led through a first recess can thus advantageously be differently use or treated tension threads being led through a second recess.

In certain embodiments, the bar is part of the sleeve. Hence, the bar is part of the sleeve wall and/or integrally formed therewith.

According to the invention, both recesses given above can also be referred to as niches, as extensions, as indentations, as furcations, as notches and so on (this can also apply for third, fourth and several more recesses). All of these terms have in common that the recesses, niches or the like branch off a common, particularly wide, area of the sleeve aperture or are connected therewith.

In certain embodiments at least the first recess (or any other recess) extends in one dimension of the apparatus (preferably in a longitudinal extension of the apparatus) differently than at least the second recess does in the same dimension of the apparatus.

In some embodiments, the shaft of the apparatus is permeable or has a passage for fluids in its interior in at least some sections of its longitudinal direction.

In certain embodiments, the shaft has a wall.

In some embodiments, the shaft includes at least one shaft aperture. The at least one shaft aperture is preferably rather arranged on a lateral area of the shaft than on the front side thereof.

In certain embodiments, the shaft of the apparatus includes a plurality of shaft apertures having been uniformly or non-uniformly distributed along one or more circumferences and/or along the longitudinal extension of the shaft.

In some embodiments, tension threads for folding and/or unfolding the implant can enter and/or leave the apparatus through the shaft aperture. During use of the apparatus, the sleeve is arranged in an interior or in an exterior of the shaft such that tension threads can (preferably unrestrictedly and/or directly) be led from the exterior of the apparatus into an interior of the apparatus.

In certain embodiments, the tension threads can be led unrestrictedly and/or directly, that means without having to be bent or redirected, from an exterior of the apparatus into an interior of the apparatus, particularly into an interior of the sleeve and/or into an interior of the shaft.

In some embodiments, the sleeve is preferably arranged in a shiftable manner.

In certain embodiments, the sleeve can preferably surround the shaft in such a way that the shaft is located inside the sleeve and the sleeve is at an outside of the shaft. The at least one tension thread can thus pass from an exterior of the sleeve through the shaft aperture into an interior of the shaft (or vice versa).

Alternatively, the sleeve can in some embodiments be located inside of the shaft such that the shaft is surrounding the sleeve. The at least one tension thread can thus pass from an exterior of the shaft through the shaft aperture and through the sleeve aperture into the interior of the sleeve (or vice versa).

In certain embodiments, the apparatus includes a pre-tensioning device which is arranged for exerting tension on the sleeve in at least one state of use.

In some embodiments, the pre-tensioning device exerts a tension on the sleeve substantially or exclusively in a longitudinal direction of the shaft.

In certain embodiments, the pre-tensioning device exerts a tension on the sleeve so as to twist the sleeve relative to the shaft.

In some embodiments of the invention, the pre-tensioning device is embodied as a spring, particularly as a coil spring, and/or is embodied from any suitable—preferably from elastic or flexible—material such as rubber.

In certain embodiments, the pre-tensioning device is arranged for build up or maintaining the pre-tension in a pushing manner.

In some embodiments, the pre-tensioning device is arranged for build up or maintaining the pre-tension in a pulling manner.

In certain embodiments, the pre-tensioning device is arranged for maintaining the separation device and/or the cutting device and/or the sleeve in a non-separating position in which no tension thread is separated from the implant and in which no tension thread is cut through.

In some embodiments, the apparatus includes a device with which the separation device and/or the cutting device and/or the sleeve is transferred into a separating position in which at least one tension thread is separated from the implant or in which at least one tension thread is cut (through).

In certain embodiments, said device which enables a transfer or transition from the non-separating position into the separating position is embodied as a pulling device as, e.g., a thread or the like. In another embodiment, said device is embodied as a pushing or twisting device.

In some embodiments, said device which enables a transfer or transition from the non-separating position into the separating position includes at least one pulling thread or pushing means or twisting means which is led from its branching off at the sleeve to an exterior of the apparatus. The end of the thread or pushing means or twisting means which is distal to or far from the sleeve can be connected to a grasping device suited for grasping the thread for the purpose of pulling or pushing or twisting it. At an exterior thereof, the apparatus can include—but does not have to include—a snapping device for releasably receiving the grasping device. The grasping device can rest on the snapping device until its use.

In some embodiments, said device is arranged such that it can only be transferred from a non-separating position into the separating position by overcoming the effect of the pre-tensioning device.

In certain embodiments, at least one recess is designed and provided in the sleeve such that at least three different openings or outlets can be formed by superposing the shaft aperture and the sleeve aperture (e.g. by shifting or moving the sleeve inside the shaft or the shaft inside the sleeve).

Whenever it is herein referred to “at least three different openings”, these openings can each have a different geometrical shape, according to some embodiments of the present invention. In other words, the openings can differ in their design.

In certain embodiments of the present invention, the “three different openings” can differ in the size of their respective area.

In some embodiments of the present invention, such an opening is defined as the open passage between an exterior of the apparatus or of the shaft of the apparatus and an interior of the apparatus or of the shaft.

In certain embodiments of the present invention, such an opening is an area in which a passage both through the shaft aperture (i.e. an opening in the wall of the shaft) and through the sleeve aperture (i.e. an opening in the wall of the sleeve) is open in the sense of a passage opening.

In some embodiments of the present invention, the “three different openings” can differ in their function.

In certain embodiments a first opening can be large enough to allow introducing one or several tension threads from an exterior of the apparatus into an interior of the apparatus, e.g., both through a wide area of the sleeve aperture and a shaft aperture adjoining or neighboring the latter.

In some embodiments of the present invention, a second opening can have the function (and the shape required thereto) not to be large enough for allowing (preferably easy) introduction of a tension thread through a wide area of the sleeve aperture. The wide area of the sleeve aperture can thus, for example, be covered by the shaft wall. The second opening can have the function to lead at least two tension threads through at least two recesses in such a way that the tension threads cannot touch each other and/or cannot get in contact with each other in the recesses and/or cannot entangle themselves.

In certain embodiments of the present invention, a second opening can have the function of not bringing a separation device or a cutting device in contact with one or all of the tension threads or of inhibiting such a contact.

In some embodiments of the invention, the separation device or the cutting device can be covered at the second opening by a portion of the wall such that the tension threads cannot get in contact with the separation device or the cutting device.

In certain embodiments of the invention, the openings can each comprise a range of geometrical shapes. Therefore, the openings need not to have an unchangeable shape or size as long as the respective function (and optionally this function alone) is possible in the respective range of the geometrical shape. However, each opening can be variable within the limits given by the corresponding range.

In some embodiments of the invention, the apparatus is designed for folding and/or unfolding an implant in form of a stent or a cardiac valve assembly.

Furthermore, the present invention may refer to a method of folding or unfolding an implant or to a method of detachably attaching an implant comprising such a step. Such a method of folding or unfolding or step may, according to the invention, comprises the use of an apparatus or of a set according to the present invention.

In certain embodiments the method or the method step may comprise the altering the tension that is exerted on an implant by using at least one tension thread. The tension may be preferably controlled by altering a length of the pulling device branching off the interior of the shaft.

In some embodiments the method or the method step may further comprise cutting or cutting through, respectively, at least one tension thread by inducing, enabling or allowing a relative movement between the shaft and the sleeve.

The advantages achievable by the apparatus as described above can also be achieved by the set according to the invention and/or by an implantation device.

Among the advantages achievable according to the present invention is the possibility of independently actuating tension threads. This is due to the self balancing design. Thus, it is possible to specifically fold and/or unfold implants having a plurality of tension threads. Parts or sections of the implant can thus be folded or unfolded though other parts or sections of the implant have already been completely folded or unfolded. This can i.a. be reasonable when an unfolded implant and the implantation site do not completely match in their dimensions, or if the implant does, e.g., not have a uniform shape over its entire length.

Furthermore, the possibility of simply connecting tension threads to the apparatus, for example, by means of and trough the wide area of the sleeve aperture in some embodiments of the invention, is one of the advantages achievable according to the present invention.

Another advantage is that the tension threads can in some embodiments of the present invention be led or guided separately from each other. A mutual interference of the tension threads can thus be avoided.

In some embodiments of the present invention, it is advantageously possible to separate at least one tension thread from the implant. The thread can particularly be cut through by means of a cutting device.

Another advantage may arise from the fact that, in some embodiments, only one tension thread (or two or more threads) can specifically be separated or detached or cut through, while other tension threads will not be separated, detached or cut through. Particularly for a sloop-like or loop-like design of the tension threads, it is thus possible to cut through a portion of the tension thread instead of the sloop as a whole. In that way, the whole material that had constitute the sloop or loop can be retracted into the shaft by pulling the non-cut parts or side of the sloop of the tension thread(s). In other words, the tension threads not cut through can be used for separating or detaching all tension threads from the implant by simply pulling the tension thread. This can advantageously be useful i.a. after an implantation.

In some embodiments, the implant comprises at least one supporting means which is suited for supporting the implant at or on an implantation site.

In certain embodiments, both the supporting means and the implant are expandable from a respective first diameter to a respective second diameter and/or are collapsible from the second diameter to the first diameter.

In some embodiments, the supporting means comprises bars which are connected to each other by means of connecting sections.

In certain embodiments, the supporting means comprises at least one post for connecting the supporting means with at least one other structure of the implant.

In some embodiments, at least two of the connecting sections differ in at least one material characteristic, for example in the thickness of the respective connecting sections, in the selection of their manufacturing material, i.e. chemically or in a combination of a different thickness in an arbitrary direction and of a different material selection.

In certain of the embodiments, the material characteristic refers to a thickness of at least one section of the connecting sections in a longitudinal direction of the implant and/or in a direction present at a right angle thereto (lateral direction). In an implant which is perfused by bodily fluids after its implantation, the longitudinal direction of the implant can correspond to the direction of the (main) perfusion. The lateral direction of the implant can be a direction in which a (main) expansion of the supporting means takes place (e.g., at the implantation site in the patient's body or before the implantation in a laboratory without any external application of force to the supporting means).

In some of the embodiments of the implant, a first connecting section having the smallest distance to a post has a first thickness d1. Thereby, this thickness is the smallest thickness of all thicknesses of the connecting sections.

In certain of the embodiments of the implant, a second connecting section has a second thickness d2. Thickness d2 is larger than thickness d1. In some embodiments, the second connecting section has the next smaller distance to the post. It is thus adjacent to the first connecting section and has a larger distance to the considered post than the first connecting section.

In some of the embodiments of the implant, a third connecting section has a third thickness d3. Thickness d3 is larger than thickness d2. It is thus adjacent to the second connecting section; however, it is not adjacent to the first connecting section and has a larger distance to the considered post than the first connecting section and the second connecting section.

In certain of the embodiments of the implant, a post is understood as a section of the supporting means which substantially is not curved, i.e., extends linearly.

In some embodiments, a connecting section is understood as a section of the connecting means which connects two or more bars with each other and which is more curved than at least one of the bars.

In certain embodiments, a connecting section is understood as a section of the supporting means being a point of curvature in the sense of a curve progression. Thereby, the point of curvature of the connecting section is the point which is ever located most next to an end of the implant—with respect to the longitudinal direction thereof.

In some embodiments, the implant is designed or embodied with cardiac valves—in particular artificial ones or ones which have been manufactured from animal tissue.

In certain embodiments, bars comprised by the supporting means which connect a rod or post with the adjacent connecting sections are shorter than other bars. The shorter bars contribute to forming a slit that reaches from an end of the supporting means to the adjacent end of the post. The slit has a length of L2 (from its open end to its closed end).

In some embodiments, from the opposite end of the slit to the adjacent end of string outlet or aperture 10 a distance having a length L3 is provided. Between the closed end of the slit and the centre of string outlet or aperture a distance having a length L4 is provided.

In certain embodiments, the open end of the slit may be spaced from the centre of the outlet or aperture by the sum of L2 and L4.

In particular embodiments, L1 is between 2.5 and 3.5 times as long as L2, preferably 3 times as long. L1 is the length of the supporting means in a distal-proximal direction thereof.

In some embodiments, L2 is 2 times (or between 1.5 and 2.5 times) the length of L4.

In certain embodiments, L2 is 3 times (or between 2.5 and 3.5 times) the length of L4.

Regarding its basic design, the implant can be constructed in some embodiments as is, for example, described in DE 10 2008 013 948 A1 by the applicant of the present invention. By way of reference, the content thereof is also subject-matter of the present invention. This particularly applies for the materials and (part) geometries given therein.

In certain embodiments, the implant can be partly self-expanding, partly by the use of an expanding means.

In some embodiments, the implant can exclusively be non-self-expanding. By using the implant according to some embodiments of the invention, an expansion thereof is possible without distorting or warping, respectively, or undulating or waving, respectively, the supporting structure. In some embodiments, this can primarily enable a multiple, however, at least a twofold, re-expansion following a happened reduction of the cross-section. The desired expansion behavior of the supporting means is maintained in such a case, too. A distortion or warping, respectively, or an undulation or waving, respectively, does not take place in such a case, too. The latter advantageously allows for a precise positioning of the implant. It can further ensure the more accurate residing of the supporting means and, thus, of the implant against the tissue of the implantation site.

Further, the likelihood of any undue or adverse stress concentration within the solid parts of the implant can advantageously be avoided or minimized.

In some embodiments, the in particular medical, implant comprises at least one foldable or collapsible or crimpable and unfoldable or expandable structure (therefore also referred to as structures) on or around or over a portion or outer surface of a catheter or of a catheter tip or of any other delivery implement or device or part thereof is proposed.

In particular embodiments, a method or method step for crimping may comprise the feature that no pressure or only a permissible, for example predetermined pressure, is exerted on the structure during and/or after crimping of the implant beyond a predetermined pressure.

In some embodiments, the set according to the invention and/or the implantation device or a device for receiving and/or supporting the implant may comprise at least one crimping device for crimping of an implant comprising at least one foldable and unfoldable structure on or around or over a portion or outer surface of a delivery or implantation device with a predetermined pressure exerted on the structure.

In certain embodiments, the predetermined pressure exerted does not exceed a predetermined pressure.

In some embodiments, no pressure is exerted on an implant or on the least one foldable and unfoldable structure during and/or after crimping of the implant beyond a predetermined pressure.

In particular embodiments in the context of the present invention, the term “crimping an implant” may mean the crimping result achieved after termination of the entire crimping process of the medical implant.

In the context of the present invention the term “crimping an implant” may mean that the implant crimped is to be understood as prepared on a delivery or implantation device or device to be inserted or implanted.

In certain embodiments, in the context of the present invention the term “crimping a medical implant” may mean that additional or further crimping it not necessary or not contemplated or not required before implanting of the implant.

In some embodiments, in the context of the present invention the term “predetermined pressure” may refer to a pressure value that has been determined and/or considered and/or selected by the person responsible for the crimping process or carrying out the same before or during the crimping process takes place.

In certain embodiments in the context of the present invention the term “predetermined pressure” may refer to a pressure value adjusted at a crimping device. The value can preferably be adjusted as a maximum pressure value exerted on certain structures of the implant, for example, heart valve replacement leaflets or commissures thereof.

In some embodiments, in the context of the present invention the term pressure exerted “during and/or after crimping of the implant” may refer to pressure exerted by means of the crimping itself.

In particular embodiments of the method of detachably attaching an implant and/or of releasing an implant according to the invention, the method may comprise the step of measuring the pressure acting on or in the structure, or between the structure and other parts of the implant, or between the structure and the delivery device (in particular, the circumferential surface or a section thereof of the delivery device), and the step of terminating the crimping procedure once the pressure measured has reached the predetermined pressure or exceeds the predetermined pressure. In some of these embodiments, the methods comprises placing a pressure or force sensor in direct contact with the structure.

In certain embodiments, in the context of the present invention the term “predetermined pressure” may refer to a pressure that exclusively results from the crimping steps as such. In those embodiments, pressure exerted on the structures at issue stemming or originating from other pressure sources than by the crimping steps is not referred to as the predetermined pressure. Such other pressure comprises the atmospheric pressure, water or fluid pressure, and the like. In certain embodiments, such additional pressure does not contribute to the determined pressure or the level thereof.

In some embodiments a predetermined pressure may be understood as a predetermined force, strain, stress and the like as well. Hence, in those embodiments, the terms pressure, force, strain, stress and the like may be understood as interchangeable.

In certain embodiments, the predetermined pressure is to be understood as a maximally allowable pressure that is measured or may be measured between the structure of the implant and a circumferential surface or an envelope of the delivery device, or equals the such measured pressure.

In particular embodiments, the structure is not the proximal or the distal ring of the implant.

In some embodiments, the structure on the implant is not intended to contribute to the temporary fixation of the implant on the delivery implement/device.

In certain embodiments, the implant comprises one or more interconnecting elements, and the pressure exerted or applying on the structure is determined between the interconnecting elements and the outer surface or the portion of the catheter.

In some embodiments, the interconnecting elements may be embodied as posts interconnecting a proximal and a distal ring or support structure.

In certain embodiments, the interconnecting elements may be embodied as radially (as regards a longitudinal axis of the implant or of the delivery device) expandable or shiftable structures, of the implant, wherein they are expanded or shifted or moved away from the upon expansion of implant.

In some embodiments, the interconnecting elements may be embodied as one or more posts.

In certain embodiments, the interconnecting elements may be embodied as structures provided for maintaining a distance between a distal ring and a proximal ring of the implant.

In some embodiments, the predetermined pressure is 0 N per square millimetre (0 N/mm2) or 1 N/mm2 or 2 N/mm2 or 3 N/mm2 or 5 N/mm2. If the predetermined pressure is 0 N/mm2 or about 0 N/mm2, the method may be called a “zero pressure crimping” method.

In certain embodiments, the predetermined pressure is 5 N per square millimetre (5 N/mm2) or 8 N/mm2 or 10 N/mm2 or 15 N/mm2 or 20 N/mm2 or 25 N/mm2 or 30 N/mm2 or any value in between.

In some embodiments, the method of crimping or folding and/or unfolding is carried out manually by the aid of non-electric tools.

In certain embodiments, the method of crimping is carried out by the aid of automatic tools. Such tools can be electric, pneumatic, hydraulic tools and the like.

In some embodiments, the crimping device comprises a pressure limiting means for limiting the pressure that is exerted or exertable on the implant and/or on the structure during and/or after crimping of the implant.

In certain embodiments, the pressure (or force) exerted or exertable on the structure may be known once the pressure (or force) exerted on the implant comprising the structure is known. For example, it might be known—e. g. from known relationships between a first and a second pressure as defined in the following—that zero pressure (being one example of a first pressure) is applied on the structure if less than a certain pressure (second pressure) is exerted on the implant during crimping. In those embodiments, it may be sufficient to limit the (second) pressure applied to the implant. As may be known in that case from earlier experiments or from a look-up-table, the (first) pressure applied to the structures or acting on the structures in question will then not be higher than a predetermined pressure or a pressure considered to be a maximum pressure that is allowed to apply to the structure.

In some embodiments, the crimping device comprises a pressure sensor (or is functionally linked with it) that reflects the pressure or force exerted on the structure at issue (e.g., the leaflets comprised by the implant) during crimping. Preferably, the pressure sensor is placed, for example, between the structure at issue (such as the leaflets of the implant) and a neighbouring structure (such as an outer surface or other part of the implantation device used). In certain embodiments, such a pressure sensor or any other suitable sensor is provided with the implantation device. In some embodiments, the pressure sensor or any other suitable sensor is located within a lumen of the implantation device or on an outer surface thereof.

In certain embodiments, the crimping device is intended and/or configured for crimping by means of a predetermined pressure exerted on the structure during and/or after crimping of the implant.

In some embodiments, the crimping device comprises a controller for limiting or controlling the pressure exerted on the structure of the implant.

In certain embodiments, the crimping device comprises an adjusting means for adjusting the pressure exerted upon crimping. The adjusting means may be connected to the controller.

In some embodiments, the crimping device is intended and/or configured for crimping medical implants, in particular for crimping only medical implants.

In certain embodiments, the crimping device comprises one or more pressure sensors that output a signal indicating the pressure applied on the structures at issue during crimping.

In some embodiments a device for receiving, supporting and/or folding or unfolding the implant is intended to be attached to or interconnected with at least a medical implant and intended to be attached to or interconnected with at least an implantation device and/or a device supporting or receiving the implant.

In certain embodiments, the device can be temporarily or permanently or detachably attachable to or interconnectable with the medical implant.

In some embodiments, the device can be temporarily or permanently or detachably attachable to or interconnectable or connected, respectively, with the implantation device.

In certain embodiments, the device can be mainly or partly tube-shaped. As such, the device can have a circular or oval cross-section. However, the device may also have any other cross-section apt for establishing a connection between the device and the implantation device.

In some embodiments, as regards the implant, the device does not have to be designed in a particular way as long as the implant can be temporarily or permanently or detachably fixed at or onto the device.

In certain embodiments, the device detachably comprises the implant.

In particular embodiments, the implant can be of any type that is known to a person skilled in the art for supporting or carrying out functions of a patient's body. Examples include implants such as heart valves, substitutes or replacement of heart valves, stents for holding vessels or other body tubes open, and the like.

In some embodiments, the implant is foldable and/or unfoldable and comprises first folding and/or unfolding means adapted and/or intended or configured for folding and/or unfolding the implant.

In certain embodiments, the implant is intended to be attached to or interconnected with the device by means of crimping. That is, the device is intended to have the implant crimped thereon, or the implant has already been crimped onto the device.

In some embodiments, the first folding and/or unfolding means are guided around certain portions of the implant that can be tightened or released.

In certain embodiments, the first folding and/or unfolding means can be arranged at or at least in connection with the implant such that it is or they are, respectively, operatively connected with the implant. The first folding and/or unfolding means can be arranged such that they can contribute to or effect the folding and/or unfolding of the implant which is attached to the device. The folding and/or unfolding of the implant by use of the first folding and/or unfolding means can be effected when a force, a tension or stress or strain is applied or put, onto the first folding and/or unfolding means or rather released from the first folding and/or unfolding means. Such a tension, stress or strain can, for example, be induced or generated by an actuating device (e.g., a pulling device) which can be operated by a user.

In some embodiments, it can be intended to use at least the first folding and/or unfolding means of the implant to establish a connection such as a form closure connection between the device and the implantation device.

In certain embodiments, the first folding and/or unfolding means of the implant can pass through an inner space of the device.

In some embodiments, the inner space is an opening that extends along the whole or entire length of the device, i.e. from a distal end to a proximal end thereof. In other embodiments, the inner space is arranged in a longitudinal direction of the device and extends at least from a front end opening of the device (be it the proximal or the distal end of the device) to a second opening which is an outlet of the first folding and/or unfolding means.

In certain embodiments, the first folding and/or unfolding means of the implant can be arranged such that they leave the device through at least one opening of the device. Such an opening may be provided at one end of the device. However, the first folding and/or unfolding means can leave the device at any other suitable position. The first folding and/or unfolding means can leave the device all through the same opening, however, some of the first folding and/or folding means can also leave the device through different openings.

In some embodiments, the first folding and/or unfolding means can comprise one or more tension threads or threads or strings or can consist thereof.

In certain embodiments, the device comprises attaching or interconnecting means. Such attaching or interconnecting means are intended and provided for attaching or interconnecting the device to an implantation device. The attaching or interconnecting means can assist or support the attachment or interconnection of the device with the implantation device.

In some embodiments, the attaching or interconnecting means are arranged at or within the device. Examples include lugs or noses or the like, but also recesses or notches or the like which are arranged in an inner space of the device in such a way that they favour the attachment or interconnection of the device with the implantation device. However, the device does not have to comprise particularly formed geometrical shapes.

In particular embodiments, in order to establish a preferably tight or firm connection, the device can comprise male faces or terminals and the implantation device can comprise female faces or terminals or vice versa. As such, the connection between the device and the implantation device can resemble or be a plug-in connection.

In certain embodiments, the device is a catheter tip. During preparation of implanting the implant attached to the device by use of the implantation device, the device, the catheter tip or the like can be attached to or interconnected with the implantation device in situ, for example, in the operating room or theatre, by, for example, merely slipping or snapping on the catheter tip onto the implantation device.

In some embodiments, the implantation device according to the present invention is suited and/or configured or prepared for receiving at least one such device.

In certain embodiments, the implantation device comprises at least one device.

In some embodiments, the implantation device comprises attaching or interconnecting means for being attached to or interconnected with the device. Such attaching or interconnecting means can include lugs or noses or the like, but also recesses or notches or the like. The interconnecting means can be arranged in an inner space of the implantation device or at an outer surface or any other part thereof. The implantation device may have any particularly formed geometrical shapes. The attaching or interconnecting means of the implantation device can form complements or counter pieces, respectively, to the attaching or interconnecting means of the device.

In certain embodiments of the invention, the implantation device comprises second folding and/or unfolding means.

In some embodiments, these second folding and/or unfolding means can contribute to or effect an interconnection or attachment of the implant to the device.

In certain embodiments, the second folding and/or unfolding means is interconnected with or attached to the first folding and/or unfolding means of the implant. The second folding and/or unfolding means can be embodied as tension threads, threads or strings or the like.

In some embodiments, the second folding and/or unfolding means is made of wire (or comprises a wire). In other embodiments according to the invention, the second folding and/or unfolding means is not made of wire (nor comprises it one).

In certain embodiments, the second folding and/or unfolding means is operatively connected to the first folding and/or unfolding means of the implant.

In particular embodiments, the second folding and/or unfolding means is operatively connected to the first folding and/or unfolding means of the implant to remain connected until the implantation device is withdrawn from the patient's body after completion of the implantation of the implant. That is, in these embodiments, the implant is released from the device and/or from the implantation device, by disconnecting the device from the implant (or by releasing the implant from the device) at a site that is different to a connection site where the first and the second folding and/or unfolding means had been connected to each other. In these embodiments, the implantation device may comprise a separating means to separate or to release (e.g., to cut or to disconnect) the implant from the first and/or the second folding and/or unfolding means at a site different to the site where the first and the second folding and/or unfolding means had been connected with each other.

In some embodiments, both the first and the second folding and/or unfolding means are intended to remain with the implantation device after release of the implant from the implantation device.

In certain embodiments, different folding and/or unfolding means are differently colour coded, or they have matching connectors of different shape, or they have different lengths or a combination of such features to avoid a wrong and possibly adverse connection.

In some embodiments, the second folding and/or unfolding means contributes to folding and/or unfolding the implant by means of the first folding and/or unfolding means in that they can transmit a force such as a tension or stress from an actuating device operated by a user to the implant. Usually, such an actuating device is arranged at a proximal end of the implantation device as regards a user, such as, for example a tool holder or a handle, wherein the implant is arranged at the distal end of the implantation device as regards the user.

In certain embodiments, the second folding and/or unfolding means are knotted or interloped with the first folding and/or unfolding means. In other certain embodiments, hook and eye connections are used to interconnect the first and the second folding and/or unfolding means. However, the design or construction of the connection between the first and the second folding and/or unfolding means is not restricted to a particular design. As long as the intended connection between the first and the second folding and/or unfolding means is achieved, any design or construction apt for this purpose is contemplated.

The method of attaching or interconnecting according to the invention serves for loading or providing a delivery implement with an implant before implantation, wherein the method comprises attaching or fixing a device according to the invention comprising an implant onto a delivery implement.

In certain embodiments, the device comprises at least an implant or is composed at least of an implant.

In some embodiments of the method, the implant is released from the device and/or from the implantation device by disconnecting the device at a site that is different from a connection site at which the first and the second folding and/or unfolding means had been connected to each other (before implantation). In some of these embodiments, appropriate means for separating the implant from the device or from the implantation device (i.e. by cutting of strings) may be used.

In certain embodiments, the attachment or fixation of the device to or onto the delivery implement such as an implantation device, in particular a catheter, can be performed at any desired or required point of time. In some embodiments, the device is attached or fixed to the delivery implement in the operation room or theatre or a the bedside.

By using the device, the present invention provides a simple option for attaching or interconnecting an implant to an implantation device at any desired or required point of time, in particular in situ in the operating room just before implanting the implant.

As medical implants can also partly or entirely consist of living tissue, such as for example, pig heart valves, it may be recommended to keep the living tissue in fluid environment during storage or transport. However, at least due to its mechanical structure, the implantation device as a whole should not be stored or transported under wet conditions.

With the described device, it is advantageously possible to store and/or transport the implant and the implantation device separate from each other in best suitable environments.

In some. embodiments, it is possible to assemble the implant and the implantation device for the purpose of implanting in a relative short time and in an uncomplicated manner. For example, in certain embodiments of the invention, a cumbersome assembling of strings and implant right before implantation, e.g., at the bedside, can advantageously be avoided.

In this way, it is advantageously possible to store and/or transport the implantation device in a, for example, dry environment suitable for the sensitive mechanical structure of the implantation device; and to store and/or transport the implant under wet or humid conditions in order to keep its biological tissue in a humid condition. Thus, possible damages of the mechanical structure of the implantation device can advantageously be avoided. The biological tissue does not dry out.

In certain embodiments of the present invention, the device can be designed or constructed such that it is not susceptible for being damaged or destroyed by fluids such as liquids surrounding the implant. As such, it is advantageously also possible to interconnect the device and the implant before storage or transportation. In particular, due to the separation of implantation device and device (tip of the implantation device, for example), both the implantation device and the device can be produced from different materials, in different processes and the like. Each can thus be manufactured to its best, independently of the need of the other part.

Due to the attaching or interconnecting means of the device, it is advantageously possible in certain embodiments to establish or achieve a simple and uncomplicated connection between the device and the implantation device.

In some embodiments, due to simply establishing a connecting between the first folding and/or unfolding means of the implant and the second folding and/or unfolding means of the implantation device, it is advantageously simply and in an uncomplicated manner possible to transfer the required force for folding and/or unfolding the implant from an actuating device of the user to the implant.

In some embodiments, the device for receiving and/or supporting the implant comprises a portion intended for folding and/or unfolding the implant.

The device can be temporarily or permanently or detachably attachable to or interconnectable with the medical implant. The device can be temporarily or permanently or detachably attachable to or interconnectable or connected, respectively, with the implantation device. The implantation device is intended for implanting the implant.

In certain embodiments, the portion intended for folding or unfolding the implant is arranged rotatably, in particular around a longitudinal axis of the device or of the implantation device.

According to the invention, the rotatability can relate or be relative, respectively, to the surroundings, an exterior, an outer layer, or the like of the device.

In some embodiments, the portion intended for folding or unfolding the implant can be supported within the device by means of a bearing, e. g., a pivot bearing.

In certain embodiments, the portion can be provided in one single component with the first portion of the means for attaching or interconnecting described further below or can be provided in force connection in any other way such that both portions are able to rotate only commonly.

In some embodiments, the portion for folding and/or unfolding the implant can be cylindrical or a rotationally symmetrical portion or comprise such a portion.

In certain embodiments, the portion for folding and/or unfolding the implant may have openings intended for guiding strings or threads therethrough.

In some embodiments, the portion intended for folding or unfolding the implant can consist of or comprise another material than a thread or string, respectively, material.

In certain embodiments, the portion for folding and/or unfolding the implant can have another form than a thread or string, respectively, form.

In certain embodiments, the portion can be provided and intended for winding a thread or a string thereon, the thread or string being intended for folding and/or unfolding the implant.

In particular embodiments, the portion intended for folding or unfolding the implant can be detachably interconnected with the device.

In some embodiments, the portion intended for folding or unfolding the implant can be provided and/or intended not to be separated or released from the device by cutting.

In certain embodiments, the portion intended for folding or unfolding the implant may not contact the implant in the state of use of the device.

In some embodiments, the portion intended for folding or unfolding the implant is completely separated from the implant by means of an outer component or layer of the device in the state of use of the device in some embodiments.

In certain embodiments, the portion is interconnected with a portion of the means for folding and/or unfolding the implant. This connection can be an interlooping connection, a screwing connection, a sticking connection, or the like.

In some embodiments, the portion comprises a guiding structure.

In certain embodiments, the guiding structure can be provided at an outer surface of the portion.

In some embodiments, the guiding structure can comprise or consist of one or more channels or grooves, respectively, or recesses that are intended for winding the means for folding and/or unfolding the implant, e. g., in form of one or more threads or strings, respectively.

In certain embodiments, the guiding structure can comprise or consist of one or more fins or noses or lugs, respectively, that are intended for winding the means for folding and/or unfolding the implant, e. g., in form of one or more threads or strings, respectively.

In particular embodiments, the device can further comprise a displacing means or an advancing means (or mechanism) by which the portion for folding and/or unfolding the implant can be moved forwards or backwards during its rotation around its longitudinal axis within the device or a portion thereof.

In some embodiments, it is contemplated to actuate the displacing mechanism or the advancing mechanism or a corresponding mechanism intended for advancing, retracting or displacing the portion intended for folding and/or unfolding the implant by hand only. In other embodiments, a motor or the like is provided and intended to be used (solely or auxiliary) for advancing or displacing the portion intended for folding and/or unfolding the implant.

In certain of the embodiments in which such a motor is provided, the motor or main parts of it can be located, for instance, at or near the tip or in or at a tip portion of the implantation device. In other embodiments, the motor or main parts of it can be located at the implantation device's handle.

In some embodiments, the folding and/or unfolding means can be provided at or at least in connection with the implant such that it is or they are, respectively, operatively connected with the implant. The folding and/or unfolding means can be arranged such that it or they, respectively, can contribute to or effect the folding and/or unfolding of the implant which is attached to the device. The folding and/or unfolding of the implant by means of the folding and/or unfolding means can be effected when a force, a tension or stress or strain is applied or put, onto the folding and/or unfolding means or rather released from the folding and/or unfolding means. Such a tension, stress or strain can, for example, be induced or generated by an actuating device (e. g., a pulling device) which can be operated by a user.

In certain embodiments, the means for folding and/or unfolding the implant can pass through an inner space of the device. The folding and/or unfolding means of the implant can be arranged such that they leave the device through at least one opening of the device. Such an opening may be provided at one end of the device. However, the folding and/or unfolding means can leave the device also at any other suitable position and/or re-enter therethrough. The folding and/or unfolding means can leave the device all through the same opening, however, some of the folding and/or folding means can also leave the device through different openings and/or re-enter therethrough.

In some embodiments, the folding and/or unfolding means can comprise one or more tension threads or threads or strings or can consist thereof.

In particular embodiments, the folding and/or unfolding means are not identical to the device.

In some embodiments, the device is not intended to be implanted itself or to remain within the patient's body after the implantation procedure has been finished.

In certain embodiments, the device is intended to be separated from the implant after the implantation procedure has been finished.

In some embodiments, the device is configured to be separatable from the implant during normal use of the device. In certain embodiments, the device is not permanently attached to the implant or linked to it.

In some embodiments, the device is arranged within a central part or through-hole of the implant.

In certain embodiments, the device and/or the implantation device comprise attaching or interconnecting means. Such attaching or interconnecting means are intended and provided for attaching or interconnecting the device to or with, respectively, an implantation device. The attaching or interconnecting means can assist or support the attachment or interconnection of the device with or to, respectively, the implantation device.

In some embodiments, the means for attaching or interconnecting comprises a first section which is arranged rotatably in or at the device, in particular around a longitudinal axis of the device or of the implantation device.

In certain embodiments, the first section is preferably rotatably supported in or at the device.

In some embodiments, the means for attaching or interconnecting comprises a second section which is not arranged rotatably in or at the device, in particular not around a longitudinal axis of the device or of the implantation device.

In certain embodiments, examples for the first and the second section include lugs or noses or the like, but also recesses or notches, toothings or coggings, dogs, tooth or gear wheel structures, clip connections, plug-in connections, or the like. However, the device does not have to comprise particularly formed geometrical shapes.

Everything that was said herein about the first section of the means for attaching or interconnecting may undiminishedly also apply for the third section. This is, however, not mandatory.

Everything that was said herein about the second section of the means for attaching or interconnecting may undiminishedly also apply for the fourth section. This is, however, not mandatory.

In some embodiments, the first and the second section are present on or in or at the device.

In certain embodiments, the third and the fourth section are present on or in or at the implantation device.

In some embodiments, in order to establish a preferably tight or firm connection between, e. g., the first and the third section and/or between the second and the fourth section, the device can, for example, comprise male faces or terminals at the first and/or the second section and the implantation device can comprise female faces or terminals at the third and/or the fourth section or vice versa. As such, the connection between the device and the implantation device can resemble or be one plug-in connection or two plug-in connections.

In certain embodiments, the device is an implantation device tip. During preparation of implanting the implant attached to the device by means of the implantation device, the device, the catheter tip or the like can be attached to or interconnected with the implantation device by merely slipping or snapping on the catheter tip onto the implantation device in situ, for example, in the operating room or theatre.

In some embodiments, the implantation device is suited and/or configured or prepared for receiving at least one such device.

In certain embodiments, the implantation device comprises at least one device.

In certain embodiments, the device can be designed or constructed such that it is not susceptible by fluids such as, for example, liquids surrounding the implant or is damaged or destroyed by those. As such, it is advantageously also possible to interconnect the device and the implant before storage or transportation. Due to the separation of implantation device and device (tip of the implantation device, for example), both the implantation device and the device can be manufactured from different materials, in different processes and the like. Each can thus be manufactured to its best and independently from the other part.

In certain embodiments, due to the attaching or interconnecting means of the device, it is advantageously possible to establish or achieve a simple and uncomplicated connection between the device and the implantation device.

In some embodiments, as, the means for folding and/or unfolding the implant are interconnected with the portion for folding and/or unfolding the implant provided in the device and as the means for folding and/or unfolding are thus provided at the device alone and not also at the implantation device, the means for folding and/or unfolding can advantageously be kept short.

Moreover, due to their shortness, the means for folding and/or unfolding do not have to be guided through an interior of the implantation device to the hand of a surgeon as it has to be the case in other solutions of the applicant of the present application as well. The present solution is thus advantageously characterized in that the means for folding and/or unfolding do not have to be diverted or deflected, respectively, at all or only less, cannot experience any shear forces, experience less friction, and the like and the possibility of being displaced, entangled or the like is advantageously reduced.

Another advantage of certain embodiments can be that, due to shorter means for folding and/or unfolding the implant, a shorter range of move is required for the mechanism used. Thus, for the example of the thread as a means for folding and/or unfolding the implant it is known that the thread is subjected to a lengthening generated due to mechanical stress. This lengthening resulting in a reduced precision of the function of the entire mechanism may advantageously be prevented or, however, significantly reduced with respectively short threads as are possible.

Another advantage of some embodiments is that, due to their short design, less forces act on the means for folding and/or unfolding that do not have to be diverted around curves, bendings, and the like. This particularly applies at bending portions of the means and/or the device or the implantation device, respectively. The means such as, e. g., the one or more threads can be manufactured thinner, more simply, cheaper. This advantageously further allows for a cutting device for cutting the threads after a successful implantation of the implant being designed in a more simple, smaller and/or cheaper way.

A still further advantage of certain embodiments is the omission of the requirement of having to connect the means for folding and/or unfolding the device that can, for example, be threads or strings, respectively, with means for their operation that can be provided in the implantation device after attaching the device at the implantation device. A connection of the means for folding and/or unfolding of, e. g., the threads with the implantation device is not required. It is sufficient for the present invention to connect the device with the implantation device. A further connection is not required. In this way, in particular a connection of threads of the device with threads of the implantation device or the like which can be time-consuming and cumbersome can be omitted. The latter can advantageously contribute to maintaining the required sterility.

In some embodiments, it is thus possible that the means for folding and/or unfolding the implant are solely present in the device, e. g., at the implantation device tip. They do not have to extend across the entire implantation device. In this way, the implantation device does also not have to be designed such that the means can penetrate therethrough.

Another advantage of certain embodiments is that—in a correspondingly designed guiding structure that can extend, for example, spirally or helically along the portion for folding and/or unfolding the implant—a winding path of the means for folding and/or unfolding the implant that is designed in form of a thread in some embodiments can disperse or extend, respectively, along the longitudinal axis of the portion for folding and/or unfolding the implant. Thus, an only small space within the device, for example, between an outer sheath and the portion for folding and/or unfolding the implant rotatably supported therein is sufficient for winding the thread as close as possible at or around the portion for folding and/or unfolding the implant. In this way, the device can advantageously be designed having a smaller diameter. The same advantage can be obtained by providing an advancing mechanism or a displacing mechanism.

In some embodiments, the implantation device comprises an aligning device for aligning the apparatus at the implantation site.

In certain embodiments, the aligning device is capable of being transferred from a non-aligning position into an aligning position.

In certain embodiments, aligning the implantation device according to the invention is alternatively or additionally understood as aligning the medical implant.

In some embodiments according to the invention, aligning is alternatively or additionally understood as orienting the implantation device or the medical implant such that a user of the implantation device is aware of the position of the implantation device and/or the implant relative to a body tissue or a body structure or an anatomic condition of the implantation site, respectively, after aligning or that the user is given a corresponding hint by means of the alignment.

In certain embodiments, aligning is understood as aligning the implantation device and/or the medical implant.

In some embodiments, the aligning device is capable of being transferred from the aligning position into the non-aligning position as well.

In certain embodiments of the implantation device, the aligning device is or comprises a wire or a filament, respectively.

According to the present invention, the term “wire” or “filament”, respectively, may also define a plurality of wires or filaments, respectively, whenever a person skilled in the art recognizes the exchangeability of the terms.

In some embodiments, the aligning device comprises a through opening or passage opening, respectively, in at least one section thereof.

In certain embodiments, the through opening extends along the aligning device or a section thereof. The through opening may be present in an interior of the aligning device. It can be intended or provided and/or serve for transporting or guiding or directing a fluid from one end of the aligning device or of the section thereof to another end portion of the aligning device or of the section.

In some embodiments, the through opening is penetrable or permeable, respectively, for fluids in its longitudinal direction. The term “penetrable” or “permeable”, respectively, hereby refers to the ability of the aligning device to be flown through by fluids and/or to guide the said.

In certain embodiments, the aligning device is movable—namely either as a whole or in sections thereof—relative to the implantation device.

According to the invention a corresponding support may be provided, but does not have to be provided. A corresponding material combination may be provided, but does not have to be provided.

In some embodiments, the implantation device is tubular (i.e., having a hollow interior), such as, e.g., a hollow cylinder having a through opening in or along its interior. The implantation device may be designed symmetrically or asymmetrically, both relative to its through direction and also in another direction, in particular in a direction or plane perpendicular to the through direction.

In certain embodiments, a shaft of the implantation device is penetrable or permeable, respectively, in its interior in at least sections of its longitudinal direction. The shaft comprises a wall. The shaft comprises at least one shaft opening or shaft aperture, respectively. The at least one shaft aperture is preferably not arranged at the front side but at or on a lateral or envelop surface of the shaft. The shaft aperture is preferably a through opening establishing a connection between the interior and an exterior of the shaft of the implantation device. In certain embodiments, the aligning device can be delivered or passed or transferred from the non-aligning position into the aligning position through the shaft aperture.

In some embodiments, the shaft of the implantation device comprises a plurality of shaft apertures that are evenly or unevenly spaced around or across a periphery or a lateral surface of the shaft. Additionally or alternatively, the shaft apertures may be dispersed along a longitudinal direction of the shaft.

In certain embodiments, sections of the aligning device may enter and/or exit through the shaft apertures.

In some embodiments, the aligning device comprises two or more aligning sections that may be actuated independently from each other such that they can contact the tissue of the implantation site or of the implantation organ or of the organ independently from each other, respectively.

For this purpose, the aligning sections are in some embodiments capable of being transferred from the non-aligning position into the aligning position independently from each other.

In particular embodiments, the aligning device comprises or consists of a memory shape material.

In certain embodiments, the aligning device of the implantation device comprises at least one open or closed loopy or wound section. The latter can have the form of a loop or sling.

In particular embodiments, the open or closed wound section may have the form of a pig tail, a spiral, a helix, or the like.

In some embodiments, the implantation device comprises a reception area for receiving the implant.

In certain embodiments, the reception area is a shaft or has the form of a shaft.

The reception area can in some embodiments receive the implant releasably such that the implant can be delivered to the implantation site by means of the implantation device. After having reached the final implantation site, the implant may be released from the reception area optionally by using appropriate means or devices, respectively. Then, the implant stays at the implantation site while the reception area may be removed from the implantation site together with the other sections of the implantation device.

In certain embodiments, the aligning device of the implantation device is connected or intended or provided and suited for a connection with a means or device, respectively, for releasing a fluid for medical imaging.

In some embodiments, the aligning device is designed or embodied as a spring, in particular as a spiral or coil spring, and/or is formed from an appropriate—preferably an elastic or flexible—material such as, e.g., a plastic material or gum.

In certain embodiments, the implantation device comprises at least two or at least three different passages or apertures, respectively.

When according to the invention at least two or three different passages or apertures, respectively, are mentioned, this can, in some embodiments, refer to a respective different geometrical shape of the passages or apertures, respectively. In other words, the passages or apertures, respectively, may differ in their design.

In some embodiments, the “three different passages or apertures, respectively” may differ in their area size.

In certain embodiments, a passage or aperture, respectively, is the open passage between an exterior of the implantation device or of the shaft of the implantation device and an interior of the implantation device or of the shaft of the implantation device.

In some embodiments, the passages or apertures, respectively, may each comprise a range of geometrical designs. Thus, the passages or apertures, respectively, do not have to have an unchangeable design or size as long as the respective function (and optionally only this function) is possible within the respective range of the geometrical design. Within the limits predetermined by means of the range, the respective passages or apertures, respectively, may therefore—to all intents and purposes—be variable.

In certain embodiments of the method of attaching and/or releasing an implant, the method may comprise transferring the aligning device from the non-aligning position into the aligning position and/or vice versa, after having inserted the implantation device at the implantation site.

In some embodiments of the methods according to the invention, the methods may comprise directing a fluid for medical imaging through a lumen of the aligning device.

Among the advantages achievable according to the invention is that the mechanical stress or impairment of the tissue of the implantation site in general is in certain embodiments at best only little.

This advantage can even be increased in that the aligning device or the aligning sections thereof are in some embodiments wires or filaments, respectively, e.g., made from or comprising Nitinol, however, in any case, sections having a certain flexibility. This can also favor a gentle handling with the tissue of the implantation site as the mechanical stress applied on the tissue is low. Bleedings, wounds, irritations, and the like can be prevented.

One of the advantages achievable is further that both implanting as well as imaging the implantation site is possible at the same time by using the implantation device in certain embodiments. In these embodiments, there is no further device or means required for this purpose in addition to the implantation device for fulfilling both functions. This can both facilitate the handling of the required instruments as well as require an only smaller access or fewer accesses than the solutions known.

Another advantage is that, in some embodiments, a proper alignment—or more proper as compared to the state of the art—to the morphology of the implantation site is possible., it is thus, e.g., possible to achieve an appropriate alignment even for two-part cardiac valves or the connection sites thereof with the heart muscle, respectively, as, due to the multiple separation of the aligning device, another number of aligning sections is used than is, e.g., provided or required for cardiac valves comprising three valves. Thus, in some embodiments, if needed, e.g., not three but only two aligning sections may be used or transferred into the aligning position. Moreover, the two aligning sections used can exit the implantation device appropriately at positions being suitably spread across the periphery of the implantation device or the shaft thereof. This allows a further advantageous flexibility during use of the implantation device in some of its embodiments. Thus, the latter can be used for, e.g., aligning any arbitrary one of the cardiac valve prostheses known during the implantation thereof.

A still further advantage of the present invention which can be achieved in certain embodiments is the simple match of the aligning device or the aligning sections thereof. This can, for example, be achieved by means of a memory shape (or shape memory, respectively,) material. Thus, the aligning device or sections hereof may consist of Nitinol wires or filaments, respectively, that can be brought into an appropriate form easily, which they are able to reassume after omitting external limitations.

In some embodiments, the implantation device and/or a device for folding and/or unfolding an implant comprise at least an apparatus configured for inserting and/or folding and/or unfolding the implant by means of at least one tension thread.

In particular embodiments, the folding and/or unfolding device, which may be called in the following folding device, comprises a shaft.

In certain embodiments, the folding device comprises at least one tensioning device for altering a form or shape, a geometry or a folding state of the foldable and/or unfoldable implant by means of the at least one tension thread.

In some embodiments, the shaft of the folding device comprises in at least one shaft section thereof a plurality of individual shaft fibers.

In some embodiments according to the present invention, altering a form or shape of the implant means reducing or increasing a diameter, in particular an outer diameter, of the implant. Alterations of the diameter may be accompanied by any kind of alteration of the implant's length or any other alteration, or may be not.

In some embodiments, the at least one tension thread is a thread or filament or yarn, respectively. It can be designed or embodied similar to a surgical sutural thread or it can be such a surgical sutural thread. It can be designed or embodied as a rope or a cord or twine or string, respectively. It can be designed or embodied as a chain comprising a plurality of chain members engaged with adjacent chain members.

According to the present invention, whenever reference is made to a thread or tension thread, the terms may include a plurality of threads or tension threads as well insofar as a person skilled in the art recognizes the exchangeability of the terms.

In certain embodiments, the shaft of the folding device is in at least one section thereof embodied rigidly. In some embodiments, the shaft of the folding device is in at least one section thereof embodied such as to be bendable in one or more directions (i. e. it may be bent in a longitudinal direction or in a direction of the shaft's width, in both directions or in any other direction). In some embodiments, the shaft is embodied extendably or stretchably. In other embodiments, the shaft is embodied stiffly or inflexibly.

In some embodiments, in the moment of unfolding or folding, the implant is loosely arranged or attached to or at or on a receiving area of the folding device. In certain embodiments, the implant is thereby connected with the receiving area only by means of the tension threads.

In some embodiments, a shaft fiber of the folding device is permeable or patent (like a blood vessel) within its interior in at least sections of its longitudinal direction or along its entire length. In those embodiments, the shaft fiber comprises a wall.

In certain embodiments, at least one of the tension threads (or all of them) is partly arranged within an inner space of the respective shaft fiber and extends from there to an outside of the shaft fiber through the shaft opening.

In some embodiments, at least one of the tension threads (or all of them) exits from an inner space of the shaft fiber through one shaft opening. In other embodiments, at least one of the tension threads (or all of them) exits from the inner space through two or more shaft openings.

In certain embodiments, the at least one shaft opening is provided at or on the front surface of the shaft fiber. In other embodiments, it is arranged at or on a circumferential surface or lateral surface area of the shaft. Preferably, the shaft opening is arranged in or within a tip area of the shaft fiber or in or within a proximal area of the shaft fiber.

In certain embodiments, the shaft fiber comprises a plurality of shaft openings uniformly or non-uniformly distributed or arranged along or about a periphery or along or about a circumferential surface or lateral surface area of the shaft or of the shaft fiber. Additionally or alternatively, the shaft openings may be dispersed along or about a longitudinal direction of the shaft or of the shaft fiber. For example, in some embodiments, shaft fibers may have two or more shaft openings which are arranged under different distances between the respective opening and the tip or one end of the corresponding shaft fiber.

In certain embodiments, at least one or all of the shaft fibers are arranged such that they do no move relatively to the folding device in a longitudinal direction of the folding device upon folding or unfolding the medical implant.

In some embodiments, tension threads for folding and/or unfolding the implant enter and/or exit through the shaft opening.

In certain embodiments, during the use of the folding device, shaft fibers of the plurality of individual shaft fibers are always present in bundled form in at least one first section of the shaft section. In contrast, in a second section, they are provided for moving or drifting away from each other during use of the folding device.

In some embodiments, the second section is closer to the tip of the folding device than the first section.

In certain embodiments, the first section directly merges with or passes over into the second section.

In some embodiments, the individual shaft fibers are arranged in contact to each other in the first section such that there is no lumen such as, e. g., a central lumen, for example a lumen usable during the use of the folding device for fulfilling particular functions, provided between the shaft fibers in the first section. Spoken differently, the shaft fibers are arranged closely or at close quarters.

In some embodiments, the term “individual shaft fibers”—when used discretely—comprises all shaft fibers present of the plurality of the entirely present individual shaft fibers; in other embodiments, it only comprises some of them.

In certain embodiments, the number of individual shaft fibers is set to two shaft fibers; in other embodiments, the number is set to three, four, five, six, seven etc. The number may be a great number; it may exceed ten or twenty and comprises every natural number up to at least 30 or 40.

In some embodiments, a great number of individual shaft fibers advantageously allows for separating the part or rim portion of the implant (for example, the periphery of the implant) which experiences an action by means of the tension threads for folding/unfolding, that exit from the individual shaft fibers into a great number of subunits. The inventors of the present invention have recognized that, for example, dividing the periphery into many but small or short sectors or rim portions upon attaching the tension threads at or on the implant, in certain embodiments, favors a uniform folding or unfolding the implant. Additionally, such dividing into a great number of sections may advantageously avoid any buckling or bulging or denting of the periphery.

In certain embodiments, a great number can be any numerical value between 3 and 40, for example, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40. Greater values are encompassed by the present invention as well.

In some embodiments, neither the individual shaft fibers nor sections thereof are arranged within the interior or material of a wall of an envelope, an outer boundary or limitation, or the like of the folding device.

In particular embodiments, neither the shaft nor sections thereof are arranged within the interior or material of a wall of an envelope, an outer boundary or limitation, or the like of the folding device.

In certain embodiments, the individual shaft fibers are provided such that they cannot be shifted or moved relative to the remainder of the folding device in a longitudinal direction thereof.

In some embodiments, the shaft or shaft fibers of the plurality of individual shaft fibers each comprise one or more shaft openings. The one or more tension threads can enter into and/or exit from the respective shaft fiber or from the shaft through the shaft openings.

In certain embodiments, such shaft openings are solely provided for allowing tension threads to enter in or into and/or leave or exit from the respective individual shaft fiber and/or from the shaft.

In some embodiments, the individual shaft fibers are designed or embodied to comprise one or more through-openings (extending into a longitudinal direction of the shaft fiber) or one or more hollow interiors. These through-openings or hollow interiors may allow guiding one or more tension threads through the shaft fiber, e. g. from the tensioning device of the folding device to a shaft opening or to an exit opening at the tip portion of the shaft fiber.

In some embodiments, the tension threads are arranged within an interior of the shaft fibers or of the shaft such that they can be shifted or moved relative to the respective shaft fibers.

In some embodiments, the shaft, the individual shaft fibers and/or the tension threads do not comprise any devices for establishing a hook engagement with the implant.

In some embodiments, some or all of the tension threads are connected with the implant by solely entangling or entwining the implant or a part or section or portion thereof.

In certain embodiments, during a state of use of the folding device, shaft fibers of the plurality of individual shaft fibers are arranged movably in or forth from (in the direction towards the tip of the implantation or folding device) at least the second section of the shaft respectively independently of each other and/or independently of the position of the implant relative to the folding device. Differently spoken, they can move away from each other and/or move towards each other in or within the second section.

In some embodiments, the shaft comprises in at least one section thereof a device for bundling individual shaft fibers of the plurality of individual shaft fibers.

In certain embodiments, there are provided more than just one device for bundling (but two, three, four, and so on, devices of this kind). Additionally or alternatively, in some embodiments, the device for bundling comprises not just one means, e. g. having the shape of a collar, but more than one means (e. g., two, three, four, and so on, means).

In particular embodiments comprising more than one device for bundling or means for bundling, the individual devices or means are provided on the shaft fibers while being spaced apart from each other. The particular space or distance chosen or set may advantageously contribute to setting or predetermining the stiffness, bendability and other mechanical features of the shaft fibers. This may be true for the shaft fibers' parts arranged between the devices or means for bundling. It may also be true for the parts of the shaft fibers that are not bundled but allowed to move freely with regard to each other.

In some of the embodiments comprising more than one device for bundling or means for bundling, a (that is, one or more) core element or a (that is, one or more) interconnecting element is provided on, at or within the bundle of shaft fibers. The core element or interconnecting element may also advantageously contribute to setting or predetermining the stiffness, bendability and other mechanical features of the shaft fibers. The core element or interconnecting element may be attached to one, two or all of the devices or means for bundling. However, it may not be attached as well. The core element or interconnecting element may be provided to be extendable and to change its length, for example, when a distance between neighbouring, adjacent or interacting devices for bundling or means for bundling is changed or adapted to need.

Both providing more than only one device for bundling or means for bundling and providing a core element or the like may in certain embodiments of the present invention allow for keeping the shaft fibers in parallel in use along a certain or even predetermined distance. Again, this may also advantageously contribute to setting or predetermining the stiffness, bendability and other mechanical features of the shaft fibers, both in the vicinity of core element or device or means for bundling and also in the second section of the shaft in which the shaft fibers are intended to move, wander or migrate freely in case of need.

In certain embodiments, the device for bundling is designed or embodied as a ring encompassing the individual shaft fibers to be bundled and inhibiting the shaft fibers from drifting or moving away from each other. In some embodiments, the device for bundling is designed or embodied as a clamp, a protrusion or a constriction of the folding device, or the like.

In certain embodiments, the device for bundling individual shaft fibers is arranged to be shiftable along a longitudinal extension of the folding device. Additionally or alternatively, the device for bundling may be alterable or manipulatable or engineerable in any other way. For example, the device for bundling may be manipulated by setting or altering a gap or play between shaft fibers and the device for bundling limiting or encircling the shaft fibers. Additionally or alternatively, the device for bundling can be provided for being used at or on different sections of the folding device along the longitudinal extension thereof. The afore-mentioned manipulations may advantageously alter or adapt to the need, respectively, the stiffness or rigidity of the individual shaft fibers in or within the second section.

In particular embodiments, a device for bundling such as specified above is not provided. In some embodiments, it is not possible to distinguish a first section from a second section (such as specified above and below) or required.

In some embodiments, individual shaft fibers are designed or embodied and provided or prepared for moving or bending or tilting, or the like, towards a rim portion of the implant when applying tension onto the implant by means of the tension thread extending through the said individual shaft fiber.

“Moving towards” is in some embodiments to be understood as a deviation of at least one section of the individual shaft fiber (mainly in the second section or in a tip area of the individual shaft fiber) from a position that is arranged closer to a center of a cross section of the folding device into a position that is arranged more radially as compared to the first position, e. g. into a rim area or towards a rim portion.

In certain embodiments the bundle of shaft fibers can be (or are) arranged in a circular manner.

In some embodiments, the shaft fibers (e. g. nine shaft fibers in total) are arranged in a circular manner in both the unfolded and/or the folded state of the medical implant.

In certain embodiments, a rim portion is a section, in particular a section of a circumference of the implant or a main part thereof, that is present in an area of an—in relation to the implant—exterior wall or envelope, for example the exterior wall or envelope.

In some embodiments, the rim portion comprises a part of the foldable material of the implant. The rim portion may be a curve-shaped part of an outer limitation or of a wall (e. g. a mesh, grid, strut or bar structure) of the implant. The rim portion may be a tart-like structure.

In certain embodiments, tension threads exiting from individual shaft fibers are connected with a rim portion of the implant for applying a force onto the implant and/or onto the rim portion. In some embodiments, tension threads exiting from at least two or more individual shaft fibers are connected with the said rim portion, or parts thereof.

In some embodiments, the rim portion which is folded or unfolded by means of a particular shaft fiber or by means of the one or more tension threads of the shaft fiber, respectively, is only a part of the foldable and/or unfoldable periphery of the implant or is the entire foldable and/or unfoldable periphery of the implant.

In certain embodiments, the rim portion does not comprise the entire periphery, whereas, in other embodiments, it indeed does.

In some embodiments, tension threads exiting from individual shaft fibers are connected with a rim portion in an overlapping manner for applying a force onto the rim portion of the implant. In those embodiments, it is intended to fold or unfold a particular rim portion or a part thereof by means of two, three or more tension threads exiting from different individual shaft fibers. In this way, an overlap of several tension threads is achieved in an area of a particular rim portion. This may advantageously contribute to a more uniform folding of the implant.

In certain embodiments, tension threads exiting from individual shaft fibers are connected with differently large, broad, long or in any other way different rim portions of the implant for applying a force on the said rim portions. Thus, a first rim portion may have a first arc or curve length x, a second rim portion may have a second arc or curve length 2x. This may advantageously allow for or contribute to a more uniform folding of the implant even in cases in which the implant does not behave in a mechanically uniform way over its entire periphery upon folding.

In certain embodiments, individual shaft fibers on the one hand and the implant on the other hand are adapted, chosen or fit to each other as regards their mechanical properties. In certain embodiments, this may be effected such that, during the process of folding the implant, a first force or tension required for moving the shaft fibers in a section thereof, in particular in an area of a shaft opening for tension threads, from their longitudinal alignment or in a direction of the radial extension of the implant is lower than a second force or tension. The second force or tension is a force or tension required for effecting a folding or the beginning or an appreciable beginning of a folding of the implant by means of the tension threads connected with the implant that exit from the shaft openings. Said in a more simple manner, in some of those embodiments, upon application of tension, the individual shaft fiber firstly moves in a—for example, radial—direction towards a rim portion of the implant upon applying tension by means of the tension threads exiting from the shaft fiber, prior to beginning any folding of the respective rim portion. This adaptation or adjustment of properties of the individual shaft fibers (such as bendability, flexibility, elasticity, or rigidity) with respect to the properties of the implant may ensure that, upon applying tension by means of the tensioning device, the individual shaft fibers firstly automatically move into a position in which the force applied or to be applied by means of the tension thread is applied onto the implant or onto the rim portion in or under a desired angle.

In some embodiments, the implant is connected or intended to be interconnected with the folding device by means of tension threads such that the tension threads (independently of each other or in an overlapping manner) interconnect with a great number of peripheral sections of the implant. A great number may be any numerical value between 3 and 40, for example, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40. Greater values are encompassed by the present invention as well.

In some embodiments of the set, the implant is connected or intended to be interconnected with the folding device by means of tension threads such that the tension threads may act and/or contact the medical implant not only at one end of the medical implant but at least at two or more sections of the medical implant which are longitudinally offset from each other.

In certain embodiments of the methods, the methods may comprise altering a tension applied onto the implant by means of at least one tension thread. The tension is controlled by means of altering a length of the one or more tension threads exiting from the interior of the shaft or of the shaft fiber.

In some embodiments of the method, the method comprises shifting or otherwise manipulating or engineering the device for bundling individual shaft fibers. In this way, the rigidity, stiffness, elasticity, or the like, of the respective individual shaft fibers may be set according to need—in particular with regard to the second section.

In some embodiments of the methods according to the present invention, prior to its implantation, the implant is connected with the folding device and/or implantation device by means of tension threads such that the tension threads (independently of each other or in an overlapping manner) interconnect with a great number of peripheral sections of the implant.

Some or all of the following advantages and the advantages mentioned above can be achieved in some, certain or all embodiments.

In some embodiments, one advantage achievable is to advantageously reduce or even completely avoid buckling or bulging or denting of the implant resulting from applying a force onto the implant—mainly onto the periphery thereof—by means of the tension threads.

Another advantage of certain embodiments is a uniform folding of the implant even in a case in which an implant is designed inhomogeneously as regards the mechanical properties of the implant along the periphery thereof.

According to yet another advantage of some embodiments—due to the capacity of the individual shaft fibers to move, migrate or wander—the tension threads' exit from exit openings of the shaft fibers may be effected in or under angles at which the tension threads will not suffer particular friction or shear stress at the exit or shaft openings. In connection therewith, a further advantage could be that the force for folding—again due to the possibility of the individual shaft fibers to move, migrate or wander—is acting on the implant under particularly advantageous angles.

In some embodiments, the folding device further includes at least one tensioning device for altering or amending a shape of the foldable and/or unfoldable implant by the tension thread or by at least one first string connected to the tension threads.

In certain embodiments, the folding device includes a clamping mechanism, a clamping device or a clamping section (hereinafter: clamping mechanism) for clamping at least one section (for example a free end) of at least one of the tension threads.

In some embodiments, the medical implant comprises a set of tension threads for folding or unfolding the medical implant or is connected or provided herewith, the set of tension threads being designed as set forth herein.

In certain embodiments, the set of tension threads comprises at least one first string, at least a first tension thread and at least a second tension thread.

In some embodiments, the first string comprises at least a first guiding element for guiding through the first tension thread and a second guiding element for guiding through the second tension thread.

In certain embodiments, both the first tension thread and the second tension thread are attached to the first string.

In some embodiments, the methods according to the present invention may comprise providing a folding device or a set of tension threads. They may also comprise clamping at least one section of at least one tension thread by a clamping mechanism.

In particular embodiments, the diameter of the implant is arranged in the reception area in a plane perpendicular to a main flow direction of the implant in case fluids flow through the implant after its implantation.

According to the present invention, the term thread or tension thread may also define a plurality of threads or tension threads whenever a person skilled in the art recognizes the exchangeability of the terms.

In some embodiments, the pulling device and the tension thread are intricate with each other.

In certain embodiments of the present invention, the transfer (or the transmittal, respectively) of force or tension between the pulling device and the tension thread is achieved by a non-form closure connection.

In certain embodiments, the pulling device is embodied as at least one pulling thread or wire or consists of at least one pulling thread or wire.

In certain embodiments, “clamp” or “clamping” means to fasten the section of the thread within or by the clamping mechanism. It may be understood as to fasten the thread to another part of the folding device such as the first or second clamping section. The term may be understood as to maintain the section of the thread within or by the clamping mechanism in a manner such that the clamped section of the thread cannot be withdrawn from the clamping mechanism by the tension device under normal use conditions unless the releasing device has been operated. The term may be understood so as press two parts towards each other strongly enough so as to keep an object (here: the section of the thread) arranged between the two parts in place. The term may be also understood as to press the section of the thread against one first clamping section by a second clamping section, wherein at least one of the clamping sections is not movable with regards to the shaft or the entire folding device.

In some embodiments, the term “clamping mechanism” relates to a part of the folding device or a set of parts thereof that work together in order to clamp the section of the thread in a releasable manner. The releasing device may be arranged to be operated by the surgeon.

In certain embodiments, the first and the second clamping section may together be understood as a clamp for the thread when clamping the section of the thread between them.

In some embodiments, the clamping mechanism consists of the first and the second clamping section. In certain embodiments, the clamping mechanism comprises the first and the second clamping section.

In certain embodiments the first clamping section and the second clamping section of the folding device are arranged such that they are inclined to the longitudinal axis of the folding device, its shaft, and/or the reception or retaining area for receiving the implant.

In some embodiments, the “releasing device” for releasing the clamp, the clamping mechanism or the clamping of the section of the thread is to be understood as a device for removing restrictions acting on the section of the thread, for removing the section of the thread from at least one of the first or second clamping section, or vice versa, for removing at least one of the first or second clamping section from a fixed position, allowing the section of the thread to move or to be withdrawn, e. g. by the pulling device or by the tensioning device.

In certain embodiments, the clamping mechanism does not form part of the tensioning device.

In some embodiments, a first end of the thread is clamped by the clamping mechanism, whereas another part, in particular a second end opposite to the first end of the thread is interconnected with the tensioning device.

In certain embodiments, the folding device comprises a releasing device for releasing the clamped section of the tension thread from the implant by releasing the clamping mechanism.

In some embodiments of the folding device the clamping mechanism is adapted for clamping the at least one section of at least one of the tension threads between a first clamping section and a second clamping section of the folding device. Alternatively, the clamping mechanism consists of such first and second clamping sections.

In certain embodiments, the first clamping section and the second clamping section of the folding device are arranged such that they may slide relatively to each other (for example, mainly or exclusively in a direction parallel to the main extent or the longitudinal direction of the folding device).

In some embodiments, the second clamping section of the folding device is arranged in an inner space of the first clamping section of the folding device.

In certain embodiments, the shaft includes at least one shaft aperture through which tension threads for folding and/or unfolding the implant may enter and/or exit the inner lumen of the shaft.

In particular embodiments, neither the first nor the second clamping section is arranged on or at the implant.

In some embodiments, the implant is a stent or a cardiac valve assembly.

In particular embodiments, the folding device comprises at least one implant connected with tension threads or with a set of tension threads for folding or unfolding at least one medical implant. The combination of an folding device and at least one implant connected thereto by tension threads or a set of tension threads for folding or unfolding the medical implant may also be called a “set” (albeit different from the set of tension threads, of course).

In some embodiments, the set of tension threads comprises at least one first string, at least a first tension thread and at least a second tension thread. The first string comprises at least a first guiding element for guiding through the first tension thread and a second guiding element for guiding through the second tension thread. Both the first tension thread and the second tension thread are attached with the first string.

In certain embodiments both the first tension thread and the second tension thread are attached with the first string such that they are fixed to the first string and/or such that the ends or portions of the threads by which they are fixed to the first string cannot move relatively to the first or second guiding element. For example, the first tension thread and the second tension thread may both be knotted to or integral with the first string. In embodiments with more than two tension threads, more than two tension threads are fixed to the first string.

In some embodiments, at least one of the first guiding element and the second guiding element are rings. In certain embodiments, the first tension thread and the second tension thread may slide through the first guiding element and the second guiding element forth and back.

In certain embodiments the first string is connected to a multitude of tension threads, for example to—at least or exactly—six tension threads of which three are guided through the first guiding element, wherein three of them are guided through the second guiding element.

In certain embodiments, neither of the first tension thread nor the second tension thread is directly connected to a tensioning device of an implantation device for altering a shape of the foldable and/or unfoldable implant. Rather, they are in direct contact with the first string. It is via the first string that they are in indirect contact with the tensioning device.

In certain embodiments at least the first tension thread is connected to the first string, preferably at a first end section of the first string. Likewise, the second tension thread is connected to the first string, preferably at a second end section of the first string.

In some embodiments the first end section and the second end section are opposed ends of the first string.

In certain embodiments the first tension thread is connected with or fixed at its first end to the first string, and wherein at least the second tension thread is connected with or fixed at its first end to the first string.

In certain embodiments, the methods may encompass releasing the clamped section of the tension thread from the implant by releasing the clamping mechanism. In some of these embodiments the tension thread may then be withdrawn from the implant, e. g. by the tensioning device or the surgeon.

In certain embodiments, the releasing device is not a wire or a thread (or a multitude thereof, respectively).

In some embodiments, the releasing device is not embodied as a lock wire or lock thread that is withdrawn from the folding device so as to allow the tension threads to be removed from the folding device after final placement of the implant.

In certain embodiments, the releasing device does not comprise hooks and/or does not comprise rings for guiding or limiting tension strings or threads.

In some embodiments, the releasing device is embodied and/or intended to stay with the folding device even after termination of the implantation of the implant.

In certain embodiments, the releasing device may not be separated from the folding device except for cleaning or the like.

In some embodiments, the releasing device is embodied such that it is intended or configured to be used, only within the patient's body.

In some embodiments, at least one of the first or the second clamping device is or includes at least one sleeve. The sleeve may preferably be made tube-like (that is, it may have a hollow inside), like a hollow cylinder, like a ring or the like. The sleeve may be manufactured symmetrically or asymmetrically, both relative to its opening direction and in another direction, particularly in a direction or plane perpendicular to the opening direction or the fluid passage direction, as well.

In some embodiments of the folding device, the releasing device is no cutting device for cutting through the tension thread, and it does not comprise one.

In certain embodiments, tension threads for folding and/or unfolding the implant may enter and/or leave the folding device through the shaft aperture.

In particular embodiments, the releasing device is identical to the clamping mechanisms or comprises part of it. In these embodiments, there is one device that may both clamp and subsequently also release the thread when being operated in a “reverse manner”.

In certain embodiments, the first or the second clamping section is preferably arranged in a shiftable manner.

In some embodiments, the first clamping section surrounds or embraces the shaft in such a way that the shaft is located inside the first clamping section and the first clamping section. Alternatively, the first clamping section may be located inside of the shaft such that the shaft is surrounding the sleeve.

In certain embodiments, the folding device includes a releasing device which is arranged for exerting tension on the first or second clamping device in at least one state of use.

In some embodiments, the releasing device exerts a tension on the first or second clamping device substantially or exclusively in a longitudinal direction of the shaft

In certain embodiments, the releasing device is arranged for pushing or for pulling the first or second clamping device in order to release the clamped section of the thread. It may be embodied as a pulling device as, e.g., a thread or the like. In other embodiments, said device is embodied as a pushing or twisting device.

In some embodiments, the releasing device enables a transition from the clamping position into the release or non-clamping position.

In certain embodiments; said releasing device is arranged such that it enables a transition from the clamping position into the release or non-clamping position independently from an operation of the tensioning device.

In certain embodiments, the methods may comprise altering the tension that is exerted on an implant by using at least one tension thread. The tension is preferably controlled by altering a length of the pulling device by which it extends out of the interior of the shaft or sections thereof.

In some embodiments, at least one of the folding device, the implant and the set of tension threads comprises exclusively, i.e. only, (one or more) materials that are MRI (short for: magnetic resonance imaging) compatible. In certain embodiments, at least one of the folding device, the implant and the set of tension threads comprises exclusively (one or more) materials that are not magnetic, ferromagnetic, or both. In some embodiments, at least one of the folding device, the implant and the set of tension threads does not comprise metal or any metal alloy.

In particular embodiments, the methods may comprise monitoring or controlling the position of the folding device or the implant, or both, by means of magnetic resonance imaging (MRI) during or after implementation, advancing or delivering of the implant.

In certain embodiments, all instruments used for implanting or advancing the implant are MRI compatible.

In some embodiments, the folding device comprises a detachable tip. In certain embodiments, the tip (and only the tip) comprises the first and the second clamping section.

In particular embodiments, the first clamping section is connected to a first connecting device of the tip or the shaft of the folding device by a thread such that the first clamping section can be moved along the shaft of the tip by rotating the first connecting device whereas the first clamping section itself is not rotated.

In some embodiments, the tip of the folding device or implantation device comprises a rotational clamping mechanism such that the first clamping section can be moved to or away from the second clamping section by rotating the first connecting device whereas the first clamping section itself is not rotated.

In particular embodiments, the first connecting device may have an crown-shaped section, it may comprise a gear pattern, it may have teeth or any other engagement device, due to space constraints preferably at its front surface (not on its sided surface), configured to be engageable with a second, rotably arranged connecting device of the apparatus in a manner such that via rotating the second connecting device the first connecting device may be rotated.

In certain embodiments, the clamping surface of at least one of the first and second clamping sections is inclined against a longitudinal axis of the folding device or the tip thereof by between 10 and 30 degree, preferably between 10 and 20 degree, most preferably about 15 degree.

In some embodiments, the entire rotational clamping mechanism, including or consisting of at least the first and the second clamping section and the first connecting device, is provided on or at the side of the tip.

In certain embodiments, the first clamping section has a clamping surface, is movably arranged, comprises a thread (preferably arranged on an inner surface thereof), has a slot or groove (which preferably is straight) into which a protrusion such as a pin is inserted, whereas the protrusion is fixed to the tip, but cannot be rotated.

In some embodiments, the the first clamping section has a clamping surface, is movably arranged, comprises a thread (preferably arranged on an outer surface thereof), has a slot or groove (which preferably is straight) into which a protrusion such as a pin is inserted, whereas the protrusion is fixed to the tip, but cannot be rotated.

What is said in here with regard to one tension thread holds also true for a multitude of tension threads whenever this does stand in contrast to the general idea of the present invention.

Further, clamping the threads used for folding or unfolding the implant allows for a safe temporary connection between the folding device with the threads. At the same time, since the clamping mechanism may be released after implantation, in some embodiments the present invention allows for an easy removal of the threads from the implant just by releasing or terminating the clamping effect and by pulling the thread out of the folding device. In particular, for removing the threads from the implant or from the folding device, no thread has to be untied, unknot, undone or even cut. Hence, at least one end of the tensioning threads does not have to be interconnected to or released from e. g. the implant. This does not only safe time and effort but also contributes to a safe handling of the implant upon implantation. Also, providing a reliable clamping mechanism is less demanding regards observing tolerances in the production process of the folding device.

In certain embodiments, providing a groove, a sliding block guiding, a slotted guide, or the like that avoids twisting or rotation of the first clamping device with regard to the second device or vice versa contributes to releasing the clamped section of the thread once the releasing device has been operated. In contrast, twisting or rotating might incur an accidental clamping of the thread between part of the apparatus other than the first and second clamping sections.

Providing in some embodiments at least one of the folding device, the implant and the set of tension threads to be MRI compatible allows advantageously for controlling the location and orientation of the folding device or the implant, or both, by MRI upon use of the folding device or implantation of the implant. No heat, sparks or artefacts are generated during MRI because of the materials chosen for the folding device or the implant.

Among the advantages achievable is the opportunity in certain embodiments of independently actuating tension threads. This is due to the self balancing design provided in which the pulling thread is entangled with the first string or wound around without being fixed to it. This way, the first string may move forth and back within the loop of the pulling thread. Thus, it is possible to specifically fold and/or unfold implants having a first and a second tension thread, or a plurality of tension threads, which act on different parts or sections of the implant upon folding the latter. Parts or sections of the implant can thus be folded or unfolded though other parts or sections of the implant have already been completely folded or unfolded. This can i.a. be reasonable when an unfolded implant and the implantation site do not completely match in their dimensions, or if the implant does, e.g., not have a uniform shape over its entire length. For more details regarding the idea of an self-balancing design it is referred to WO 2011063972 A8. See, in particular, FIGS. 2 and 2A thereof. The entire respective content of WO 2011063972 A8 is incorporated herewith by reference.

In some embodiments, some or all of the individual shaft fibers have each at least one shaft opening for passing through the tension threads.

In certain embodiments, the implant comprises apertures through which tension threads are guided or passed.

In particular embodiments, tension threads which pass through (i.e., enter into or exit from) one particular (or many) of the shaft openings of a particular individual shaft fiber also pass together through (i.e., enter into or exit from) a common aperture of the implant.

In certain embodiments, unfolding means the opposite of folding.

In some embodiments, a common aperture is to be understood as a single aperture through which more than one tension thread are commonly guided or passed through. However, in other embodiments according to the invention, a common aperture means a particular site where a pair of tension threads are guided through the circumference of the implant. That common aperture may be subdivided into two or more sub-apertures which, however, all belong to a functionally ‘common’ passage through the circumference of the implant. Two or more sub-apertures may even then be referred to as a common aperture within the sense of the present invention, as long as an angle formed between the pair of tension threads is not substantially changed by the fact the common aperture is comprised by not by one but by a multitude of apertures.

In certain embodiments, the term ‘one (particular) common aperture’ encompasses all apertures that are arranged on one (particular) post or strut at one and the same spot or in either a proximal or distal section thereof.

In some embodiments, one or each of the common apertures are provided in one or more posts or struts of the implant which extends in parallel to a longitudinal axis of the implant (in an unfolded or folded state outside of the body).

In certain embodiments, all of the common apertures are provided only in such posts or struts of the implant.

In some embodiments, the posts or struts are arranged to connect a distal ring-shaped element of the implant with a proximal ring-shaped element thereof. The posts or struts may be arranged to maintain a distance or an unchangeable distance between the distal and the proximal ring-shaped elements.

In certain embodiments, the at least one opening of the shaft fiber is an opening in the end face of the shaft fiber.

In some embodiments, the at least one opening of the shaft fiber is an opening in the shaft or circumference or envelope of the shaft fiber.

In certain embodiments of the set, the shaft openings of the shaft fibers and the apertures of the implant are matched such that tension threads which pass through one or more shaft opening(s) and which also pass together through a common aperture do not—or not substantially—change the angle between them during folding and/or unfolding.

In some embodiments, the implant, shaft fibers and pairs of tension threads are arranged such that in an area of the shaft openings the respective tension threads belonging to one pair of tension threads do not diverge upon folding and/or unfolding the implant.

In some embodiments according to the invention, some or all of the tension threads, i. e. at least one tension thread, do not encompass the whole circumference of the implant. In certain embodiments according to the invention, one or more of the tension threads re-enter the lumen of the implant through apertures provided within the circumference or rim of the implant that are, for example, adjacent to the aperture through which the respective tension thread has exited from the lumen. In some embodiments according to the invention, some or all of the tension threads are provided to re-enter the lumen by an aperture provided in the rim that is different from the aperture through which the particular tension thread has exited from the lumen to an outside of the implant. In particular, any tension thread may re-enter the lumen by the next aperture, the next but one, next but two, next plus three, or the like, on the circumference or on the rim.

Please note that in any embodiment according to the present invention the number of apertures may always be identical to the number of fibers. However, the number of apertures may as well differ from the number of fibers.

According to yet another advantage of some embodiments, the tension threads which pass through one or more shaft opening(s) of a particular individual shaft fiber and which also pass together through a common aperture of the implant are guided more or less in parallel between shaft opening and aperture. Further, even upon folding or unfolding of the implant, the pair of tension threads do remain parallel (or the angle between does not change or not substantially change). This way, in particular weaker stents or implants (weak being related to the force required for folding or crimping the implant) may be folded or crimped without buckling, even if only a small number of apertures is provided. Otherwise, upon folding or crimping, the implant may just buckle. With the present invention, single sections of the circumference or rim portions are compressed by forces applied onto the rim portions between the respective apertures. Hence, the force applied acts advantageously also in a circumferential direction.

In some embodiments, at least one of the implant delivery device and the implant comprises a releasing string knotted to tension threads by at least one knot.

In certain embodiments, the knot is configured such that it can be released or opened upon pulling the releasing string.

The methods according to the present invention may comprise in some embodiments releasing the tension thread by pulling the releasing string in order to unknot, untie or untangle the knot.

In some embodiments, at least one of the implant delivery device and the implant further comprises a pulling string also knotted to tension threads by the knot.

In certain embodiments, the implant delivery device comprises a releasing mechanism allowing a user to pull, or selectively pull, the releasing string, in particularly in order to disengage, interrupt or cease the force transmission between the tension threads and the implant such that the implant cannot be folded any longer by means of the delivery device.

In some embodiments, the implant delivery device further comprises a mechanical reversing device arranged to reverse or amend the direction in which the releasing string extends.

In certain embodiments, the implant delivery device further comprises a balance limiter.

In some embodiments, the knot comprises at least, or exactly, two loops, wherein the first loop is inserted into the second loop and can be pulled out of the second loop upon or by pulling the pulling string.

In certain embodiments, the knot is embodied or knotted as shown in FIG. 7.

In certain embodiments, the set of tension threads comprises at least or exactly three tension threads each of which is preferably wound around at least parts of the implants periphery on at least two heights or longitudinal levels of the implant.

In some embodiments, the implant comprises a first guiding element and a second guiding element. The tension threads are each guided through or along at least sections of both the first and the second guiding elements.

In certain embodiments, at least one of the first guiding element and the second guiding element are hollow rings or channels that are preferably partly open in a cross section thereof.

In certain embodiments of the implantation device, the tensioning device includes at least one pulling string (or thread or wire or the like). The pulling string is arranged and/or provided in such a way that it may indirectly or directly apply a tension on the implant for altering the shape of the implant by the tension thread if the pulling string is pulled or activated by an operator (e. g., by the surgeon).

In certain embodiments of the implantation device, the pulling device is a pulling string.

In certain embodiments, the pulling string is arranged and/or provided such that it may interact with the tension thread in order to transfer force or tension.

In some embodiments of the implantation device, the pulling string and the tension thread are intricate with each other at at least one site.

In some embodiments, the term “releasing mechanism or device” relates to a part of the implantation device for releasing the tension tread(s) from the implant, or the tension applied by the tension threads onto the implant, by unknotting the knot. The releasing device may be arranged to be operated by the surgeon.

In certain embodiments a first string is connected to a multitude of tension threads, for example to—at least or exactly—three or six tension threads.

In some embodiments, none of the tension threads is directly connected to the tensioning device of the implant delivery device, which may be a catheter, for altering a shape of the foldable and/or unfoldable implant. Rather, they are in direct contact with the first string. It is via the first string that they are in indirect contact with the tensioning device.

In certain embodiments, the methods encompasse releasing the knot by pulling the pulling string. In some of these embodiments the tension thread may then be withdrawn from the implant, e. g. by the tensioning device or the surgeon himself.

In certain embodiments, the releasing device is a wire or a thread (or a multitude thereof, respectively).

In certain embodiments, the releasing or separating device may be separated from the implantation device.

In some embodiments of the implantation device, the releasing device is not a cutting device for cutting through the tension thread, and it does not comprise one.

In certain embodiments, the releasing device exerts a tension on the knot via the releasing string substantially or exclusively in a longitudinal direction of the shaft.

In some embodiments, the releasing device enables a transition from the knotted state into the release or unknotted state.

In certain embodiments of the implantation device, said releasing device is arranged such that it enables a transition from the knotted state into the release or non-knotted sate independently from an operation of the tensioning device.

In certain embodiments, the set of tension threads comprises a self-balancing design. For more details regarding the idea of a self-balancing design it is referred to WO 2011063972 A8. See, in particular, FIGS. 2 and 2A thereof. The entire respective content of WO 2011063972 A8 is incorporated herewith by reference.

In some embodiments, the shaft of the implant delivery device is arranged within the center of the implant and/or of the valve at all times.

In certain embodiments, the implant is evenly folded or unfolded by the implant delivery device along its entire length (or longitudinal direction or extension).

Among the advantages achievable with some embodiments is the opportunity of releasing threads from the implant by the releasing string that is arranged for ceasing the stated in which the thread(s) are knotted.

Further, in certain embodiments, the knot allows for a safe but temporary connection between the implantation device, and/or the implant, with the tension threads. At the same time, since the knot may be released after implantation, in some embodiments the present invention allows for an easy removal of the threads from the implant just by opening the knot and by pulling the thread out of the implantation device. In particular, for removing the threads from the implant or from the implantation device, no thread has to be unclamped or even cut. This contributes to a safe handling of the implant upon implantation since every device, such as a cutter, might fail, whereas in the absence of devices such a cutter with the present invention there are less devices that might fail. Also, providing a reliable mechanism such as a cutter always requires observing tolerances in the production process of the implantation device. In contrast, with the solution, less tolerances are to be observed since the knot is not a technical device which function relates on technical tolerances.

Further, in some embodiments, the knot allows for a very easy release of the tension threads.

In certain embodiments, in contrast to cutting, for example, knotting does not require the use of tension threads that provide sufficient resistance to the cutter blade. Rather, for knotting any tension thread will do, in particular also the very flexible ones which provide additional advantages by themselves. Among those advantages, a flexible tension thread may be withdrawn from the implant once it has been release without unintentionally crimping or folding the implant again (because of the resistance between the tension thread and the implant) that can be observed when a stiff(er) tension thread is withdrawn from the implant.

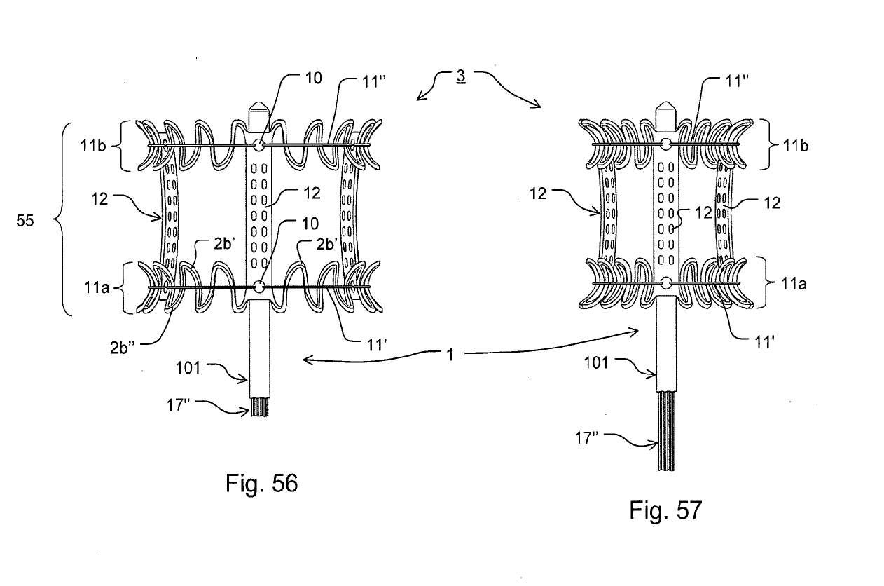

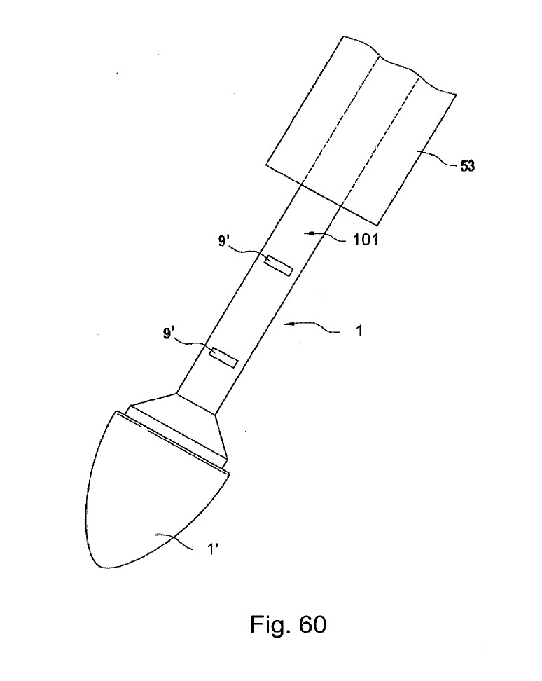

In some embodiments, the knot suggested in FIG. 7 is designed in a way such that the tension thread's ends are bent at a minimum. This has the advantage that the ends of the tension threads are crinkled as little as possible so that they can be easily withdrawn through the apertures of both the implant and the delivery device, see, e. g. FIGS. 56 and 60.

Also, the solution according to certain embodiments which is based on a knot allows using implants comprising tensions threads connected by a knot with all kinds of delivery devices. In particular, the features of the tension threads that come along with the implant do not have to match with a cutter or a clamping device already arranged on the delivery device since the cutter or the clamping device will simply not be used.

If, as contemplated in particular embodiments, one particular tension thread is wound around several sections of the implant, whereas the sections are at different longitudinal heights of the implant, and if both ends of that tension thread are tightened in the knot, then the crimping of the implant may be carried out in a very smooth and easy manner since the tension threads are pulled from both ends. At the same time, the crimping force is applied to the implant in a very homogeneous manner resulting in a very homogeneous folding.

Also, in some embodiments, using a knot instead of a clamping device, a cutter or the like advantageously allows to build a delivery device which is smaller (e. g., in diameter) than prior art devices comprises such a releasing device (i. e. cutter, clamp, and the like).

Furthermore, in certain embodiments, using a knot having two loops as disclosed supra and infra means very little slippage which makes the knot a particularly safe connection.

In certain embodiments, the knot is configured such that it can not be released or opened upon pulling the pulling string, for example at least or solely by pulling in a particular, determined direction.

In some exemplary embodiments according to the present invention, the knot may be tightened and/or maintained knotted by pulling the pulling string and/or applying a tension thereof.

In certain exemplary embodiments according to the present invention, the pulling string and the releasing string are distinct parts of the same string, for example two strings arranged or connected to each other, or constitute the same string.

In some exemplary embodiments, the knot is formed by a string being both a releasing and a pulling string, the one end of which may be pulled for folding the implant and the other end of which may be pulled to release the knot and/or in particular to unfold the implant.

In some embodiments according to the present invention, the releasing string is knotted to the tension threads without additional device or element, for example without strings and/or wire and/or posts, in particular without one or more elongated and/or rigid element, for retaining the knot.

In certain embodiments according to the present invention, the knot may be released from the at least one tension thread without cutting the pulling and/or releasing string and/or without sliding at least one open end of the string through one sling or loop of a tension thread.

In some embodiments, the catheter or implant delivery device for folding or unfolding at least one medical implant by means of at least one tension thread comprises at least a handle assembly.

In certain embodiments, the handle assembly comprises a drum for winding the tension thread thereon by rotating the drum.

In some embodiments, the handle assembly further comprises a knob to be rotated—or being arranged to be rotatable relative to another section of the handle assembly—by a user of the handle assembly in order to fold or unfold the medical implant by tightening or winding the tension thread or by releasing or unwinding the tension thread.

In certain embodiments, the knob is arranged with or interconnected with the drum such that the drum is rotated when the knob is rotated.

In some embodiments, the handle assembly further comprises a displacement limiter for limiting the length or displacement by which the tension thread may be at least one of wound onto or unwound from the drum by rotating the knob.

In certain embodiments, the handle assembly further comprises a force limiter for limiting the maximum force or tension that may be applied or is applicable to the tension thread or to the drum by rotating the knob.

In some embodiments, the handle assembly further comprises a brake frame assembly comprising at least one brake element and at least one spring arranged to act on the brake element such that the brake elements contact one surface of the rear knob.

In certain embodiments, the handle assembly's knob comprises a gear pattern or teeth, e. g. on an inner rim of the knob. In these embodiments, the force limiter comprises or consists of a first ring element, e. g. a rush gear, comprising a gear pattern or teeth matching or corresponding to the gear pattern or teeth of the knob. Further, the force limiter comprises at least one spring element arranged for pressing the first ring element against the knob in a manner such that when a user rotates the knob, in an assembled state of the handle assembly the first ring element is also rotated because of the interaction between the gear pattern or teeth of the knob on the one side and the gear pattern or teeth of the first ring element on the other side.

In some embodiments, the handle assembly's first ring element comprises teeth on an inner surface thereof.

In certain embodiments, the force limiter further comprises a second ring element, e. g. a clutch stopper or a drive wheel. In these embodiments, the spring element is interposed between the first ring element and the second ring element and has contact to both of them.

In some embodiments, one of the first and the second ring element comprises protrusions, and the other one comprises or receptions arranged for receiving the protrusions so as to establish at least one of a form fit and a force disclosure between the first and the second ring elements.

In certain embodiments, some or all of the receptions and the protrusions, respectively, are each arranged at on lower surface of the first ring element and on the upper surface of the second ring element.

In some embodiments, the force limiter further comprises an internal retaining ring attached within the inner lumen of the knob.

In certain embodiments, the drum and the knob are interconnected by a rush gear. The rush gear is arranged to rotated inside—and preferably in engagement with—a gear stopper. The gear stopper, which is preferably arrange inside the rear knob but preferably not fixed or interconnected to the rear knob resulting in that the gear stopper and the rear knob can rotate independently from each other, is moved along or by means of a gear pattern or teeth when being rotated. The rush gear is interconnected to the drum or to part of it such that a rotation of the rush gear results in a rotation of the drum. The displacement limiter comprises or consists of a ring-shaped or tube-shaped element—e.g. a gear stopper—having an inner surface. The inner surface comprises at least one of a second section having an inner surface different to the inner surface of the first section or a radial width (being the distance between the outer surface of the section and the inner surface thereof) smaller than that of the first section, and a third section having an inner surface different to the inner surface of the first section or a radial width smaller than that of the first section.

In some embodiments, at least one of the second section and the third section does not comprising teeth or a gear pattern.

In certain embodiments, at least one of the second section and the third section is arranged in contact with the first section. Alternatively, at least one of them is arranged adjacent to the first section.

In some embodiments, the ring-shaped or tube-shaped element also comprises at least a fourth section on its inner surface. Preferably, the fourth section does not comprise teeth. The fourth section preferably does not comprise teeth or a gear pattern. The fourth section is separated or delimited from at least one of the first or second section by an inclination, an edge, a stop or a protrusion configured to prevent the rush gear to be rotated further towards to or onto the fourth section.

In certain some embodiments, the inner surface of at least one of the second section and the third section has a radial distance to the center of the ring-shaped or tube-shaped element that is larger than a radial distance between the tips of one, some, or the majority of the teeth and the center of the ring-shaped element.

In some embodiments, the ring-shaped or tube-shaped element comprises at least a first protrusion arranged to interfere with a second protrusion of the casing assembly or any other element of the handle assembly so as to limit the rotation of the rush gear within the ring-shaped or tube-shaped element.

In certain embodiments, the at least one first protrusion is arranged so as to protrude into an inner lumen of the ring-shaped or tube-shaped element.

In some embodiments, the at least one first protrusion is arranged between the second and the third sections or at the fourth section or opposite the first section of the ring-shaped or tube-shaped element or has its base at one of the aforementioned sites.

In certain embodiments, the second section has at least one of a length, width (in a radial direction) and inclination such that the rush gear may be positioned inside the second section such that at least one tooth of the rush gear is engaged with at least one tooth of the teeth of the first section while at least one tooth of the rush gear is at the same time in contact with the inclination, the edge, stop or protrusion delimiting the second section from the fourth section or arranged within the second section.

In some embodiments, the third section has at least one of a length, width (in a radial direction) and inclination such that the rush gear may be positioned inside it such that one tooth of the rush gear is only half-engaged with at least one tooth of the teeth of the first section while other teeth of the rush gear are at the same time in contact with the inclination, the edge, stop or protrusion delimiting the third section from the fourth section or arranged within the second section. “Half-engaged” within the meaning of the present embodiments may be such that rotating the drum in one direction may not result into rotating the gear stopper as well, whereas rotating the drum in the opposite direction would necessarily result in an functional engagement of the teeth of the drum with the teeth of the first section of the gear stopper again. “Half-engaged” may be understood such that one tooth of the drum and the last tooth of the first section 1911 contact each other temporarily but slip over each over if the rush gear 16 is rotated in a first direction whereas the rush gear 16 and the gear stopper 19 get into normal teeth engagement if the rush gear 16 is rotated in the counter-direction.

In certain embodiments, the drum is simultaneously engaged (or at least half-engaged as defined above) both to the rush gear and the gear stopper, or is in contact with both, preferably always.

In some embodiments, the brake frame assembly is configured and arranged to brake a rotation of the rear knob or to prevent the unintended rotation thereof.

In certain embodiments, the at least one brake element is arranged on a frame of the brake frame assembly such that the brake element may pivot with respect to the frame.

In some embodiments, the brake frame assembly is arranged inside the rear knob.

In certain embodiments, the brake frame assembly has two brake elements arranged opposite to each other.

In some embodiments according to the present invention, unfolding is to be understood as increasing a diameter of the implant or as allowing the implant to increase in diameter by, for example a shape memory capability plus a sufficient release of the tension thread that would otherwise hinder the implant from returning into its original shape.

To “unfold” may, hence, in some embodiments be understood as actively releasing the tension acting on tension threads biasing the implant.

In some embodiments, the shaft may comprise individual shaft fibers as it is disclosed in WO 2012/084178, the respective disclosure is expressly incorporated herein by reference thereto. In particular embodiments, some or all features mentioned herein with respect to the shaft may also be comprised by some or all of the individual shaft fibers.

In some embodiments, one achievable advantage is that the force applied on the tension threads by which the implant is folded or unfolded may not be exceeded. Hence, the risk of a tension thread rupture because of undue operation of the handle assembly is minimized or even completely avoided because of the force limiter. In fact, the tension the tension threads have to stand upon winding them onto the drum is limited to a pre-set maximum value.

Also, in particular embodiments, the tension threads will stretch under tension. By using the force limiter, one can take up the slack caused by this stretching, thereby ensuring complete folding.

Also, in certain embodiments, the force limiter is designed as a component allowing patency because of its overall ring or tube shape. Hence, the force limiter can be designed to fit into very little space while allow the room it takes to used for other purposes as well. For example, a guide wire may be advanced through the inner lumen established by the elements forming the force limiter.

In some embodiments, one achievable advantage is that the implant must not be overly unfolded. In fact, because of the displacement limiter the tension threads cannot be released more than it was pre-set as the length by which the tension threads may be unwind is limited to a pre-set value by means of the displacement limiter. That way, the tension threads will not be over-released beyond the maximum diameter of the stent. Over-releasing causes the stent/valve to bounce up and down in the blood flow of the beating heart making positioning difficult. Over-releasing also poses a risk where the strings may come off the stent/valve. This may be advantageously avoided by the present invention.

In certain embodiments of the present invention, the force limiter, the knob and/or the first ring element of the force limiter, in particular the inner rim of the knob, for example the gear pattern or teeth matching or corresponding to the gear pattern or teeth of the first ring element, do not comprise deformable projections or deformable teeth.

In some embodiments of the present invention, the spring element of the force limiter comprises at least two or more springs or two or more any other elastic elements.

In certain embodiments of the present invention, the at least two or more springs or two or more any other elastic elements are arranged, formed and/or disposed, in particular uniformly distributed, in or onto the first and/or the second ring element, in particular on a peripherical ring or portion of the first and/or the second ring element.

In some embodiments of the present invention, the first and/or the second ring element is not configured, designed, formed and/or shaped as a plate or disk.

In certain embodiments of the present invention, the ring elements are not connected and/or intended to be connectable to each other through a central shaft and/or the spring element is not supported and/or arranged or disposed around a central shaft connecting the ring elements.

In certain embodiments according to the present invention, one achievable advantage is that the force applied to the force limiter may be distributed homogeneously through and/or within the spring element.

In some embodiments according to the present invention, one achievable advantage is that a tilting movement of the first and/or the second element, in particular relating to a longitudinal direction of the handle assembly and/or to a direction going through the centers of both ring elements, may advantageously be reduced or even avoided.

In particular embodiments a sliding of the first and/or of the second ring element, in particular in a direction perpendicular to the longitudinal direction of the handle assembly and/or to a direction going through the centers of both ring elements, may be advantageously prevented or avoided.

In certain embodiments according to the present invention, one achievable advantage is that the spring constant of the individual springs or corresponding suitable elastic elements can advantageously be reduced compared to or may be lower than the spring constant of a spring element with a single spring (or corresponding single suitable elastic element) of similar properties.

In some embodiments according to the present invention, the force limiter may be of light-weight type, for example in embodiments without central shaft joining the ring elements.

In some embodiments according to the present invention, the handle assembly does not comprise an indicator for indicating the position of an actuation element, for example of a tension thread, in particular no indicator to be aligned with some markings of the handle assembly.

In certain embodiments according to the present invention, the displacement limiter is not designed and/or does not comprise a Geneva-gear mechanism or a Maltese-cross, in particular not of the circumscribed type.

In some embodiments according to the present invention, the displacement limiter does not comprise a band, in particular of a predetermined length, intended and/or configured to be wound around an axis or spool configured to rotate when the drum is rotated and/or during the winding/unwinding of the tension thread.

In certain embodiments according to the present invention, the brake assembly is not configured and/or arranged to brake and/or prevent rotation of the knob in a single direction only and/or does not comprise a device or assembly for releasing, at least temporarily and/or reversibly, the braking force of the brake element applied on the surface of the knob.

In some embodiments according to the present invention, the brake element does not contact the drum and/or may be urged over the outer periphery or circumference of the brake assembly.

In certain embodiments according to the present invention, the surface of the knob being in contact, in particular permanently, with the brake element is an inner surface of the knob.

In some embodiments according to the present invention, the brake element is not and/or does not comprise a detent and/or ratchet and/or may not be actuated by a user and/or its braking force or intensity may not be changed during use of the handle assembly, in particular by a user.

In certain embodiments according to the present invention, the surface of the brake element contacting the knob and/or the corresponding knob surface does not present or comprise teeth.

In some embodiments, the handle assembly comprises at least one of a force limiter, a displacement limiter and a brake frame assembly.

In some embodiments, the implant comprises at least two heart valve leaflets.

In certain embodiments, the implant comprises at least one crown piece (also referred to as ‘triangle’ hereinafter) interconnected to the leaflets.

In some embodiments, the crown piece is preferably intended to be interconnected, directly or indirectly, for example sewed, to a frame (also referred to as the support or the body of the implant or a stent by way of example hereinafter).

In certain embodiments, the implant comprises a top cuff and a bottom cuff.

In some embodiments, the crown piece, the top cuff and the bottom cuff are each rings or ring-shaped, and both the top cuff and the bottom cuff are interconnected with the crown piece.

In certain embodiments, the implant has a frame or support or stent comprising at least or exactly one first guiding structure for guiding or comprising at least one tension thread for folding and/or unfolding the frame around or along the frame, support or stent, for example at an outside or an circumference thereof.

In some embodiments, the frame, support or stent comprises at least or exactly one second guiding structure different from the first guiding structure also for guiding or comprising at least one tension thread for folding and/or unfolding the implant around or along the frame, for example at an outside or an circumference thereof.

In certain embodiments, the frame further comprises at least two, preferably three, posts. The posts are arranged between the first and the second guiding structure such that they interconnect the first and the second guiding structure with each other and/or maintain the distance between them.

In some embodiments, at least one of the top cuff and the bottom cuff is formed from a stripe (or strap) or comprises a stripe (or strap, e.g. a thin band that is longer than broad). The stripe is (in its flat state) curved along its length, preferably or at least in a plane of its width.

In certain embodiments, the top cuff has a width that is smaller than a width of the bottom cuff.

In certain embodiments, the ‘width’ refers to an average width of the stripe.

In some embodiments, the top cuff and the bottom cuff are equally long (or almost equally long).

In particular embodiments, the top cuff is arranged closer to the leaflets than the bottom cuff.

In some embodiments, all leaflets are sewed to the crown piece by means of one or exactly one suture or stitch.

In particular embodiments, a suture is a filament or a thread or yarn. In these embodiments, ‘one suture’ means one filament (or thread or yarn) used for sewing two pieces together. In these embodiments, no second filament is used and, in consequence, only one knot is required.

In some embodiments, both the top cuff and the bottom cuff are sewed to the crown piece by means of one or exactly one suture.

In particular embodiments, the crown piece is sewed to the posts, preferably using (or via or through) through holes or eyelets of the posts, preferably through at least three or four, preferably consecutive through holes, preferably by means of tabs being small extensions of the crown piece or by leaving out the tabs, preferably by means of one or exactly one suture per post.

In some embodiments, the suture for interconnecting the crown piece to one post was started from an outer side of that post to an inner side of the post. Preferably, the single knot that interconnects both ends of the suture is arranged on an outside of the post.

In particular embodiments, the posts are arranged inside a circle or an area circumscribed by the crown piece.

In some embodiments, the posts are arranged outside the circle or the area circumscribed by at least one of the top cuff and the bottom cuff.

In particular embodiments, at least one of the first and the second guiding structure comprises or consists of bars (can be struts instead) that are interconnected to each other so as to form a zig-zag pattern or an undulating or meandering pattern. Neighbouring or adjacent or contacting bars are provided for moving relative to each other or for changing a distance or an angle between them (or between sections thereof, respectively) upon folding or unfolding of the implant or frame. The bars are preferably arranged outside the circle or the area circumscribed by at least one of the top cuff and the bottom cuff. In preferred embodiments the bars are covered on their inner side (being the side towards the inner space of the frame or implant) at least in part(s) by at least one of the top cuff and the bottom cuff.

In some embodiments, the leaflets have a first tab and a second tab arranged at opposite ends of the respective leaflet. The tabs are sewed onto the post of the frame.

In certain embodiments, the tabs extend from the body of the leaflet.

In particular embodiments, tabs of two adjacent leaflets are sewed to one post in an overlapping manner.

In certain embodiments, the tab of a first leaflet is or was sewed onto a post first, and wherein the tab of a second leaflet was sewed onto both the tab of a first leaflet and the post the tab of the first leaflet had been sewed to, all in one running stitch or with one suture.

In some embodiments, the medical implant comprises exactly three posts.

In certain embodiments, the heart valve comprises exactly three leaflets.

In particular embodiments, the implant is a heart or cardiac valve assembly.

In certain embodiments, the crown piece may have up to three sections that are triangle in shape (at least once the crown piece's free ends are put together such that the crown piece forms a ring).

In particular embodiments, the frame (or support or body) of the implant is made of or comprises a metal or a shape memory material.

In certain embodiments, the top cuff and the bottom cuff are originally separate pieces, directly or indirectly interconnected with each other by sewing.

In some embodiments, the crown piece may have sections ending in a tip of a triangle section of the crown piece or in a tab extending from the tip of the triangle, the tab having a free end (before being fixed to, for example, the leaflets).

In certain embodiments, the crown piece is interposed between the top cuff and the bottom cuff.

In certain embodiments, at least one of the top cuff and the bottom cuff is made from porcine pericardium or is a fabric.

In some embodiments, the leaflets are interconnected with, preferably glued or sewed to, the crown piece.

In particular embodiments, the frame, support or stent has features as described in WO 2011/063972 A1 or WO 2009/109348 A1 with respect to the implant. The respective disclosures of WO 2011/063972 A1 and WO 2009/109348 A1 are incorporated in its entirety into the present specification by reference.

In certain embodiments, the implant is configured to have or has tension applied to it by using at least one tension thread. The tension is preferably controlled by altering a length of the pulling device by which it extends out of the interior of the shaft or an implantation device or sections thereof.

In certain embodiments, at least one of the heart valve and the frame comprises exclusively (one or more) materials that are not magnetic, ferromagnetic, or both.

In some embodiments, at least one of the heart valve and the frame does not comprise metal or any metal alloy.

In certain embodiments, eat least one of the posts has at least two openings through which tension threads are guided from an inside or inner space of the implant to an outside of the implant and back from the outside to the inside. The tension threads are guided to the outside through a first opening of a first one of the posts and back to the inside—or vice versa—through any second first opening of any second post, the first opening being different from the second opening, and the first post being different from the second post.

In particular embodiments, the crown piece is interconnected to the frame of a medical implant or a heart valve assembly.

In certain embodiments, both the top cuff and the bottom cuff are interconnected to bars of a guiding structure of the frame, preferably by sewing, preferably in direct contact to the bars.

According to some embodiments, the top cuff and the bottom cuff may have different widths. If the top cuff and the bottom cuff is now everted to the outside face of the bars both at an upper end and an lower end of the bars by an equal distance (the equal distance is usually equal since it takes the same amount of material or overlap to secure the cuffs on the tips of both the upper parts and the lower parts of the bars), a suture interconnecting the two cuffs (plus the crown piece) will not be positioned in a middle line of the bars. That way, the suture will not be damaged by the bars in a folded state of the implant in which the middle of the bars will usually have to face the highest pressure. At the same token, the suture does not contribute to applying pressure on the leaflets starting about the height of the bars of the guiding structure as the suture will not contribute to narrowing the space about the middle line of the bars due to its position beyond the middle line.

Further, in certain embodiments, sewing parts by just one suture may contribute in avoiding knots which in turn require space and are prone to damaging neighboring structures such as leaflets.

If, as in particular embodiments, the inner side of the top cuff is interconnected to the outer side of the bottom cuff, the resulting geometrical shape will show a profile that extends with a middle portion thereof into the inner space it circumscribes. In a front cut the geometrical shape may be called concave. That shape may fit best to the also concave shape of the bent bars and the resulting concave shape of the guiding structure which is another advantage.

In some embodiments, the implant is or comprises a heart valve assembly comprising a frame and a heart valve.

In certain embodiments, the frame supports the heart valve or is interconnected thereto, preferably by sewing or sewing alone.

The interconnecting tissue or parts thereof, respectively, is interconnected to at least one of the guiding structures such that it covers the guiding structure or parts thereof at an inner circumference of the guiding structure and such that it also covers both an upper part and a lower part of the outer circumference of the guiding structure.

In certain embodiments, at least one of the first and the second guiding structure comprises bars or consist of bars. The bars have top portions and bottom portions (herein, ‘top’ and ‘bottom’, like ‘upper’ and ‘lower’, refer to the orientation of the drawings or to the main orientation of the heart valve assembly or to the main flow through direction of the heart valve assembly in use). The interconnecting tissue or parts thereof are interconnected to the bars such that the interconnecting tissue covers the bars at an inner circumference of the guiding structure. It also covers the bars at both an upper part and a lower part of the outer circumference of the guiding structure.

In some embodiments, the interconnecting tissue comprises or consists of at least one crown piece interconnected to the leaflets, a top cuff and a bottom cuff each of which is ring-shaped, and both the top cuff and the bottom cuff are interconnected with the crown piece.

In certain embodiments, at least one of the top cuff and the bottom cuff is formed from a stripe or comprises a stripe, wherein the stripe is curved along its length.

In some embodiments, the top cuff has a width that is smaller than the width of the bottom cuff.

In certain embodiments, the crown piece or an section thereof is interposed between the top cuff and the bottom cuff.

In some embodiments, the frame is foldable and/or unfoldable.

In certain embodiments, bars of at least one of the first and the second guiding structure are interconnected to each other so as to form a zig-zag pattern or an undulating or meandering pattern. The bars are preferably arranged outside the circle or the area circumscribed by at least one of the interconnecting tissue, the top cuff and the bottom cuff.

In particular embodiments, the heart valve assembly or the frame may be folded or unfolded upon implantation by using one or several tension threads or filaments wound around the assembly.

In certain embodiments, folding the implant means reducing the diameter of the implant. Folding also covers “re-folding” of an once expanded implant.

In some embodiments, unfolding should be understood as increasing the diameter of the implant, or as expanding.

In some embodiments, at least one of the top cuff and the bottom cuff is formed from a stripe (or strap) or comprises a stripe (or strap, e.g. a thin band that is longer than broad). The stripe is (in its flat state) curved along its length, preferably or at least in a plane of its width.

In certain embodiments, the top cuff has a width that is smaller than a width of the bottom cuff.

In certain embodiments, the ‘width’ refers to an average width of the stripe.

In some embodiments, the top cuff and the bottom cuff are equally long (or almost equally long).

In particular embodiments, the top cuff is arranged closer to the leaflets than the bottom cuff.

In some embodiments, all leaflets are sewed to the crown piece by means of one or exactly one suture or stitch.

In particular embodiments, a suture is a filament or a thread or yarn. In these embodiments, ‘one suture’ means one filament (or thread or yarn) used for sewing two pieces together. In these embodiments, no second filament is used and, in consequence, only one knot is required.

In some embodiments, both the top cuff and the bottom cuff are sewed to the crown piece by means of one or exactly one suture.

In particular embodiments, the crown piece is sewed to the posts, preferably using (or via or through) through holes or eyelets of the posts, preferably through at least three or four, preferably consecutive through holes, preferably by means of tabs being small extensions of the crown piece or by leaving out the tabs, preferably by means of one or exactly one suture per post.

In some embodiments, the suture for interconnecting the crown piece to one post was started from an outer side of that post to an inner side of the post. Preferably, the single knot that interconnects both ends of the suture is arranged on an outside of the post.

In particular embodiments, the posts are arranged inside a circle or an area circumscribed by the crown piece.

In some embodiments, the posts are arranged outside the circle or the area circumscribed by at least one of the top cuff and the bottom cuff.

In particular embodiments, at least one of the first and the second guiding structure comprises or consists of bars that are interconnected to each other so as to form a zig-zag pattern or an undulating or meandering pattern. Neighbouring or adjacent or contacting bars are provided for moving relative to each other or for changing a distance or an angle between them (or between sections thereof, respectively) upon folding or unfolding of the implant or frame. The bars are preferably arranged outside the circle or the area circumscribed by at least one of the top cuff and the bottom cuff. In preferred embodiments the bars are covered on their inner side (being the side towards the inner space of the frame or implant) at least in part(s) by at least one of the top cuff and the bottom cuff.

In some embodiments, the leaflets have a first tab and a second tab arranged at opposite ends of the respective leaflet. The tabs are sewed onto the post of the frame.

In certain embodiments, the tabs extend from the body of the leaflet.

In particular embodiments, tabs of two adjacent leaflets are sewed to one post in an overlapping manner.

In certain embodiments, the tab of a first leaflet is or was sewed onto a post first, and wherein the tab of a second leaflet was sewed onto both the tab of a first leaflet and the post the tab of the first leaflet had been sewed to, all in one running stitch or with one suture.

In some embodiments, the frame comprises exactly three posts.

In certain embodiments, the heart valve comprises exactly three leaflets.

In certain embodiments, the crown piece may have up to three sections that are triangle in shape (at least once the crown piece's free ends are put together such that the crown piece forms a ring).

In particular embodiments, the frame (or support or body) of the implant is made of or comprises a metal or a shape memory material.

In certain embodiments, the top cuff and the bottom cuff are originally separate pieces, directly or indirectly interconnected with each other by sewing.

In some embodiments, the crown piece may have sections ending in a tip of a triangle section of the crown piece or in a tab extending from the tip of the triangle, the tab having a free end (before being fixed to, for example, the leaflets).

In certain embodiments, the crown piece is interposed between the top cuff and the bottom cuff.

In certain embodiments, at least one of the top cuff and the bottom cuff is made from porcine pericardium or is a fabric.

In some embodiments, the leaflets are interconnected with, preferably glued or sewed to, the crown piece.

In particular embodiments, the frame has features as described in WO 2011/063972 A1 or WO 2009/109348 A1 with respect to the frame. The respective disclosures of WO 2011/063972 A1 and WO 2009/109348 A1 are incorporated in its entirety into the present specification by reference.

In certain embodiments, the implant is configured to have or has tension applied to it by using at least one tension thread. The tension is preferably controlled by altering a length of the pulling device by which it extends out of the interior of the shaft or a catheter or sections thereof.

In certain embodiments, at least one of the heart valve and the frame comprises exclusively (one or more) materials that are not magnetic, ferromagnetic, or both.

In certain embodiments, at least one of the posts has at least two openings through which tension threads are guided from an inside or inner space of the implant to an outside of the implant and back from the outside to the inside. The tension threads are guided to the outside through a first opening of a first one of the posts and back to the inside—or vice versa—through any second first opening of any second post, the first opening being different from the second opening, and the first post being different from the second post.

Some or all embodiments may provide for one, several or all of the advantages named above and/or hereafter.

In some of the embodiments, the interconnecting tissue or element forms two sealing tissues or elements. Having two such elements instead of only one has proven to contribute to achieving a superb sealing effect.

Since, as in certain embodiments, the sealing elements are ring-shaped structures which circumference is supported by only the tips of bars forming an undulating pattern without any support by the frame between the sealing elements' contact with the tips of the bars, the sealing elements are free to adapt to the native tissue surrounding the heart valve assembly. This way, sealing may be improved when compared to the results achieved by the state of art hitherto.

According to the present invention, the top cuff and the bottom cuff may have different widths. If the top cuff and the bottom cuff is now everted to the outside face of the bars both at an upper end and an lower end of the bars by an equal distance, a suture interconnecting the two cuffs (plus the crown piece) will not be positioned in a middle line of the bars. That way, the suture will not be damaged by the bars in a folded state of the implant in which the middle of the bars will usually have to face the highest pressure. At the same token, the suture does not contribute to applying pressure on the leaflets starting about the height of the bars of the guiding structure as the suture will not contribute to narrowing the space about the middle line of the bars due to its position beyond the middle line.

Further, sewing parts by just one suture may contribute in avoiding knots which in turn require space and are prone to damaging neighboring structures such as leaflets.

If, as in particular embodiments, the inner side of the top cuff is interconnected to the outer side of the bottom cuff, the resulting geometrical shape will show a profile that extends with a middle portion thereof into the inner space it circumscribes. In a front cut the geometrical shape may be called concave. That shape may fit best to the also concave shape of the bent bars and the resulting concave shape of the guiding structure which is another advantage.

In the following, examples of the present invention will be described with reference to the accompanying figures wherein similar or identical assemblies or elements may be denoted by same reference numerals.

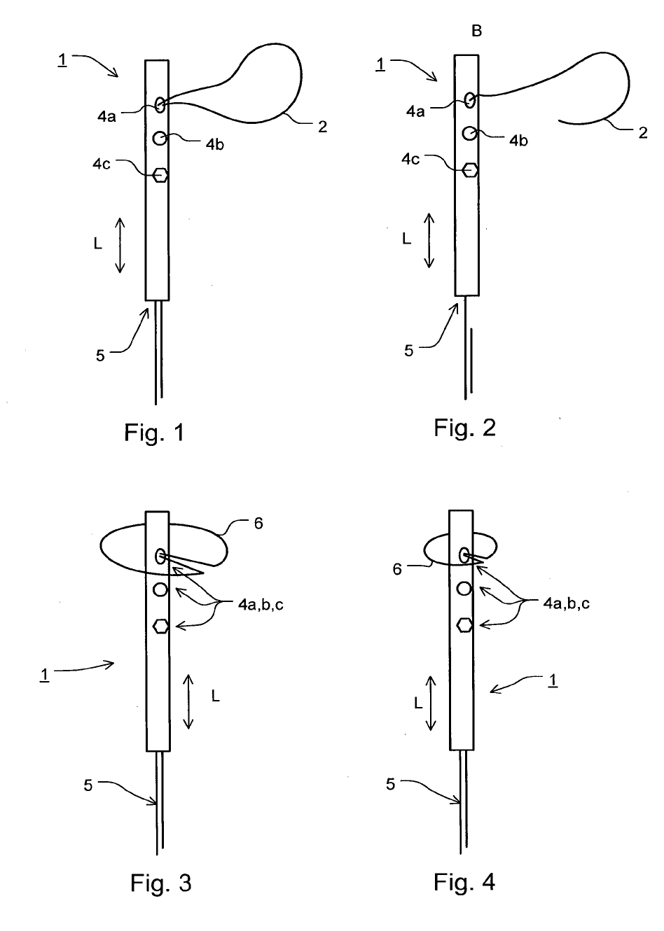

FIG. 1 shows in a schematically simplified view, an implantation device which comprises guiding units;

FIG. 2 shows the arrangement of FIG. 1;

FIG. 3 shows the arrangement of FIG. 1;

FIG. 4 shows the arrangement of FIG. 1;

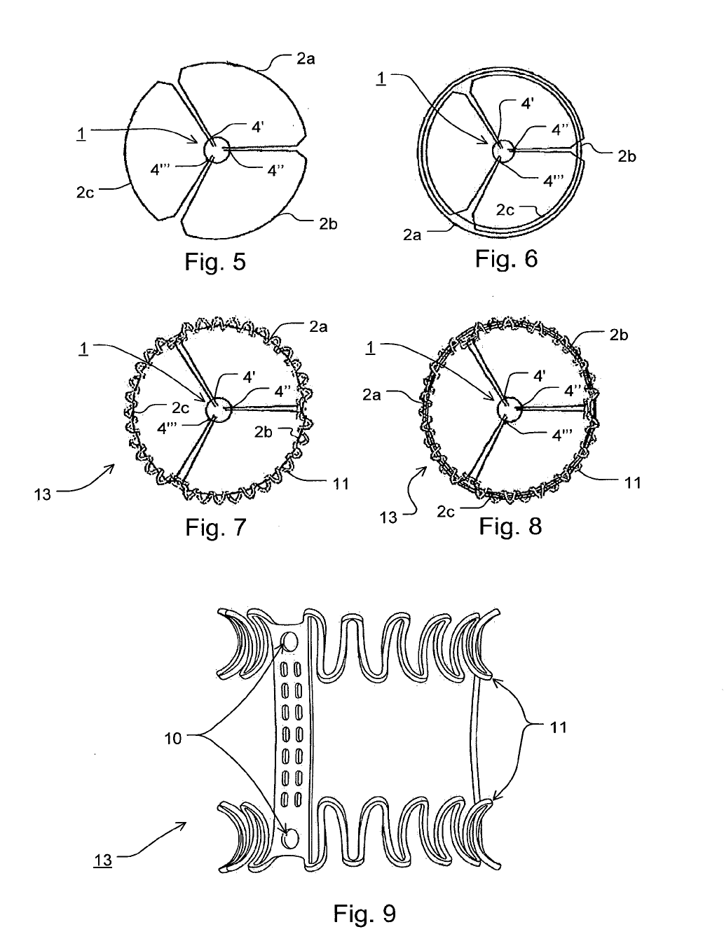

FIG. 5 shows an exemplarily run or extension, respectively, of three tension threads each about ⅓ of the periphery of a stent;

FIG. 6 shows the run or extension, respectively, of tension threads along the complete periphery of a stent;

FIG. 7 shows the implantation device of FIG. 5;

FIG. 8 shows the implantation device of FIG. 6 comprising the stent of FIG. 7;

FIG. 9 shows an expandable stent which is reducable in its diameter again by the use of a means;

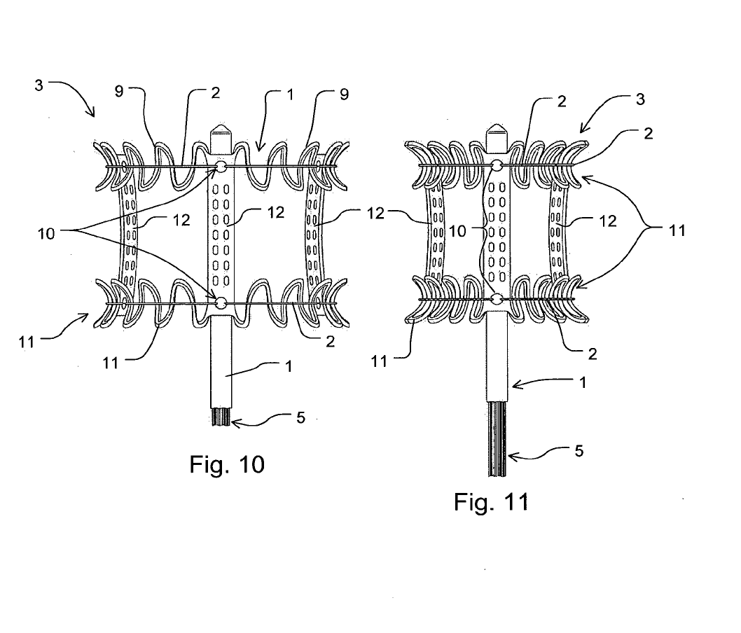

FIG. 10 shows the stent of FIG. 9;

FIG. 11 shows tensioned tension threads and a reduced diameter of the stent;

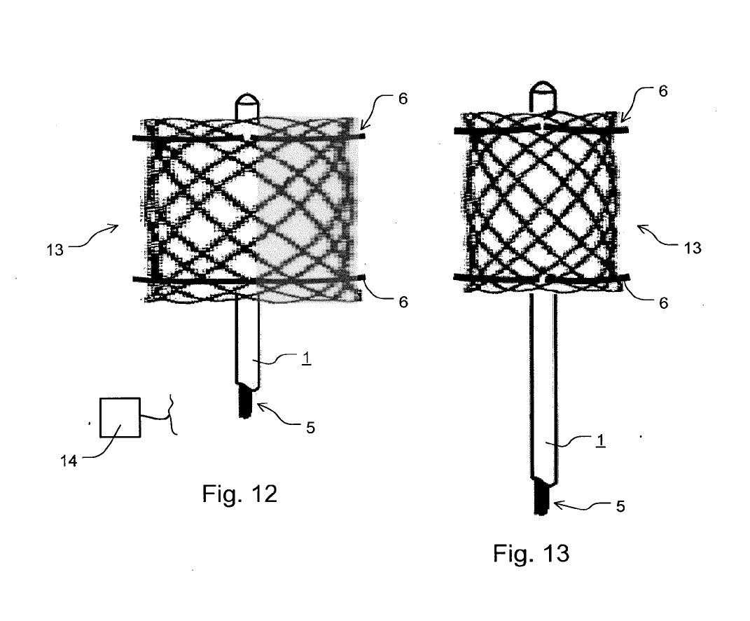

FIG. 12 shows a stent in the representation of FIG. 9;

FIG. 13 shows a stent in the representation of FIG. 10;

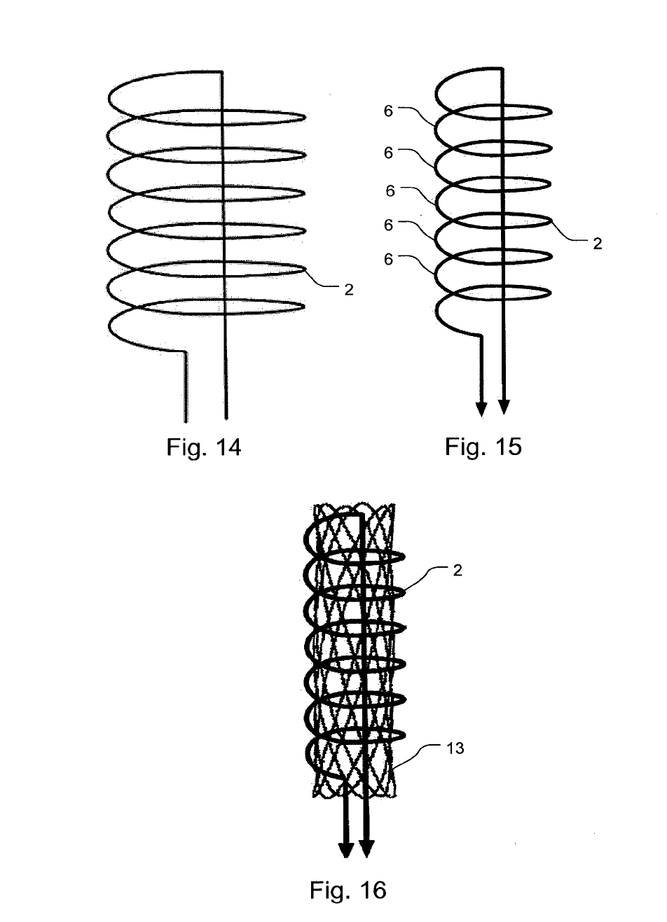

FIG. 14 shows an embodiment in which the tension threads are guided around the stent in a spiral form;

FIG. 15 shows a tension thread which is tensioned or stressed, respectively, on both ends, wherein the diameter of the spirally guided tension thread has been reduced;

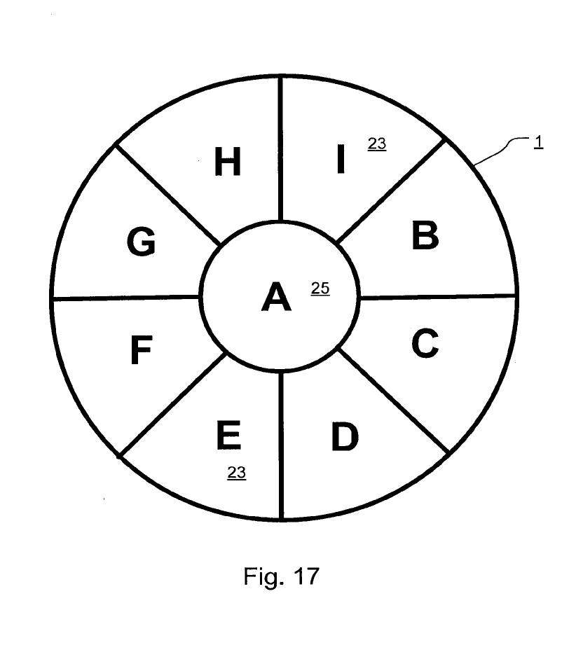

FIG. 16 shows the state of the tension thread of FIG. 15 about or around a stent;

FIG. 17 shows an implantation device according to the invention having a cross-section comprising a plurality of lumina;

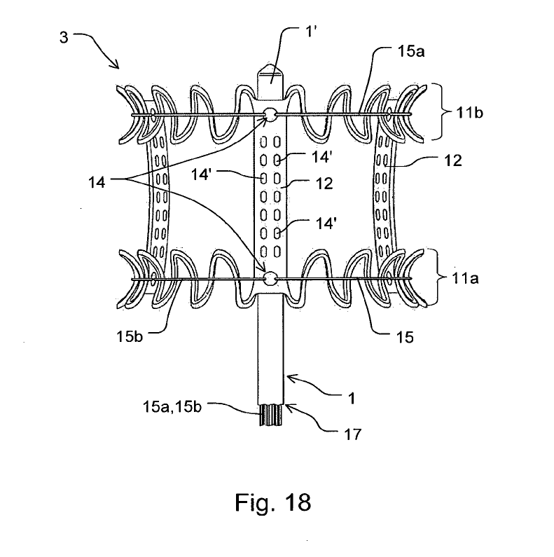

FIG. 18 shows how an implant with an implantation device may look like in a side view during expansion or in an expanded state;

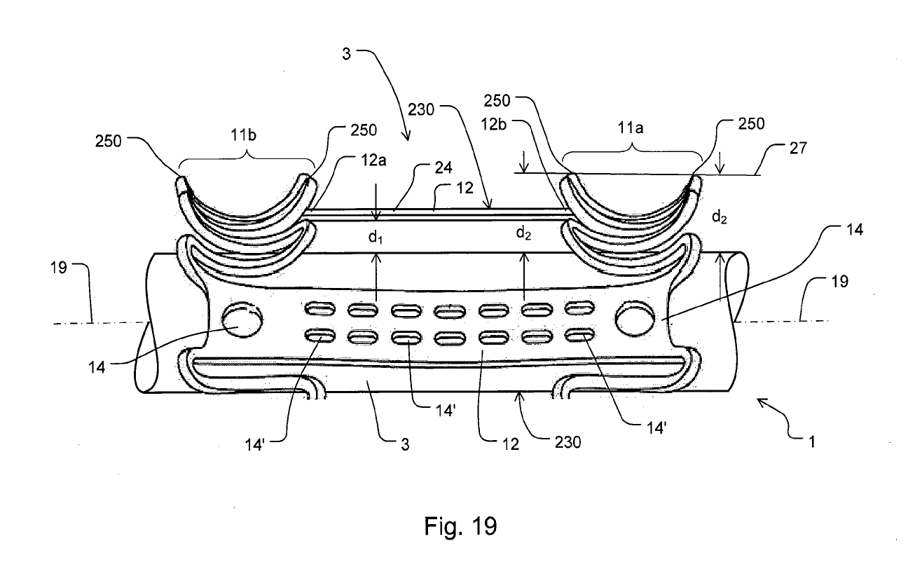

FIG. 19 shows an implant according to the invention, partly cut, crimped on a implantation device;

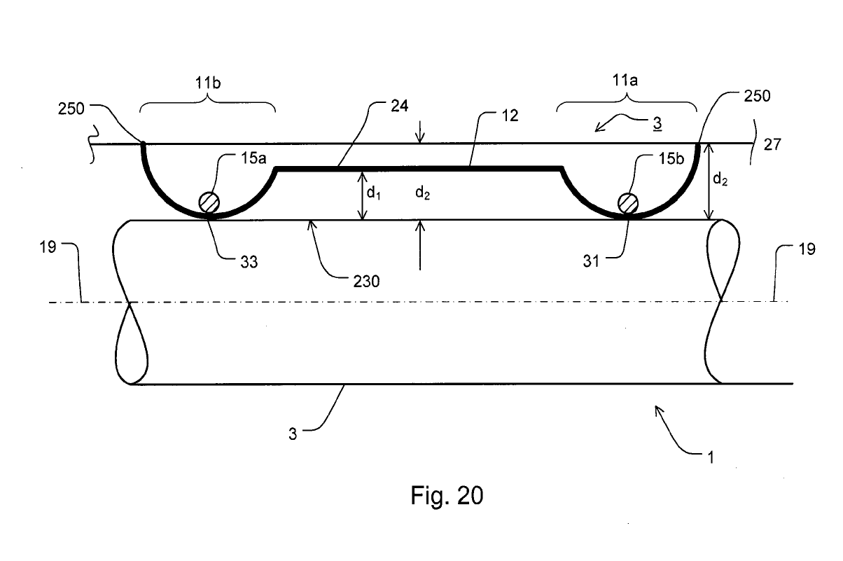

FIG. 20 shows a schematic illustration of an implant of the invention in a longitudinal section;

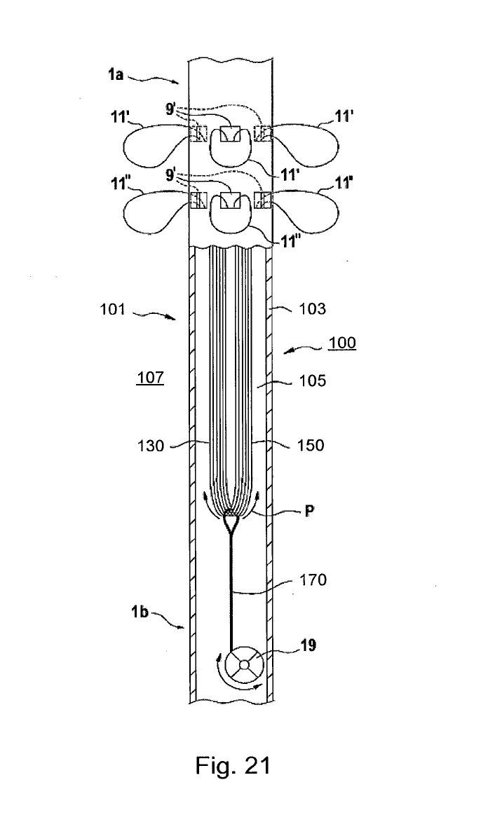

FIG. 21 shows a longitudinal part section through a schematically simplified cutaway view of an implantation device;

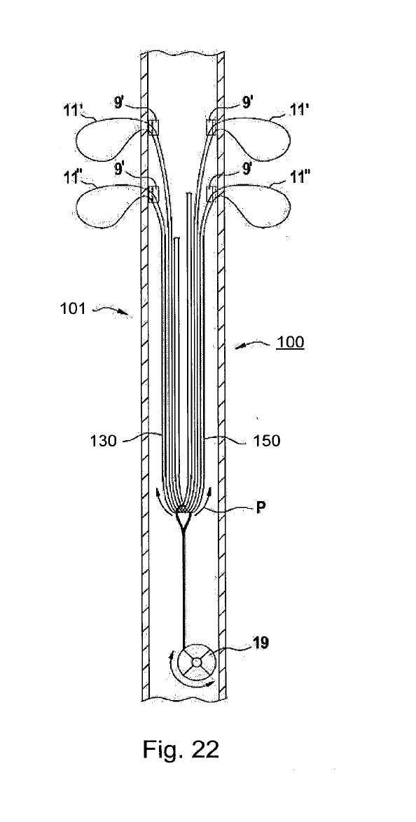

FIG. 22 shows the object in FIG. 21 in a full longitudinal section;

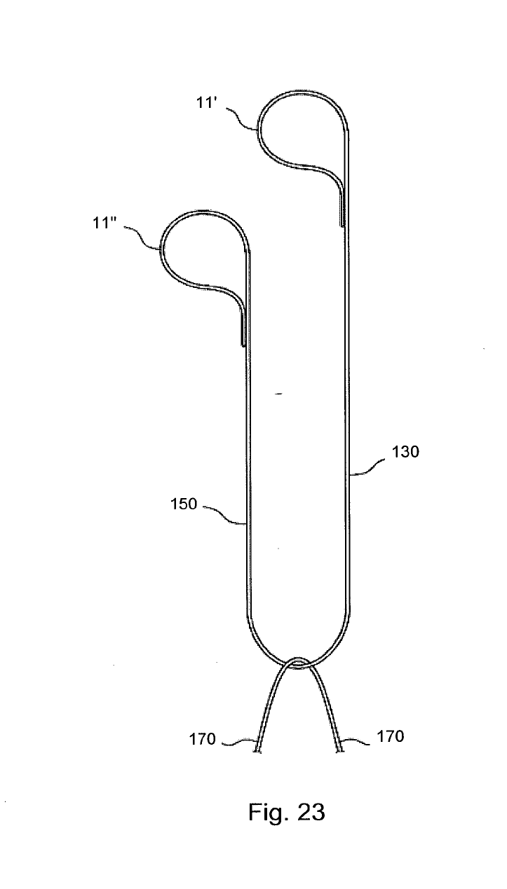

FIG. 23 shows the self-balancing design shown in FIG. 21 and FIG. 22 in a schematically simplified manner;

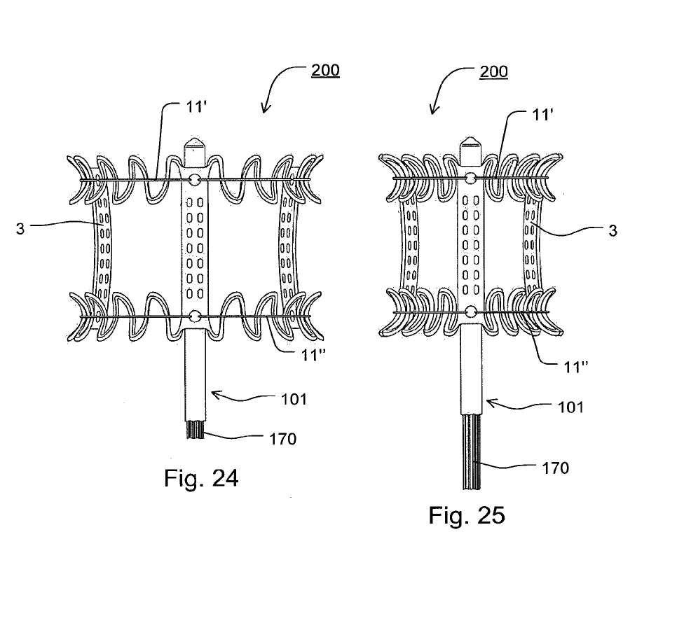

FIG. 24 shows schematically simplified and in part section, a set according to the invention with an implant expanding though the action of the implantation device;

FIG. 25 shows the set of FIG. 24 with the implant in a further (partly) folded condition by means of the implantation device and/or folding device;

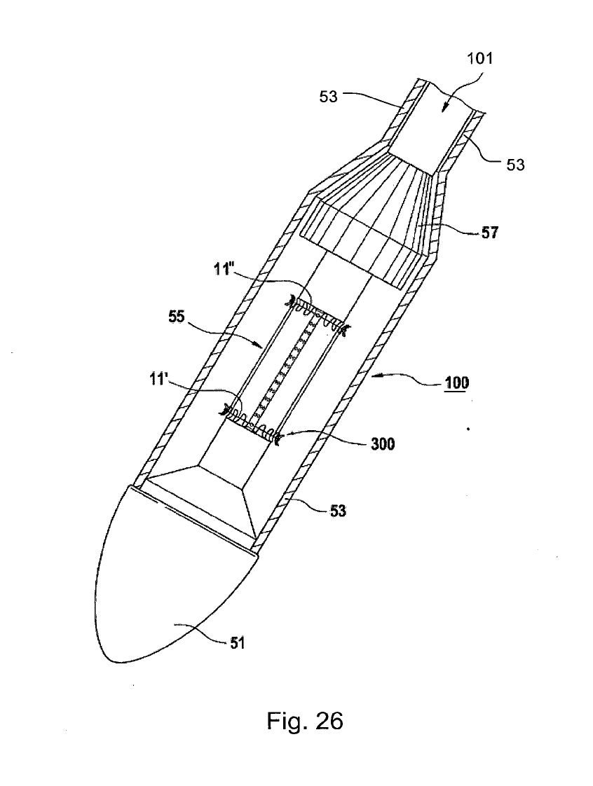

FIG. 26 shows the tip of an implantation device shown in a closed condition prior to implantation;

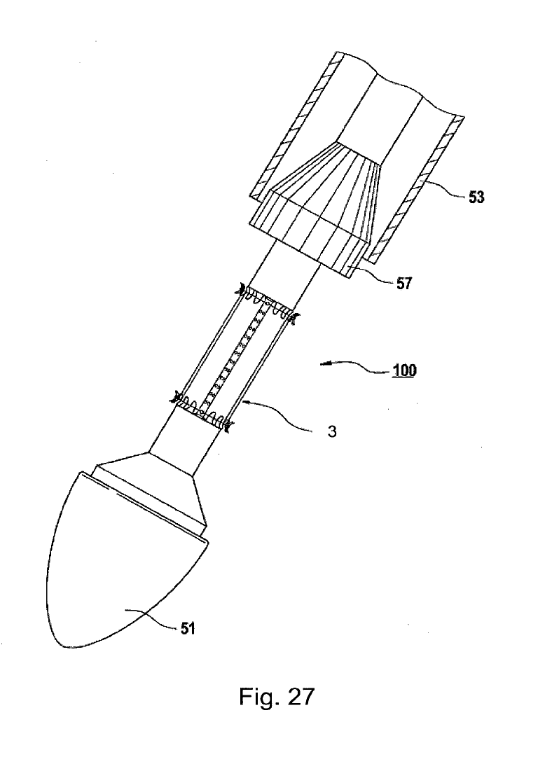

FIG. 27 shows the tip of the implantation device as in FIG. 26 prior to implantation with partially withdrawn outer sleeve;

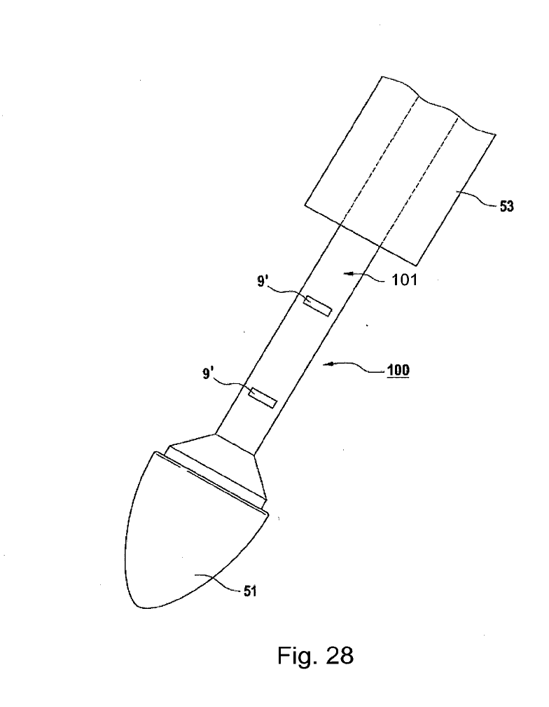

FIG. 28 shows the tip of an implantation device in FIG. 27 without implant;

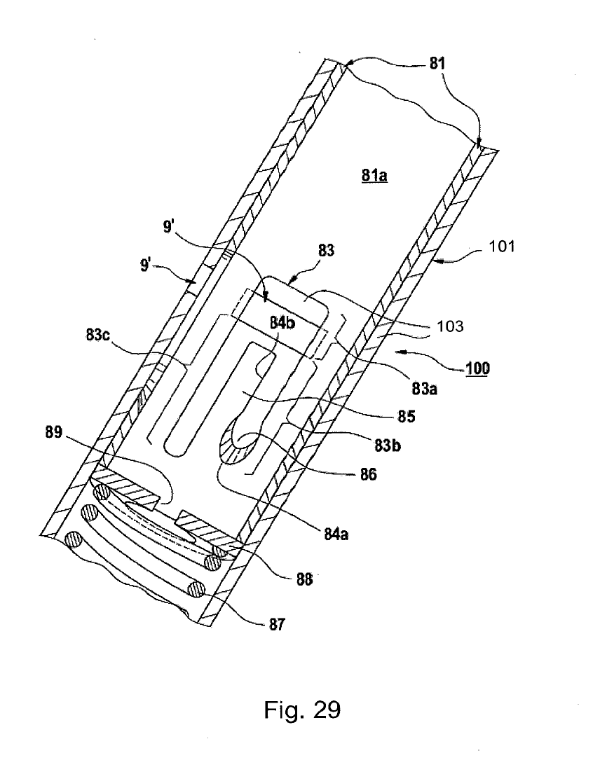

FIG. 29 shows the shaft and the sleeve of the implantation device of FIG. 27 in part view, in a part section and with a first opening;

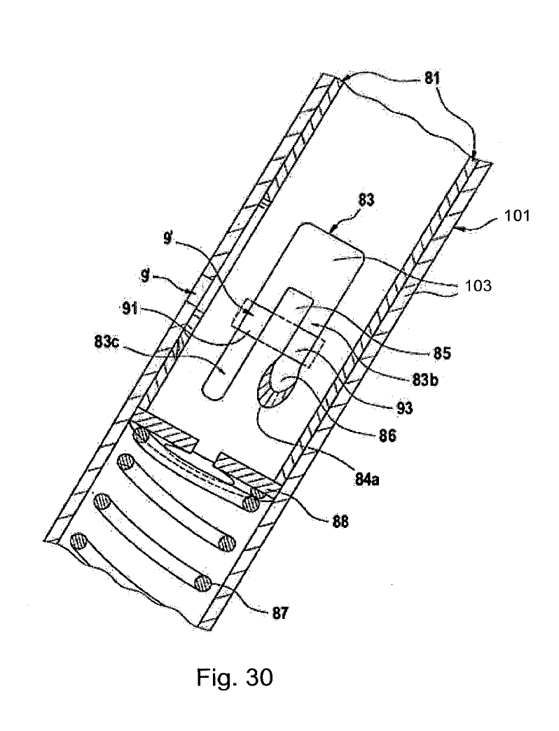

FIG. 30 shows the shaft and the sleeve of the implantation device of FIG. 27 in part view, in a part section and with a second opening;

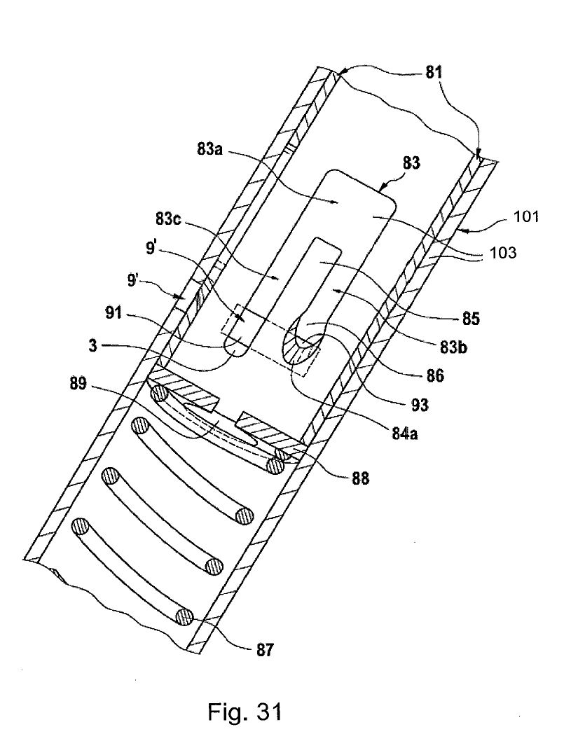

FIG. 31 shows a shaft and sleeve of the implantation device of FIG. 27 in part view, in a part section and in a third opening; and

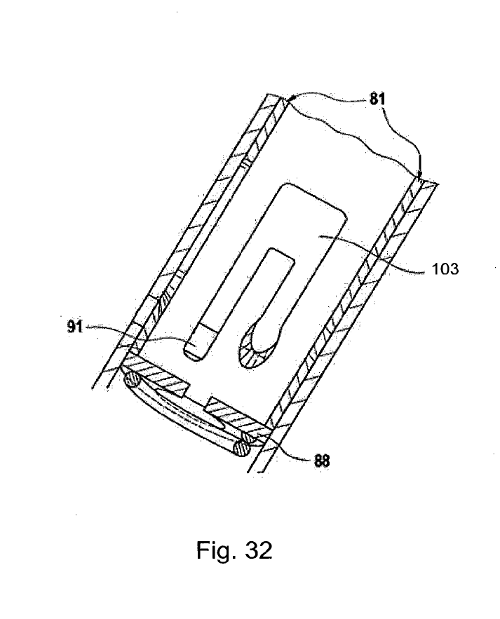

FIG. 32 shows a shaft and sleeve of the implantation device of FIG. 27 in part view, in a part section after passing through the third opening.

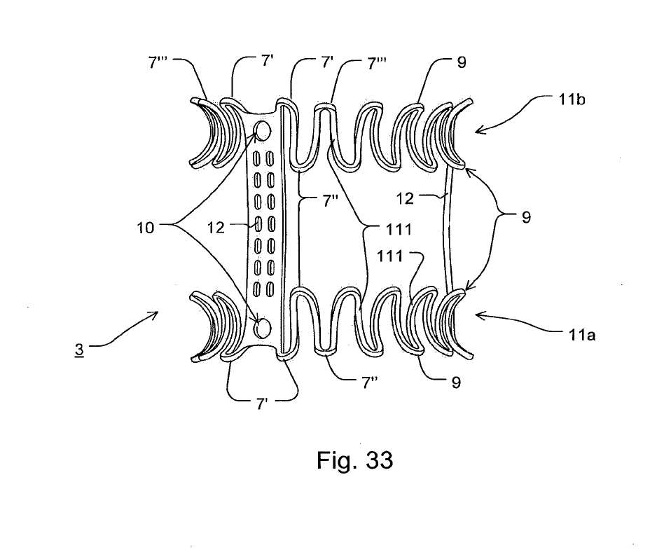

FIG. 33 shows enlarged sections of the medical implant of FIG. 10;

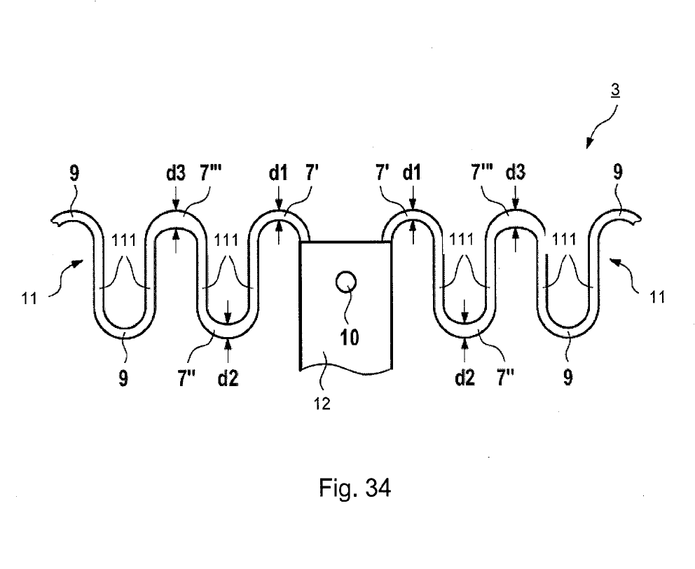

FIG. 34 shows enlarged sections of a supporting means of the implant of FIGS. 10, 11 and 33;

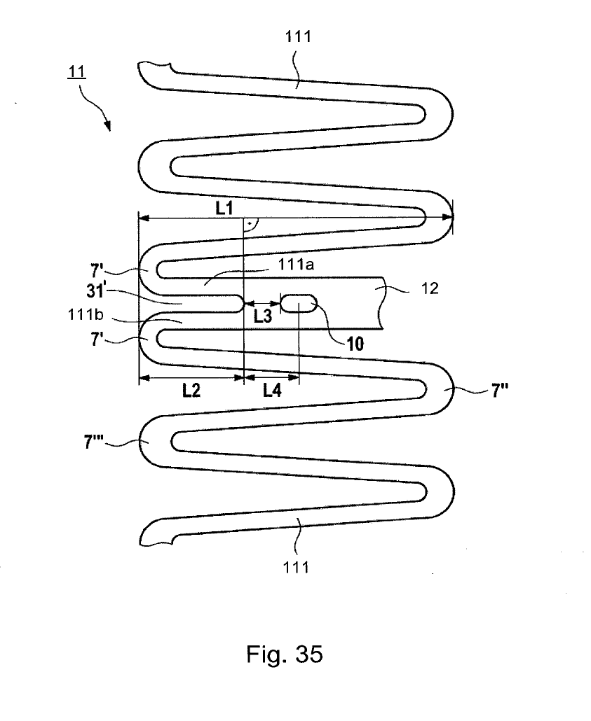

FIG. 35 shows an enlarged section of a supporting means of an implant;

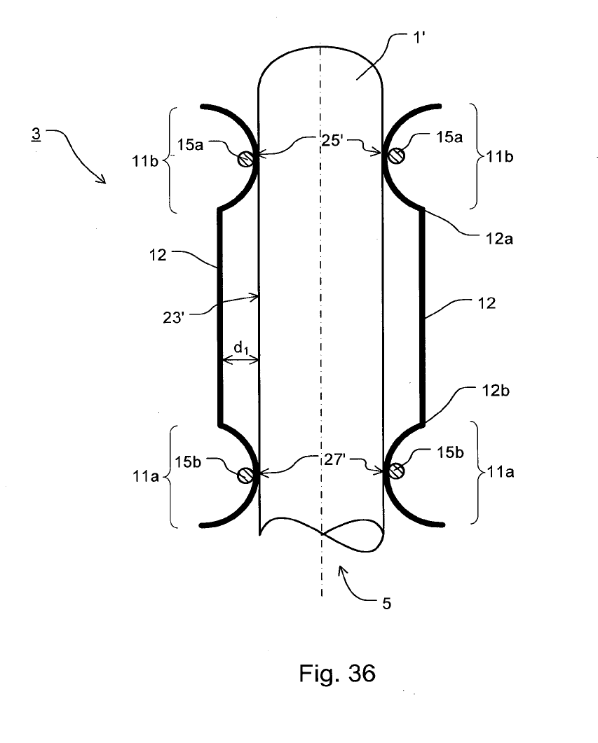

FIG. 36 shows a schematic illustration of of an implant;

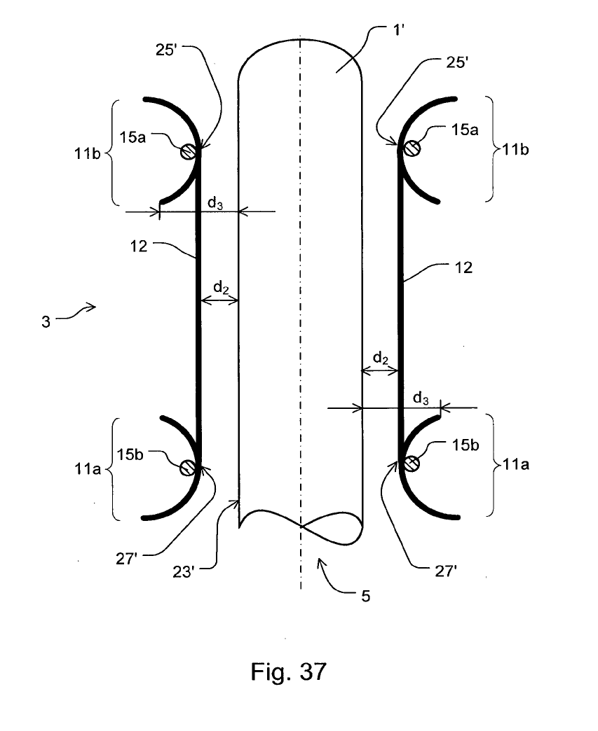

FIG. 37 shows a schematic illustration of another embodiment of an implant;

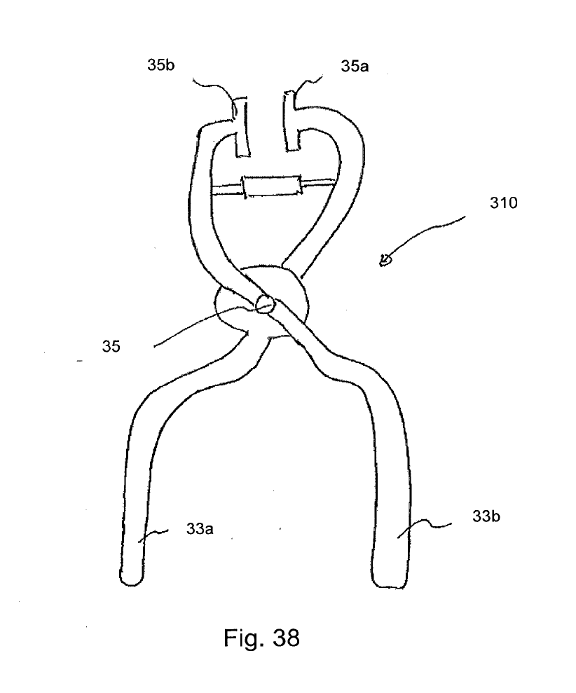

FIG. 38 shows a schematic illustration of a crimping device;

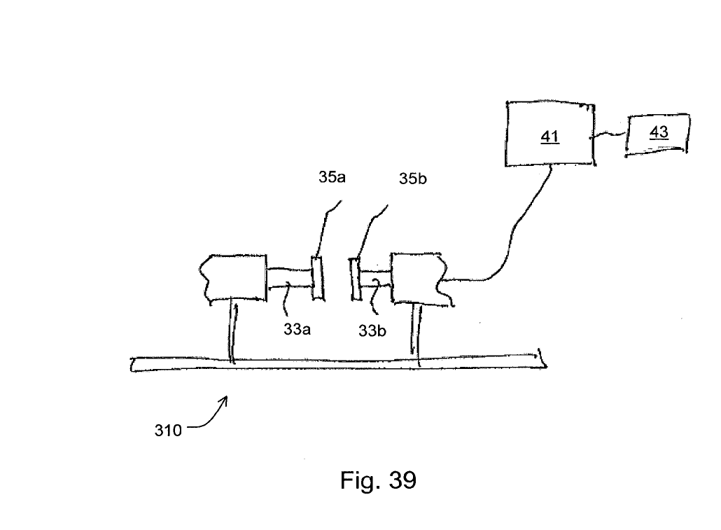

FIG. 39 shows a schematic illustration of another embodiment of a crimping device;

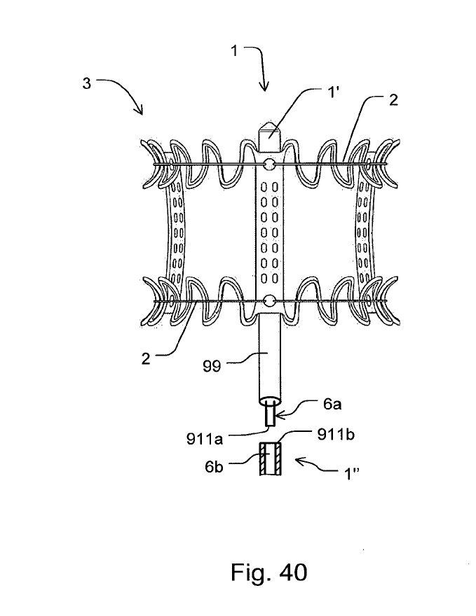

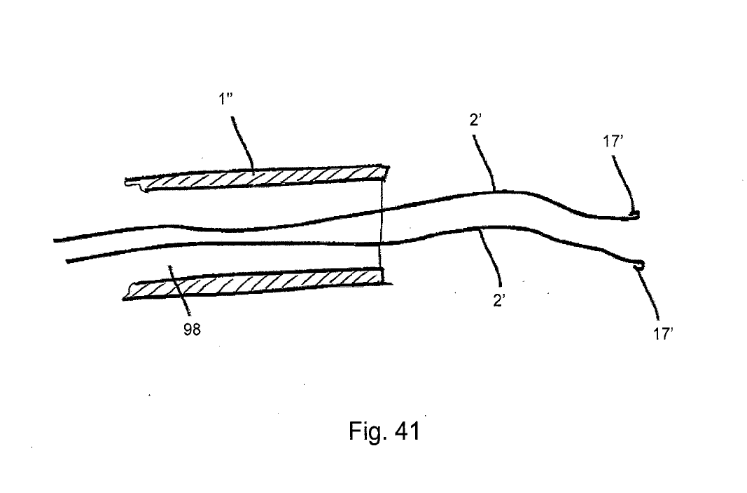

FIG. 40 shows an implantation device comprising an implant;

FIG. 41 schematically shows a catheter or implantation device comprising second folding and/or unfolding means; and

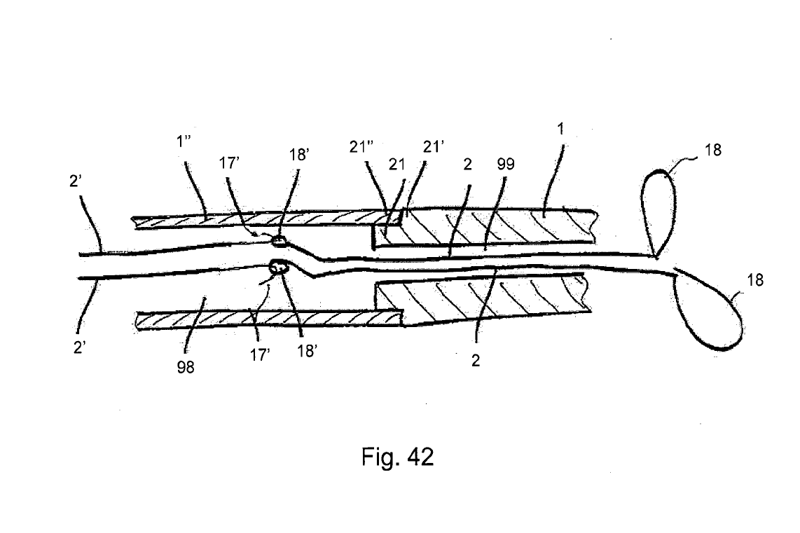

FIG. 42 schematically shows a connection state between a catheter and a catheter tip;

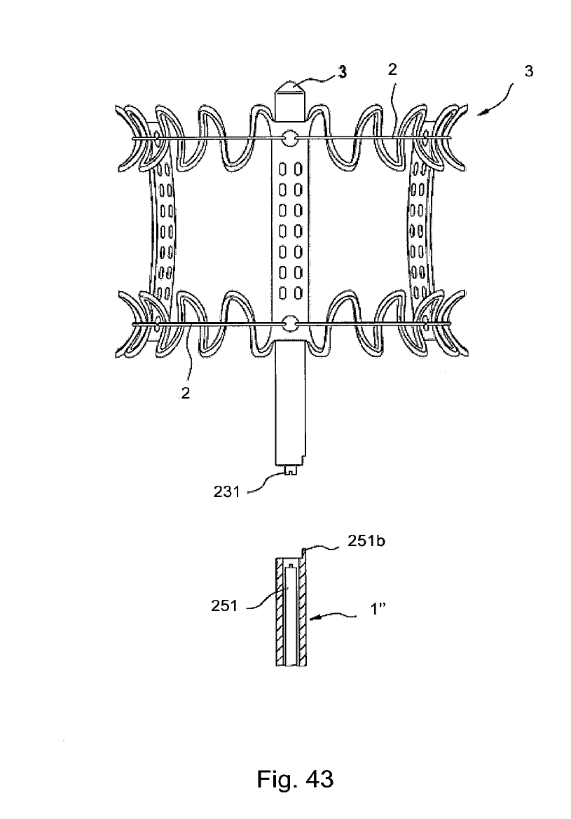

FIG. 43 shows an implantation device comprising an implant;

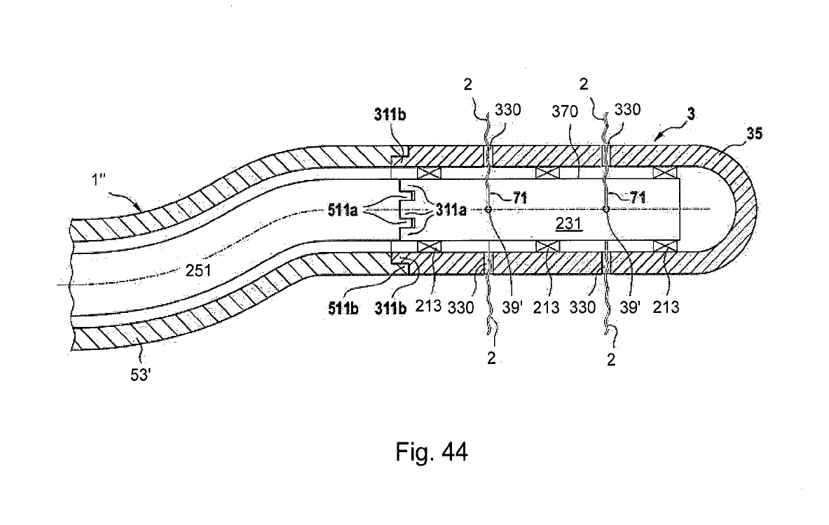

FIG. 44 schematically shows a part of a catheter comprising a folding device of a first embodiment designed as a catheter tip in a longitudinal section;

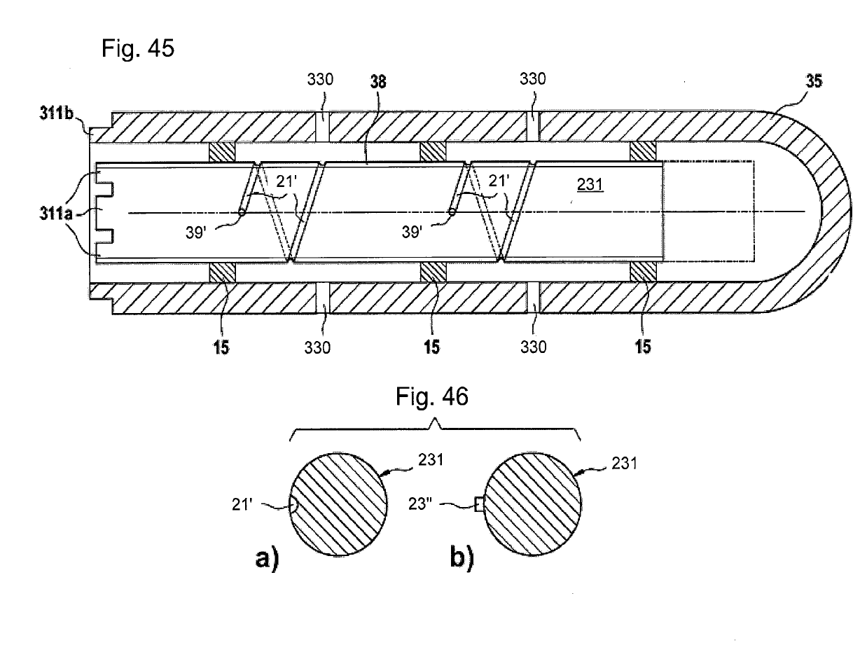

FIG. 45 schematically shows a catheter of a second embodiment designed as a catheter tip in a longitudinal section;

FIG. 46a, b shows different embodiments of the portion for folding and/or unfolding the implant in cross-section.

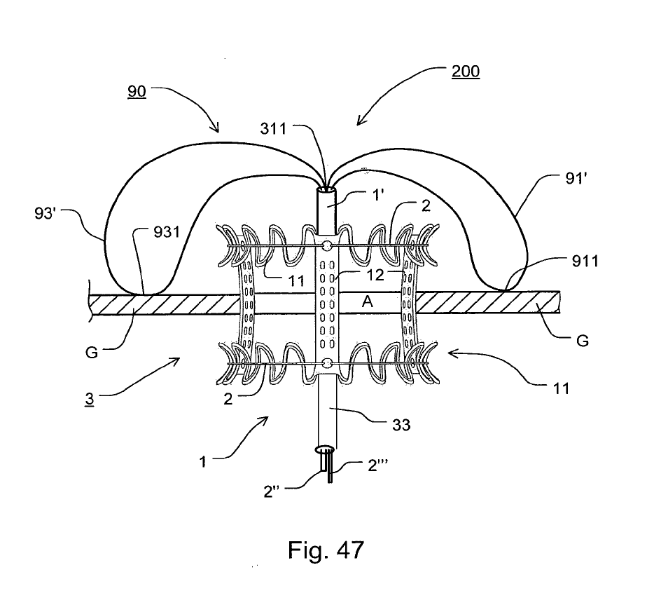

FIG. 47 shows a first embodiment of a set comprising an implantation device, a medical implant and an aligning device;

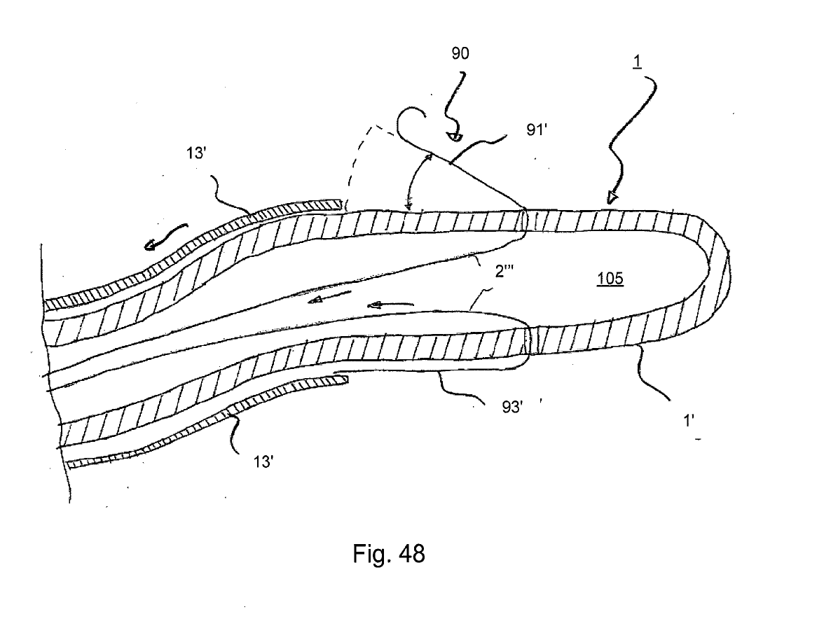

FIG. 48 shows a second embodiment of an implantation device comprising a medical implant and an aligning device;

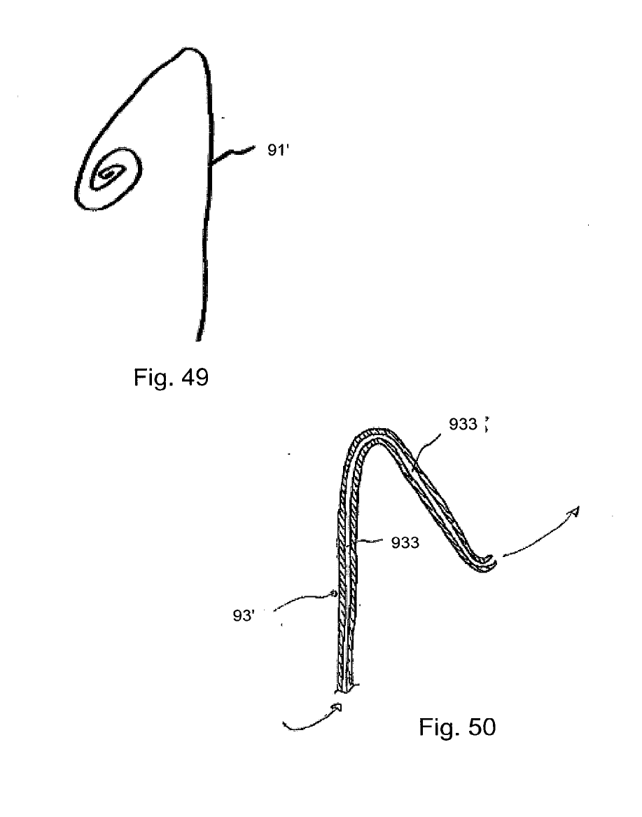

FIG. 49 shows an aligning section of an implantation device in a further embodiment;

FIG. 50 shows an aligning section of an implantation device in a still further embodiment;

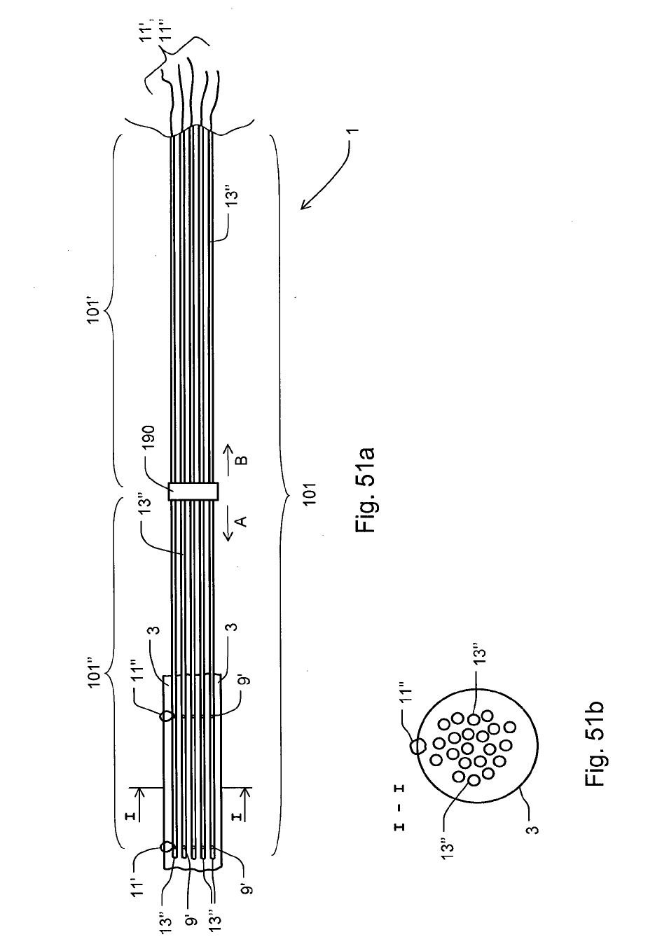

FIG. 51a shows a partial longitudinal section through an implantation device according to the present invention, a section of which is shown in a schematically simplified manner, prior to unfolding an implant;

FIG. 51b shows a section along the line I-I of FIG. 51a;

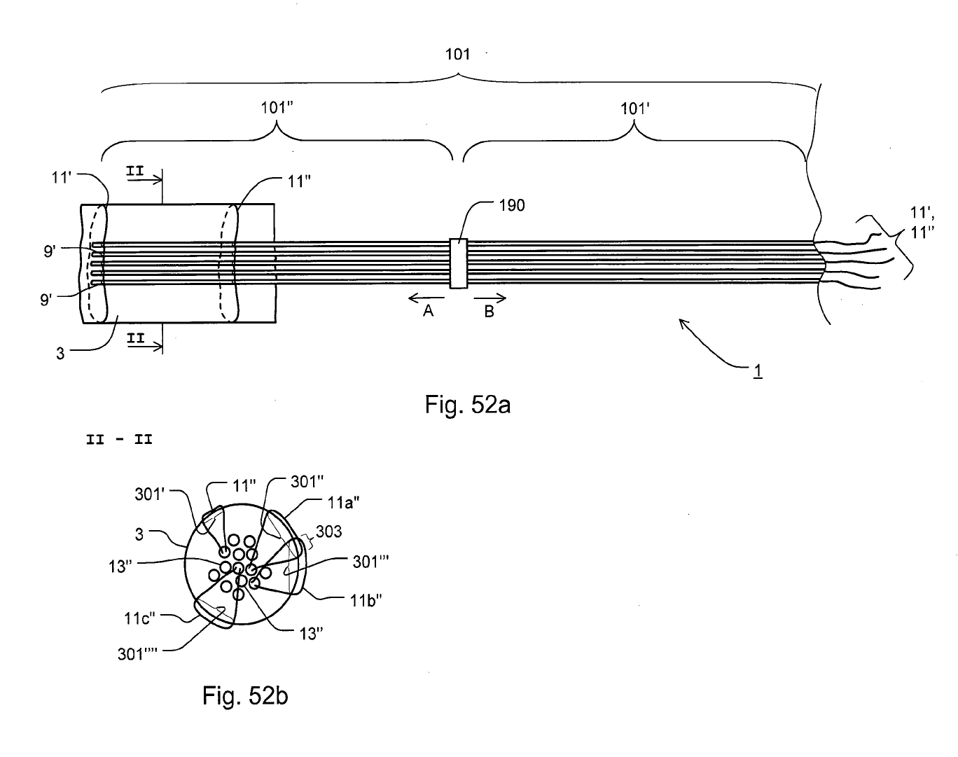

FIG. 52a shows a partial longitudinal section through the implantation device of FIG. 51a, a section of which is shown in a schematically simplified manner, after unfolding the implant, with tension-free tension threads;

FIG. 52b shows a section along the line II-II of FIG. 52a;

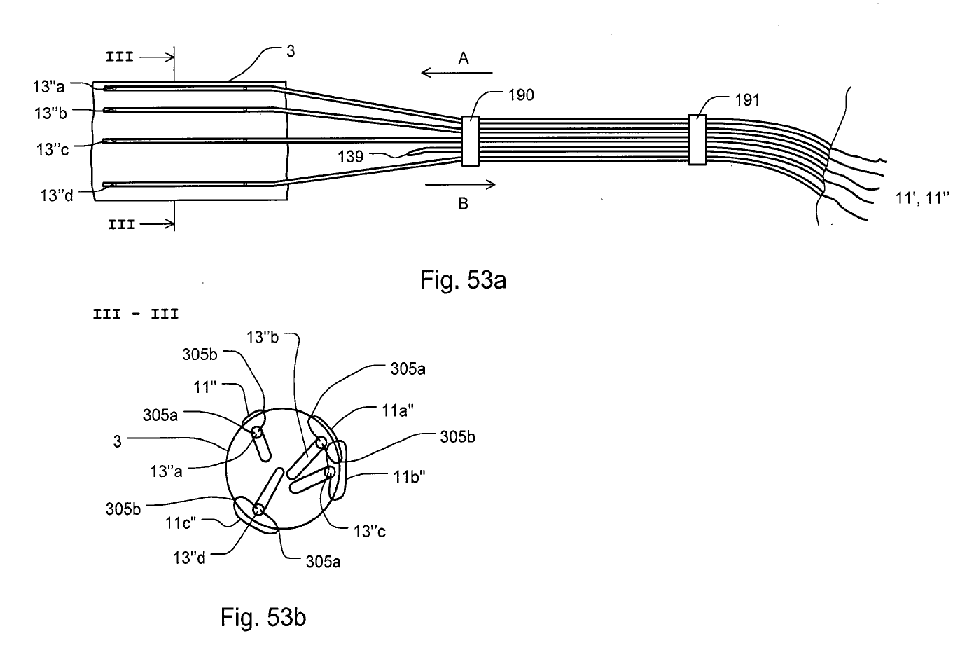

FIG. 53a shows a partial longitudinal section through the implantation device of FIG. 51a, a section of which is shown in a schematically simplified manner, after unfolding the implant with tensioned tension threads;

FIG. 53b shows a section along the line of FIG. 53a;

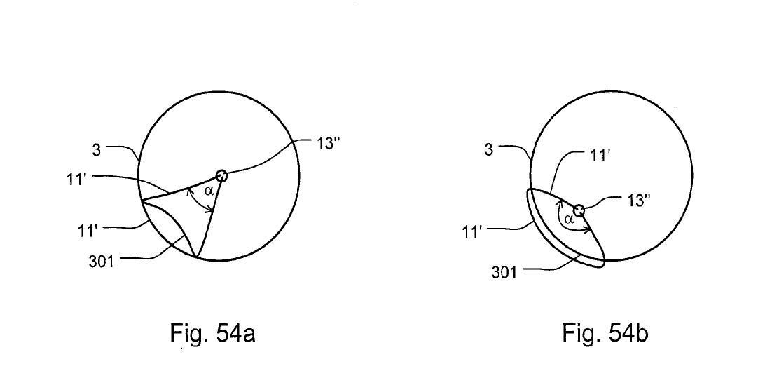

FIG. 54a, 54b show in synopsis an advantage achievable by means of certain embodiments of FIGS. 51a to 53b;

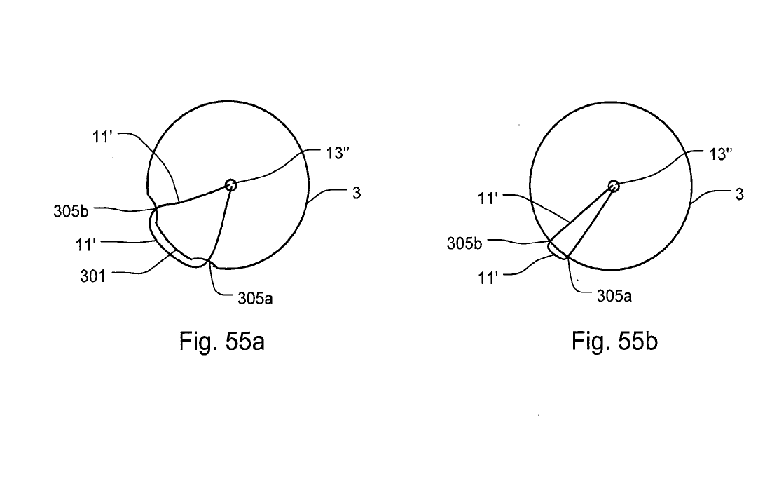

FIG. 55a, 55b show in synopsis a further advantage achievable by means of certain embodiments of FIGS. 51a to 53b.

FIG. 56 shows schematically simplified and in part section an implantation device with an expanded implant;

FIG. 57 shows the implantation device of FIG. 56 with the implant in a further (partly) folded condition;

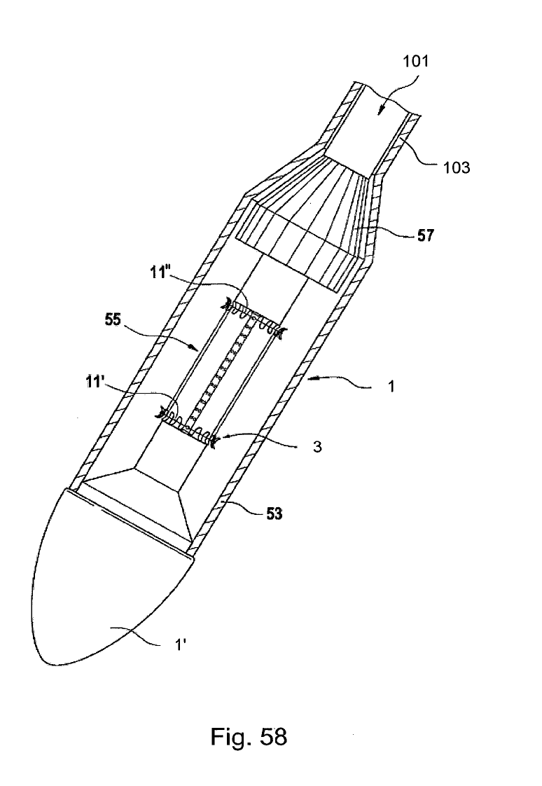

FIG. 58 shows the tip of an implantation device shown in a closed condition prior to implantation;

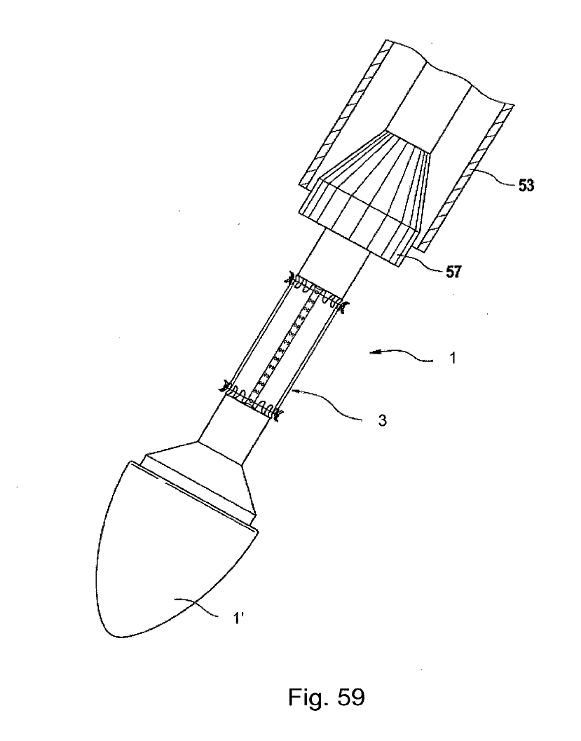

FIG. 59 shows the tip of an implantation device as in FIG. 58 prior to implantation with partially withdrawn outer sleeve;

FIG. 60 shows the tip of an implantation device as in FIG. 59 without implant;

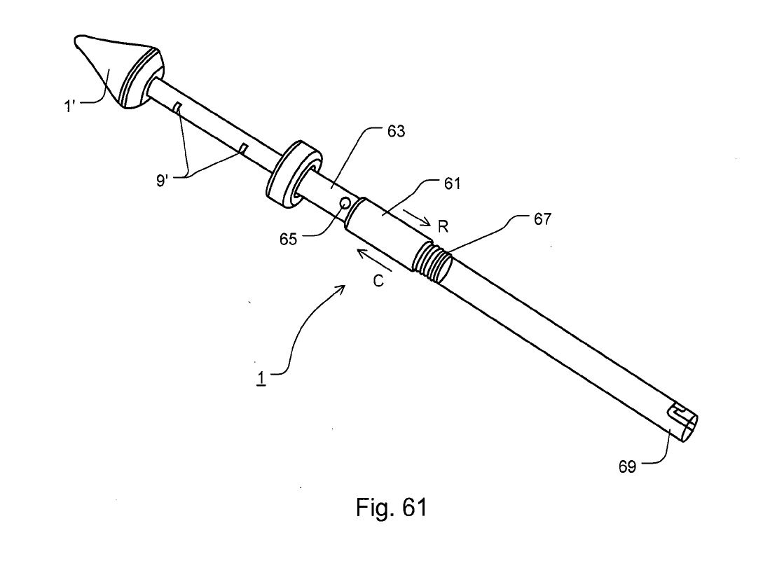

FIG. 61 shows a longitudinal part section through a schematically simplified cutaway view of the implantation device;

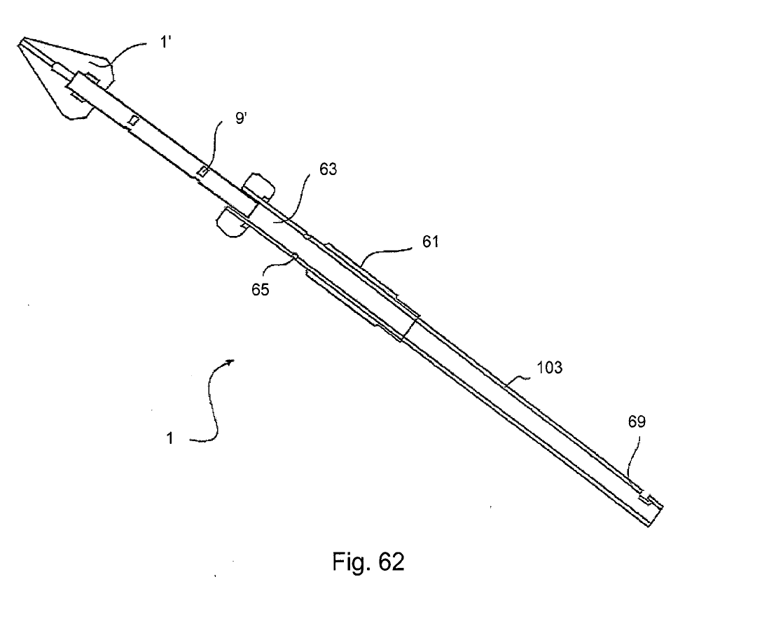

FIG. 62 shows the implantation device of FIG. 61 in a longitudinal section;

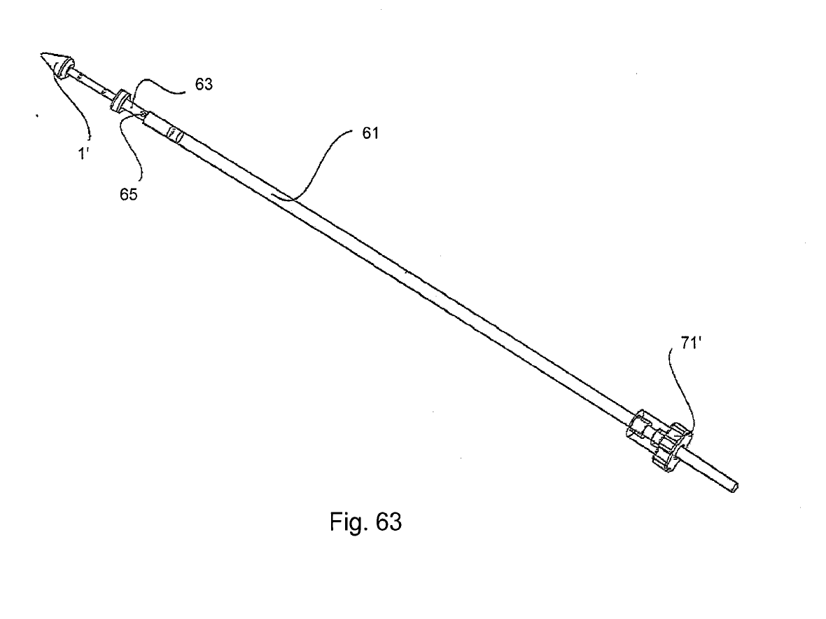

FIG. 63 shows another embodiment of an implantation device;

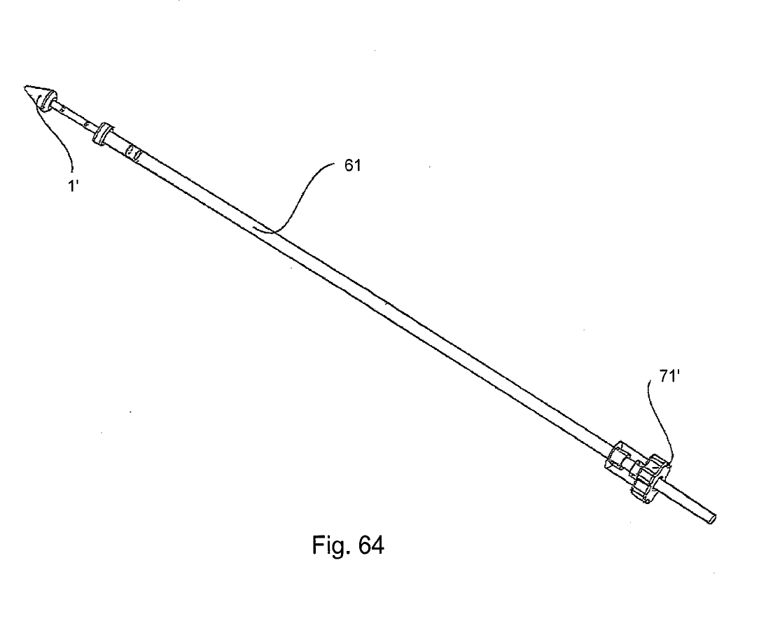

FIG. 64 shows the implantation device of FIG. 63 in a clamping position or state;

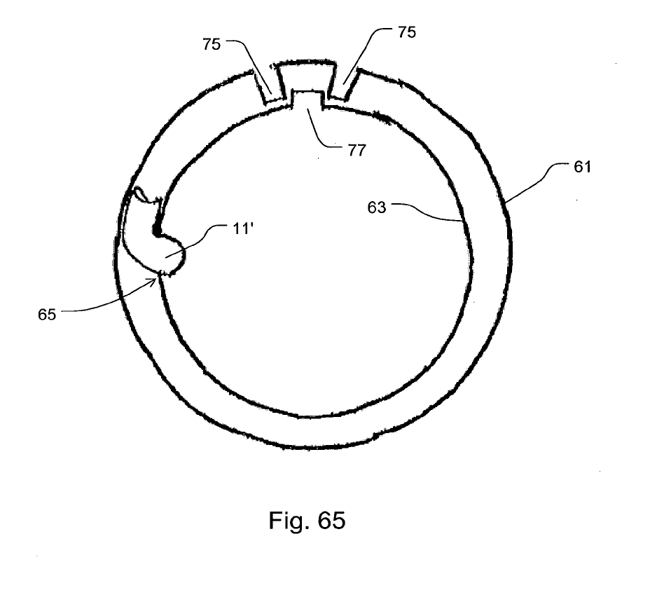

FIG. 65 shows a schematically simplified cross section through the first and the second clamping sections of an implantation device;

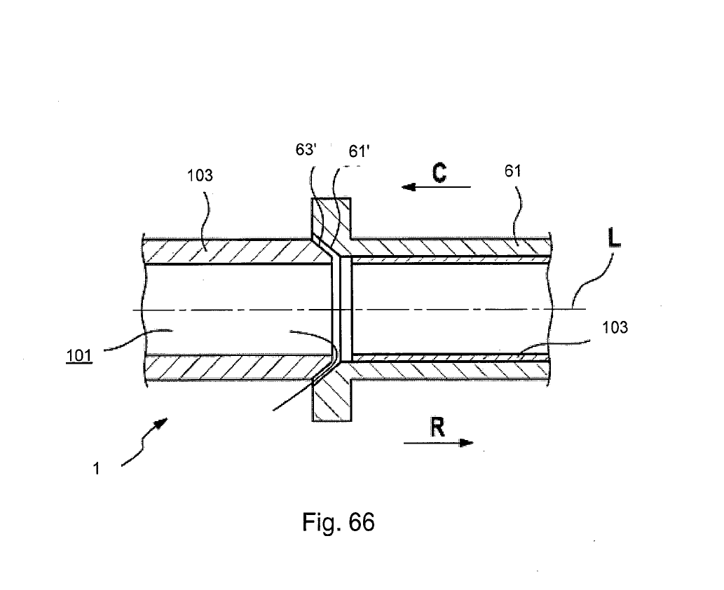

FIG. 66 shows a longitudinal cut, in section, of an implantation device in another embodiment revealing the first and the second clamping sections of that embodiment;

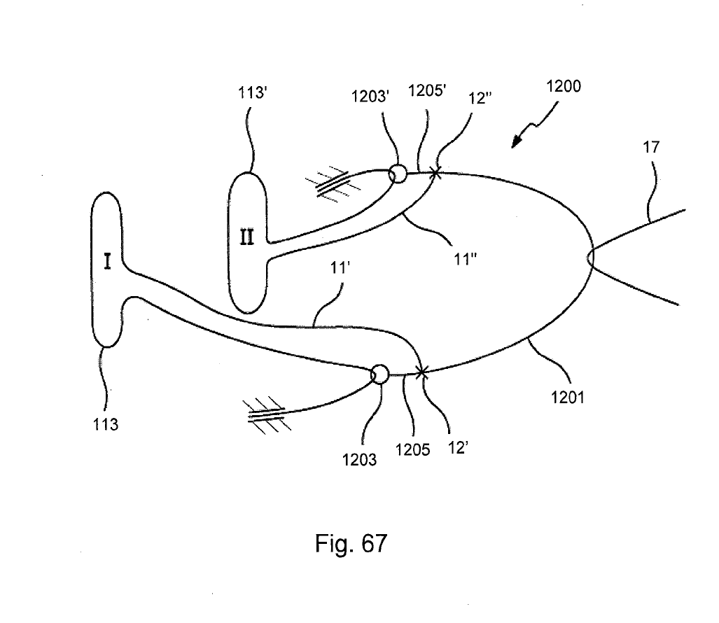

FIG. 67 shows a set of threads for folding an implant;

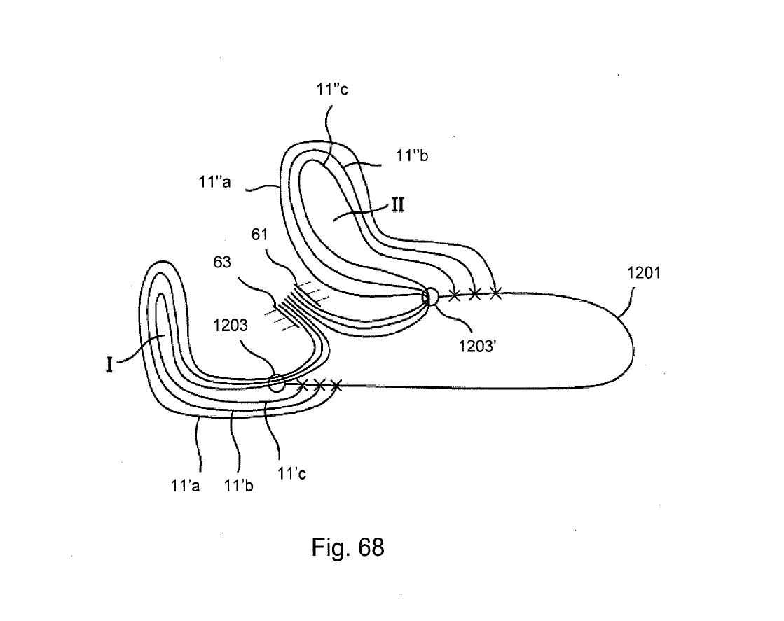

FIG. 68 shows another set in a second embodiment;

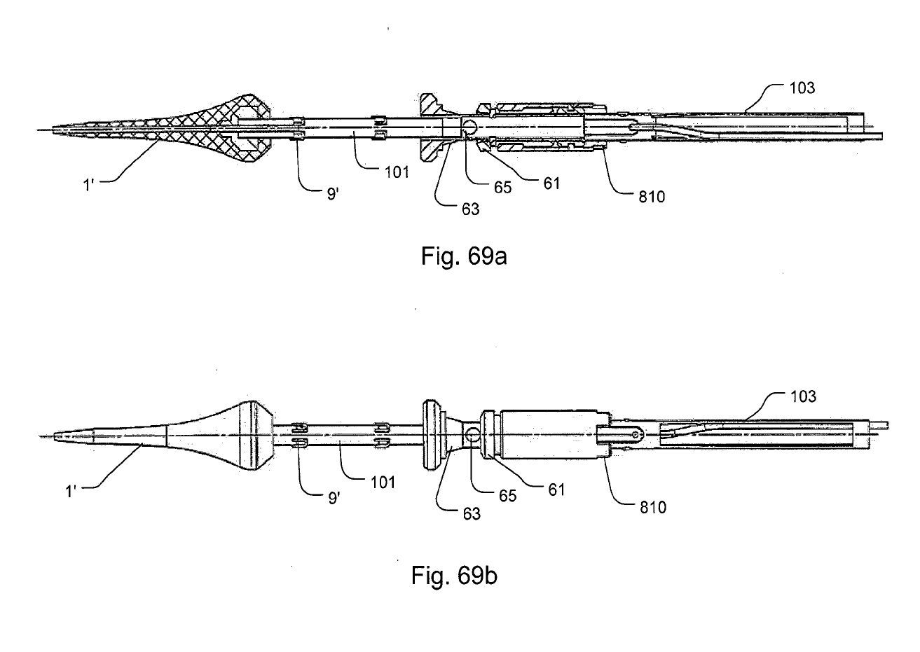

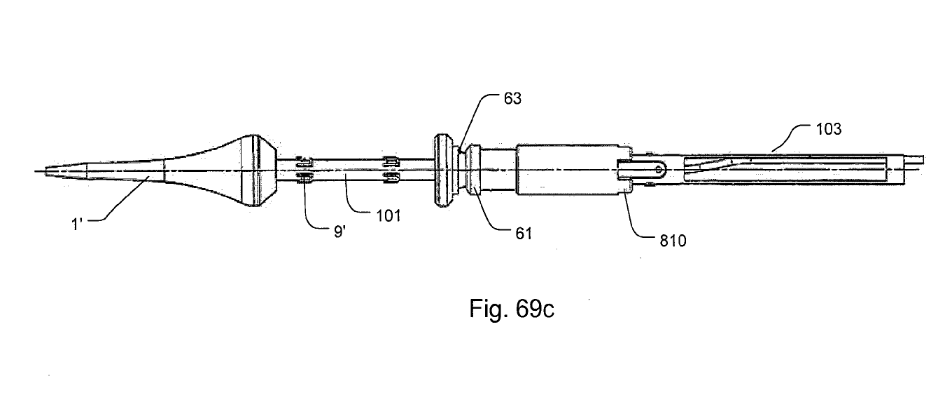

FIG. 69a shows a longitudinally cut tip of an implantation device in yet another embodiment, in an unclamping state revealing the first and the second clamping sections of that embodiment;

FIG. 69b shows the tip of the implantation device of FIG. 69a, not cut;

FIG. 69c shows the tip of the implantation device of FIGS. 69a and 69b in a clamping state;

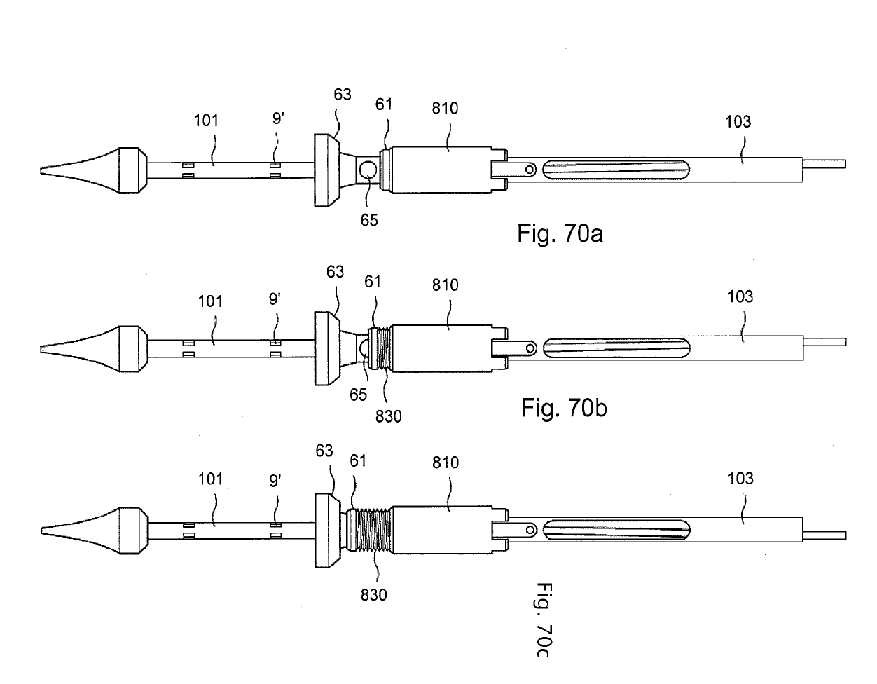

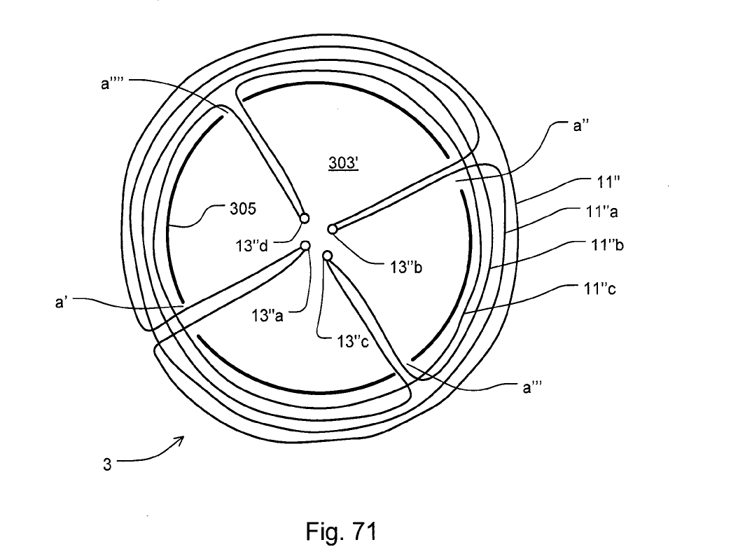

FIG. 70a shows the tip shown in FIGS. 69a-c in a unclamped state;

FIG. 70b shows the tip shown in FIG. 70a in another unclamped state;

FIG. 70c shows the tip shown in FIGS. 70a-b in a clamped state;

FIG. 71 shows a section along the line II-II of FIG. 52a;

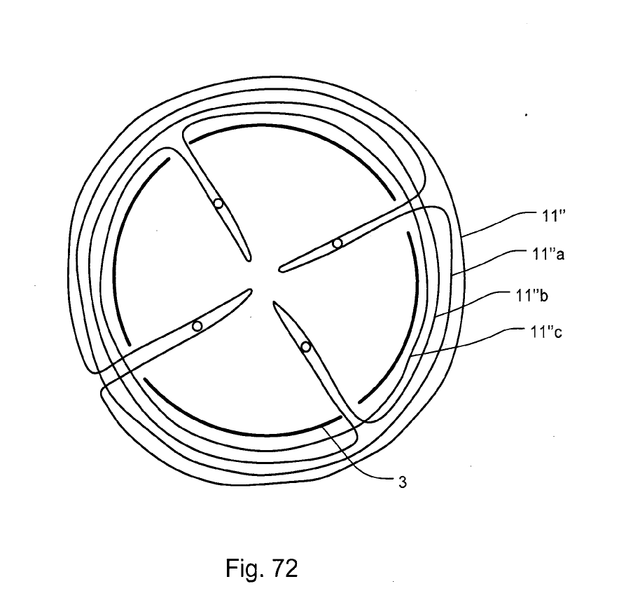

FIG. 72 shows a section along the line of FIG. 53a.

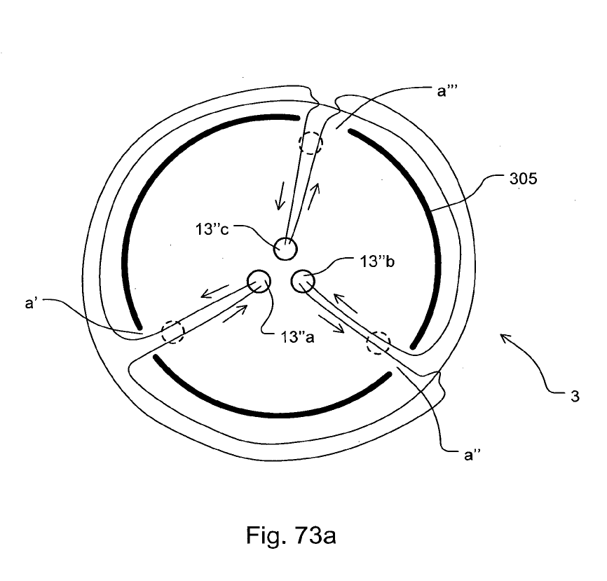

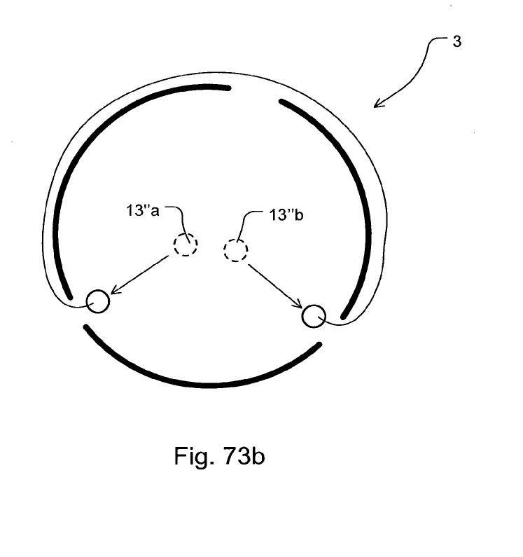

FIG. 73a shows a partial longitudinal section through the implantation device of FIG. 51a similar to FIG. 71, a section of which is shown in a schematically simplified manner, after unfolding the implant, with tension-free tension threads;

FIG. 73b shows part of what is seen in FIG. 73a;

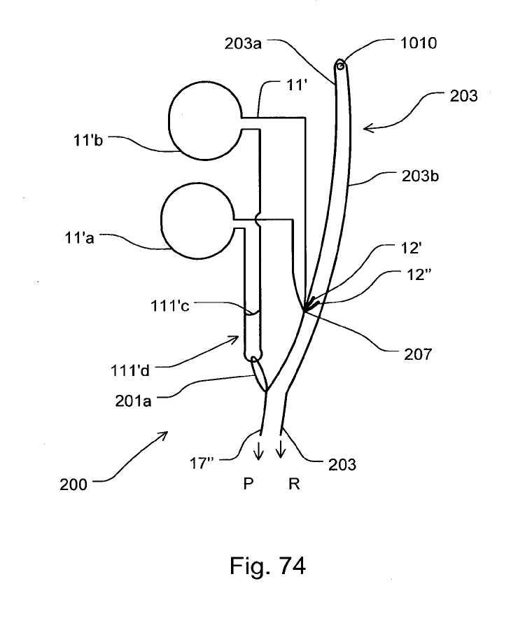

FIG. 74 shows a set of threads of the delivery device comprising a knot;

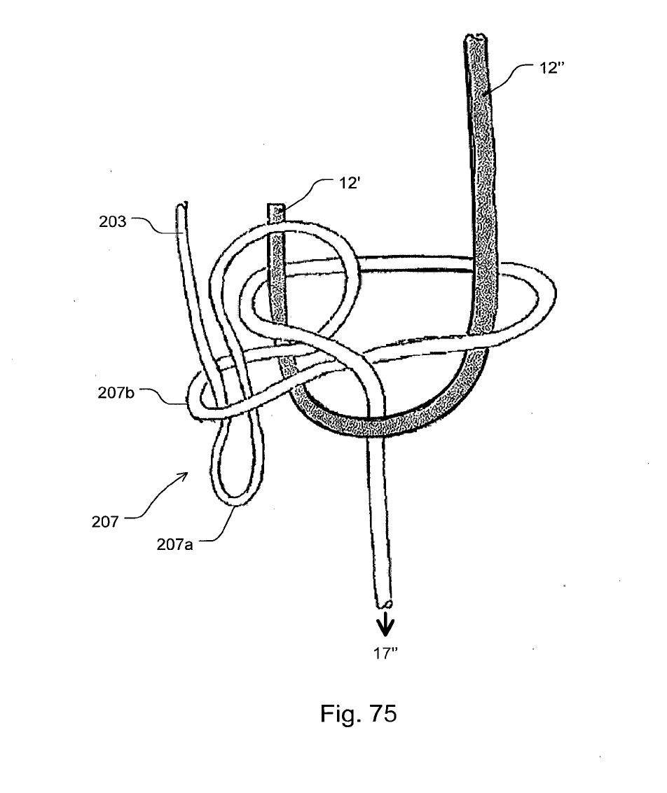

FIG. 75 shows the knot of FIG. 74 in more detail;

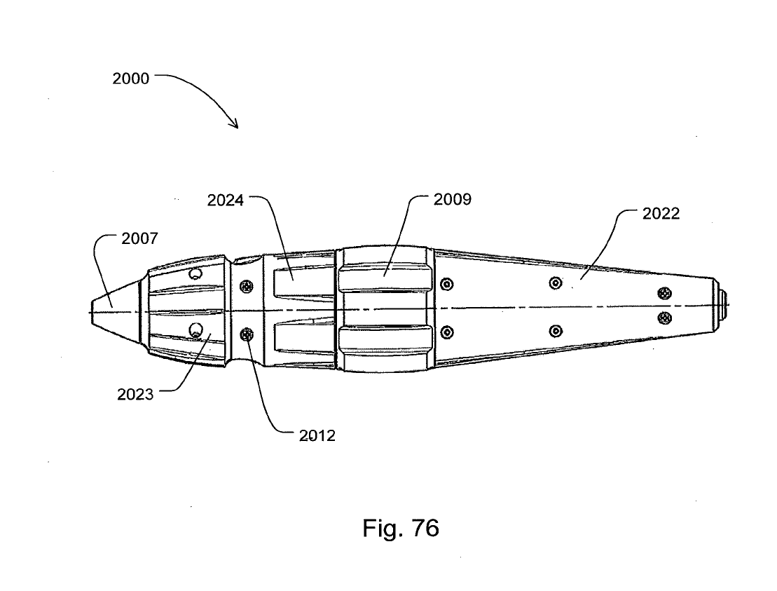

FIG. 76 shows a handle assembly in a side view;

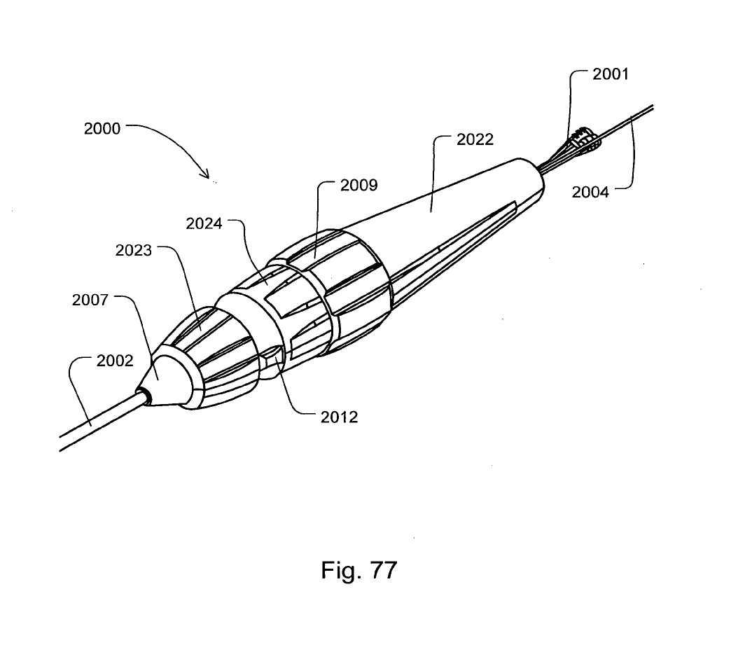

FIG. 77 shows the handle assembly of FIG. 76 in a perspective view;

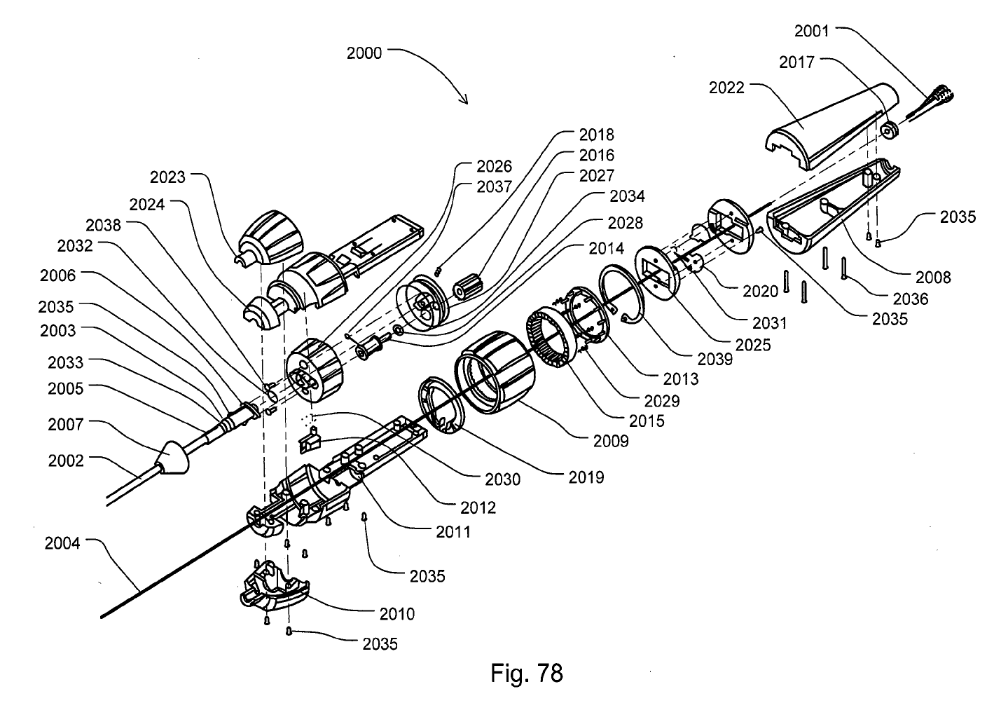

FIG. 78 shows the handle assembly of FIGS. 76 and 77 in an explosion view;

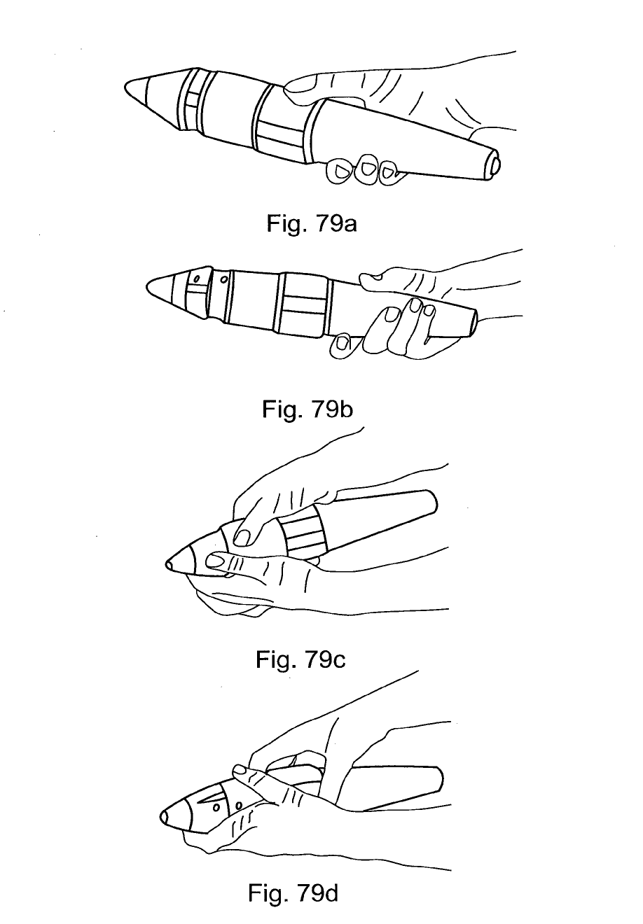

FIGS. 79a-d show different operating modes of the handle of FIGS. 76 to 78;

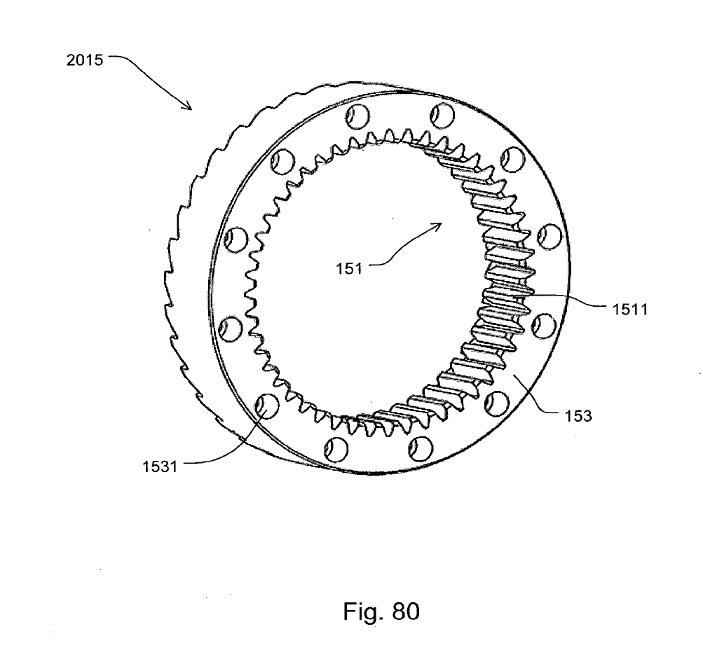

FIG. 80 shows a rush gear as part of the force limiter of the handle assembly of FIG. 76;

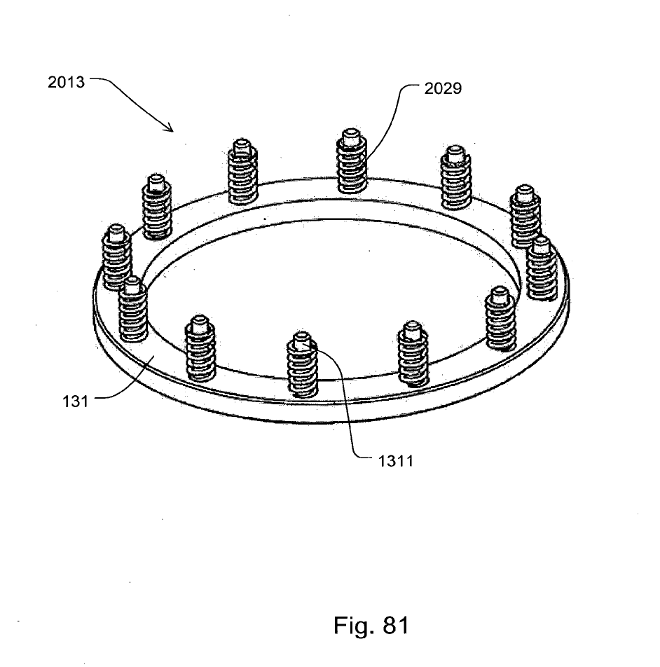

FIG. 81 shows a clutch stopper as part of the force limiter of the handle assembly of FIG. 76;

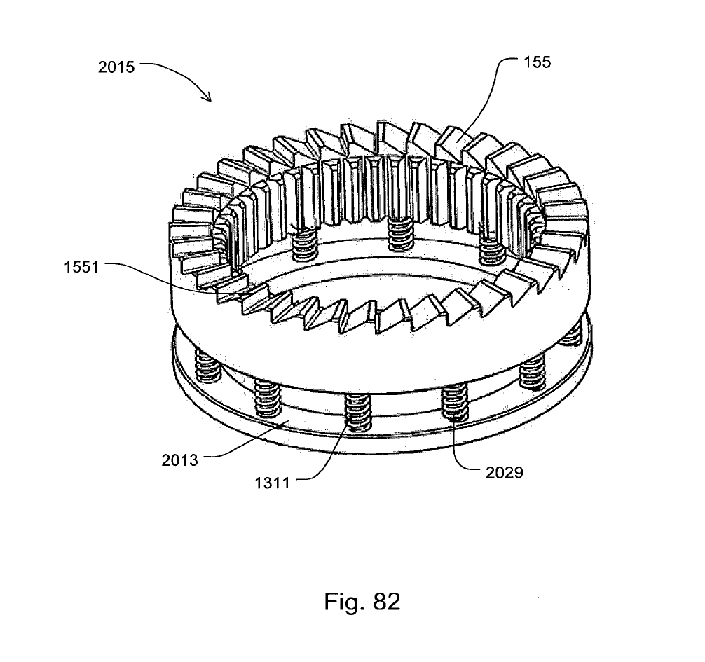

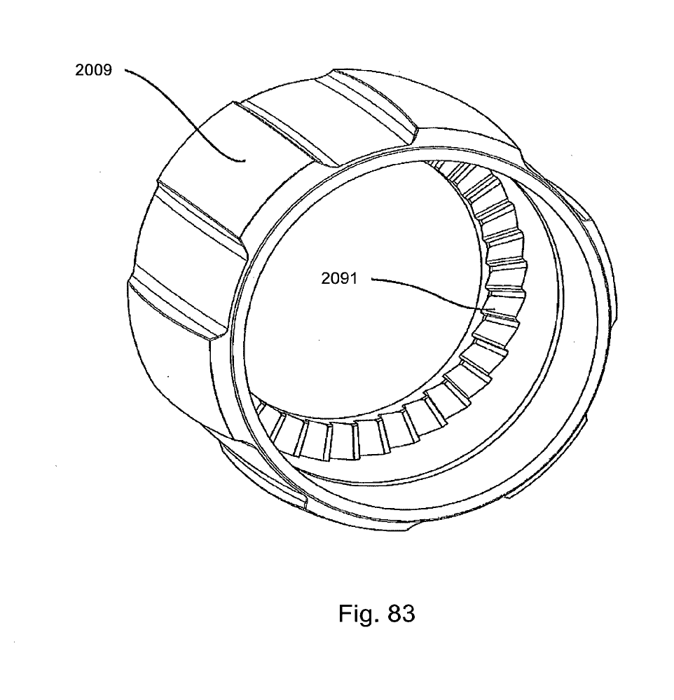

FIG. 82 shows the rush gear of FIG. 80 together with the clutch stopper of FIG. 81;

FIG. 83 shows the rear knob as part of the force limiter of the handle assembly of FIG. 76;

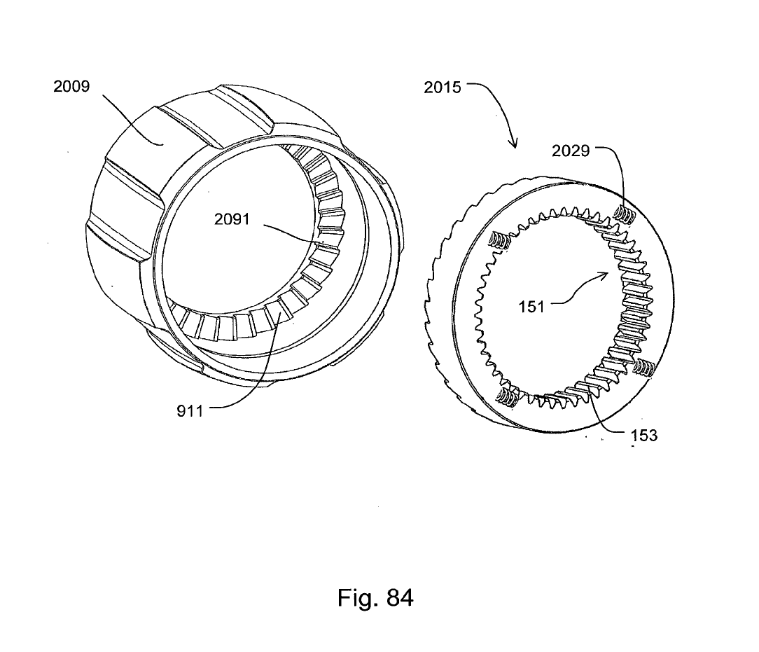

FIG. 84 shows the rear knob and the rush gear of the force limiter of the handle assembly of FIG. 76 in a first perspective view;

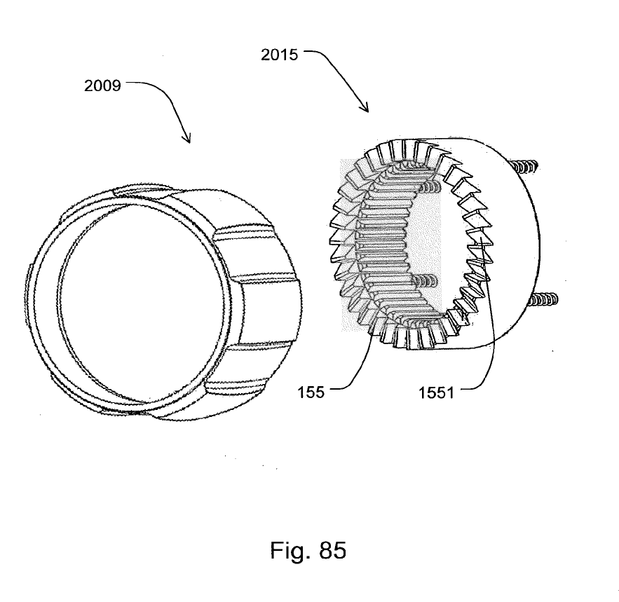

FIG. 85 shows the rear knob and the rush gear of FIG. 84 in a second perspective view;

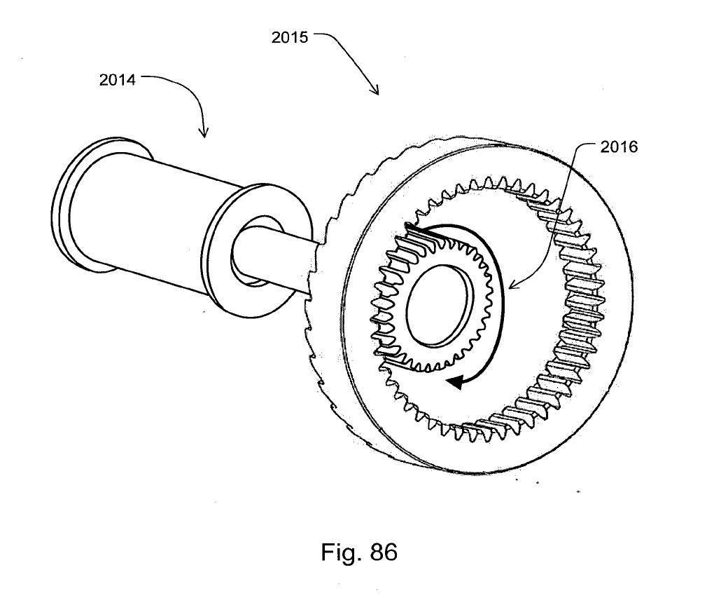

FIG. 86 shows the rush gear of the force limiter of the handle assembly of FIG. 76 in connection with a drum for winding a tension thread in a perspective view;

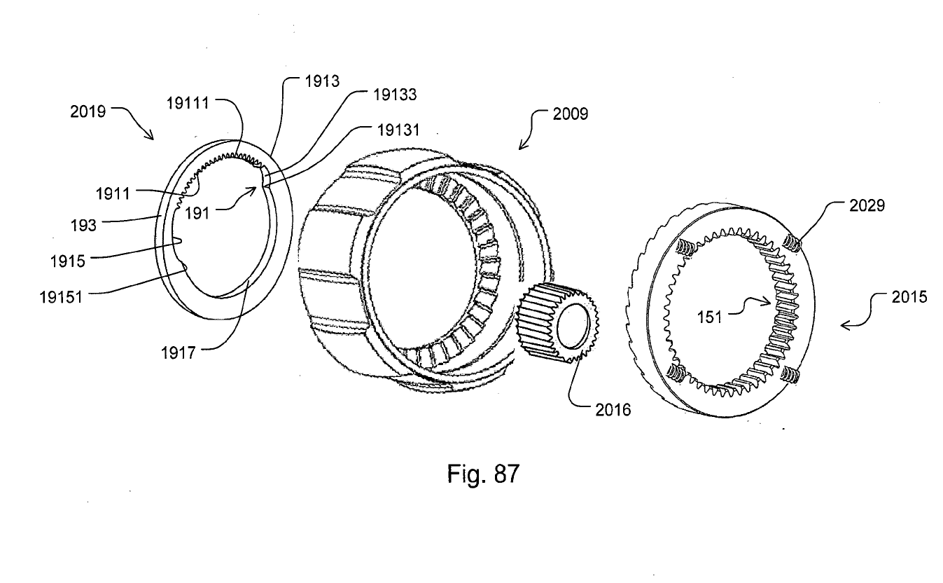

FIG. 87 shows a stopper wheel or gear stopper as part of the displacement limiter of the handle assembly of FIG. 76 in a first embodiment;

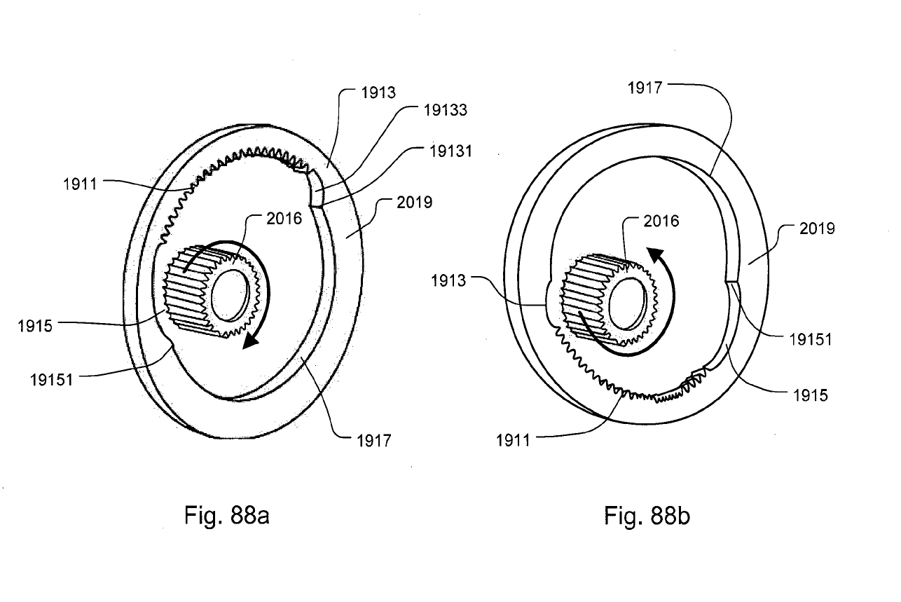

FIG. 88a, b show the stopper wheel of FIG. 87 in engagement with a rush gear demonstrating the function of the stopper wheel;

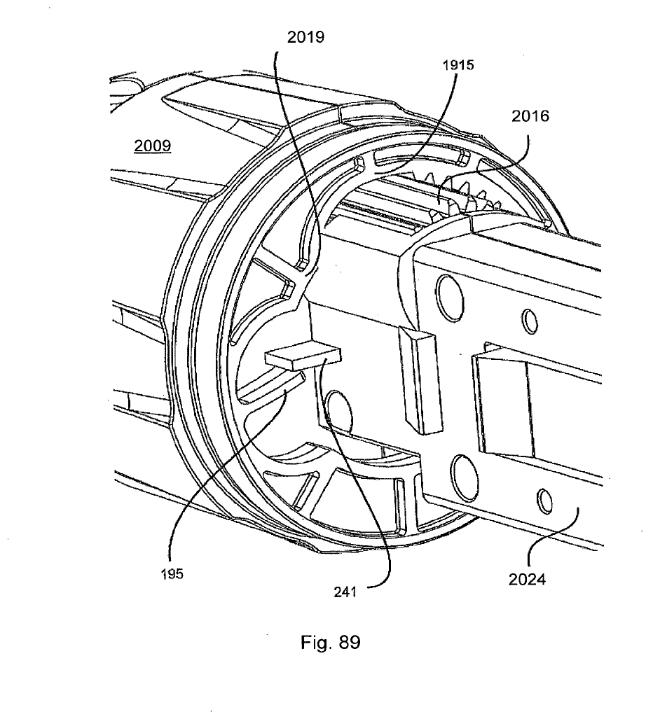

FIG. 89 shows the stopper wheel or gear stopper of FIG. 76 in a second embodiment in a first state;

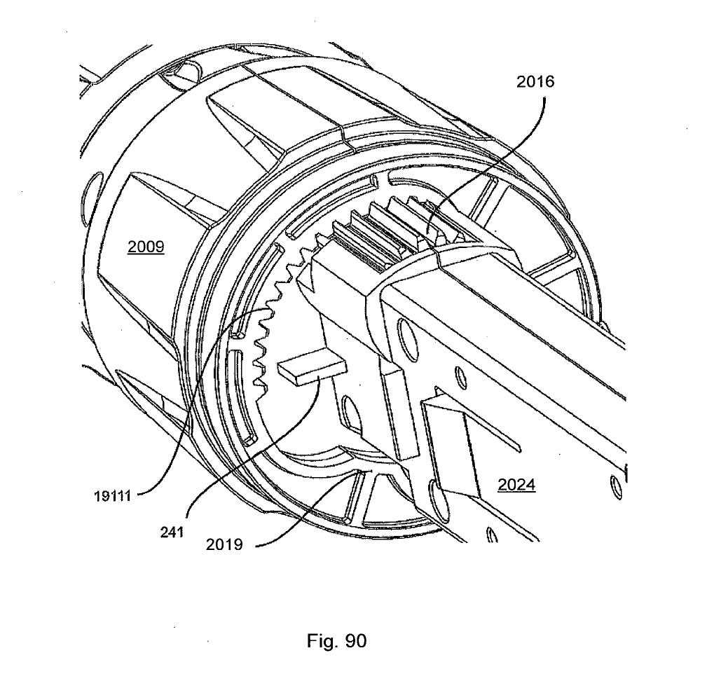

FIG. 90 shows the stopper wheel or gear stopper FIG. 89 in a second state;

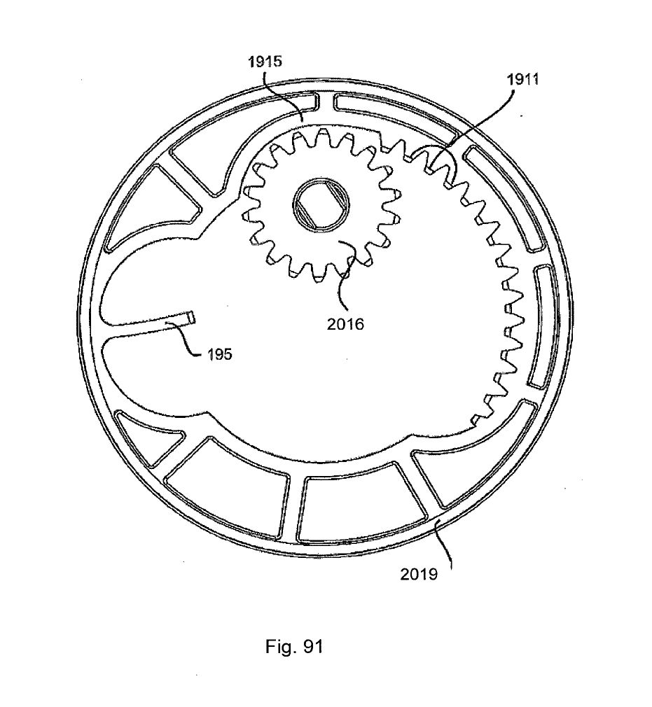

FIG. 91 shows the stopper wheel of FIGS. 89 and 90 in a first plan view;

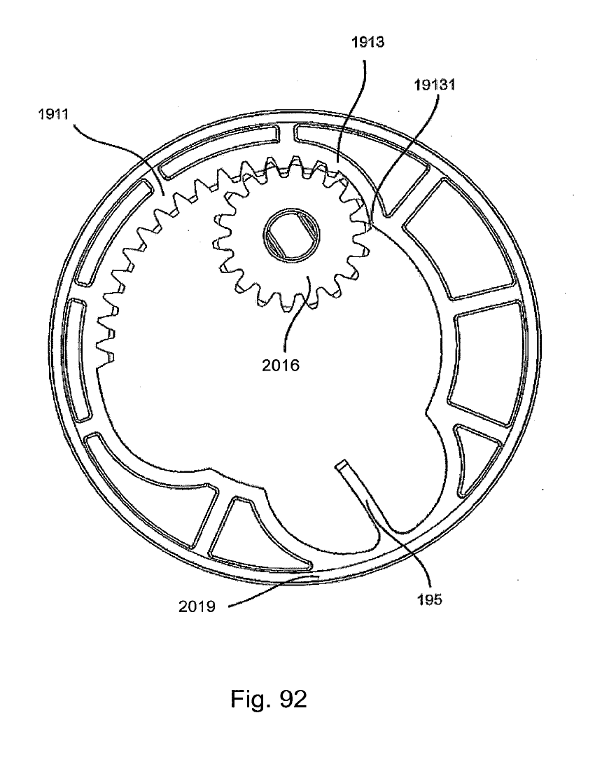

FIG. 92 shows the stopper wheel of FIGS. 89, 90 and 91 in a second plan view;

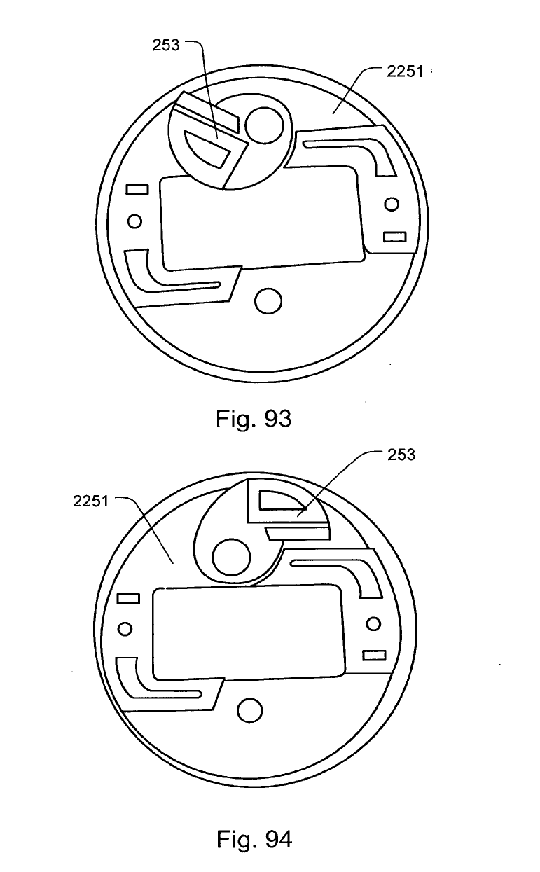

FIG. 93 shows parts of a brake frame assembly of the handle assembly of FIG. 76 in a first state;

FIG. 94 shows the parts of FIG. 93 in a second state;

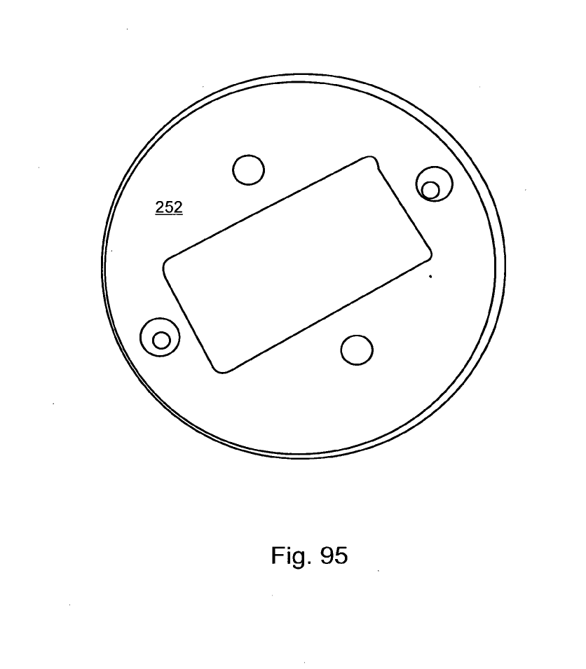

FIG. 95 shows a cover to the parts of FIG. 93;

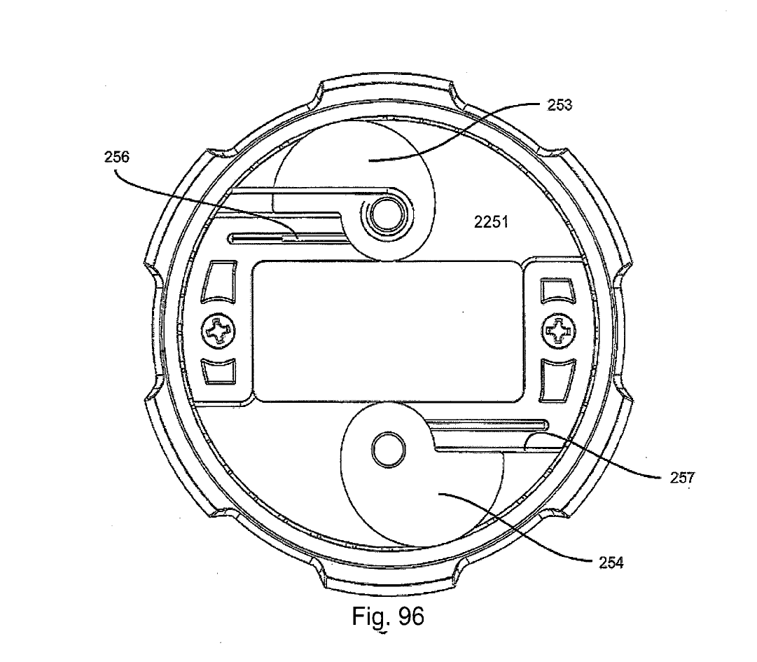

FIG. 96 shows the assembled brake frame assembly, however without a cover;

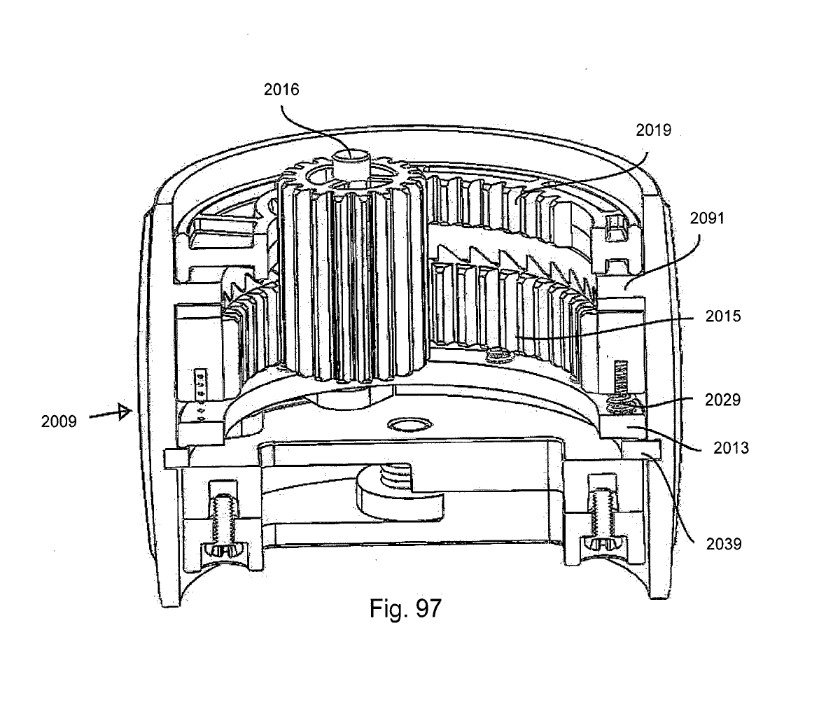

FIG. 97 shows a slightly perspective view of a longitudinal section of the rear knob of the handle assembly according to the present invention;

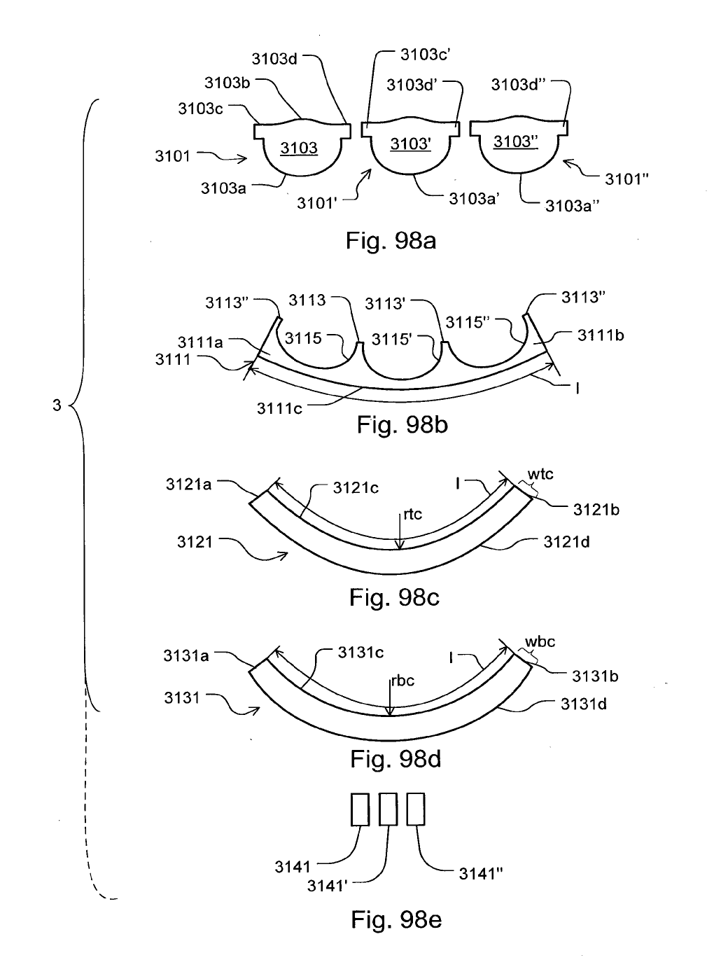

FIG. 98a shows three leaflets of a heart valve according to an exemplary embodiment of the present invention;

FIG. 98b shows a crown piece of a heart valve according to the exemplary embodiment of FIG. 98a;

FIG. 98c shows a top cuff of a heart valve according to the first exemplary embodiment of FIG. 98a;

FIG. 98d shows a bottom cuff of a heart valve according to the first exemplary embodiment of FIG. 98a;

FIG. 98e shows three pledges of a heart valve according to the first exemplary embodiment of FIG. 98a;

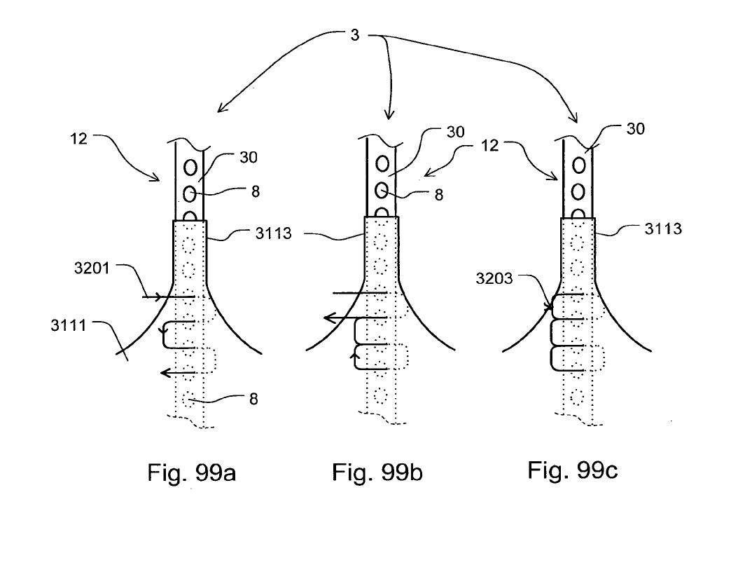

FIGS. 99a-99c show how the heart valve of FIG. 98a to 98d or 98e is being fixed or secured to a frame 55 according to FIG. 56 or 57 or supporting and/or guiding structure 11 according to FIG. 10 or 11;

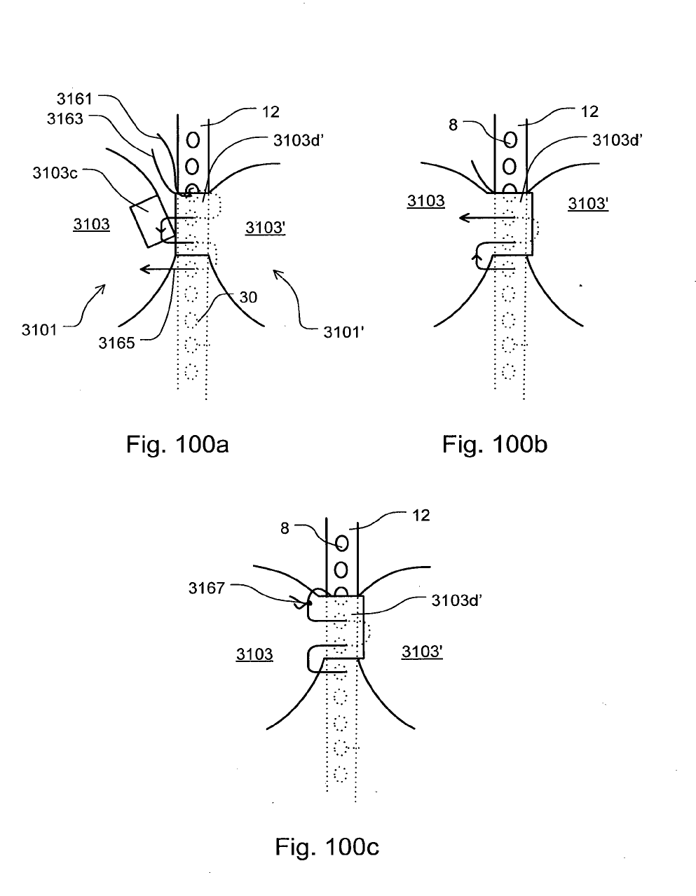

FIGS. 100a-100c show how tabs of adjacent leaflets of an heart valve are commonly attached to one post of the frame or supporting and/or guiding structure;

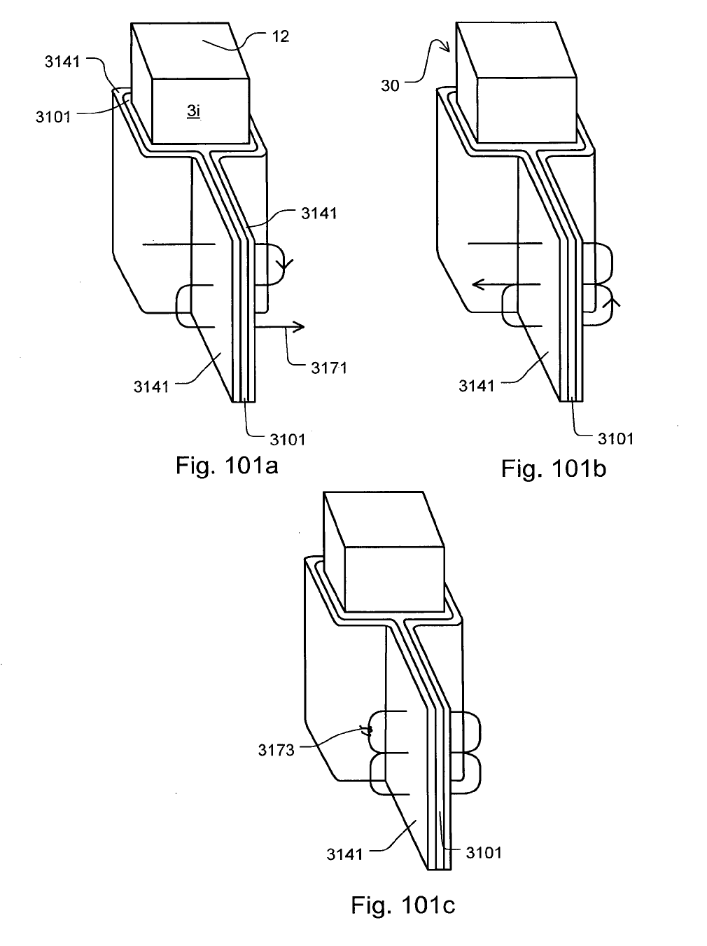

FIGS. 101a-101c show how a pledge is used for reinforcing the connection of the heart valve;

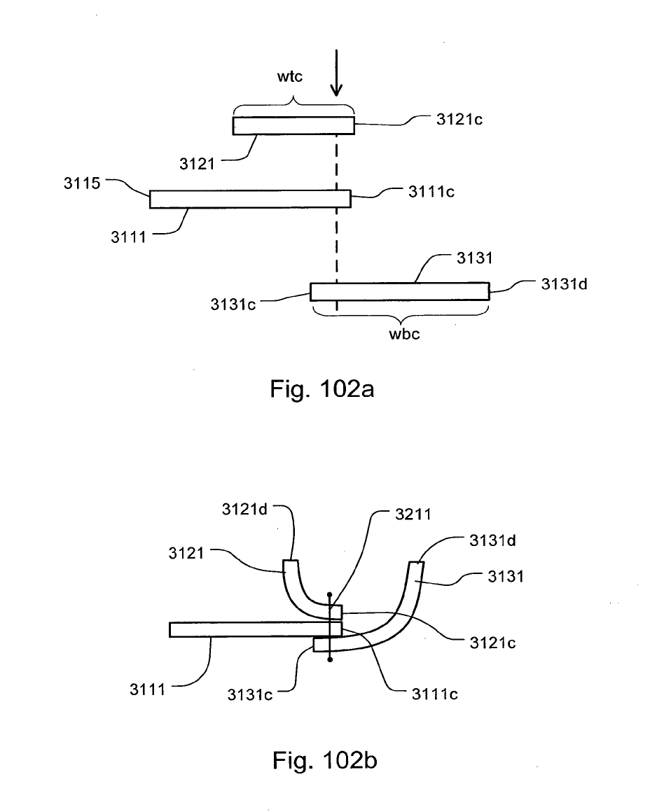

FIGS. 102a-102b show a top cuff and a bottom cuff sewed to a crown piece;

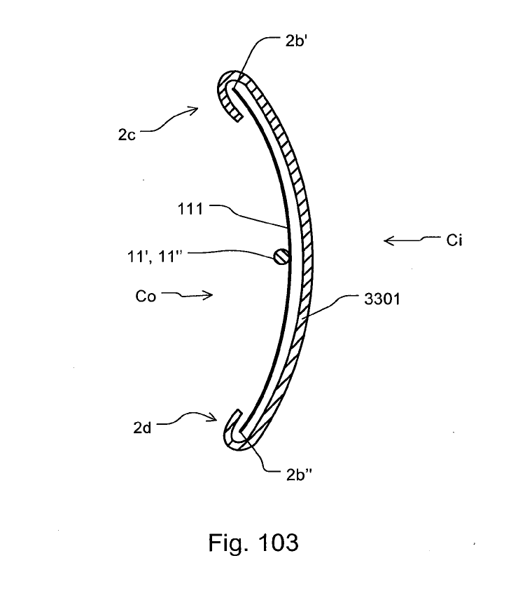

FIG. 103 shows one embodiment of a bar that is shown at the left-hand border of the second guiding structure shown in FIG. 56;

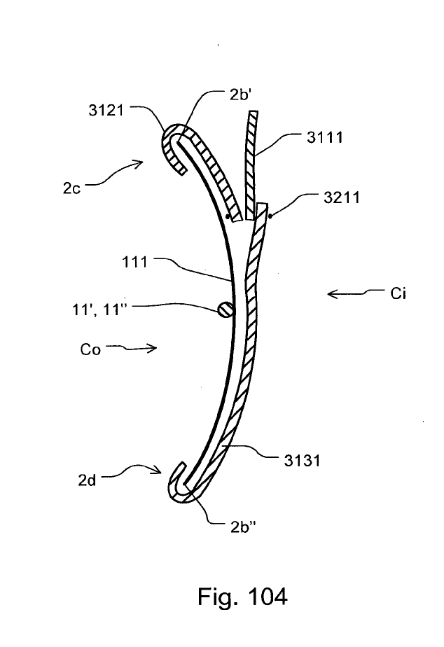

FIG. 104 shows another embodiment of a bar that is shown at the left-hand border of the second guiding structure shown in FIG. 56;

FIG. 105 shows one embodiment of a heart valve assembly (in parts);

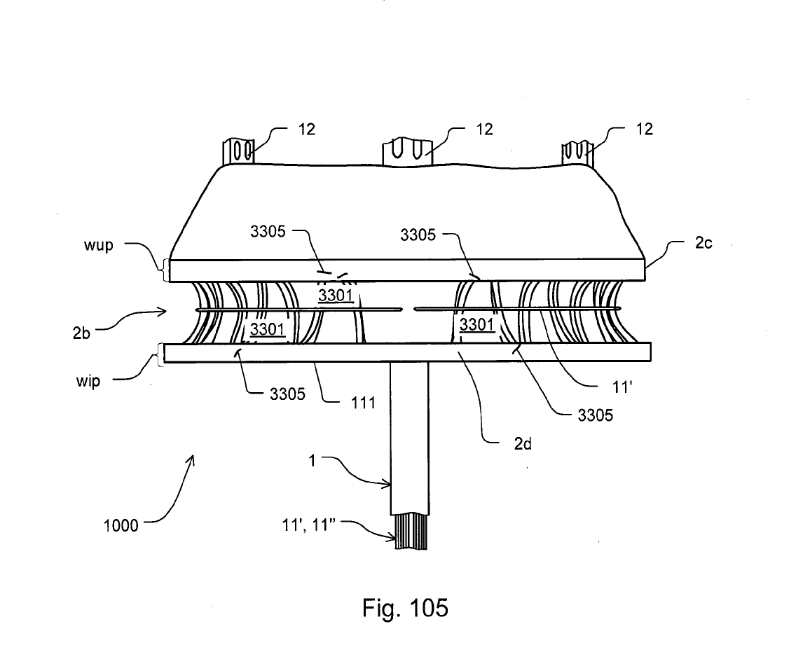

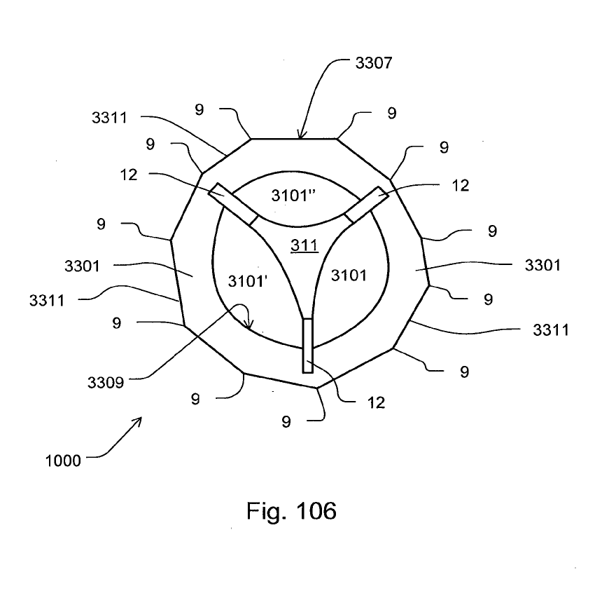

FIG. 106 shows the heart valve assembly of FIG. 105 in a bottom view.

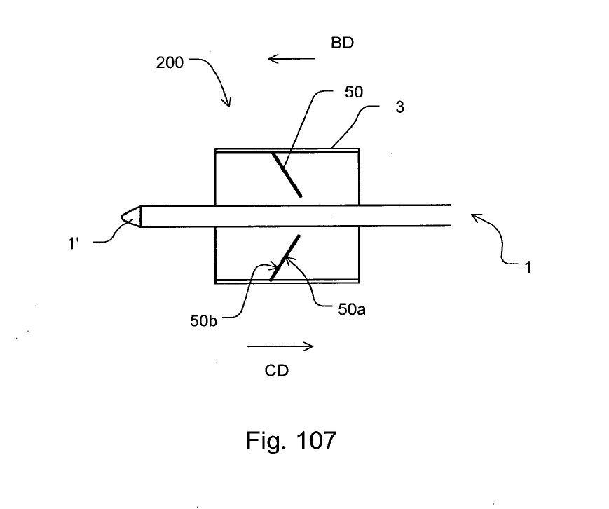

FIG. 107 shows in a schematically simplified view, a set according to the present invention comprising an implantation device, an implant and a check valve;

FIG. 1 shows an implantation device 1 comprising a tension thread 2 which enters into the implantation device 1 in the direction of a longitudinal axis L of the implantation device 1 and leaves or gets out through passage means 4a, 4b and 4c, which can also serve for guiding the tension thread 2 which can, for example, be designed as a thread, on one or several planes or sections of the implantation device 1. The passage means 4 can be symmetrical or asymmetrical. They can be designed round (4b), oval (4a), square (4e) or in any other suited form. The tension thread 2 which in the example of FIG. 1 enters through a longitudinal opening 5 into the implantation device 1, can form a closed loop after leaving or emerging the passage means 4a, wherein the other end of the closed loop re-enters the implantation device 1 at or through the same passage means 4a which hereby serves as an entry means and as an exit means at the same time, and leaves the implantation device 1, for example, through the longitudinal opening 5 of the implantation device 1. Between the tip (at the top of FIG. 1) and the longitudinal opening 5 there is located a void 9 of the implantation device 1 through which the tension thread or tension threads 2 can pass. The void 9 can extend at least from the longitudinal opening 5 to the (in FIG. 1) uppermost passage opening 4a.

Independent of any other feature, i. e., without having to comprise any other feature in combination, the implantation device 1 can comprise a mechanically enforced or reinforced section, in particular in a tip area of the implantation device and in particular in a section which also comprises at least one of the passage means.

FIG. 2 shows the arrangement of FIG. 1, wherein the tension thread 2 has been cut or torn through and can now be pulled back from a stent not shown here and out of the implantation device 1.

FIG. 3 shows in turn the arrangement of FIG. 1 wherein the tension thread 2 is guided in form of a curve 6 around the stent (not shown) and returns through the same passage means 4a into the implantation device 1. The tension thread 2 has no or only little tension at which the stent can be unfolded.

FIG. 4 shows in turn the arrangement of FIG. 1 and in particular that of FIG. 3, wherein the tension thread 2 is tensioned or stressed in FIG. 4. The diameter of the stent (not shown) has been reduced again due to the effect of the curve 6. In case of a foldable stent, the stent is partly or completely re-folded.

FIG. 5 shows an run or extension of three tension threads 2a, 2b and 2c each around ⅓ of the periphery of the stent. Each of these tension threads leaves the implantation device through a passage means 4′, 4″ or 4′″ and returns back through a different passage means.

FIG. 6 shows the run or extension of the tension threads 2a, 2b and 2c around the complete periphery. Each of the tension threads emerges from the implantation device 1 out of a passage means 4′, 4″ or 4′″ and re-enters into the implantation device 1 through the same passage means.

FIG. 7 shows the implantation device 1 of FIG. 5. The tension threads 2a, 2b and 2c leave the implantation device 1 and are guided at around ⅓ of the periphery in a guiding means 11 of the stent 13 (which is hereby exemplified by a half-open channel in form of a C).

FIG. 8 shows the implantation device 11 of FIG. 6 comprising the stent 13 of FIG. 7. The tension threads 2a, 2b and 2c leave the implantation device 1 and are each guided back through the guiding means 11 along the complete circumference or the complete periphery of the stent 13 to the same passage means at or on the implantation device 1.

FIG. 9 shows an expandable stent 13 which is reducable in its diameter (in a plane perpendicular to a longitudinal axis of the stent which substantially corresponds to a laminar flow direction of the blood in an unbent or uncurved, respectively, vessel into which the stent has been inserted; the longitudinal direction also corresponds to the direction of the largest spatial extension of the stent before its unfolding outside the patient's body as well as after its unfolding in a comparable linear vessel section) again by the use of a means according to the invention which is not shown here. This stent 13 comprises two circular guiding means 11 each in form of a channel half-open to the outside—that can also be open to the stent—and two passage means 10 in form of round passages (thereby, the passage means 10 can also in any embodiment and independent of any other feature be designed non-round and can be present alone, in pairs, in a threesome or in a multi-some). Furthermore, the stent 13 can also comprise a number of guiding means 11 other than two, for example, one, three, four or more. The guiding means 11 can be arranged circularly, they can, however, also be provided in a non-circular manner. The guiding means 11 can be formed integrally with the stent, they can, however, also be fabricated separately. The passage means 10 can likewise be formed integrally with the stent or likewise be fabricated separately. The guiding means 11 can be designed in wave form, they can, however, also be fabricated in any other form, in particular in a non-wavy form. The passage means 10 are arranged in sections of rods. They can be arranged at opposite ends of the rods, but also in every other section, for example, in a central area or section and not at the end of the rods. They can furthermore be arranged at a position of the stent 13 other than in or at the rods. Tension threads not shown here can be guided from an interior of the stent 13 through the passage means 10 to the outside and back again.

As an example of an implant, the stent 13 can comprise an arbitrary number of rods which are all designed in the same way or have at least two different designs. The rods can thereby be spaced apart from each other with the same distance. They can, however, also be arranged with at least two different distances from each other, respectively. The rods can indeed comprise the passage openings 10, the latter can, however, also be provided separately from the rods. Likewise, the rods can have openings, they can, however, also be designed without any passage openings. The stent 13 can be designed having rods which do not have any passage openings 10. The stent can further comprise at least one rod having passage openings and at least one rod not having any passage openings. The stent can comprise at least one rod which does not have any passage openings at all. The stent can comprise at least one rod which is arranged in or at the stent in a manner in which it is inclined to the longitudinal direction of the stent. The rods can thereby extend in a manner in which they are bent or curved at both ends to the outside. Regardless of any other features, they can, however, extend such that they are not or at least not at both ends curved or bent to the outside. The rods can be connected with or at their both ends to a wavy structure of the stent. Regardless of any other features, they can, however, not or at least not at both ends be connected to wavy structures.

Independent of any other feature, the stent 13 can be manufactured from flat material, for example, a material which has been cut with a laser, wherein, e.g., after having designed a pattern in the flat material, the material is reformed into a tube (optionally by connecting, such as welding, longitudinal sides of the former flat material lane or web, respectively). However, the stent 13 can also be fabricated from a tubular material directly.

FIG. 10 shows the stent 13 of FIG. 9. Two tension threads 2 have been guided around the stent 13 and return back to the implantation device 1 through the same guiding means 10, respectively. The tension threads 2 exert no or only a small tension or stress on the stent 13 and the stent 13 is unfolded.

In FIG. 11, the tension threads 2 are tensioned or stressed. The diameter of the stent 13 has been reduced. The stent 13 is re-folded at a larger extent. The tension threads 2 are guided in the guiding means 11 of the stent 13.

FIG. 12 and FIG. 13 show a stent 13 in a representation corresponding to the representation of FIG. 9 and FIG. 10, respectively. Thereby, FIG. 9 shows the stent 13 in an expanded state, FIG. 10 shows the stent 13 with a reduced diameter again. The stent shown in FIG. 9 and FIG. 10 can thereby correspond to any stent known (with or without having valves). The stent 13 can in particular correspond to any unfoldable stent known.

In addition, in a schematically simplified manner, FIG. 12 shows a control unit for unfolding or expanding and in turn folding or reducing the stent 13 in its diameter in a controlled manner.

FIG. 14 shows that the tension threads 2 can also be guided in a spiral form around the stent 13. Here, only one tension thread 2 is shown which is guided in the interior of the stent 13 to the section in the front thereof. Then, the tension thread 2 is guided over the stent 13 at the outside and re-enters the implantation device 1 (not shown here) again. The tension thread 2 is not or only a little tensioned or stressed and the stent (not shown here) is unfolded.

In FIG. 15, the tension thread 2 is pulled up at both ends in the arrow direction and the diameter of the curve 6 of the spirally guided tension thread 2 has been reduced.

FIG. 16 shows the state of the tension thread 2 of FIG. 15. The tension thread 2 (which is here also referred to as a thread) has been tensioned or stressed and the stent 13 has been folded or collapsed by use of the spirally guided tension thread 2.

FIG. 17 shows an implantation device 1 according to the invention comprising a plurality of lumina or channels 23 for guiding through tension threads (not shown in FIG. 17) in a cross-section thereof. These channels 23 for the tension threads are designated by the letters B, C, D, E, F, G and H.

The lumina can be suited and prepared for guiding through tension threads for using the implantation device.

The implantation device 1 has a further lumen 25 in its center which is designated by the letter A and which is, in the embodiment shown, provided for receiving a means, such as a guiding wire, which is likewise not shown in FIG. 17.

The further lumen 25 which is shown in the center of the cross-section of implantation device 1 in FIG. 17 is not limited to this arrangement. (One or more) lumen/lumina or channel(s) can also be arranged at the edge of the cross-section; two of them can be opposite to each other due to handling reasons, etc. Moreover, the implantation device can comprise more than one lumen 25 for one or several further means, respectively.

The further means can be a means other than a tension thread and/or not assuming a function in changing the diameter of the implant.

The further means can be a means for cutting or tearing through the tension threads.

FIG. 18 shows an implant 3, viewed from the side, during implantation. The implant 3 is still connected with the tip 1′ of an implantation device 1. As can be seen from FIG. 18, the implant 3 has a first structural element embodied as proximal ring 11a and a second structural element embodied as distal ring 11b.

The proximal ring 11a and the distal ring 11b are interconnected with each other by means of three interconnecting elements which are embodied in the implant 3 of FIG. 18 by way of example as posts 12.

As can further be seen from FIG. 18, the posts 12 each comprise two circular apertures 14 (which may have any other shape such as elliptic, oval, rectangular, and the like), through which strings 15a and 15b are routed form an inner space of the implant 3 in the centre of which the tip 1′ of the implantation device 1 is placed to an outside of the implant 3 for controlling the expansion and re-folding of the implant 3 as is explained in great detail in WO 2008/029296 A2 (“Minimally invasive heart valve replacement”, filed on Feb. 15, 2007) to the inventors of the present invention. For further general details on the implant and the catheter or implantation device it is referred to that document, the respective disclosure of which is herewith incorporated by way of reference.

Strings 15a and 15b are directed to an inside of the implantation device 1, which inside of the implantation device 1 the strings 15a and 15b leave opposite its tip 1′ as is shown at the lower part 17 of the implantation device 1.

Due to the central position of implantation device 1 within the inner space of the implant 3, in the representation of FIG. 18 the implantation device 1 may be seen as representing the longitudinal axis 19 of the implant 3. In the particular embodiment of FIG. 18 (and also in those of FIG. 19 and FIG. 20) the posts 12 extend in a plane that is parallel to another plane encompassing the longitudinal axis 19 of both the implantation device 1 and the implant 3.

Posts 12 comprise a number of apertures 14′, arranged in two parallel rows extending in a longitudinal direction of the implant 3. As is explained in WO 2008/029296 A2 in detail, the apertures 14′ may be used for passing chords or ties through the posts 12 to secure lateral edges of the leaflets in place with the interior of the implant 3 to create a working valve, for example. It has to be noted that according to the present invention, one row of apertures 14′ (of any shape and size thereof) is also contemplated. Having one row instead of two rows advantageously allows for designing posts having a smaller width. A smaller width of the post 12 allows in turn that the implant can be designed to be more open, even more flexible, that more space is left for the functionally effective part of the implant and the like.

As has been said above, FIG. 18 shows how one particular embodiment of the implant according to the invention may look like seen from the side. It is, however, to be noted that due to the perspective chosen, FIG. 18 does not show the particularities of the present invention. Those can be seen from FIGS. 19 and 18.

FIG. 19 shows an implant 3 according to the invention, partly cut, crimped on a tip 1′ of an implantation device 1. For the sake of enhanced readability, strings are omitted in FIG. 19. Implant 3 contacts an outer surface 230 of the tip 1′ of the implantation device 1 at a first portion and a second porting both of which cannot be seen in FIG. 19 but in FIG. 20. What can be seen from FIG. 19, however, is the fact that the posts 12—being the interconnecting elements—comprise a third portion 24 each that is more radially arranged compared to the first and second portions at which the distal and proximal rings 11a, 11b, contact the implantation device 1.

As can be seen from FIG. 19, due to the positioning of the ends 12a and 12b of the posts 12 on the distal and proximal rings 11a, 11b, in the crimped state of the implant 3 as shown in FIG. 19, there remains a first gap d1 between each post 12 and the outer surface 230.

Similarly, due to a fourth portion 250 of the distal and proximal rings 11a, 11b, a sleeve indicated with reference numeral 27 without being actually shown in FIG. 19, can be provided on the implant without applying undesired pressure on the implant or, more important, flexible structures thereof such as heart valve leaflets.

Such leaflets, mentioned here by way of example, find enough space or room underneath the sleeve 27 between sleeve 27 and implantation device 1. That room may be provided by first gap d1 and/or by second gap d2.

FIG. 20 shows a schematic illustration of the implant 3 according to the invention in a longitudinal section. Although implant 3 has only been reproduced in the upper half of FIG. 20, for symmetry reasons a mirrored representation of the implant 3 should also be found in the lower half of FIG. 20. The missing part of the implant 3 has only been omitted as it comprised no additional information.

In contrast to FIG. 19, in FIG. 20 strings 15a and 15b are depicted in cross section each. Also, in FIG. 20, first portion 31 and second portion 33 are shown.

FIG. 21 shows a part longitudinal section through a part-sectioned, schematically simplified view of the folding device 100 according to the invention. The folding device 100 has a shaft 101 with wall 103. Shaft 101 is in the upper area 1a of FIG. 21 shown not sectioned and in the lower area 1b longitudinally sectioned in such a way that an open view of the interior 105 of shaft 101 is possible.

The wall 103 separates the interior 105 of shaft 101 from an exterior 107 of shaft 101, the exterior 107 of shaft 101 can be an outer part of folding device 100 (so an external layer). The shaft 101 can however still be surrounded by a further structure (not shown in FIG. 21).

Shaft 101 features apertures 9′. In FIG. 21, 6 such shaft apertures 9′ are shown. This quantity is purely an example to assist explanation.

The shaft apertures 9′ can thereby be evenly spaced from each other around the circumference of shaft 101. They can be divided with at least 2 different distances from each other around the circumference.

The shaft apertures 9′ can as shown in FIG. 21 pass through the total thickness of wall 103 of shaft 101 so be developed as connecting openings.

Through the shaft apertures, a tension thread or several tension threads 11″ and 11′ can be threaded from the interior 105 of shaft 101 to the exterior 107 of shaft 101 and/or threaded-in from the opposite direction. In FIG. 21 all the threads 11″ and 11′ pass both in and out of the shaft apertures 9′ in loop-form.

The threads 11″ and 11′ are arranged to hold an implant not shown in FIG. 21 so that the implant diameter can be altered through varied tension in the threads 11″ and 11′.

As can from the sectioned lower area 1b of the shaft 101 in FIG. 21 be understood and with reference to FIG. 22 becomes clearer, a number of threads 11′ are collected together to a first bundle 130 and the other threads 11″ to bundle 150. In the bundle, the threads can be provided to be effected or pulled separately from each other.

In FIG. 21 as an example the upper 3 threads 11′ are grouped together in bundle 130 while the lower 3 threads 11″ are grouped together in a second bundle 150. This arrangement is purely for example and can be defined in any other order.

In a lower area of shaft 101, bundles 130 and 150 are joined together.

In a lower area of shaft 101, a pulling thread 170 loops over bundles 130 and 150 for being engaged with them such as to be capable to transmit a pulling force on threads 130 and 150.

By virtue of the sliding connection of bundles 130 and 150 to pulling thread 170, force in the direction of the lower area of FIG. 21, can bring to bear tension or force on bundle 130 or bundle 150. Thereby can increased tension be brought to bear on bundle 130 when tension on bundle 150 can be allowed to increase no further (or reduce of course). Likewise the tension on the first bundle 130 can be reduced when the pulling thread 170 is brought back in the direction of upper end of FIG. 21, also when by means of the pulling thread 170 no more tension can be brought to bear on (or reduced from the second bundle 150. That is, usually force or tension on bundle 130 and bundle 150 are the same at each particular point of time. It is always the same force on both bundle 130 and 150, only when there is less resistance on one side, this side will elongate, until forces become the same again. The same situation applies during pulling. Even when bundle 130 and bundle 150 have different lengths, or different pulling distance, the force and tension should be always balanced, i.e. the same on both sides.

This effect of independently operated bundles (here bundles 130 and 150) together with the invention here presented will be designated as “self balancing design”. It is achievable through the special connection of bundles 130 and 150 with the pulling thread 170 with a free through movement of bundles 130 and 150 which is enabled by the loop design of pulling thread 170. This connection allows a sliding movement of bundles 130 and 150 through the loop of the pulling thread 170 in both the directions indicated by arrow P.

The tension of the pulling thread 170 is in turn adjustable by means of a rotation or tensioning device.

It is clear that this “self balancing design” is not limited to two bundles which can moreover be developed as single threads and not to a further thread—here the pulling thread 170.

Tension or pull exerted by means of the pulling thread 170 will be carried through to the threads 11″ and 11′ by means of bundles 130 and 150. In this manner an operation of the pulling thread 170 can bring about a change in one or more cross-section dimensions of the implant not shown in FIG. 21.

FIG. 22 shows the object in FIG. 21. In FIG. 22 shaft 101 is actually sectioned or cut open over the complete length shown. There is no difference shown between the two areas 1a and 1b, Hence in FIG. 22 only 4 of the 6 thread loops 11″ and 11′ indicated in FIG. 21 are shown.

FIG. 22 is to illustrate how the bundles 130 and 150 of tensioning threads being separately provided from each other devolve into threads 11″ and 11′ with respect to the grouping together of the latter in the bundles 130 and 150.

FIG. 23 shows the self-balancing design shown in FIGS. 21 and 22, in a schematically simplified manner for only one bundle 130/150, ending with two loops 11″ and 11′, respectively. It is obvious from FIGS. 21, 22 and 23 that each loop of tensioning threads 11′ enters the shaft 101 through a first aperture 9′ for joining the downwards directed bundle 130, passing through the tensioning thread 170 for going up again as a part of bundle 130 so as to go out through a second aperture 9′ (being arranged below the first aperture 9′). That is, each tensioning thread 11″ surrounding a portion of the stent (implant) near the base thereof is integrally formed with one tensioning thread 11′, with the tensioning thread 11′ surrounding a portion of the stent (implant) near the tip thereof.

FIG. 24 shows a set 200 according to the invention with an implant 3 expanding through an folding device 100 according to the invention. The expansion can benefit in the present through the internal stress of implant 3. An implant of this specification can expand itself although only after a corresponding release of the pulling thread 170.

As to be seen in FIG. 24 (and likewise in FIG. 25) set 200 is shown only with one upper thread 11′ and one lower thread 11″. This reduction (simplification) is used for improved clarity. It is therefore clear that any arbitrary number of upper and lower threads 11″ and 11′ can be provided.

FIG. 25 shows a set 200 according to the invention from FIG. 24 with an implant 3 by means of folding device 100 according to the invention in a partly folded condition, through the further folding of the implant (in comparison with the condition in FIG. 24) the pulling threads 170 protrudes further out of shaft 101 in the direction of lower part of the view as in FIG. 25.

FIG. 26 shows in part section the tip 51 of a folding device 100 shown in a closed condition prior to implantation.

Shown in part section is an exterior protective sleeve which gives protection to a retaining area 55 for the implant 3, in this case a stent which is stored between the tip 51 and a collar 57. The collar 57 may advantageously guide the sleeve over the implant 3, e.g. when being a crimped stent.

The implant 3 is held in a restrained state in which the implant 3 is not expanded, by means of the threads 11″ and 11′.

FIG. 27 shows the tip 51 of an folding device 100 according to the invention in FIG. 26 prior to implantation, with a partly withdrawn external protective sleeve 53. Through the withdrawal of the outer protective sleeve the implant is released for implantation. The restrained state is substantially or fully maintained through the tension of the circumferential threads 11″ and 11′.

FIG. 28 shows the tip 51 of an folding device 100 according to the invention of FIG. 27 without implant, to be noted now are the shaft apertures 9′ through which the threads 11″ and 11′ not shown in FIG. 28 (see FIGS. 26 & 27) exit and enter shaft 101.

FIG. 29 shows the shaft 101 and herein arranged movable sleeve 81 of a folding device 100 according to the invention of FIG. 27, in a part sectioned part view in the first position of sleeve 81 on shaft 101.

The sleeve 81 features an opening 83 of the sleeve 81 comprising a wide area 83a, a first recess 83b and a second recess 83c. The first recess 83b and the second recess 83c are separated from one another by a bar 85.

Recesses 83b and 83c can be of different lengths as shown in FIG. 29. They can alternatively have different lengths in another spatial envelope.

Bar 85 is able to separate two threads from one another, one of which runs through the first recess 83b and the second runs through the second recess 83c.

As can be seen in FIG. 29, located on the recess 83b and/or on the bar 85 on the side facing the recess 83b or 83c may optionally be an initial area 84a with a cutting device 86. Located on the recess and on bar 85 on the side facing the recess 83b and/or the recess 83c may optionally be a second area 84b without a cutting device.

In the first position the threads (not shown in FIG. 29) can be threaded from the outside of shaft 101 through the shaft aperture 9′ and the wide area 83a of sleeve 81 into the inside of sleeve 81.

This first position is suitable for the insertion of the threads into folding device 100. This position can be achieved by bringing a pre-tensioning device 87 under an increased tension.

The pre-tensioning device is by way of example shown as a spring, or more precisely a coil spring.

Further there is a sealing device 88 with an opening 89 for a guide wire that is not shown in this view.

A second shaft aperture 9′ with a second sleeve aperture 83 is shown by a cutaway section on the outer sleeve of shaft 101 on the left side. By means of this through section an otherwise obscured view through shaft 101 and sleeve 81 of the inside shaft 101 is made possible.

FIG. 30 shows the arrangement as in FIG. 29 in a second position.

In the second position the load in the pre-tensioning device 87 is reduced from that in FIG. 29. It is clearly no longer under load in FIG. 30. In FIG. 30 the sleeve 81 is slid further up to the right (ref FIG. 30). This also applies to the sleeve apertures 83. The bar 85 thereby divides the shaft apertures to the extent that the shaft aperture 9′ with sleeve aperture 83 now give a passage from the outside of shaft 101 to the inside sleeve 83 through two shaft part apertures 91 and 93. It is possible, pertaining to the invention, that the transition from the first to the second position may be brought about solely or with the assistance of the pre-tensioning device or indeed the be brought about manually.

In the second position are two threads (not shown in FIG. 30) one of which is for example passed through the shaft part aperture 91 and the other passed through shaft part aperture 93 separated from one another by bar 85. This division can be advantageous in forestalling a tangling or functional obstruction of the two threads, or in the area of the shaft apertures at least reduce these. Lastly, it is advantageous chiefly if the threads are to be operated separately from one another.

FIG. 31 shows the arrangements of FIG. 29 and FIG. 30 in a third position.

In the third position the shaft part aperture is further reduced to such an extent that a thread (not shown) which is running through the shaft part aperture 93 comes into contact with the cutting device in the second area 84b.

By sliding the sleeve 81 further up to the right (in FIG. 210) relative to shaft 101, further towards the nearer end of shaft 101, the shaft part aperture will become smaller and the thread (not shown) finally cut through. This position which is continued on from the third position is shown in FIG. 211.

In FIG. 32 it can be seen that the shaft part aperture 91 no longer exists. The shaft part aperture 91 of FIGS. 30 and 31 is closed by the wall 103 of shaft. The thread is completely severed.

FIG. 10 shows an implant 3 which is expandable and can be reduced in its diameter. The diameter thereby refers to a plane perpendicular to a longitudinal axis of the medical implant 3. The longitudinal direction also corresponds to the direction of the extension of the implantation device 1 shown in FIG. 10. The implant 3 comprises two circular supporting means or rings 11. The supporting means 11 are connected to rods or posts 12. In some embodiments, the supporting means 11 can—additionally or alternatively or exclusively—fulfill the function of a guiding means for tension threads 2. The tension threads 2 form part of an implantation device 1 and serve for applying force or tension or stress, respectively, to the supporting means 11 for the purpose of expanding or folding the implant in a targeted manner. In the example of FIG. 10, the supporting means 11 are each designed in form of an outwardly half-open channel, through which the tension threads 2 are guided. The half-open channel is opened in a direction away from the center of the implant 3. However, the channel can also be designed in a form open to the implant or to another direction.

In the example of FIG. 10, the supporting means 11 are interrupted by posts 12, i.e. the posts 12 are integrated into the supporting means 11 such that they form sections of the supporting means 11.

In the embodiment of the implant 3 according to the invention of FIG. 10, the supporting means 11 have (round or differently shaped, e.g., oval, rectangular, elliptic, and so on) passage means or apertures 10. In the embodiment of the implant 3 according to the invention, these serve as a passage for the tension threads 2.

Furthermore, the implant 3 can also comprise a number of guiding means other than two, for example, one, three, four or more guiding means. The supporting means 11 can be arranged circularly, however, they can also be arranged non-circularly. The supporting means 11 can be formed integrally with the implant; however, they can also be fabricated separately. The supporting means 11 can have the form of a wave or undulation, respectively; however, they can also be fabricated in any other form, in particular, a non-wavy or non-undulating form.

Independent of all other features, the implant 3 can be fabricated from flat material, e.g., a material which has been cut with a laser, wherein, e.g., after having designed a pattern in the flat material, the material is reformed into a tube (optionally by connecting, such as welding, longitudinal sides of the former flat material lane or web, respectively). However, the implant 3 can also be fabricated from a tubular material directly.

The supporting means 11 of the implant 3 consist of a plurality of bars which are each connected to another by means of connecting sections 9. According to the invention, the connecting sections 9 differ in their design. However, as the latter is not shown in FIG. 10, reference is made to FIG. 34.

FIG. 11 shows the implant 3 of FIG. 10. Two tension threads 2 have been led around the implant 3 and return back to the implantation device 1 through the respectively same passage means or apertures 10. The tension threads 2 apply a tension or stress, respectively, on the implant 3 and the implant 3 is not completely expanded or unfolded, respectively. The diameter of the implant 3 has been reduced.

FIG. 33 shows an enlarged section of the medical implant of FIG. 10. In this enlargement, connecting sections 7′, 7″, 7′″ and 9 can be seen. All of them connect bars 11 which are arranged there between.

FIG. 34 shows a detail of a supporting means 11 of the implant 3 according to the invention. For illustration purpose, this detail of the supporting means 11 is shown as an even structure in FIG. 34.

Connecting means 7′ are shown which are each followed by a bar 111 and subsequent connecting sections 7″. Those are again followed by another bar 111 which is in turn followed by another connecting section 7′″. Respective bars 111 and, finally, connecting sections 9 follow the connecting sections 7′″ at both ends of the post 12.

FIG. 34 shows that the connecting sections 7′, 7″ and 7′″ each have widths d1, d2 and d3. Thereby, in the presentation of FIG. 34, width d1 is smaller than width d2 and width d2 is smaller than width d3. That means: d1<d2<d3.

In the embodiment according to the invention shown in FIG. 34, the connecting section is an apex of curvature or comprises such an apex of curvature.

The difference of the widths d1, d2 and d3 is present in a direction which extends in parallel to a longitudinal axis of the medical implant 3.

In other embodiments according to the invention of the supporting means according to the invention, the differences are present in another direction, e.g., in a direction which does not extend in parallel to a longitudinal axis of the medical implant during a state of use (e.g., before an extracorporeal expansion) or in turn in another direction. This other direction can be a direction perpendicular to a longitudinal axis of the medical implant 3. Moreover, this other direction can be any other direction.

The widths of the connecting sections 9 can, e.g., correspond to width d3. However, the connecting sections 9 can also have any other width. In particular, the connecting sections 9 can have a uniform width.

FIG. 35 shows an enlarged section of the supporting means 11 of the implant 3 according to one embodiment. In the example of FIG. 35, the supporting means has a width of L1 (from the distal end to the proximal end).

As can be seen from FIG. 35, the supporting means 11 comprises bars 111a and 111b which connect a rod or post 12 comprising an (oval) string outlet or aperture 10 with corresponding adjacent connecting sections 7′, respectively. Bars 111a and 111b which can be considered as to merge with the post 12 (in contrast to other bars 111) are shorter than other bars 111. In fact, bars 111a and 111b contribute to forming a slit 31′ that reaches from an end of the supporting means 11 shown at the left-hand side of the representation of FIG. 35 to the left-hand end of rod or post 12. In FIG. 35, the slit has a length of L2.

As can further be derived from FIG. 35, between the right-hand end of slit 31′ and the left-hand end of string outlet or aperture 10 a distance having a length L3 is provided. The distance L3 may be filled with a solid part of post 12.

Between the right-hand end of slit 31′ and the centre of string outlet or aperture 10 a distance having a length L4 is provided.

The left-hand end of the supporting means 11 of FIG. 35 may be spaced from the centre of the outlet or aperture 10 by the sum of L2 and L4.

As regards to the relation of L1, L2, L3, and L4, it is noted that in one preferred embodiment of the invention, L1 is between 2.5 and 3.5 times as long as L2, preferably 3 times as long.

In some embodiments, L2 is 2 times (or between 1.5 and 2.5 times) the length of L4.

In certain embodiments, L2 is 3 times (or between 2.5 and 3.5 times) the length of L3.

As regards FIG. 35, it is noted that the left-hand end of the supporting means 11 may be the distal or proximal end of the supporting means 11 and/or of the implant 3.

It is to be noted that the features described with reference to FIG. 35 may be embodied in an implant according to the invention without necessarily comprising also features described with regards to FIGS. 10 to 34.

FIG. 36 shows a schematic illustration of an implant 3. The implant 3 is crimped onto the outer surface 23′ of the tip 1′ of an implantation device 1. The implantation device 1 has a proximal ring 11a, a distal ring 11b and posts 12 with proximal and distal ends 12a, 12b. Strings 15a, 15b are guided by means of the distal ring 11b and the proximal ring 11a, respectively. The strings 15a, 15b may be used for folding and unfolding of the implant 3 in a controlled manner.

The implant 3 may be a heart valve replacement as is described in WO 2008/029296 A2 or in WO 2009/109348 A1 as referred to above.

As can be seen from FIG. 36, the implant 3 is tightly crimped onto the implantation device 1 such that ring-shaped portions 25′ and 27′ are in contact with the outer surface 23′ of the implantation device 1. As can also be seen, at least a first gap d1 between the post 12 and the outer surface 23′ of the implant 3 is created and/or maintained during crimping. In certain embodiments according to the invention, the first gap d1 has the shape of a tube. In the embodiment of FIG. 36 it is due to the first gap d1 that structures comprised by the implant such as heart leaflets or commissures (both not shown in the figures) may be left unstressed, unpressed unforced and the like upon and after crimping of the entire implant 3 or the implant as such, respectively.

FIG. 37 shows a schematic illustration of an implant 3 in a second embodiment.

In the second embodiment, in contrast to the crimping state shown in FIG. 36 in which the implant 3 is in contact or form fit with the outer surface 23′ of the implantation device 1 along ring-shaped portions 25′ and 27′, the implant 3 does not have contact with the outer surface 23′ at all. Rather, after completion of the crimping process of implant 3, a second gap d2 remained between the implant 3 (e.g., its post 12 or its ring-shaped portions 25′ and 27′ of the distal and proximal rings 11b, 11a) and the outer surface 23′ of the implantation device. A interconnection between the implantation device 1 and the implant 3 needed for delivery of the implant 3 to its implantation site may be achieved by means of the strings 15a, 15b, which are connected to the implantation device 1 (interconnection is not shown in FIG. 36 or 37; it can, however be seen in all detail in WO 2008/029296 A2 or in WO 2009/109348 A1 as referred to above). A connection may also be achieved by means of a sleeve (not shown) covering the implant during delivery.

The interconnection between implant 3 and implantation device 1 is a more loose one when compared to that achieved by the crimping the result of which is shown in FIG. 36.

As is obvious to the skilled person, structures of the implant 3 such as (not shown) heart valve leaflets may be comprised and housed by the implant 3 during and after crimping of the implant 3 without being stressed, crushed, forced, pushed and/or the like. Gaps d1, d2 and d3 provide sufficient space for such structures such that the implant can be crimped without any adverse effect happening to said structures.

As can be seen from FIG. 37, in contrast to the implant shown in FIG. 36, at least one (or all) of the posts 12 of the implant 3 are arranged such that it is level with the distal and proximal rings 11a, 11b. Hence, as can be derived from FIG. 37, the method according to the present invention can be carried out with any type of implant. The benefit of the method according to the invention does not depend on the concrete or specific design or embodiment of the implant.

FIG. 38 shows a schematic illustration of a hand-held and hand-operated crimping device 310 according to a first embodiment.

The crimping device 310 comprises actuators 33a, 33b comprising brackets 35a, 35b for receiving the (not shown) implant for crimping same. The actuators 33a, 33b are connected to each other by means of an articulation or a joint 37. They are further connected to each other by means of a pressure limiting means 39. The pressure limiting means 39 may be adjustable. It limits the pressure exerted to the structure at issue of the implant to the predetermined pressure.

FIG. 39 shows a schematic illustration of a crimping device according to a second embodiment.

Like the crimping device of FIG. 38, the crimping device 310 comprises actuators 33a, 33b comprising brackets 35a, 35b for receiving the (not shown) implant for crimping the same.

In contrast to the first embodiment, in the second embodiment the crimping device comprises pressure limiting means embodied as controller 41. The controller 41 may be interconnected to an adjusting means 43 for adjusting the maximum pressure exerted to the structure in question of the implant in correspondence to the predetermined pressure.

It is noted that the crimping device according to the invention may have in any embodiment thereof (that is, irrespective of any further features of the crimping devices 31 shown in FIG. 38 or 39) a sensor for measuring the pressure or force exerted on the structure during crimping. FIG. 40 shows an implant 3, viewed from the side, in an expanded state. The implant 3 is connected to an implantation device 1 according to the invention. The implantation device 1 is designed in form of a catheter tip 1′.

At the lower end of FIG. 40, a part of a catheter 1″ is shown. The catheter 1″ is detached from the implantation device 1 or catheter tip 1′.

The implantation device 1 comprises first interconnection means 6a. The catheter 1″ comprises second interconnection means 6b.

The implantation device 1 and/or the implant 3 comprise first folding and/or unfolding means 2. The first folding and/or unfolding means 2 can be embodied as strings.

The first folding and/or unfolding means 2 of the implant 3 pass through an inner space 99 of the implantation device 1. The first folding and/or unfolding means leave (not shown) the implantation device 1 through an opening 911a. In FIG. 40, opening 11a is arranged at the lower end of the implantation device 1 according to the invention which is directed to the catheter 1″. The first folding and/or unfolding means may enter into the catheter 1″ through an opening 911b of catheter 1″.

FIG. 41 shows a catheter 1″ comprising second folding and/or unfolding means 2′. The second folding and/or unfolding means 2′ are arranged in an inner space 98 of catheter 1″.

The second folding and/or unfolding means 2′ can be embodied as strings. At one end (in FIG. 41 at the right-hand end), the second folding and/or unfolding means 2′ comprise hooks 17′. The hooks 17′ are provided for establishing an interconnection to the first folding and/or unfolding means 2 of the implant (not shown in FIG. 41). It is noted that FIG. 41 shows a state of the catheter 1″ before being connected to the device (not shown in FIG. 41).

FIG. 42 shows a connection state between the catheter 1″ and the implantation device 1 or catheter tip 1′.

In the inner space 99 of implantation device 1, the first folding and/or unfolding means 2 of implant 3 (not shown here) are arranged. The first folding and/or unfolding means 2 comprise loops 18 for surrounding the implant (not shown in FIG. 42) and also loops or eyes 18′ provided for establishing an interconnection with the second folding and/or unfolding means 2′ of catheter 1″. As exemplified here, the connection between the first and the second folding and/or unfolding means 2, 2′ could be established by hooking hooks 17′ into eyes 18′.

As is shown in FIG. 42, implantation device 1 comprises attaching or interconnecting means such as a nose 21 and a recess 21′ forming an offset for receiving a blunt end 21″ of catheter 1″. Blunt end 21″ of catheter 1″ is an example for an attaching or interconnecting means of catheter 1″.

The connection between implantation device 1 and catheter 1″ can be achieved by simply slipping on implantation device 1 onto catheter 1″ such as a plug-in connection. An additional frictional closure may be provided.

As is obvious to the skilled person, the connection is, of course, not limited to plug-in or slipping or snatching connections as exemplified here. Any other suitable interconnection is also contemplated.

FIG. 43 shows an implant 3, viewed from the side, in an expanded state. The implant 3 is connected to an implantation device 1. The implantation device 1 is designed in form of a catheter tip.

At the lower end of FIG. 43, a part of a catheter 1″ is shown. The catheter 1″ is detached from the catheter tip.

The implantation device 1 comprises a portion 231 for folding and/or unfolding the implant 3. The catheter 1″ comprises a heart or a cord 251 and a fourth section 251b for its connection with the implantation device 1.

The implantation device 1 and/or the implant 3 comprise first folding and/or unfolding means 2. The folding and/or unfolding means 2 can be embodied as strings.

FIG. 44 shows an implantation device 1 according to the invention having an inner portion 231 for folding and/or unfolding the implant that is rotatably supported on three bearings 213 in an outer sheath 35. The implantation device 1 which is shown in a first embodiment in FIG. 44 is connected with a flexible catheter 1″ in the representation of FIG. 44. The catheter 1″ comprises a flexible cord 251 or heart rotatably supported in a catheter sheath 53′. In the representation of FIG. 44, the cord 251 can be rotated within and relative to the catheter sheath 53′. This can be effected by means of respective bearings (not shown in FIG. 44 and not even mandatorily required). However, instead of those bearings, there can be provided corresponding geometrical embodiments of the catheter 1″ and/or corresponding surface characteristics or treatments of the cord 251 and/or of the inner surface or the periphery surface, respectively, of the catheter sheath 53′.

In the example of FIG. 44, the outer sheath 35 as well as the catheter sheath 53′ are interconnected by means of a second section 311b and a fourth section 511b both embodied across the entire periphery of the sheath 35 and the catheter sheath 53′. However, in other embodiments according to the invention, the second section 311b and the fourth section 511b can also solely be provided across certain portions of the periphery.

In the example of FIG. 44, the second section 311b and the fourth section 511b are plug-in connections that do not permit a rotation of the outer sheath 35 of the implantation device 1 relative to the catheter sheath 53′ of the catheter 1″ during normal use of the implantation device 1 and/or the catheter 1″. Thus, the implantation device 1 as a whole can only be rotated around its longitudinal axis even if the catheter 1″ is rotated around the longitudinal axis thereof in at least the portion receiving the implantation device 1.

The opposite applies for portion 231 for folding and/or unfolding the implant (which is not shown in FIG. 44). The portion 231 is arranged rotatably within the outer sheath 35 of the implantation device 1 by means of the bearings 213. The portion 231 can be actuated by means of a rotational move of cord 251—relative to the catheter sheath 53′ of the catheter 1″—to perform a rotation around the longitudinal axis of portion 231 indicated by the dot dash line.

Such a coupling between cord 251 of the catheter 1″ and portion 231 for folding and/or unfolding the implant of implantation device 1 is, for example, possible by means of the first and third sections 311a and 511a represented in FIG. 44. The first section 311a and the third section 511a can be frictional and/or form closure connection devices.

It can be further seen from FIG. 44 that threads or strings that represent examples for the means 2 for folding and/or unfolding the implant not shown in FIG. 44 are guided from an interior of the implantation device 1 through openings 330 to an exterior of the outer sheath 35 in order to be in contact with the implant not shown here. As the threads are connected with the portion 231 for folding and/or unfolding the implant in a portion 71 thereof (for example, by means of interlooping, sticking or the like, in each case by means of a frictional and/or form closure connection), the threads are wound up during the rotational move of the portion 231 around the longitudinal axis thereof around an outer surface 370 or an outer periphery, respectively, of the portion 231 for folding and/or unfolding the implant.

FIG. 45 shows an implantation device 1 according to a second embodiment. The embodiment shown in FIG. 45 differs from the embodiment of FIG. 44 at least in that the portion 231 for folding and/or unfolding the implant comprises grooves 21′ that are wound spirally or helically along the longitudinal axis (the dot dash line of FIG. 45) around the portion 231 for folding and/or unfolding the implant. A thread (as an example for a means 2 for folding and/or unfolding the implant which is also not shown in FIG. 45), that is, for example, attached at an attachment site 39′ of the portion 231 for folding and/or unfolding the implant that is not shown in FIG. 45, may—after having been wound around the portion 231, be provided within the outer sheath 35 such that the thread is received within the groove 21′. The thread thus extends across the longitudinal axis of the portion 231 for folding and/or unfolding the implant. In this way, an agglomeration of thread material at a closely limited periphery portion of the portion 231 due to winding the thread upon folding the implant is prevented. A space between portion 231 and outer sheath 35 can thus be embodied marginally or small.

In order to favour such a winding of the thread along the longitudinal extension of the portion 231, in the embodiment shown in FIG. 45 opposite to the implantation device 1 according to the one of FIG. 44, the portion 231 is embodied having a worm or thread 38 which is engaged with engagements 15. In this way, the portion 231 can be displaceable into the position indicated by the dot dash line to the right, relative to the plane of projection of FIG. 45, during its rotation within the outer sheath 35 of the implantation device 1. In the embodiment of FIG. 45, the worm or thread 38 and the engagements 15 act together as one example of an advancing mechanism or a displacing mechanism. In FIG. 45, the engagements 15 replace the bearing 213 represented in the embodiment shown in FIG. 44. Alternatively, instead of the engagements 15 a worm can be provided at the outer sheath 35 as well. Moreover, the engagements 15 can alternatively be provided on the portion 231.

It is further obvious that it is contemplated according to the invention to provide an engagement on an outer surface of the portion 231 and a worm as being part of the outer sheath 35 instead of the outer worm or thread 38 of the portion 231 and the engagement 15. The present invention encompasses both embodiments.

By means of worm and/or engagements, a mechanism for effecting a longitudinal displacement of the portion 231—relative to the outer sheath 35—is given in the embodiments of FIG. 45. However, the said can of course be embodied in another way than the one given in the example of FIG. 45.

It is obvious that the provision of grooves 21′ can be provided or can also not be provided independently from the provision of a mechanism for effecting a longitudinal displacement of the portion 231, relative to the outer sheath 35 in the implantation device 1.

Furthermore, a person skilled in the art will recognize that a fin 23″ can be provided instead of groove 21′ as shown in FIG. 45 and as can also be seen in FIG. 46a in the cross-section of portion 231 along which, for example, the thread can be guided as a means 2 for folding and/or unfolding the implant.

As is obvious to the skilled person, the present invention is, of course, not limited to plug-in or slipping or snatching connections as exemplified here. Any other suitable interconnection is also contemplated.

FIG. 47 shows a set 200 according to the invention comprising an implantation device 1. The implantation device 1 having an end portion and a tip 1′ comprises a medical implant 3 and an aligning device 90.

The implant 3 comprises two ring structures 11 that can be expanded or folded by means of threads 2. The threads 2 are guided through an interior of the implantation device 1 and can be actuated at a further end portion of the implantation device 1 as a continuation 2″. The ring structures 11 are distanced from each other by means of struts 12 that are arranged between the ring structures 11. As can be recognized by a person skilled in the art, the implant 3 shown in FIG. 47 is just an arbitrary example of an implant selected for illustrative purposes. For details of the implant shown in FIG. 47 it is referred to WO 2009/109348 A1.

In the example of FIG. 47, the aligning device 90 comprises two aligning sections 91′ and 93′; however, according to the invention, more or fewer than two aligning sections could also be provided or intended.

The aligning sections 91′, 93′ of the representation of FIG. 47 are designed or embodied as wires or filaments, respectively, and in form of loops or slings, respectively, i.e., in form of closed structures. However, according to the invention, a closed structure is not required.

Each of the aligning sections 91′, 93′ is supported by the tissue G at a section 911 or 931 and thereby contacts tissue G. As the aligning sections 91′, 93′ show a stiffness resulting from their material and/or shape, they show a resistance during pulling back or retrieving, respectively, the implantation device 1 guided from the bottom through the tissue aperture A in FIG. 47. They hereby align the implantation device 1 relative to the tissue G or the tissue aperture A thereof by requiring an increased force for retrieving the implantation device 1 out of the tissue aperture A (i.e., in a downward direction in FIG. 47) or by even preventing such a retrieval.

After aligning the implantation device 1 as described above and thus the accompanying alignment of the implant 3 relative to the tissue G or the tissue aperture A, the implant 3 can be expanded by means of the threads 2 or the continuations 2″ thereof, respectively. Subsequently—as well as at any other point of time —, the aligning sections 91′ and 93′ may be pulled into an interior of the implantation device 1 by pulling a continuation 2′″ of the aligning sections 91′ and 93′. The loops or slings, respectively, of the aligning device 90, i.e., the aligning sections 91′ and 93′, shown in FIG. 47 are thus not present outside the implantation device 1 anymore. Therewith, the alignment is cancelled or offset. The implantation device 1 can be pulled downwards out of the tissue aperture A (after having released the implant 3 from the implantation device 1).

FIG. 48 shows an implantation device 1 of a second embodiment comprising an end portion 1′ and an interior 105. The implantation device 1 comprises an aligning device 90 comprising two aligning sections 91′ and 93′. The aligning sections 91′ and 93′ are continued within the interior 105 of the implantation device 1 in form of continuations 2′″ and can be pulled into, e.g., the interior 105 by means of the latter.

As compared to the implantation device 1 of FIG. 47, the implantation device 1 shown in FIG. 48 comprises a flexible or stiff sheath 13′. The latter is movable relative to the shaft of the implantation device 1 by, e.g., being retrieved in the direction of the arrow (to the left border of FIG. 48).

When the sheath 13′ is in a non-retrieved state, the aligning sections 91′ and 93′ are kept from lifting off the implantation device 1 or an outer surface thereof, respectively, which they would do otherwise due to their shape memory characteristics. By retrieving the sheath 13′ in the direction of the arrow, the limitation on the moving or unfolding capability of the aligning sections 91′ and 93′ relative to the implantation device 1 is offset.

In FIG. 48 a state is shown in which the aligning section 91′ could already be released from the implantation device 1 and transferred into its memory shape state after the sheath 13′ having been retrieved. In the shape memory state predetermined by the manufacturing process, one end of the aligning section 91′ lifts off the implantation device 1 and winds up in a C shape manner. According to the invention, any other shape into which the aligning section could wind up instead of a C shape is contemplated as well.

In the state shown in FIG. 48, the sheath 13′ is not retrieved to a sufficient extent for also releasing the other aligning section 93′. The latter still unchangedly contacts the implantation device 1 or an outer surface thereof, respectively. Only after the sheath has been pulled back a little bit further—not shown in FIG. 48 —, also the aligning section 93′ will deform in an intended manner and be released from the outer surface of the implantation device 1.

FIG. 49 shows an aligning section 91′ of an apparatus according to the invention in a further embodiment. The aligning section 91′ has a still further shape. The said may be referred to as spirally.

FIG. 50 shows an aligning section 93′ of an apparatus according to the invention in a still further embodiment.

The aligning section 93′ has a still further geometrical outer shape.

The aligning section 93′ further comprises a continuous lumen 933 within its interior. The latter extends along the whole length of the aligning section 93′. According to the invention, in other embodiments, the lumen can, however, also only extend across a (part) portion of the aligning section 93′.

In some embodiments, the Lumen 933 is provided or intended to administer a fluid therethrough. The fluid can be a drug, a contrast agent, e.g., for imaging methods, or the like. The fluid can be introduced in and discharged out of the lumen along the directions of the arrows of FIG. 50. By means of the lumen, it is advantageously possible to use the aligning device both for aligning and for administering at the same time. In this way, introducing or inserting, respectively, an additional instrument for aligning or administering may advantageously be omitted.

As the aligning device is inserted together with the apparatus which is also used for inserting the implant into the body, inserting one or more further instruments for an alignment or an administration of an agent in addition to the apparatus may advantageously be omitted.

FIG. 51 shows a partial longitudinal section through an implantation device 1 according to the present invention that is shown in a schematically simplified manner and only in a section thereof. The implantation device 1 comprises a shaft 101 comprising a first section 101′ and a second section 101″. A plurality of individual shaft fibers 13″ extends along or about both the first section 101′ and the second section 101″. The first and the second section 101′, 101″ may be referred to as shaft section.

The individual shaft fibers 13″ comprise shaft openings 9′. Merely exemplarily, FIG. 51a shows two tension threads 11′ and 11″ each which exit from a shaft opening 9′, twining or looping around a rim portion of the implant 3 shown in a folded state in FIG. 51a and—to be understood merely exemplarily as well—which re-enter into the same or into another shaft opening 9′ of the same shaft fiber 13″.

One tension thread or more tension threads 11′ and 11″ may exit from an interior of the shaft 101 towards the exterior of the shaft 101 through the shaft openings 9′ and/or may enter in the opposite direction. In FIG. 51a, all threads 11′ and 11″ both exit and enter in a loop manner through the shaft openings 9′.

The tension threads 11′ and 11″ are provided or intended to encompass an implant not shown in FIG. 51a such that the implant will have an altered diameter when altering the tension applied onto the threads 11′ and 11″ in sections thereof.

In the state of the implant 3 shown in FIG. 51a, the tension threads 11′ and 11″ are arranged at the implant 3 under tension by means of which they inhibit an undesired opening, unfolding or expansion of the implant 3 (the latter resulting, e. g., from a memory shape property of the implant).

FIG. 51a shows only two tension threads 11′ and 11″. This serves for the purpose of clarity. However, a person skilled in the art will recognize that every shaft fiber 13″ can comprise one or more of such tension threads. The latter can exit from the shaft fibers 13″ at different heights thereof, wherein this applies both for one the same shaft fiber 13″ and, e. g., adjacent shaft fibers 13″.

As can be seen from FIG. 51a, the individual shaft fibers 13″ arranged on the right side (i. e. away from the tip of the implantation device 1 or within the second section 101″, respectively) of a device 190 for bundling individual shaft fibers 13″ are combined or concentrated in a bundle. In the second section 101″ (i. e. on the left side of the device 190 or towards the tip of the implantation device 1, respectively), the shaft fibers 13″ are arranged freely movably—relative to each other, although, in the state of the implant 3 shown in FIG. 51a, they also contact each other—as they do also in section 101″.

On the one hand, the device 190 for bundling allows for the bundled shaft fibers 13″ in FIG. 51a to move freely in radial direction on the left side of the device 190. Thereby, they can follow the motion or movement or geometry of the unfolded implant 3. The device 190 is arranged not to hamper that movement.

On the other hand, the device 190 for bundling allows for setting the rigidity or stiffness of the shaft fiber 13″ on the left side of the device 190. By shifting the device 190 along the shaft 101 to the left as indicated by arrow A in FIG. 51a, the rigidity or stiffness of the individual shaft fibers 13″ beyond the device 190 can be increased. By shifting the device 190 in the direction indicated by arrow B (i. e. to the right side in FIG. 51a), the rigidity or stiffness of the shaft fiber 13″ on the left side of the device 190 can be reduced. In this way, the implantation device 1 may advantageously be adapted to different features or behaviour of different implants.

It is obvious that the implant is represented in a very schematic and simplified manner. The present invention may be carried out with any implant designed or embodied to be foldable and/or unfoldable by means of tension threads upon implantation.

FIG. 51b shows a section along line I-I of FIG. 51a.

FIG. 52a shows a partial longitudinal section through the implantation device 1 according to the present invention of FIG. 51a that is shown in a schematically simplified manner and only in a section thereof after having entirely unfolded the implant 3 with the tension threads 11′ and 11″ being completely or substantially released or free from tension.

The individual shaft fibers 13″ are present in a bundled form on both sides of the device 190 (i. e. on the left side and on the right side of the device 190, i. e. both in the first section 101′ and in the second section 101″). In any case they are provided in a bundle in which the individual shaft fibers 13″ are close to each other or even contact each other. Due to their form which they take on while not experiencing any external tension or force, the shaft fibers 13″ are present in an extended or stretched form in the second section 101″. This is possible because the tension threads 11′ and 11″ are getting longer or stretch upon (or after) opening or unfolding of the implant 3. The latter can be achieved by correspondingly actuating the tensioning device not shown in the figures.

FIG. 52b shows a section along the line II-II of FIG. 52a. As can be recognized, the individual shaft fibers 13″ are present in a bundle such as shown in FIG. 51b; however, they are only bundled loosely and are not pressed against each other by an external force.

Tension threads 11″, 11″a, 11″b and 11″c that are shown by way of example each encompass a rim portion 301′, 301″, 301′″ or 301″″. In an area 303 of overlap both the tension thread 11″a and the tension thread 11″b are provided. According to the present invention, such an overlap can be provided at any position, in particular along the periphery, of the implant 3. Moreover, it can be designed in any arbitrary way: based on two tension threads, three tension threads etc. In some embodiments, a more uniform application of tension force for folding the implant may be achieved by means of such an overlap.

As is illustrated in FIG. 52b, the rim portions encompassed by tension threads may have different widths or lengths, yielding the advantages mentioned above.

FIG. 53a shows a partial longitudinal section through the implantation device 1 according to the present invention of FIG. 51a that is shown in a schematically simplified manner and only in a section thereof after unfolding the implant 3 using tensioned tension threads. For example, four shaft fibers 13″a, 13″b, 13″c and 13″d are present within the implant 3. As can be seen from FIG. 53b in which an additional cut shaft fiber 139 is shown, the number of four shaft fibers is merely chosen for improved clarity and more than four shaft fibers may be present. However, a person skilled in the art will recognize the latter when considering the above specification.

FIG. 53b shows a section along the line of FIG. 53a. It can readily be seen that, due to the tension applied by means of the respective tension threads 11″, 11″a, 11″b and 11″c, the shaft fibers 13″a, 13″b, 13″c and 13″d have moved from the center of the implant 3 towards a rim area of the implant 3 or at least in a radial direction. One effect of this motion or movement is explained in detail with respect to FIGS. 54 and 55 below. However, without any further explanation, a person skilled in the art will yet be aware from FIG. 53b that the respective tension threads exit from the shaft fibers 13″a, 13″b, 13″c and 13″d via the shaft openings in a substantially diametrical manner.

Further, it can be recognized that the respective tension threads each extend between two penetration openings 305a and 305b that are present within the implant's periphery and at which the tension threads penetrate from the interior to the exterior through the “envelope” of the implant, on a more or less straight curve—together with the cross section of the shaft fiber.

In FIG. 53a, only by way of example, a second device for bundling depicted as reference numeral 191 is shown. As can be seen from FIG. 53a, the shaft fibers' portions situated between the two devices 190 and 191 are kept in parallel by means of the devices 190 and 191. On the right side of device 191 for bundling, the shaft fibers can flex or bend again.

In synopsis with FIG. 54b, FIG. 54a shows one advantage achievable by means of some embodiments according to the present invention using a sectional view similar to that of FIGS. 51b, 52b and 53b.

FIG. 54a shows how a rim portion 301 may bulge or dent inwardly when the tension thread 11′ entangled around it is guided by means of a shaft fiber 13″ arranged in the center of the implant—as is often the case with conventional arrangements in certain constellations or apparatus-implant-arrangements. The inventors of the present invention have realized that such a bulging or denting—both inwardly and outwardly—including any undesired non-uniform folding of the implant as well largely depends on the angle α shown in FIG. 54a.

In contrast, FIG. 54b shows the alteration of angle α when the shaft fiber 13″ is allowed to move, wander or migrate to or towards an outer area or the rim portion 301 of the implant 3 upon biasing or tensioning the tension thread 11′. Bulging or denting inwardly or outwardly or non-uniformly folding the implant may hereby in certain settings under otherwise unchanged conditions advantageously be reduced or even excluded.

In synopsis with FIG. 55b, FIG. 55a shows a further advantage achievable by means of certain embodiments according to the present invention using a sectional view similar to that of FIGS. 51b, 52b, 53b and 54b.

FIG. 55a shows the effect a tension thread 11′ may have onto the periphery or the rim portion 301 of an implant 3 when applying tension onto the periphery or the rim portion 301 by means of the tension thread 11′.

In FIG. 55a, as in FIG. 53b, penetration areas 305a and 305b are shown. The tension thread 11′ penetrates through the penetration areas 305a and 305b from a center of the implant to the exterior thereof, or vice versa. As can be seen in FIG. 55a, the penetration areas 305a and 305b may, due to the tension, bend or dent towards the center of the implant 3 or may fold non-uniformly with respect to the remaining periphery of the implant 3. As shown in FIG. 55a, this effect can be seen when the shaft fiber 13″ is arranged in the center of the implant 3, an arrangement that is common in the state of the art. In certain embodiments, the same effect can also be seen when the shaft fiber 13″ is arranged more radially.

A solution to the problem shown in FIG. 55a is shown in FIG. 55b. If the rim portion encompassed by the tension thread is set or determined to be sufficiently narrow or short (i. e. if the penetration openings 305a and 305b are arranged sufficiently close) such as proposed with respect to some embodiments according to the present invention, denting or bulging or a non-uniformly folding of the implant 3 can advantageously be avoided.

FIG. 56 shows schematically simplified and in part section an implantation device 1 according to the present invention with an expanded implant 3 according to a first exemplary embodiment of the present invention (the combination of implantation device 1 and implant 3 also being referred to as “set” herein).

A first tension thread 11′ and a second tension thread 11″ are arranged around the implant 3. As can be seen from FIG. 56, the implant 3 comprises a first guiding structure 11a for guiding the first tension thread 11′ and a second guiding structure 11b for guiding the second tension thread 11″.

In the exemplary embodiment of FIG. 56, the first guiding structure 11a and the second guiding structure 11b are designed as rings or channel-like ring structures. These structures are optionally radially open but medially closed as it is exemplarily also shown in FIG. 56.

Also by way of example, two, three or more posts 12 are arranged between the first guiding structure 11a and the second guiding structure 11b. The posts 12 each comprises one, two or more openings 10 for letting pass the first or second tension threads 11′, 11″ from an inside of the implant 3 to on outside thereof.

The posts 12 may be configured to keep the distance between the first guiding structure 11a and the second guiding structure 11b.

In the example of FIG. 56, the threads 11′ and 11″ are provided for holding the implant 3 with regards to the implantation device 1. In any case, the diameter of the implant 3 or its folding state may be altered by varying the tension of the threads 11′ and 11″ as will be explained in more detail below.

The implantation device 1 has a shaft 101 having a lumen covered by a wall (depicted with reference numeral “103” in, e.g., FIGS. 58 and 68). In the lower area of FIG. 56, the wall of shaft 101 is longitudinally cut. Pulling threads 17″ arranged within the lumen of the shaft 101 extend therefrom.

The pulling threads 17″ are integral with or interconnected to threads 11′ and 11″ which are guided along the circumference of implant 3 at different levels—by the first and the second guiding structure 11a and 11b—thereof such that pulling or releasing the pulling threads 17″ makes the threads 11′ and 11″ to exert more or less force on the implant 3 as it is also described in the patent application published under WO 2011/063972 A1. This way, operating the pulling threads 17″ may provide for a change in one or more cross-section dimensions of the implant 3. The respective disclosure of WO 2011/063972 A1 is incorporated into the present specification by reference.

The threads 11′ and 11″ enter into the lumen of shaft 101 by apertures 9′ not shown in FIG. 56 (but shown in FIG. 5) and they exit shaft 101 from such apertures again.

The expansion of implant 3 may benefit in the present exemplary embodiment from the internal stress or from shape-memory capacities of implant 3. The implant 3 may be manufactured from Nitinol or comprise such material. In order to expand the implant 3, the pulling threads 17″ need, however, to be sufficiently released. For folding the implant 3 again, the pulling threads 17″ are tightened again.

FIG. 57 shows the implantation device 1 of FIG. 56. The implant 3 is in a partly folded condition (also referred to herein as “folded” or “refolded”). Since folding of the implant 3 has to be achieved by pulling the pulling threads 17″, in FIG. 57 the pulling threads 17″ protrude further out of shaft 101 than in FIG. 56.

In FIG. 56 (and likewise in FIG. 57), implantation device 1 is shown with only one upper (“second”) thread 11″ and one lower (“first”) thread 11′. This reduction (simplification) is used for improved clarity. It is therefore clear that any arbitrary number of upper and lower threads 11′ and 11″ may be provided (“upper” and “lower” relate to the upright position of the implant shown in FIG. 57). A corresponding number of apertures 9′ may be provided.

FIG. 58 shows an implantation device 1 according to the present invention with an implant 3 attached at or within an implantation device 1 according to the present invention.

FIG. 58 shows in part section a tip 1′ of an implantation device 1 according to the present invention in a closed condition prior to implantation.

Shown in part section is an outer protective sleeve 53 which gives protection to a retaining area 55 for the implant 3. In the example of FIG. 58, the implant 3 is a stent which is arranged between the tip 1′ and a collar 57. The collar 57 may advantageously guide the sleeve over the implant 3 which may be, e. g., a crimped stent, as in the example of FIG. 58.

The implant 3 is held by the threads 11′ and 11″ in a restrained or folded state in which it is not expanded.

FIG. 59 shows the tip 1′ of FIG. 58 prior to implantation. The outer protective sleeve 53 is partly withdrawn. By withdrawing the outer protective sleeve 53, which is only provided by way of example, the implant 3 is ready for implantation. The restrained state is still maintained, substantially or fully by the tension of the circumferentially wound threads 11′ and 11″.

FIG. 60 shows the tip 1′ of the implantation device 1 of FIG. 59 without the implant 3. In FIG. 61, the wall apertures or shaft apertures 9′ through which the threads 11′ and 11″—which are also not shown in FIG. 60—exit and enter shaft 101.

Shaft 101 features an arbitrary number of apertures 9′, at one, two (as shown in FIG. 5) or more longitudinal heights of the axis.

The shaft apertures 9′ extend through the entire thickness of the wall of shaft 101 and, hence, interconnect the lumen or inner space of shaft 101 with the exterior of shaft 101.

The shaft apertures 9′ may be evenly spaced from each other around the circumference of shaft 101. Alternatively, they may be divided with at least two different distances from each other around the circumference.

FIG. 61 shows an implantation device 1 according to another exemplary embodiment of the present invention. FIG. 61 does not show an implant. It does not show threads and neither a section thereof to be clamped.

In FIG. 61 a first clamping section 61 and a second clamping section 63 are shown. In the example of FIG. 61, the first and the second clamping sections 61, 63 are both arranged as tube sections. Also by way of example, in FIG. 61 the first and the second clamping sections 61, 63 are arranged with respect to each other in a coaxial manner. Further, in the exemplary embodiment of FIG. 61, the first clamping section 61 which is arranged around the second clamping section 63 may be slid or moved along and relative to the second clamping section 63. For example, the first clamping section 61 may be slid or moved towards the tip 1′ into a clamping position in a direction indicated by the arrow C (for clamping). In this position, the section of the not shown thread would be clamped between the first and the second clamping section. In this particular and exemplary embodiment, the second clamping section 63 may be slid away from the tip 1′ into a release position in a direction indicated by the arrow R (for releasing). In this position, the section of the not shown thread would not be clamped any longer between the first and the second clamping section. In the particular exemplary embodiment of FIG. 5, the first clamping section 61 is in any case moved parallel to the longitudinal extension or the main extension of the shaft 101.

As discussed above, in FIG. 61 the implantation device 1 is shown in a release position or state. Also, the section of the threads 11′ and 11″ to be clamped is not shown in FIG. 61. However, as is readily been understood by one skilled in the art, a section of threads 11′, 11″, for example the end section thereof, may be clamped between the first and the second clamping sections 61, 63. This is easily accomplished by moving the second clamping section 61 towards the tip 1′ into the clamping position not shown in FIG. 61. The section in question may than be clamped between the first clamping section 61 (for example, the inner surface thereof) and the second clamping section 63 (for example, the outer surface thereof).

In FIG. 61, the second clamping section 63 comprises one or more clamping holes 65 through which the section of the threads 11′ and 11″ to be clamped may be guided from inside of shaft 101 (that is, from its lumen) to the outer surface of the second clamping section 63 and in between the first and the second clamping. sections 61, 63.

By way of example, for the ease of moving the first clamping section 61 into the release position shown in FIG. 61 during surgery, the first clamping section 61 may be provided with a thread 67 or any other coupling for interconnecting the first clamping section 61 with a retracting device, or release device, for pulling or retracting the first clamping section 61 in the direction indicated by arrow R. In other exemplary embodiments according to the present invention, the first clamping section 61 is integral with a releasing device.

It goes without saying that everything that has been stated herein for the first clamping section 61 may in further exemplary embodiments according to the present invention which are not shown in the figures also hold true for the second clamping section 63, and vice versa. For example, it may be the second clamping device 63 that is retractable or movable (in whatsoever direction) with regard to the first clamping device 61, in contrast to what is depicted in FIG. 61.

In the example of FIG. 61, the shaft 101 comprises a bayonet coupling 69 for connecting the shaft 101 with further parts of the apparatus. However, many other couplings such as screw threads may do as well and are, therefore, also encompassed by the present invention.

Further, the shaft 101, the first clamping section 61 or any other part of the implantation device 1 and the retracting device, or release device interconnected with the first clamping section 61 by the thread 67 (shown in FIG. 61) or any other coupling may be provided with a click-release lock or the like in order to avoid an unintended release of the first clamping section 61 from the clamping position.

Furthermore, the shaft 101 may have a further groove, sliding block guiding, slotted guide or the like to avoid twisting or rotation of retracting device, or release device with regard to the shaft.

FIG. 62 shows the implantation device 1 of FIG. 61 in a longitudinal section.

FIG. 63 shows the implantation device 1 according to a further exemplary embodiment of the present invention down to its handle 71′ of the of the releasing device. In FIG. 63, the releasing device is integral with the first clamping device 61. It may be retracted by pulling the handle 71′.

In FIG. 63, the implantation device 1 is shown in the release or open position or state in which no clamping can occur. However, no implant and no threads are shown.

FIG. 64 shows the implantation device 1 of FIG. 63. In FIG. 63, the implantation device 1 is shown in the clamping position or state.

FIG. 65 shows a schematically simplified cross section through the first and the second clamping sections 61, 63 of a first embodiment thereof.

The first clamping section 61 comprises two indentations 75 which together form a groove in which a bulge 77 may be moved in a direction perpendicular to the plane of projection of FIG. 65. The indentations 75 and the bulge 77 disallow, however, a rotation of the first and the second clamping sections 61, 63 relative to each other. That way, the indentations 75 and the bulge 77 act as a mechanism for disabling or for limiting rotation between the first and the second clamping sections 61, 63. Of course, the mechanism for disabling rotation may be embodied in any other suitable manner as well. It may comprise or exist of extensions, indentations, furcations, notches, oval cross sections of one or both clamping sections 61, 63, and so on. The invention is not limited to the exemplary embodiment shown in FIG. 65.

FIG. 66 is a cut view of an implantation device 1 according to the present invention in another embodiment in which the first clamping section 61 and the second clamping section 63 of the implantation device 1 are arranged such that they (or respective surfaces or planes thereof) are inclined to the longitudinal axis L of the implantation device 1, its shaft 101, and/or the reception or retaining area 55 for receiving the implant.

In the exemplary embodiment of FIG. 66, the first and the second clamping sections 61, 63 are inclined under the same angle. This way, upon moving the first clamping section 61 along array C (C for “clamping”), an inclined surface 61′ is eventually contacted or abutted by an inclined surface 63′ of the second clamping section 63 or of wall 103. This way, the clamping may advantageously take place along a longer distance than, e.g., the width of the shaft wall would allow.

The inclined surface 63′ of the second clamping section 61′ may be part of a collar 79 of the second clamping section 61′.

In the particular embodiment of FIG. 66, arrow C points towards the tip of the implantation device 1.

FIG. 67 shows a set 1200 of tension threads, according to the present invention.

The set 1200 of tension threads comprises in the exemplary embodiment of FIG. 67 one first string 1201. The first string 1201 is connected to the first tension thread 11′ and to the second tension thread 11″.

The first string 1201 comprises a first guiding element 1203 for guiding through the first tension thread 11′ and a second guiding element 1203′ for guiding through the second tension thread 11″. Both the first tension thread 11′ and the second tension thread 11″ are attached to the first string 1201.

In the exemplary embodiment of FIG. 67, both the first tension thread 11′ and the second tension thread 11″ are attached to the first string 1201 by their respective first end sections 12′ and 12″. They are attached such that their first end sections are fixed to the first string 1201 such that upon withdrawing the first string 1201 from the lumen of the implantation device 1 to an outside thereof, the first and second end sections have to follow the first string 1201 to the outside once the first and second tension threads 11′, 11″ are not clamped any more.

For example, the first tension thread and/or the second tension 11′, 11″ thread may be knotted to or integral with the first string 1201.

Both the first and the second tension thread 11′, 11″ are folded into loops 113 and 113′. By these loops 113, 113′ the threads 11′, 11″ may be inserted into the guiding structures 11a and 11b (not shown in FIG. 67) and hence, wound around the whole circumference of the implant or parts thereof. The implant 3 may, hence, be hold by the threads inside the space I and II.

In the exemplary embodiment of FIG. 67, both the first and the second guiding element 1203, 1203′ are rings. However, any other shape that allows the first and second tension threads 11′, 11″ to slide forth and back through it (which is a precondition for folding and unfolding the implant 3 not shown in FIG. 67) are suitable and, hence, encompassed by the present invention as well.

As can be seen in FIG. 67, in certain embodiments according to the present invention neither of the first tension thread 11′ nor the second tension thread 11″ is directly connected to the pulling thread, the tensioning device of a catheter (not shown in the figures) or any other apparatus for altering the shape of the foldable and/or unfoldable implant. Rather, they are in direct contact with the first string 1201. It is via the first string 1201 that they are in indirect contact with the pulling thread 17″ and, hence, the tensioning device as well.

As can be further seen in FIG. 67, in some embodiments according to the present invention the first tension thread 11′ is connected to the first string 201 at a first end section 1205 of the first string 1201. Likewise, by way of example only, the second tension thread 11″ is connected to the first string 1201 at a second end section 1205′ of the first string 1201. As in FIG. 67, the first end section 1205 and the second end section 1205′ may be opposed ends of the first string 1201.

Finally, as can also be seen in FIG. 67, in certain embodiments according to the present invention the first tension thread 11′ is connected with its first end section 12′ to the first string 1201, and/or the second tension thread 11″ is connected with its first end section 12″ to the first string 1201.

FIG. 68 shows another embodiment according to the present invention in which the first string 1201 is connected to at least six tension threads 11′a, 11′b, 11′c, 11″a, 11″b, 11″c. Three of them (11′a, 11′b and 11′c) are guided through the first guiding element 1203. Three of them (11″a, 11″b′ and 11″c′) are guided through the second guiding element 1203′.

As can be seen, all free ends of the six tension threads 11′a, 11′b, 11′c, 11″a, 11″b′, 11″c′ (i.e., those ends which are not fixedly connected with the first string 1201) are clamped by the common first and second clamping sections 61, 63. However, some of the tension threads may as well be clamped by a first clamping mechanism that is different from a second clamping mechanism as this is shown, e.g., in FIG. 67. Of course, more than two clamping sites may as well be contemplated. In fact, each tension thread might even be clamped by one clamping mechanism (as described herein or in a different design) for itself. Providing a sufficient number of clamping mechanisms is subject-matter of certain embodiment according to the present invention.

FIG. 69a shows an longitudinally cut tip of an implantation device 1 according to the present invention in yet another embodiment. It is shown in an unclamping state revealing the first and the second clamping sections 61, 63 of that embodiment.

FIG. 69b shows the tip of the apparatus of FIG. 69b, slightly rotated, but not cut.

As can be seen in FIGS. 69a and 69b, the first clamping section 61 is connected to the tip of the implantation device 1 by a thread such that the first clamping section 61 can be moved along the shaft or wall 103 of the tip by rotating it. The first clamping section 61 can only slide up and down but is not rotatable.

For moving the first clamping section 61 along the longitudinal axis, a first connecting device 810 is provided. The first connecting device 810 may have a crown-shaped end, it may comprise a gear pattern, it may have teeth or any other engagement device, due to space constraints preferably at its front surface (not on its sided surface), configured to be engageable with a second, rotably arranged connecting device (not shown) of the implantation device 1 (not of the tip) in a manner such that via rotating the second connecting device the first connecting device 810 may be rotated.

The first connecting device 810 comprises threads on an outer surface thereof. Also, there are matching threads on an inner surface of the first clamping section 61.

Any rotation of the first clamping section 61 is precluded or avoided by a longitudinal, straight slot provided in a (preferably inner) side or on a circumferential surface of the first clamping section 61 (e. g. by cutting) and a protrusion such as a pin that is arranged within this slot: the first clamping section 61 can only pass by the pin while the pin is guided inside the slot. That way, the first clamping section 61 can be moved to or away from the second clamping section 63 simply by rotating the first connecting device 810. The last named element may be considered as a rotational clamping mechanism. By the rotational clamping mechanism, the clamping surfaces do not rotate in relation to each other. The advantage that comes along with this is that the tension threads to be clamped do neither become damaged nor displaced because of any rotation of the clamping surfaces.

Instead of the slot, a groove might also be provided.

Also, the protrusion such as the pin might as well be a recession whereas the instead of the slot a protrusion might be arrange. In other words, it does not matter whether the first clamping section 61 comprises the slot and the wall 103 comprises the pin, or the other way round.

In the particular exemplary embodiment of FIG. 69a, the pin may be welded onto the inner main tube that can be seen in FIGS. 69a-c, or the wall 103 thereof. The first clamping section 61 is a (preferably short) tube with outer threads and a (preferably) straight slot cut along its length. The first clamping device 61 slides over the wall 103 and its slot is aligned with the pin which in turn is fixed to the wall 103. It is the pin and the slot acting like a crank or a compulsory guiding that prevents the first clamping section 61 from rotating.

The first connecting device 810 comprising the crown is a tube with inner threads that engages the outer threads of 61. When the first connecting device 810 is rotated, it remains at its place with regard to the longitudinal axis of the tip. Its distance to the second clamping section 63 does never change. It does not move in translation. Only, because of the threads the first clamping section 61 is moved towards the clamping hole 65 or away from it. The first connecting device 810 is arranged on an outside of the first clamping section 61, whereas the first clamping section 61 is arranged on an outside of the wall 103.

In other words, the tip of the implantation device 1 comprises a rotational clamping mechanism while the clamping surface as such are arranged so as not to be rotated.

In FIGS. 69a and 69b, the first and the second clamping sections are moved apart from each other such that they would not clamp any tension thread between them (if provided). The clamping hole 65 is open, i. e., not covered by the first clamping section 61.

As can be seen in FIG. 69a, the clamping surface of at least one of the first and second clamping sections 61, 63 is inclined against a longitudinal axis of the implantation device 1 or the tip thereof shown in FIGS. 69a, 69b. The inclination may be between 10 and 30 degree, preferably between 10 and 20 degree, most preferably about 15 degree, since the latter value has been proven to ensure the best clamping effect.

As can be seen in FIGS. 69a, 69b, the entire clamping mechanism is arranged on the tip shown in these figures. Hence, both the first and the second clamping sections 61, 63 are arranged on the tip which is, in some embodiments, detachable from the remaining parts of the apparatus/implant delivery device. The first connecting device 810 may be embodied (as, e. g., in FIG. 69a) such that it has not to be actively connected with the main parts of the apparatus upon assembling tip and apparatus. Rather, there are a number of designs such as the one shown in FIG. 69a that allows that the first connecting device 810 is automatically being connected to the second connecting device upon putting the tip onto the apparatus. This advantageously safes time and effort. Also, connecting the first and second connecting devices together cannot be forgotten.

FIG. 69c shows the tip of the apparatus of FIGS. 69a and 69b in a clamping state. The first and the second clamping sections 61, 63 have been move towards each other and, thus, into contact with each other. This defines the clamping state. The clamping hole 65 is no longer visible. It is covered by the first clamping section 61.

FIG. 70a shows the tip shown in FIGS. 69a-c in an unclamped state.

FIG. 70b shows the tip of FIG. 70a in another unclamped state. One can now see the thread 830 of the first clamping section 61. In preferred embodiments of the present invention, the slot (not shown in the figures) is arranged within the section of the first clamping section 61 that carries the thread 830.

FIG. 70c shows the tip FIGS. 70a and 70b in a clamped state.

By means of the rotational mechanism any longitudinal actuations by the user in order to unclamp the tension threads can be avoided. This is of advantage because longitudinal operations hampers precise positioning of the device: If one of the clamping sections has to be push or pulled (instead of rotated), a counteracting force on the apparatus or the main catheter is required. That counteracting force may result in that the desired position of the implant will change due to this action. This is avoided by the rotational clamping. FIG. 71 shows a section along the line II-II of FIG. 52a. As can be recognized, the four individual shaft fibers 13″a, 13″b, 13″c and 13″d are present in a bundle such as shown in FIG. 51b; however, they are only bundled loosely and are not pressed against each other by an external force.

Tension threads 11″, 11″a, 11″b and 11″c that are shown by way of example each encompass the whole circumference of the implant after having left the lumen 303′ of the implant 3 through apertures a′, a″, a′″ and a″″ provided in the rim 305 thereof. In all sections of the circumference the tension threads 11″, 11″a, 11″b and the tension thread 11″c are provided.

In other embodiments than the one shown in FIG. 71 some or all of the tension threads 11″, 11″a, 11″b and 11″c do not encompass the whole circumference of the implant. Rather, one or more of the tension threads may re-enter the lumen 303′ of the implant 3 through apertures a′, a″, a′″ and a″″ (also referred to as penetration openings herein) provided within the circumference or rim 305 of the implant 3 that are, for example, adjacent to the aperture through which the respective tension thread has exited from the lumen 303′.

In fact, some or all of the tension threads 11″, 11″a, 11″b and 11″c are provided to re-enter the lumen 303′ by an aperture a′, a″, a′″ and a″″ provided in the rim 305 different from the aperture through which the particular tension thread has exited. In particular, any tension thread may re-enter the lumen 303′ by the next aperture, the next but one, next but two, next plus three, or the like. For example, the tension thread 11″ that exits from aperture a′ may re-enter again through aperture a″, a′″ or a″″ Generally said, any tension thread that exits from an aperture or penetration opening a′ may re-enter through an aperture a′+n, n comprising all natural numbers ranging from 1 (defining one of the two apertures directly neighboring aperture a′) to m (m being the overall number of all apertures but one which are provided along the circumference).

FIG. 72 shows a section along the line of FIG. 53a.

It can readily be seen that, due to the tension applied by means of the respective tension threads 11″, 11″a, 11″b and 11″c, the shaft fibers 13″a, 13″b, 13″c and 13″d have moved from the center of the implant 3 towards a rim area of the implant 3 or at least in a radial direction. One effect of this motion or movement is explained in detail with respect to FIG. 73 below.

In FIG. 53a, only by way of example, a second device for bundling depicted as reference numeral 21 is shown. As can be seen from FIG. 53a, the shaft fibers' portions situated between the two devices 190 and 191 are kept in parallel by means of the devices 190 and 191. On the right side of device 191 for bundling, the shaft fibers can flex or bend again.

FIG. 73a shows a partial longitudinal section through the apparatus of a set according to the present invention in a schematically simplified manner. Again, the tension threads are shown after unfolding the implant in a tension-free state.

In contrast to the set shown in FIG. 71, only three shaft fibers 13″a, 13″b and 13″c and only three apertures a′, a″ and a′″ are shown. Another difference between the set of FIG. 71 and the set of the FIG. 73b is that the tension threads exiting from a particular shaft fiber, e. g. shaft fiber 13″a, does not enter into the same shaft fiber 13″a again. Rather, in the example of FIG. 73b, each fiber covers two thirds of the circumference 305 of the implant 3. That is, in the example of FIG. 73b, each pair of two tension threads extending parallel to each other between a particular shaft fiber and a common aperture of the implant's circumference do not belong to a common tension thread. A pair of two tension threads extending parallel to each other between a particular shaft fiber and a common aperture do not—with respect to what is shown in FIG. 73a—constitute start and end of one particular tension thread.

In FIG. 73a, there are possible positions of the shaft fibers 13″a, 13″b and 13″b marked as dotted circles. Those circles are located closer to the circumference 305 the non-dotted positions in the center area of the implant 3.

As can be derived from the dotted positions which related to a non tension-free state of the tensions threads, which state is not shown in FIG. 73a, even in the non tension-free state pairs of tension threads extending parallel to each other between a particular shaft fiber and a common aperture of the implant's circumference remain parallel to each other even during folding of the implant.

The arrows shown in FIG. 73a are provided for easier reference only.

FIG. 73b shows part of what is seen in FIG. 73a. In particular, it shows only two shaft fibers 13″a and 13″b connected by one tension thread. Also otherwise not necessarily derivable from FIG. 73b, the position of the shaft fibers 13″a and 13″b indicate that the tension thread shown in FIG. 73b is not in a tension-free state. Rather, it requires some tension acting on the tension tread and via the latter on the shaft fibers to move them from the center section of the implant 3 where the shaft fibers are only indicated in dotted lines towards the circumference 305 as shown in FIG. 73b.

FIG. 74 shows a set 200 of threads of the delivery device 1 (the device 1 is not shown in FIG. 74). The set 200 comprises a first tension thread 11′. The first tension thread 11′ comprises two loops 11′a and 11′b indicating that the one tension thread 11′ is wound in two loops 11′a, 11′b around the implant (not shown in FIG. 74), preferably through the first and second guiding structures 11a, 11b.

The tension thread 11′ comprises a first end section 12′ and a second end section 12″.

The set 200 further comprises a pulling string or pulling thread 17″. Pulling it results in a folding of the implant 3 by reducing the loops' 11′a, 11′b diameters.

The pulling string 17″ is interconnected with the tension thread 11′. This may be by an optionally provided interconnecting loop 201a, the knot 207, another knot, or the like. Also, the pulling string 17″ and the tension thread 11′ may be intricated with each other.

The set 200 further comprises a releasing string 203. In the exemplary embodiment of FIG. 74, the releasing string 203 comprises an upwardly directed section 203a and a downwardly directed section 203b. Between them there is a reversing device 1010, or redirector, which is an element of the delivery device 1. The reversing device 1010 is part of the implantation device 1 and arranged to redirect the effect of the releasing string 203 when pulling it.

As can be seen in FIG. 74, the set 200 further comprises a knot 207. By the knot 207, both ends 12′, 12″ of the tension thread 11′ are interconnected to the pulling string 17″ such that pulling the pulling string 17″ may result also in pulling the tension thread 11′. For details regarding the knot 207, it is referred to FIG. 7. As can be seen there, the releasing string 203 is also part of the knot 207.

The releasing string 203 is configured such that it unties the knot 207 if it is sufficiently strong pulled in the direction indicated by the arrow R in FIG. 74. Pulling may be effected by hand or by any suitable device that is part of the implant delivery device.

The pulling string 17″ is tied to at least one of the first and the second end sections 12′, 12″ of the tension thread 11′ by the knot 207.

In the exemplary embodiment shown in FIG. 74, both the first and the second end sections 12′, 12″ of the tension thread are entangled in a knot 207. In other embodiments, only one end 12′, 12″ is entangled by the knot 207.

Also, in FIG. 74, only one tension thread 11′ is shown. However, instead of only one tension thread 11′ the set 200 may comprise a multitude of tension threads. In that case, they all might be tangled with one knot 207. Alternatively, the multitude of tension threads might as well be tied by more that one knot 207. In case of several knots, they might all be identical or similar to the one shown in FIG. 74 or 75. However, several knots might, of course, be knotted in different ways.

In the exemplary embodiment of FIG. 74, the tension thread 11′ comprises a balance limiter 111′c. The balance limiter 111′c may be fixedly connected at two sites with the tension thread 11′ defining a loop 111′d of the tension thread 11′ of fixed length. That way, after the knot 207 is released, the balance limiter 111′c is there to allow the tension thread 11′ to be retrieved completely from the catheter by pulling on 17″. It may ensure that the tension thread 11′ is pulled out from the implant in a desired manner. The balance limiter 111′c may be a simple string interconnecting the two halfs or arms or the like of the tension thread 11′. It may be a knot between the two halfs or arms.

The pulling string 17″ is connected fixedly to the tension thread 11′ such that after untying the knot 207 the then loosened tension thread 11′ may be separated from the implant 3 by pulling the pulling string 17″. The pulling string 17″ may be connected with the tension thread 11′ not only in the knot 207 but additionally, directly or as shown in FIG. 74 in an indirect manner, e. g. by means of the loop 201a or any other section of the pulling string 17″. Since the pulling string 17″ is connected to the tension thread 11′, the latter may be withdrawn from the implant 3 when the pulling string 17″ is withdrawn by the user.

In one exemplary embodiment, as much as three tension threads are provided in the identical way as the tension thread 11′.

Similarly, more than one pulling string 17″, more than one releasing string 203 and more than one knot 207 may be provided.

Also, the two loops 11′a, 11′b of tension thread 11′ may well be distributed to two separate tension threads, one comprising loop 11′a, the other comprising loop 11′b.

Finally, in certain embodiments according to the present invention, as much as, e. g., three or six knots 207 are provided.

FIG. 75 shows an exemplary knot 207 in more detail.

As can be seen in FIG. 7, the knot 207 comprises a first loop 207a which enters and returns from a second loop 207b. The first loop 207a extends into or is part of the releasing string 207.

The knot 207 is configured to tighten if one pulls the pulling string 17″ and to untie if one pulls the releasing string 203. FIG. 76 shows a handle assembly 2000 according to the present invention in a side view. In FIG. 76, a nose 2007, a front knob assembly 2023, a middle casing assembly 2024 having a button 2012, a rear knob 2009, and a rear casing assembly 2022 of the handle assembly 2000 can be seen.

FIG. 77 shows the handle assembly 2000 of FIG. 76 in a perspective view. In addition to what is shown in FIG. 76, in FIG. 77 the handle assembly 2000 is connected a hub dummy 2001, an outer tube 2002 and an inner tube 2004.

The inner tube 2004 is in its front section arranged within the outer tube 2002. The outer tube 2002 may comprise further elements besides the inner tube 2004 such as tension threads or strings (not shown) for folding and unfolding the implant (also not shown).

In certain embodiments according to the present invention, the outer tube 2002 comprises a first connection device (not shown in the figures) configured to engage with a second connection device (also not shown in the figures) forming part of a detachable catheter tip carrying the implant. The first and the second connection device may be configured as plug-in connectors, as crests or crowns or the like, in all cases configured to engage with each other.

It goes without saying that with respect to the present invention, the handle assembly does not need all elements shown in FIG. 76 or 77. For example, the present invention can also be carried out with a handle assembly (not shown) which comprises just the rear knob 2009, and a rear casing assembly 2022. All other elements described herein are optional. For that reason, whenever it is referred to the hub called “rear” knob 2009 herein, it is to be understood that the term “rear” has been added in order to distinguish the (rear) knob 2009 from the (front) knob 2023. Hence, the rear knob 2009 could also be simply referred to as “knob” (without “rear”). The same applies to the rear casing assembly 2022 which could as well be addressed as “casing assembly 2022”.

FIG. 78 shows the handle assembly 2000 of FIGS. 76 and 77 in an explosion view. As stated with regard to FIGS. 76 and 77, the handle assembly 2000 may comprise all or only some of the elements shown in FIG. 78. It may even consist of those elements. However, the handle assembly 2000 may as well comprise further elements in addition to the ones disclosed in FIG. 78.

Further, according to the embodiment according to the invention shown in FIG. 78, some or all of the elements shown in FIG. 78 may be arranged in the handle assembly 2000 in the order or relation to each other shown in FIG. 78. However, the order may be amended in any arbitrary manner as long as the function of the handle assembly 2000 or certain sections thereof is still ensured according to the understanding of the skilled person.

As can be seen from FIG. 78, the front knob assembly 2023, the middle casing assembly 2024, the rear knob 2009, and the rear casing assembly 2022 each are comprised or accompanied by further elements.

In particular, the front knob assembly 2023 comprises a front knob 2010 covering a first rod fitting 2005 and a second rod fitting 2006, the first one being larger than the second one. It comprises another tube 2003, a first o-ring 2032. (may be metric), a second o-ring 2033 (may be metric), several pan heads 2035, also known as pan-head screws, (may be M2×0.4), and a sealing pan head 2038 (may be M3×0.5).

The front knob 2010 may also cover sections of the middle casing assembly 2024. The middle casing assembly 2024 comprises a middle casing 2011, a button 2012, a torsion spring 2030, sealed chamber assembly 2026, a hex socket set screw 2037 (may be M3×0.5), a drum 2014 for winding thereon the tension threads used for folding/unfolding of the implant (not shown), a shaft seal 2028, a seal chamber pin 2018, an o-ring 2034 (may be metric), a sealed chamber cover assembly 2027, and a rush gear 2016.

Parts of the casing assembly 2024 are also covered by the rear knob 2009. The rear knob 2009 comprises a gear stopper 2019, a rush gear 2015 as an example of the first ring element mentioned above and below, some compression springs 2029 as one example of a spring element, a clutch stopper 2013 (or drive wheel) as an example of the second ring element mentioned above, and an internal retaining ring 2039. It also comprises a groove for receiving the retaining ring 2039 in its inner surface.

The rear casing assembly 2022 comprises a rear casing 2008 covering an end 2017, a brake frame assembly 2025, a torsion spring 2031, two brake pads 2020, a brake frame 2021, some pan heads 2035 (may be M2×0.4), and some pan heads 2036 (may be M2×0.4).

As is evident to the skilled person, the feature combination described with respect to FIG. 78 is not the only possible one. In fact, some elements shown in FIG. 78 may be waived upon manufacturing a handle assembly according to the present invention as long as the invention as defined in its most general way, see above, or by the appended claims is still reflected by the so composed handle assembly. For that reason, the number of the single elements and their arrangement relative to each other shown in FIG. 78 is to be understood as just one possible embodiment according to the present invention. Thus, the handle assembly according to the present invention may comprise any arbitrary combination of features shown in FIG. 78 or even not shown.

Also, whenever some elements have been attributed to a particular component discussed with reference to FIG. 76 it has to be understood that certain elements may as well have been attributed to another component shown in FIG. 76. Also, some elements certainly may be attributed to at least two adjacent components at the same time as they extend through at least two neighboring components.

FIGS. 79a-d show different modes for operating of the handle of FIGS. 76 to 78 according to certain embodiments of the invention.

FIG. 79a shows how the handle assembly 2000 can be held while simultaneously rotating the rear knob 2009 by the operator's thumb. That way, the implant can advantageously be folded or unfolded by using just one hand.

FIG. 79b shows how the handle assembly 2000 may be held without effecting the operation sections thereof. By rotating the rear casing assembly 2022 about its longitudinal axis as is indicated in FIG. 79b, the implant (not shown but connected to the outer tube 2002) is also being rotated. Hence, the implant may be properly arranged at the site of its implantation, for example within the heart, by rotating the handle assembly 2000.

FIG. 79c shows how the button 2012 is being pressed (by the thumb of the right hand). Pressing the button 2012 allows the front knob assembly 2023 to be rotated (by, for example, the left hand as shown in FIG. 79c) about its longitudinal axis while the button 2012 is being pressed or once the button 2012 was pressed. As long as the button 2012 is not depressed or was not pressed, the front knob assembly 2023 may not be rotated. The outer tube 2002 comprising the first connection device is interconnected with the front knob assembly 2023 such that rotating the latter results in simultaneously rotating the outer tube 2002 and the first connection device as these elements are fixed to each other in a compulsory guiding such that one cannot rotate while one of the other elements does not rotate. In particular embodiments, rotating the outer tube 2002 results in de-clamping of at least one tension thread (not shown) and in releasing it from the implant and/or from the implant delivery device. Hence, the button 2012 prevents accidental rotation of the outer tube 2002 and, hence, in said particular embodiments, unintended de-clamping of the tension thread. In other embodiments according to the present invention, rotating the outer tube 2002 may have a different effect. For example, rotating may activate a cutter used for cutting the tension thread.

It is obvious to the skilled person that any other activation or deactivation device that allows or forbids rotation of the front knob assembly may be provided instead of the button 2012 which only serves as an example.

FIG. 79d shows an alternative way of holding the handle assembly 2000 with two hands.

FIG. 80 shows a perspective view onto the lower surface of a rush gear 2015 forming part of the force limiter of the handle assembly 2000 of FIG. 76. The rush gear 2015 is a ring-shaped element comprising teeth 1511 arranged at its inner surface 151.

The lower or bottom surface 153 of the rush gear 2015 comprises the openings of at least two receptions 1531 which extend in a direction perpendicular to the lower surface 153.

FIG. 81 shows a perspective view onto the clutch stopper 2013 as part of the force limiter of the handle assembly 2000 of FIG. 76.

On its upper surface 131 the clutch stopper 2013 comprise a number of pins 1311 (or extrusions or protrusions) extending (preferably perpendicularly) from the upper surface 131. The pins 1311 (whose number may assume, for example, any value between two and 20, preferably 11 or 12) are provided to protrude into the receptions 1531 of the lower surface 153 of the rush gear 2015 shown in FIG. 80.

Compression springs 2029 (or any other elastic element or material) are provided in the upper surface 131 of the clutch stopper 2013. In particular embodiments according to the present invention, the compression springs 2029 are arranged over all or some of the pins 1311.

The embodiment of FIG. 81 comprises six compression springs 2029. However, their number may vary according to need. Internal tests have shown that twelve compression springs 2029 like the ones shown in FIG. 81 are able to provide an appropriate clutch opening force of 25 N.

In practice, the number and size of the compression springs 2029 will depend on the surface finish and the clearance between the matching parts.

FIG. 82 shows the rush gear 2015 of FIG. 80 together with the clutch stopper 2013 of FIG. 81. As can be seen from FIG. 82, the pins 1311 of the clutch stopper 2013 fit into the receptions 1531 of the rush gear 2015.

As can also be seen from FIG. 82, an upper surface 155 of the rush gear 2015 comprises teeth 1551 or saw-like or wedge-shaped elements acting as clutch elements.

FIG. 83 shows the rear knob 2009 (also referred to as hub) as part of the force limiter of the handle assembly 2000 of FIG. 76, again in a perspective view revealing the inner space or the inside of the rear knob 2009.

As can be seen from FIG. 83, the rear knob 2009 comprises an inner rim 2091 protruding into the inner space or the lumen of the patency of the rear knob 2009. On its lower surface the inner rim 2091 comprises teeth or saw-like or wedge-shaped elements acting as clutch elements.

In use, i.e., in the assembled stated, the rush gear 2015 will be inserted into the lumen of the rear knob 2009 such that the teeth 1551 of the rush gear 2015 will contact the teeth 911 (see FIG. 85) of the inner rim 2091 of the rear knob 2009 as is shown in FIGS. 84 and 85. That way, the teeth 911 of the rear knob 2009 and the teeth 1551 of the rush gear 2015 will form together a clutch. The force needed to release the teeth 911 of the rear knob 2009 from the teeth 1511 of the rush gear 2015 or to disengage them from each other such that no rotation will be transmitted any more by contact of the teeth of the contacting elements depend on the strength of the compression springs 2029 discussed with respect to FIG. 82. In any case, the teeth concerned are formed such that the clutch opens beyond a certain resistance against rotation.

FIG. 84 shows the rear knob 2009 and the rush gear 2015 (also referred to as a drive wheel) of the force limiter of the handle assembly of FIG. 76 in a first perspective view. In the embodiment of FIG. 84, the compression springs 2029 are attached to the rush gear 2015.

FIG. 85 shows the rear knob 2009 and the rush gear of FIG. 84 in a second perspective view. As in FIG. 84, in FIG. 85 the two elements are shown in a sort of an explosion drawing showing how they are to be arranged to each other upon assembling the handle assembly 2000.

The rush gear 2015 and the rear knob 2009 are linked together with the flat gear pattern or teeth 911 comprised by the inner rim 2091 at the lower surface thereof, and the teeth 1551. In use, the rush gear 2015 is pushed against the inner rim 2091 by the compression springs 29 (or any other type of springs or elastic element) strong enough to maintain the connection until a pre-set threshold force is exceeded (for example, 25 N or 40 N). Above that, the compression springs 2029 are not strong enough and the rush gear 2015 disengages from the inner rim 2091 to avoid breaking the tension thread (or string or cable or wire). This way, the force limiter limits the force or tension applied or applicable onto the tension thread(s).

The force limiter may include the internal retaining ring 2039 shown in, e. g., FIG. 78 and FIG. 97.

FIG. 86 shows the rush gear 2015 in engagement with another rush gear 2016 (also referred to as a pinion). The pinion comprises teeth on its outer surface which engage with teeth 1511 on the inside surface 151 of the rush gear 2015 and is rotated when the rush gear 2015 rotates. The drum 2014 is in turn connected to the rush gear 2016 such that it is rotated once the rush gear 2016 rotates.

At least one of the drum 2014 and the rush gear 2016 are arranged so as to rotated within the rear knob 2009 or within the gear stopper 2019 in an excentric manner (see also FIGS. 89-92).

The drum 2014 is arranged to wind the at least one tension thread (not shown in the figures).

FIG. 87 shows the stopper wheel or gear stopper 2019 in a first embodiment thereof as part of the displacement limiter of the handle assembly 2000 of FIG. 76.

The gear stopper 2019 is adapted to fit into the lumen of the hub or rear knob 2009. It may be arranged within the rear knob 2009 such as to rest on the upper surface of the inner rim 2091.

The gear stopper 2019 may have a ring or a tube shape enclosing an inner lumen or section by an inner surface 2191. The inner surface 2191 has at least two different sections or surface qualities or surface features. In other words, the inner surface 2191 is not homogeneous.

The gear stopper 2019 comprises a rib extending from its inner surface towards the inner lumen or section of the gear stopper 2019. However, also present, the rib is not shown in FIG. 87 or 88. It is, however, shown in FIG. 78, and also in FIGS. 89-92.

In the example of FIG. 87, at a first section 1911, the inner surface 2191 comprises teeth 19111. In a second section 1913, the surface 2191 comprises no teeth. In the example shown in FIG. 87, the second section 1913 merely optionally has a width (extending from the inside surface 2191 to an outside surface 193 of the gear stopper) that is the same (or almost the same) as the width of the first section 1911 (measured from the outside surface 193 to the bottom or origin of the teeth 19111. The teeth 19111 correspond to the teeth of the rush gear 2016 such that the rush gear 2016 may be moved along the first section 1911 or rotated by the first section 1911. That way, the rush gear 2016 also shown in FIG. 87 may as well be moved over from the first section 1911 where its teeth are in contact with the teeth 19111 to the second section 1913 where only some teeth of the rush gear 2016 are in contact with the teeth 19111 of the first section 1911, while some teeth of the rush gear 2016 are not in contact any more. Since some teeth of the rush gear 2016 are still in contact with the teeth 19111 of the gear stopper 2019 when the rush gear 2016 has reached the second section 1913, the rush gears 2016 can be brought back from the second section to the first section by means of the matching teeth by simply changing its rotational direction.

The second section 1913 is adjacent to the first section 1911 or contacts it (as a neighboring section).

As can be seen from FIG. 87, the second section 1913 is more or less a recess 19133 or inclined surface which is delimited or bordered by the last tooth of the first section 19111 on one side and by an inclination or edge 19131 delimiting the opposite side of the recess 19133 defining the second section 1913.

Instead of the edge 19131 or protrusion or the like, any device might be used for stopping a further movement of the rush gear 2016. For example, the second section 1913 might as well (or alternatively) have a stop, an inclination, a broader (in a radial direction) subsection, or the like, as long as the stop, an inclination, a broader (in a radial direction) subsection, or the like engages with the teeth of the rush gear 2016 such that it prevents further rotation (even without lateral movement) of the rush gear 2016. That way, the edge, the stop, the inclination, the broader (in a radial direction) subsection, or the like does engage with at least one of the rush gears' teeth.

The second section 1913 has a length (in a circumferential direction) such that the diameter of the rush gear 2016 is large enough to engage with at least one (preferably more that one) teeth 19111 of the first section 1911 and, at the same time, to contact or to reach the edge 19133 delimiting the recess 19133. That way, once the rush gear 2016 is positioned within the second section 1913, a further movement of the rush gear 2016 away from the first section 1911 (i.e., in the clockwise direction in FIG. 87) is prevented, either by the edge or by the inclination shown in FIG. 87. At the same time, since it is still in contact with at least one tooth of the first section 1911 as explained above, it can always be rotated by the teeth 19111.

In the particular and exemplary embodiment of FIG. 87, the inner surface 2191 additionally comprises a third section 1915 and a fourth section 1917.

The third section 1915 may be designed like the second section 1913 in that it has the same width (or radius) and/or an inclination and/or also no teeth. However, the second section 1913 and the third section 1915 may differ in their length and/or other geometrical features. Also, like the second section 1913 the third section 1915 may also contact the first section 1911, for example as shown in FIG. 87 (the second and the third sections are arranged at opposite ends of the first section).

The third section 1915 may be longer than the second section 1913.

Also, like the second section 1913, the third section 1915 may have an inclination, edge 19151, protrusion or the like, or any device might be used for stopping a further movement of the rush gear 2016. For example, the second section 1915 might as well (or alternatively) have a stop, an inclination, a broader (in a radial direction) subsection, or the like.

Like the second section 1913, the third section 1915 limits the movement of the rush gear 2016 by means of an edge or the like contacting the rush gear teeth. As can be seen in FIG. 87, the further movement (in a lateral or circumferential direction along the inner rim of the stopper gear 2019) of the rush gear 2016 is restricted or stopped by a stop, an inclination, a broader (in a radial direction) subsection, or the like, that does not contact the teeth of the rush gear 2016. At the same time, the third section 1915 is not long enough to allow the teeth of the rush gear 2016 to become disengage from all teeth 19111 of the first section 1911. Rather, the dimensions of the third section 1915 and the rush gear 2016, are chosen such that at least or only the last tooth of the teeth 19111 of the first section 1911 will always remain half-engaged with the teeth of the rush gear 2016. That way, the rush gear 2016 can always be returned or moved back towards the first section 1911, again by simply changing the direction of rotation.

In contrast to the when the rush gear 2016 is definitely one of blocked and inmobilized in the second section 1913, the rush gear 2016, when positioned in the third section 1915, may still be rotated. When it is rotated away from the first section 1911, the last tooth will be repeatedly overleaped which results in a funny, machinery noise of teeth that do not properly engage with each other while being moved with respect to or along each other, also known from improper operation of car gear boxes upon changing gears.

The fourth section 1917 is also optional. It may be arranged opposite the first section 1911. It may or may not contact the first section 1911. It may have teeth or no teeth.

FIG. 88a shows the wheel stopper or gear stopper 2019 in engagement with the rush gear or pinion 2016. The rush gear 2016 is positioned within the third section 1915, which is toothless. The gear stopper 2019 is positioned within the rear knob or hub 2009 without being fixed. Rather, the gear stopper 2019 is held within the rear knob 2009 acting as a casing for the gear stopper 2019. The gear stopper 2019 may, however, rotate within the rear knob 2009 and relative thereto. Hence, when being further rotated in the direction indicated by the arrow by operating the rear knob 2009, for lack of engaging teeth, the rush gear 2016 cannot be rotated any further down. Also, the optionally provided inclination of the third section 1915 hinders the rush gear 2016 to move further on along the inner surface of the gear stopper 2019. This limits the rotatability of both the gear stopper 2019 and the rear knob 2009.

FIG. 88a shows how the gear stopper 2019 and the rush gear or pinion 2016 are positioned relative to each other in a state in which the implant is maximally folded.

In the state shown in FIG. 88a, the rib of the gear stopper 2019 (only shown in FIGS. 89 and 90) abuts the rib of the rib of the casing, or the gear stopper 2019 is stopped by the rib touching some element of the casing other than the rib, such that the gear stopper 2019 can not be rotated any further. Hence, the rush gear 2016 also can not be rotated any further. Thus, any intention of the user to rotate the knob any further must be in vain in that the rush gear 2016 and, thus, the drum 2014 will not be rotated any further and the tension thread will not be wound any further. Rather, what happens once the rush gear's rib has come to a halt is that the force limiter of the handle assembly 2000 will come into play and the clutch comprised by the rear knob 2009 will open against the force of the springs 2029. In consequence, the rear knob 2009 might still be rotated even if the rush gear 2016 has entered the third section 1915. However, its rotation is no longer transmitted onto the drum 2014 and the tension thread is no longer wound or further tensioned.

FIG. 88b shows how the gear stopper 2019 and the rush gear or pinion 2016 are positioned relative to each other in a state in which the implant is maximally unfolded. The rush gear 2016 is positioned inside the second section 1913, which is toothless. Hence, when being further rotated in the direction indicated by the arrow by rotating the rear knob 2009, for lack of teeth, the rush gear 2016 cannot be rotated any further.

In the state shown in FIG. 88b, if the user should intend to further rotate the rear knob 2009 once the rush gear 2016 has entered the second section 1913, the rush gear 2016 would not get over the inclination or edge 19133. Rather, the inclination or edge 19133 would block any further rotation of the rush gear 2016 and the drum 2014, wherefore the tension thread can not be released any further. That way, the displacement of the tension thread is limited. The rush gear 2016 would not slip or turn freely as it does inside the third section 1915 as described supra. The ribs discussed above are not in contact with each other in this state. Also, the force limiter or its clutch does not open. No noise of slipping teeth will be heard.

FIG. 89 shows a stopper wheel as part of the displacement limiter of the handle assembly of FIG. 76 in a second embodiment and in a first state.

In contrast to the FIGS. 87, 88a and 88b, the protrusion of the gear stopper 2019 which may be a rib, is shown in FIG. 89. It is depicted with reference numeral 195. The rib, also referred to as spring rib, protrudes into the lumen of the gear stopper 2019.

As can be seen in FIG. 89, in this embodiment only, the rear part of the middle casing assembly 2024 also comprises a protrusion, e. g. a rib, referred to hereinafter as second rib 241. The second rib 241 is arranged on the casing, and the first rib 195 is arranged on the gear stopper 2019 such that the first rib 195 is placed at the left side (or above the second rib 241) when the gear 2019 has reached the third section 1915 of the gear stopper 2019.

It goes without mentioning that instead of the first and second protrusion any other form and shape a stop or a pair of matching stops will do as well, which is also encompassed by the present invention.

FIG. 89 shows the displacement limiter in a state in which the second rib 241 hinders the gear stopper 2019 from being further rotated in the anticlockwise direction (related to the illustration of FIG. 89) because of the contact between the ribs 195 and 241. Hence, the tension thread cannot be wound any further by rotating in the anticlockwise direction. In FIG. 89, the rush gear 2016 is positioned in the third section 1915. When positioned in the third section 1915 of this exemplary embodiment, the teeth of the rush gear 2016 do not contact an inclination or stop or the like of the third section 1915. The movement of the gear stopper 2019 is stopped only by the contacting ribs 195, 241. The rush gear 2016 may rotate freely in one direction, but it cannot create relative movement between itself and the gear stopper 2019.

FIG. 90 shows the stopper wheel or gear stopper 2019 of FIG. 89 in a second state.

FIG. 90 shows the displacement limiter in a state in which the second rib 241 does not hinder the gear stopper 2019 from being further rotated in the anticlockwise direction (related to the illustration of FIG. 89). Hence, the tension thread can not be unwound further by rotating in the clockwise direction. However, it cannot be released any more since the rush gear 2016 is blocked in the second section 1913 as describe above. In FIG. 90, the rush gear 2016 is positioned in the second section 1913.

FIG. 91 shows the stopper wheel of FIGS. 89 and 90 in a first plan view. In FIG. 91, the gear stopper 2019 takes the position relative to the rush gear 2016 it also takes in FIG. 89. As can be seen, the teeth of the rush gear 2016 are not in full contact with the last tooth of the first section 1911. Rather, the last tooth is half-engaged so that upon rotating the rush gear 2016 in the clockwise direction, its teeth will automatically get engaged again with the first tooth of the first section 1911 first and, in consequence, with the remaining teeth 19111 of the first section 1911 as well.

FIG. 92 shows the stopper wheel of FIGS. 89, 90, and 91 in a second plan view. In FIG. 92, the gear stopper 2019 takes the position relative to the rush gear 2016 it also takes in FIG. 90. As can be seen, the teeth of the rush gear 2016 are still in full contact with not only the last tooth of the first section 1911. At the same time, the rush gear 2016 contacts the edge 19131 of the second section 1913. Hence, the rush gear 2016 that moves on a constant radium about a rotation axis extending through the center of the gear knob 2019 may, not be rotated further in the clockwise direction. It is blocked in the state shown in FIG. 92 regarding any further rotation in the clockwise direction. It may, however, be rotated in the anticlockwise direction.

FIG. 93 shows parts of a brake frame assembly 2025 of the handle assembly of FIG. 1 in a first state which is a state before the brake frame assembly 2025 has been fully assembled.

The parts brake frame assembly 2025 which are shown in FIG. 93 are a first frame 2251 and a first half-wheel 253, the latter being an example of a brake element. Other examples of the brake elements also encompassed by the present invention include a brake pad and a brake shoe.

The first half-wheel 253 is interconnected with the first frame 2251 in manner such that the first half-wheel 253 may pivot relative to the first frame 2251.

FIG. 93 shows not all parts of the brake frame assembly 2025. In the exemplary embodiment shown only in parts in FIG. 93, the complete brake frame assembly 2025 comprises not only the first frame 2251 and the first half-wheel 253 but also a second frame (not shown in FIG. 93 but in FIG. 96 as second frame 252) and a second half-wheel (also not shown in FIG. 93 but in FIG. 96 as second half-wheel 254). Moreover, the assembly 2025 comprises two springs 256, 257 which are also not shown in FIG. 93 but indicated in FIG. 78 and shown in FIG. 96. Finally, the first frame 2251 and the second frame 252 are interconnected to each other by screws as shown in FIG. 78 and in FIG. 96.

In the assembled state, the first and second frames 2251, 252 serve as covers that sandwich the two half-wheels 253, 254 and the two springs 256, 257 between them.

In the particular embodiment of FIG. 93, the first and second frames 2251, 252 have a round periphery. Also, the brake frame assembly 2025 has an through-opening in its center, which may be rectangular in shape. The rectangular shape shown in FIG. 93 is configured to correspond to the cross section of the middle casing assembly 2024.

As stated above, none of the two springs 256, 257 (or other elastic elements) of the brake frame assembly 2025 are shown. In practice, one of these springs is attached between the first half-wheel 253 and the first frame 2251 such that the spring keeps the half-wheel 253 in the position relative to the first frame 2251 as shown in FIG. 93. The other spring will do the same with the second half-wheel (not shown in FIG. 93) in the lower part of FIG. 93.

In the exemplary embodiment of FIG. 93, the springs are inserted into grooves seen in FIG. 93. However, any suitable protrusion or the like will do as well.

In the example of FIG. 93, the springs are selected and arranged so as to stay open as it is shown in FIG. 93 such that it takes effort to close its spring arms (whereas with other springs it needs effort to open them). That means it takes effort to bring the first half-wheel 253 shown in FIG. 93 into its position shown in FIG. 94. The position of the first half-wheel 253 shown in FIG. 93 does not reflect its position in a fully assembled state of the brake frame assembly 2025.

It goes without explanation that instead of two springs and two brake elements one of each will also do.

FIG. 94 shows the parts of FIG. 93 in a second state. The second state shows the position of the half-wheel 253 in a fully assembled state of the brake frame assembly 2025.

Although no springs are shown in FIG. 94, it can easily be understood that the curved surface of the half-wheel 253 may be urged over the outer shape or the circumference of the brake frame assembly 2025, here exemplarily over the outer shape or the circumference of the first frame 2251, and, in a full assembly state of the handle assembly 2000, against an inner surface of the rear knob 2009 inside of which the brake frame assembly 2025 is arranged during use. Since the brake assembly 2025 is arranged in a rotationally stable manner with respect to the middle casing assembly 2024 such that the brake frame assembly 2025 cannot rotate with respect to the handle assembly 2000, whereas the rear knob 2009 can, the first (and, if provided, also the second) half-wheel 253 causes friction and brakes the rotation of the rear knob 2009. The degree of the braking effort certainly depends on the spring force and on the combination of the materials of the braking partners (i.e. the brake element and the inner surface of the rear knob 2009). In any case, the braking efficiency will be chosen to be small enough so that the rear knob 2009 still may be rotated by hand, and at the same time strong enough so that the memory shape effect of the implant or other forces applying to the implant or the tension thread(s) wound onto the drum 2014 may not rotate the rear knob 2009 by themselves.

FIG. 95 shows a cover to the parts of FIG. 93. It may be referred to as a second frame 252 in the sense discussed above with respect to the FIGS. 93 and 94.

FIG. 96 shows the almost fully assembled brake frame assembly 2025 of the preceding figures. What is missing is the second frame 252. The assembly 2025 is arranged within the rear knob 2009. The springs 256, 257 press the half-wheels 253, 254 against the inner surface of the rear knob 2009.

FIG. 97 shows a slightly perspective view of a longitudinal section of the rear knob 2009 of the handle assembly 2000 according to the present invention. The front part of the rear knob 2009 is in the upper part of FIG. 97.

As can be seen in FIG. 97, the rush gear 2016 is arranged within the rear knob 2009 such that it engages with both the gear stopper 2019 and the rush gear 2015 of the force limiter.

FIG. 98a through FIG. 98e show parts of a heart valve or medical implant 3 according to a first exemplary embodiment of the present invention.

FIG. 98a shows three leaflets 3101, 3101′ and 3101″ of the heart valve 3 of the first exemplary embodiment of the present invention. Instead of three leaflets 3101, 3101′ and 3101″ the heart valve 3 according to the present invention may comprise any other number of leaflets, for example two. In the exemplary embodiment of FIG. 98a, all leaflets 3101, 3101′ and 3101″ are identical. In other embodiments according to the present invention, at least two of them may, however, be different from each other.

In the exemplary embodiment of FIG. 98a, each leaflet 3101, 3101′, 3101″ has a body 3103, 3103′, 3103″, respectively, having a round or curved bottom section 3103a, 3103a′, 3103″ and a rim section 3103b, 3103b′, 3103b″ opposing the corresponding curved section 3103a, 3103a′ or 3103a″. The rim section 3103b, 3103b′, 3103b″ extends into opposing tabs 3103c, 3103c′, 3103″ and 3103d, 3103d′, 3103d″ which form the outmost portions to the opposing sides of the respective body 3103, 3103′ or 3103″ (i. e. left and to the right of in the illustration of FIG. 98a).

FIG. 98b shows a crown piece 3111 of the heart valve 3. In use, the crown piece 3111 is formed to a ring by connecting together the free ends 3111a and 3111b of the stripe shown in FIG. 98b with each other.

The crown piece 3111 optionally comprises small tabs 3113, 3113′, 3113″ and round portions 3115, 3115′ 3115″.

The round portions 3115, 3115′ 3115″ are shaped such that their curved rims correspond to the curved sections 3103a, 3103a′ or 3103a″ of the leaflets 3101, 3101′, 3101″.

In FIG. 98b, the reference number 3113″ is used twice. In practice, both small (half-)tabs 3113″ will be contact each other so as to form one single small tab afterwards.

In certain embodiments according to the present invention, the leaflets 3101, 3101′ and 3101″ and/or the crown piece 3111 (and, if applicable, also the pledges (also referred to a pledgets, these terms being, hence, synonyms) 3141, 3141′, 3141″, see FIG. 98e) are cut (e. g. laser cut) from a (e. g. jib-fixed) bovine pericardium having a preferred thickness between 0.35 and 0.55 mm.

In some embodiments according to the present invention, the leaflets 3101, 3101′ and 3101″ are all of identical or similar stiffness.

FIG. 98c shows a top cuff 3121 of the heart valve 3. As can be seen from FIG. 98c, the top cuff 3121, which is used in a ring-shaped form after having united the top cuff's ends 3121a and 3121b with each other, is formed from a flat stripe. The same applies to a bottom cuff 3131 discussed below with reference to FIG. 98d showing a bottom cuff 3131 with ends 3131a, 3131b of the heart valve 3 according to the first exemplary embodiment of the present invention.

The width of the top cuff 3121 is denoted with wtc. The width of the bottom cuff 3131 is denoted with wbc. In certain embodiments according to the present invention, wtc is smaller than wbc.

The length of the top cuff 3121 is denoted with 1. The width of the bottom cuff 3131 is denoted with 1 as well since in the exemplary embodiment shown in the figures the top cuff 3121 and the bottom cuff 3131 are of the same length, at least with respect to a first side 3121c of the top cuff 3121 and a second side 3131c of the bottom cuff 3131. “1” also denotes the length of the lower rim or side 3111c of crown piece 3111. All lengths denoted with 1 are identical in certain embodiments according to the present invention.

The reference numerals rtc and rbc of FIG. 98c and 1d denote the radius of the curvature of the top cuff 3121 and the bottom cuff 3131, respectively. The radius rtc and the radius rbc indicate that the stripes shown in FIG. 98c and 1d are not straight but bent within the drawing plane of FIGS. 98c and 98d. rtc and rbc may be identical, without being limited hereto.

The inner side of top cuff 3121 is denoted with 3121c, the outer side with 3121d.

The inner side of bottom cuff 3131 is denoted with 3131c, the outer side with 3131d.

Because of the radius of top cuff 3121 and bottom cuff 3131, their inner sides 3121c, 3131c are shorter than their outer sides 3121d, 3131d, respectively.

In particular embodiments according to the present invention, the inner side 3121c of top cuff 3121 is interconnected with the outer side 3131d of bottom cuff 3131. This way, the resulting structure will be generally cylindrical with a middle (or about middle) portion that protrudes into the inner space formed by the resulting structure.

In certain embodiments according to the present invention, top cuff 3121 and the bottom cuff 3131 are cut (e. g. laser cut) from a (e. g. surface-tension) porcine pericardium having a preferred thickness between 0.15 and 0.25 mm.

The small tabs 3113, 3113′, 3113″ may be used for a temporary stitch for temporarily securing the crown piece 3111 to the frame or implantation device 1. Both the provided temporary stitch and the small tabs 3113, 3113′, 3113″ may be cut off and disposed later on.

FIG. 98e shows three pledges 3141, 3141′ and 3141″ of the heart valve 3 according to the first exemplary embodiment of the present invention. The pledges 3141, 3141′ and 3141″ are optional. The benefit of the potential pledges 3141, 3141′ and 3141″ are discussed with regards to FIGS. 101a to 101c. The number of the pledges may correspond to the number of posts 12.

The medical implant according to some embodiments of the present invention comprises a heart valve 3, for example the one discussed with reference to FIG. 98a to 98d or 98e and a frame or supporting structure, for example the one discussed with reference to FIGS. 56 and 57. In certain embodiments according to the present invention, the medical implant consists of the heart valve 3 and the frame 1.

FIG. 56 shows a frame of an exemplary implant according to the present invention. The frame is expandable and can be reduced again in its diameter. The diameter refers to a plane perpendicular to a longitudinal axis of the frame. The longitudinal direction also corresponds to the direction of the extension of the implantation device 1 shown in FIG. 56.

Frame comprises at least a first or upper—preferably circular—guiding structure or ring 11a and a second or lower—preferably also circular—guiding structure 11b. The guiding structures 11a, 11b are connected to rods or posts 12. In some embodiments, the guiding structures 11a, 11b can—additionally or alternatively or exclusively—fulfill the function of guiding structures for tension threads 11′, 11″. The tension threads 11′, 11″ form part of an implantation device 1 and serve for applying force or tension or stress, respectively, to the guiding structures 11a, 11b for the purpose of expanding or folding the frame in a targeted manner. In the example of FIG. 56, the guiding structures 11a, 11b are each designed having the shape of an outwardly half-open channel through which the tension threads 11′, 11″ are guided. The half-open channel is opened in a direction away from the centre of the frame. However, the channel can also be shaped to be open to the implant or to any other direction.

In the example of FIG. 56, the guiding structures 11a, 11b are interrupted by posts 12, i.e. the posts 12 are integrated into the guiding structures 11a, 11b such that they form sections of the guiding structures 11a, 11b.

In the embodiment of the frame according to the invention shown in FIG. 56, the posts 12 and/or the guiding structures 11a, 11b have (round or differently shaped, e. g., oval, rectangular, elliptic, and so on) passage means or apertures 10. In the embodiment shown in FIG. 56, they serve as a passage for the tension threads 11′, 11″. The posts 12 also have through openings 8, for example eyelets, which can be arranged in two parallel rows as in FIG. 56, in one row as in FIGS. 99a to 99c, or in any other arrangement.

Furthermore, the frame can also comprise a number of guiding means 11a, 11b other than two, for example, one, three, four or more guiding means.

The guiding structures 11a, 11b can be arranged circularly, however, they can also be arranged non-circularly.

The guiding structures 11a, 11b can be formed integrally with the implant; however, they can also be fabricated separately.

The guiding structures 11a, 11b can have the shape of a wave or undulation, respectively; however, they can also be fabricated in any other form, in particular, a non-wavy or non-undulating form.

Independent of all other features, frame or parts thereof can be fabricated from flat material, e. g., a material which has been cut with a laser, wherein, e. g., after having designed a pattern in the flat material, the material is reformed into a tube (optionally by connecting, such as welding, longitudinal sides of the former flat material lane or web, respectively). However, frame can also be fabricated from a tubular material directly.

The guiding structures 11a, 11b of frame comprise or consist of a plurality of bars 111 which are each connected to another by means of connecting sections 9. The plurality of bars 111 may be arranged in a zig-zag pattern or an undulating or meandering pattern as is exemplary shown in FIG. 56.

FIG. 57 shows the frame of FIG. 56. Two tension threads 11′, 11″ have been led or guided around the frame and return back to the implantation device 1 through the respectively same passage means or apertures 10. The tension threads 11′, 11″ apply a tension or stress on the frame, and, in consequence, frame is not completely expanded or unfolded. Rather, the diameter of the frame has been reduced or is being hindered from expanding in a free manner.

At least one of the top cuff 3121 and the bottom cuff 3131 can be secured to the bars 111 of the second or lower guiding structure 11b, for example by using a whip stitch, with, e. g. four stitches per bar 111, preferably evenly spaced. At some or all of the top portions of bars 111, indicated by 2b′ in FIG. 56 and FIG. 57, and/or at some of all of the bottom portions of the bars 111, indicated by 2b″ in FIG. 56 and FIG. 57, the curved bottom sections 3103a, 3103a′, 3103a″ of the leaflets 3101, 3101′, 3101″ are additionally secured to the frame, for example once again by means of one or more surgeon's knots. Care should be taken to secured the body 3103a, 3103a′, 3103a″ only at its rim or seam section.

FIGS. 99a-99c show how the heart valve 3 of FIG. 98a to 98d or 98e is being fixed or secured to an exemplary post 12 of a frame according to FIG. 56 or 57 in temporal subsequence.

As can be seen in FIG. 99a, a suture 3201 is pierced through the crown piece 3111 and then let through a through hole 8 of the post 12. In doing so, the suture is guided from the outside of the heart valve 3 to the inside of the heart valve 3, leaving a suture tail of at least 2 cm on the outside of the heart valve 3. This tail will later be used to make a knot. Next, a running stitch is created down the post 12 going in and out of the through holes 8 (eyelets) until the suture has been guided through four (preferably neighbouring) through holes 8. The suture is then returned back up the post 12 in and out of the through holes 8 until the through hole 8 below the suture tail is reached, see FIG. 99b. The suture is tied off using the starting suture tail using a surgeon's knot 3203 or any other knot or fixture. The knot 3203 should be on the outside of the heart valve 3 and/or on an outer side 30 of the post 12 as in FIG. 99c.

FIGS. 100a to 100c show how a tab 3103c and a tab 3103d′ of adjacent leaflets 3101 and 3101′ are attached to a common post 12 of the frame.

As can be seen from FIG. 100a, tab 3103c of leaflet 3101 is folded over onto the body 3103 of leaflet 3101, and a knot 3161 is created at the top of tab 3103d′. Knot 3161 secures the heart valve 3 in place on the frame. A suture tail 3163 of the suture is not cut. With a running stitch 3165 going in and out of preferably each eyelet 8 downwards on the tab 3103d′ which is placed on an outside of the post 12 the tab 3103d′ is secured more and more to the post 12 until the bottom of tab 3103d′ is reached.

Now, as is shown in FIG. 100b, tab 3103c is folded back over tab 3103d′ (and hence also on an outside of post 12 although not in contact with the post 12) and the stitch 3165 is continued back up the tabs 3103c and 3103d′, again while going in and out of the eyelets 8 until the top of the tabs is reached.

Tail 3163 is used for tying off using a suitable knot such as a surgeon's knot 3167, see FIG. 100c.

FIGS. 101a to 101c show how one of optional pledges 3141, 3141′, 3141″ as described above is used for reinforcing the connection of the heart valve 3 to the frame.

FIGS. 101a to 101c show a post 12 having an outside face 3o and an inside face 3i. Outside face 3o is directed to an outside of frame whereas inside face 3i is directed to an inside of frame.

A section of post 12 which is enwrapped by a leaflet 3101 is now also enwrapped by a pledge 3141 such that the free ends of pledge 3141 are put above each other or superposed (only separated from each other by the leaflet 3101 arranged between the free ends of the pledge 3141) in a inner space of frame, i. e. at the inside face 3i of post 12.

Starting from the top of pledge 3141, a running stitch 3171 is run down to the bottom of the pledge (see FIG. 101a) and then back up to the top (see FIG. 101b). A knot 3173 is made using the beginning suture's tail (see FIG. 101c). Preferably, the knot 3173 is on the side of the pledge 3141 and does not contact the leaflet 3101.

FIGS. 102a, 102b shows the top cuff 3121 and the bottom cuff 131 before being interconnected, e. g. sewed, to the crown piece 3111, in an exploded view.

If sewed, the arrow indicates the direction of how a first stitch of a suture 3211 (see FIG. 102b) may be made.

As can be seen from FIG. 102a, the crown piece 3111 may be posed between the top cuff 3121 and the bottom cuff 3131.

Also, the three elements 3111, 3121, 3131 may be interconnected such that an end of the inner rim or side 3121c of the top cuff 3121 and an end of the lower rim or side 3111c of the crown piece 3111 are aligned at one side (see the right hand side in FIG. 102a) such that they end on a common level, whereas an end of the inner rim or side 3131c of the bottom cuff 3131, which extends remarkably beyond the ends of inner rim 3121c and lower side 3111c to the right in FIG. 102a, is not aligned with the ends of the lower rim or side 3111c and the inner side 3121c.

FIG. 102b shows the elements of FIG. 102a interconnected with each other, here by means of an exemplary suture, denoted by reference numeral 3211. As mentioned above with respect to certain exemplary embodiments according to the present invention suture 3211 may run along the entire length 1 (see FIGS. 98b to 98d). Suture 3211 may be the sole or only suture used for interconnecting these three elements.

Both the top cuff 3121 and the bottom cuff 3131 are bend upwards (with regards to FIG. 102b) to assume a c-shape which is open towards the top of FIG. 102b. The c-shape is very similar to the shape bars 111 assume in FIG. 56. Hence, the c-shape is well-suited if the combination of top cuff 3121 and bottom cuff 3131 is to cover bars 111 in practise from in inner side of the guiding structure 11a, 11b shown in FIG. 56.

The expansion of frame may benefit in the present exemplary embodiment from the internal stress or from shape-memory capacities of frame. The frame may be manufactured from Nitinol or comprise such material.

FIG. 103 shows the bar 111 that is shown at the left-hand border of the second guiding structure 11b in FIG. 56. Although not parallel to the drawing plane, no part of bar 111 is not cut in FIG. 103.

In FIG. 103, ci denotes the inner circumference the second guiding structure 11b and co the outer circumference of the latter. The bar 111 is shown in FIG. 103 from its side, and more or less in the position its takes in FIG. 56 as well.

In contrast to the illustration of FIG. 56, the second guiding structure 11b and hence also bar 111 is covered by an interconnecting tissue 3301. Within the disclosure of the present specification, interconnecting tissue 3301 may also be referred to a sealing tissue or element as in certain embodiments, sealing is one of the functions provided by that tissue.

Both the interconnecting tissue 3301 and the tension thread 11′, 11″, which runs over the bar 111 but not also over the interconnecting tissue 3301, are cut in the illustration of FIG. 103.

As can be seen from FIG. 103, the interconnecting element or tissue 3301 covers in the inner circumference ci of the second guiding structure 11b and, hence, in this exemplary embodiment also of bar 111. Additionally, it also covers an upper part 2c and a lower part 2d of the second guiding structure 11b at the outer circumference co thereof.

FIG. 104 shows the bar 111 of FIG. 103. As in FIG. 103, bar 111, being part of the second guiding structure 11b, is covered by the interconnecting tissue 3301.

However, in the exemplary embodiment of FIG. 104, the interconnecting tissue 3301 is comprised by or consists of the top cuff 3121, the crown piece 3111 and the bottom cuff 3131.

In the exemplary embodiment of FIG. 104, the top cuff 3121, the crown piece 3111 and the bottom cuff 3131 are all connected to each other by a suture 3211. As can be seen from FIG. 104, in this exemplary embodiment, the top cuff 3121 and the bottom cuff 3131 are arranged at opposite sides of the crown piece 3111.

FIG. 105 shows one embodiment of the heart valve assembly 1000. The reader looks onto the outer circumference co.

The heart valve assembly 1000 differs from what is shown in FIG. 56 by the interconnecting tissue 3301 covering the outer circumference of the second guiding structure 11b by its upper part 2c and its lower part 2d. Hence, FIG. 104 also shows at least parts of the heart valve 100, namely the interconnecting tissue 3301.

Both the upper part 2c and the lower part 2d may have a circular shape and, hence, cover the entire length of the second guiding structure 11b.

Both the upper part 2c and the lower part 2d may be interconnected to the top portions 2b′ of bars 111 and the bottom portions 2h″ as indicated by a number of sutures 3305, a couple of which are shown in FIG. 104.

Both the upper part 2c and the lower part 2d may have a similar or identical width wup or wlp.

Both the upper part 2c and the lower part 2d may act as sealing devices or elements during use and seal between the heart valve 3 and the wall of the orifices into which the heart valve assembly has been inserted or implanted.

In FIG. 105, the lower parts of the posts 12 are covered by heart valve 3 material which also lines the bars 111 from the inside or lumen of the heart valve assembly 1000.

FIG. 106 shows the heart valve assembly 1000 in a bottom view.

As can be seen, an open heart valve lumen 3311, formed by the open leaflets 3101, 3101′, 3101″ is surrounded by the leaflets and, further to an outside of the heart valve assembly 1000, also by the interconnecting tissue 3301.

The outermost rim 3307 of the interconnecting tissue 3301 is, in contrast to an inner rim 3309 thereof (corresponding to the lower part 2d), not round but has dentations or protrusions in the area of tips or interconnecting sections 9. Since the outermost rim 3307 or the lower part 2d is not supported between neighbouring interconnecting sections 9 by any metal or otherwise hard structure of the frame 1 but comprises only relatively soft tissue, sections 3311 of the outer rim 3309 which are arranged between neighbouring interconnecting sections 9 are quite deformable. That way, the overwhelming portion of the outermost rim 3307 or the upper and lower parts 2c, 2d of the interconnecting tissue 3301 may contribute to sealing the heart valve assembly 1000 against the native heart tissue.

The expansion of frame 1 may benefit in the present exemplary embodiment from the internal stress or from shape-memory capacities of frame 1. The frame 1 may be manufactured from Nitinol or comprise such material.

FIG. 107 shows in a schematically simplified view, a set 200 according to the present invention comprising at least an implantation device 1, an implant 3 and a check valve 50.

The implantation device 1 may be configured and/or embodied as described before and/or may comprise additional devices, such as e.g. guiding means, folding means and/or clamping, crimping, tensioning and/or bundling device and so on, as mentioned and described in further details before. The same may apply for the implant 3 in a similar manner.

The implantation device 1 is in this embodiment optionally arranged in the center of the cross-section of the implant 3 and of the valve 50.

The implantation device 1 discloses an end or tip 1′ intended to be introduced to an implantation site. The tip 1′ defines the distal end of the implantation device 1. The proximal end of the implantation device 1 is the end opposite to the distal end defined by the tip 1′.

The valve 50 is configured to fulfil a check valve function and therefore has a blocking direction (BD) and a conducting direction (CD) which are schematically represented by the arrows in the distal and in the proximal direction respectively. The blocking side 50a of the check valve 50 is defined as the side of the check valve which will retain or prevent a fluid to flow in the blocking direction (BD) of the valve 50. The conducting side 50b of the check valve 50 is defined as the side of the check valve which will let pass or allow the flow of a fluid in the conducting direction (CD) of the valve 50. The conducting side 50b of the valve 50 is orientated to the distal end of the implantation device. In other words, the conducting side 50b is located at a smaller distance to the distal end of the implantation device 1 as the blocking side 50a of the valve 50.

In an alternative embodiment which is not illustrated, the check valve may be arranged relative to the implant 3 and/or the implantation device 1 in such a way, that its blocking side 50a is orientated to the distal end of the implantation device 1.

1. A set (200), comprising:
at least one expandable and/or decollapsible or unfoldable implant (3) and at least one implantation device (1) for detachably receiving the implant (3) or a device comprising at least the implant (3),
wherein the implant (3) comprises at least a valve (50), and
wherein the valve (50) is configured so as to fulfill a check valve function with one conducting direction (CD) and one blocking direction (BD), in which a blocking element may move for opening and/or closing the valve (50),
wherein the valve (50) is arranged on the implantation device (1) or on the device with at least one conducting side (50b) being located at a smaller distance from the distal end or from the distal tip of the implantation device (1) than one blocking side (50a).

2. A set (200) according to claim 1, wherein the implant (3) is configured as a valve supporting implant (3) or is composed of at least one valve (50).

3. A set (200) according to claim 1, wherein the implantation device (1) is arranged in the center of a cross-section of the implant (3) and/or of the valve (50).

4. A set (200) according to claim 1, wherein the implant (3) comprises at least one foldable or collapsible or crimpable and unfoldable or expandable structure on or around or over a portion or outer surface of the implantation device (1) or of part thereof.

5. A set (200) according to claim 1, wherein the implant (3) is a stent (13).

6. A set (200) according to claim 5, wherein the stent is a heart valve supporting stent or is configured as a heart valve prosthesis.

7. A set (200) according to claim 1, wherein the implantation device (1) comprises at least one means configured for controlling the expansion, the folding and/or the unfolding of the implant (3), or is prepared for receiving such a means.

8. A set (200) according to claim 7, wherein the means configured for controlling the expansion, the folding and/or the unfolding of the implant (3) comprises at least one tension thread.

9. A set (200) according to claim 1 comprising further at least one crimping, pushing, pulling, twisting, cutting, separating, tensioning, pre-tensioning, grasping, snapping, actuating, aligning, bundling, clamping, connecting and/or releasing device.

10. A method of detachably attaching an implant (3) or a device comprising the implant (3) with an implantation device (1) comprising at least the following steps:
providing an implantation device (1) and an implant (3) comprising at least a valve (50) configured to fulfill at least a check valve function;
detachably attach the implant (3) or the device comprising the implant (3) on or in or onto the implantation device (1) so that at least one conducting side (50b) of the valve (50) is located at a smaller distance from the distal end or from the distal tip of the implantation device (1) than one blocking side (50a).

11. Method according to claim 10, wherein the method comprises additionally at least one of the following steps:
crimping or folding the implant (3);
altering a tension exerted on at least one tension thread;
covering the crimped implant (3) with a sleeve;
clamping at least one section of at least one tension thread.

12. Method for releasing an implant (3) from an implantation device (1) and/or from a device comprising the implant (3), comprising the following steps:
expanding and/or unfolding or decollapsing, respectively, of the implant (3) from a first diameter to a second diameter;
disconnecting the device comprising the implant (3) and/or the implant (3) at a site that is different from a connection site at which the implant (3) or the device comprising the implant (3) has been connected or attached on the implantation device (1).

13. Method according to claim 12, wherein the method comprises additionally at least one of the following steps:
removing an outer sleeve, which is arranged over or about the folded or collapsed implant (3);
releasing at least one tension thread and/or at least a clamped section of at least one tension thread from the implant (3);
altering a tension exerted on at least one tension thread;
releasing, cutting or cutting through at least one tension thread.

14. A set (200) according to claim 2, wherein the implantation device (1) is arranged in the center of a cross-section of the implant (3) and/or of the valve (50).

15. A set (200) according to claim 2, wherein the implant (3) comprises at least one foldable or collapsible or crimpable and unfoldable or expandable structure on or around or over a portion or outer surface of the implantation device (1) or of part thereof.

16. A set (200) according to claim 2, wherein the implant (3) is a stent (13).

17. A set (200) according to claim 16, wherein the stent is a heart valve supporting stent or is configured as a heart valve prosthesis.

18. A set (200) according to claim 2, wherein the implantation device (1) comprises at least one means configured for controlling the expansion, the folding and/or the unfolding of the implant (3), or is prepared for receiving such a means.

19. A set (200) according to claim 18, wherein the means configured for controlling the expansion, the folding and/or the unfolding of the implant (3) comprises at least one tension thread.

20. A set (200) according to claim 2, comprising further at least one crimping, pushing, pulling, twisting, cutting, separating, tensioning, pre-tensioning, grasping, snapping, actuating, aligning, bundling, clamping, connecting and/or releasing device.
